(19) United States
(12) Patent Application Publication
(10) Pub. No.: US 2019/0092877 A1
(43) Pub. Date: Mar. 28, 2019

(54) ANTI-PCSK9-GLP-1 FUSIONS AND METHODS FOR USE

(71) Applicants: MedImmune, LLC, Gaithersburg, MD (US); MedImmune Limited, Cambridge (GB)

(72) Inventors: Anthony CELESTE, Gaithersburg, MD (US); Matthieu CHODORGE, Cambridge (GB); Andrew BUCHANAN, Cambridge (GB); Cristina RONDINONE, Gaithersburg, MD (US); Joseph GRIMSBY, Gaithersburg, MD (US); Peter RAVN, Cambridge (GB); Jonathan SEAMAN, Cambridge (GB); David FAIRMAN, Cambridge (GB)

(21) Appl. No.: 16/133,025

(22) Filed: Sep. 17, 2018

Related U.S. Application Data
(63) Continuation of application No. 15/117,985, filed on Aug. 10, 2016, now Pat. No. 10,077,319.
(60) Provisional application No. 61/994,550, filed on May 16, 2014.
(60) Provisional application No. 61/943,300, filed on Feb. 21, 2014.

(51) Int. Cl. C07K 16/40; C07K 14/605; A61K 38/26; A61K 39/395; A61K 39/00; A61K 38/00

(52) U.S. Cl. CPC C07K 16/40; C07K 14/605; A61K 38/26; A61K 39/3955; C07K 2317/94; A61K 39/00; A61K 38/00; C07K 2319/00; C07K 2317/24; C07K 2317/92; A61K 2039/505

(57) ABSTRACT

This application provides anti-PCSK9˜GLP-1 fusions and methods for use.

### FIELD

Anti-PCSK9˜GLP-1 Fusions and Methods for Use

### BACKGROUND

Diabetes is associated with higher cardiovascular morbidity and mortality. Hypertension, hyperlipidemia, and diabetes are independently associated with increased risk of cardiovascular disease. Subjects with Type 2 diabetes are at two- to four-fold increased risk of cardiovascular disease compared to those without diabetes.

Glucagon-like peptide-1 (GLP-1) is known as a pleiotropic peptide with metabolic and cardiovascular benefits. It is derived from pre-proglucagon, a 158 amino acid precursor polypeptide that is processed in different tissues to form a number of different proglucagon-derived peptides, including glucagon, glucagon-like peptide-1 (GLP-1), glucagon-like peptide-2 (GLP-2) and oxyntomodulin (OXM), that are involved in a wide variety of physiological functions, including glucose homeostasis, insulin secretion, gastric emptying, and intestinal growth, as well as the regulation of food intake. GLP-1 is produced as a 37-amino acid peptide that corresponds to amino acids 72 through 108 of proglucagon (92 to 128 of preproglucagon). The predominant biologically active form is a 30-amino acid peptide hormone (GLP-1(7-37) acid) that is produced in the gut following a meal and rapidly degraded by an abundant endogenous protease-DPP4. Baggio, L. and Drucker, D., Gasteroenterology, 132:2131-2157 (2007).

GLP-1 and GLP-1 analogs, acting as agonists at the GLP-1 receptor, have been shown to be effective hypoglycemic control, e.g., type-2 diabetes. Certain GLP-1 analogs are being sold or are in development for treatment of type-2 diabetes including, e.g., liraglutide (Victoza® from Novo Nordisk), dulaglutide (Eli Lilly), Bydureon (AZ/BMS), Aliblutide (GSK) and Exenatide (Byetta® from Eli Lilly/Amylin).

One of the primary side effects following the initiation of GLP-1 therapy is gastrointestinal side effects, particularly nausea. This side effect is transient, resolves over time and can be mitigated by dose escalation. However, therapy is limited to patients that can tolerate the gastrointestinal side effects.

PCSK9 is a nonenzymatic target for LDL cholesterol reduction and PCSK9 mutations correlate with reductions in LDL cholesterol and coronary heart disease. Cohen J C, N Engl J Med, 354:1264 (2006). PCSK9 antibodies have been shown to reduce LDL cholesterol in statin-treated patients and multiple candidates are undergoing clinical review.

While there are a plurality of individual treatments for diabetes and cardiovascular diseases, there is a need for a single pharmaceutical composition to address both disease states (and the relationship between diabetes and cardiovascular disease). Providing a single pharmaceutical compound that has dual activities will reduce side effects, difficulties with patient compliance, and will increase beneficial outcomes to individual patients and will decrease costs incurred by the health care system.

### SUMMARY

In accordance with the description, disclosed is a dual active fusion molecule for the treatment of diabetes comprising an anti-PCSK9 antibody stably fused to a GLP-1 peptide, wherein the anti-PCSK9 antibody binds a PCSK9 polypeptide and the GLP-1 peptide binds a GLP-1 receptor.

In one aspect, wherein the GLP-1 peptide is fused to the PCSK9 antibody via a linker peptide.

In a further mode, the linker peptide is fused to the C-terminus of the GLP-1 peptide.

In one embodiment, the GLP-1 peptide comprises the amino acid sequence of SEQ ID NO: 36.

In one embodiment, the GLP-1 peptide comprises the amino acid sequence of SEQ ID NO: 3.

In one mode, wherein the Cys18 of the GLP-1 molecule forms a disulfide bridge with the linker peptide or with the GLP-1 peptide itself.

In one aspect, the fusion molecule controls glucose and/or reduces LDL in an animal. The animal may be human.

Disclosed also is a dual active fusion molecule for the treatment of diabetes comprising an anti-PCSK9 antibody stably fused to a GLP-1 peptide comprising the amino acid sequence of SEQ ID NO: 3, wherein C-terminus of the GLP-1 peptide is fused via a peptide linker to the light chain of the anti-PCSK9 antibody, and wherein the anti-PCSK9 antibody binds a PCSK9 polypeptide and the GLP-1 peptide binds a GLP-1 receptor.

Another embodiment disclosed is a method of treating Type 2 Diabetes comprising administering to a subject in need thereof, a fusion molecule described herein.

A further aspect comprises administering to a subject in need thereof, a fusion molecule described herein.

In one mode, a method of reducing low density lipoprotein (LDL) in a subject comprises administering to a subject in need thereof, a fusion molecule described herein.

Another aspect encompasses a method of controlling glucose and reducing LDL in a subject comprising administering to a subject in need thereof, a fusion molecule described herein.

Another aspect encompasses a method of promoting weight loss and reducing LDL in a subject comprising administering to a subject in need thereof, a fusion molecule described herein.

In one embodiment, the subject has Type 2 diabetes.

In another embodiment, the subject has metabolic syndrome.

An additional aspect is a dual active fusion molecule comprising an antibody stably fused to a GLP-1 peptide comprising the amino acid sequence of SEQ ID NO: 3, wherein C-terminus of the GLP-1 peptide is fused via a peptide linker to the light chain of the antibody, and wherein the antibody binds a target polypeptide and the GLP-1 peptide binds a GLP-1 receptor.

A further aspect is a dual active fusion molecule comprising an antibody stably fused to a GLP-1 peptide comprising the amino acid sequence of SEQ ID NO: 36, wherein C-terminus of the GLP-1 peptide is fused via a peptide linker to the light chain of the antibody, and wherein the antibody binds a target polypeptide and the GLP-1 peptide binds a GLP-1 receptor.

In some aspects, the GLP-1 molecule comprises the amino acid sequence of SEQ ID NO: 36. In some aspects, the antibody is an anti-PCSK9 antibody.

In certain modes, the light chain of the anti-PCSK9 antibody is at least 90% identical to the amino acid sequence of SEQ ID NO: 2. In other modes, the light chain of the anti-PCSK9 antibody comprises the amino acid sequence of SEQ ID NO: 2. In yet further modes, the heavy chain of the anti-PCSK9 antibody is at least 90% identical to the amino acid sequence of SEQ ID NO: 1. In some aspects, the heavy chain of the anti-PCSK9 antibody comprises the amino acid sequence of SEQ ID NO: 1.

In some embodiments, the peptide linker comprises the amino acid sequence of SEQ ID NO: 4. In some embodiments of the dual active fusion molecule, the Cys18 of the GLP-1 molecule forms a disulfide bridge with the C terminus of the GLP-1 peptide.

In some embodiments, the dual active fusion molecule is for the treatment of diabetes. In some embodiments, the dual active fusion molecule controls glucose and/or reduces LDL in an animal.

In some modes, the animal is a human.

Some embodiments include a dual active fusion molecule for the treatment of diabetes comprising an anti-PCSK9 antibody stably fused to a GLP-1 peptide that has reduced potency at the human GLP-1 receptor compared to a GLP-1 peptide comprising the amino acid sequence of SEQ ID NO: 29, wherein the C-terminus of the GLP-1 peptide is fused via a peptide linker to the anti-PCSK9 antibody, and wherein the anti-PCSK9 antibody binds a PCSK9 polypeptide and the GLP-1 peptide binds a GLP-1 receptor. In some embodiments, the GLP-1 peptide comprising the amino acid sequence of SEQ ID NO: 28 or SEQ ID NO: 29 is fused using a linker comprising SEQ ID NO:4 to the amino acid sequence comprising SEQ ID NO: 416. In further embodiments, the dual active fusion molecule of claim 25 or 26, wherein the potency at the human GLP-1 receptor is reduced by 30 to 60 fold compared to a GLP-1 peptide comprising the amino acid sequence of SEQ ID NO: 28 or SEQ ID NO: 29.

Some embodiments include an isolated polynucleotide encoding the fusion molecule described herein.

In certain modes, encompassed is a vector comprising the polynucleotide described herein. In other modes, a host cell comprises the polynucleotide or vector described herein.

In some embodiments, a method of making the fusion molecule comprises culturing the host cell under conditions allowing expression of the fusion molecule, and recovering the fusion molecule.

In some modes, a pharmaceutical composition comprises the fusion molecule described herein and a carrier. In some modes, a kit comprises the composition described herein.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1A is a schematic for a dual action fusion molecule as described herein. FIGS. 1B-D show various schematics of certain embodiments of antibody-peptide fusion molecules.

FIG. 1E shows alignment using numerical numbering of PC9#2 variable heavy chain with germline sequence 1-46 (DP-7). Discrepancies are shown in white with a black background.

FIG. 1F shows alignment using numerical numbering of PC9#2 variable light chain with germline sequence VK1 O18 O8 (DPK1). Discrepancies are shown in white with a black background.

FIGS. 2A-G show inhibition of human PCSK9 binding to anti-PCSK9 mAbs by anti-PCSK9 antibody/GLP-1 peptide fusions at the heavy chain N-terminus.

FIGS. 3A-G show inhibition of human PCSK9 binding to anti-PCSK9 mAbs by anti-PCSK9 antibody/GLP-1 peptide fusions at the light chain N-terminus.

FIGS. 4A-B show activation of human GLP1-Receptor by anti-PCSK9 antibody/GLP-1 peptide N-terminus fusions at the antibody heavy chain (A) and light chain (B).

FIG. 5A illustrates stability in rat for a GLP-1 analogue in heavy chain fusion with the control antibody NIP228.

FIG. 5B demonstrates stability in rat for an Exendin-4 GLP-1 analogue in light chain fusion with the anti-PCSK9 antibody PC9#2.

FIG. 5C demonstrates stability in mice for a GLP-1 analogue in fusion with human IgG4 Fc fragment.

FIG. 5D demonstrates stability in mice for a GLP-1 analogue in light chain fusion with the anti-PCSK9 antibody PC9#2.

FIG. 6 demonstrates a potential target profile to guide PCSK9 affinity and GLP-1 potency.

FIG. 7A provides peptide and linker amino acid sequences for eight compounds with an incorporated N-glycosylation consensus motif.

FIG. 7B provides peptide amino acid sequence for three compounds incorporating a disulphide bridge.

FIG. 8 shows a visual representation of the PC9#2_GLP1 molecule and the anti-PC9#2 antibody and GLP-1Fc(G4) used as a benchmark control.

FIG. 9 shows stability in rat of NIP228_GLP1_VH, a GLP-1 analogue in heavy chain fusion with the control antibody NIP228).

FIG. 10 shows stability in mice of PC9#2_GLP-1_VL, a GLP-1 analogue in light chain fusion with the anti-PCSK9 antibody PC9#2.

FIG. 11 shows stability in mice of PC9#2_DSB#3, Exendin-4 analogue DSB#3 in light chain fusion with the anti-PCSK9 antibody PC9#2.

FIG. 12 shows stability in mice of PC9#2_NGS#7, GLP-1 analogue NGS#7 in light chain fusion with the anti-PCSK9 antibody PC9#2.

FIG. 13A illustrates stability in mice for GLP-1 analogue NGS#7 in light chain fusion with the anti-PCSK9 antibody PC9#2.

FIG. 13B shows stability in mice for Exendin-4 analogue DSB#1 in light chain fusion with the anti-PCSK9 antibody PC9#2.

FIG. 13C illustrates stability in mice for Exendin-4 analogue DSB#3 in light chain fusion with the anti-PCSK9 antibody PC9#2.

FIGS. 14A shows stability in mice for benchmark compound GLP-1-Fc fusion Open squares: concentration of test molecule in serum over time; open diamonds concentration of “active” test molecule (as measured by GLP-1 activity) over time for the same samples.

FIGS. 14B shows stability in mice for Exendin-4 analogue DSB#1 in light chain fusion with the anti-PCSK9 antibody PC9#2. Open squares: concentration of test molecule in serum over time; open diamonds concentration of “active” test molecule (as measured by GLP-1 activity) over time for the same samples.

FIG. 15A provides a preparative SEC chromatogram for Exendin-4 analogue DSB#1 in light chain fusion with the anti-PCSK9 antibody PC9#2 after initial protein A purification.

FIG. 15B provides a preparative SEC chromatogram for Exendin-4 analogue DSB#3 in light chain fusion with the anti-PCSK9 antibody PC9#2 after initial protein A purification.

FIG. 16 shows an analytical SEC-HPLC profile of Exendin-4 analogue DSB#1 in light chain fusion with the anti-PCSK9 antibody PC9#2 after initial protein A purification.

FIG. 17 shows the amino acid sequence of additional Exendin-4 variant peptides incorporating a cysteine bridge. Cysteine residues are shown in black, other mutated residues are shown as underline and additional glycine residues at the C-terminus cap are shown in grey.

FIG. 18 provides a preparative SEC chromatogram for Exendin-4 analogue DSB#7 in light chain fusion with the anti-PCSK9 antibody PC9#2 after initial protein A purification.

FIG. 19 provides a preparative SEC chromatogram for Exendin-4 analogue DSB#9 in light chain fusion with the anti-PCSK9 antibody PC9#2 after initial protein A purification.

FIG. 20 shows stability in rat for Exendin-4 analogue DSB#7 in light chain fusion with the anti-PCSK9 antibody PC9#2.

FIG. 21 illustrates stability in rat for Exendin-4 analogue DSB#9 in light chain fusion with the anti-PCSK9 antibody PC9#2.

FIG. 22A shows simulation in human of the impact PCSK9/GLP-1 fusion for PCSK9 suppression.

FIG. 22B illustrates simulation in human of PCSK9/GLP-1 fusion molecule for GLP-1 agonism activity compared to Dulaglutide.

FIG. 23 shows the improved monomeric profile for PC9_2_DSB#7.

FIG. 24 illustrates the pharmacokinetic profile of peptide/antibody molecules in rats after a single i.v. injection.

FIG. 25 demonstrates inhibition of human PCSK9 binding to LDL receptor using competition ELISA.

FIG. 26 shows inhibition of human PCSK9-dependent loss of LDL uptake in HepG2 cells.

FIG. 27 shows the engineered reduction of potency at the human GLP-1 receptor.

FIG. 28A show a human GLP1r cAMP assay for Exendin-4 GLP-1 analogue DSB7_V19A in light chain fusion with the anti-PCSK9 antibody HS9.

FIG. 28B illustrates the stability in rat for Exendin-4 GLP-1 analogue DSB7_V19A in light chain fusion with the anti-PCSK9 antibody HS9.

FIG. 29A-D show specificity of the fusion molecule HS9_DSB7_V19 for GLP-1 receptor determined by cAMP assay.

FIGS. 30A-B show superior glucose control (A) and weight loss (B) over time, including at day 7 post dose.

FIG. 31 shows binding to human PCSK9 of HS9 anti-PCSK9 antibody in fusion with GLP-1 analogue peptide DSB7_V19A using different linkers.

FIG. 32 shows human GLP-1 receptor activation using GLP-1 analogue peptide DSB7_V19A in fusion with anti-PCSK9 antibody HS9 using different linkers.

FIG. 33 illustrates binding to human PCSK9 of anti-PCSK9 antibodies in fusion with GLP-1 analogue peptide DSB7_V19A

FIG. 34 illustrates human GLP-1 receptor activation using GLP-1 analogue peptide DSB7 V19A in fusion with anti-PCSK9 antibodies

FIG. 35 demonstrates binding to human B7-H1 of anti-B7-H1 antibody 2.7A4 in fusion with GLP-1 analogue peptide DSB7_V19A

FIG. 36 shows human GLP-1 receptor activation using GLP-1 analogue peptide DSB7_V19A in fusion with anti-B7-H1 antibody 2.7A4.

FIG. 37 describes stability in rat for the fusion molecule HS9_DSB7_V19A following a single i.v. injection at 60, 30 or 10 mg/kg.

FIG. 38 shows free PCSK9 concentration in rat following a single i.v. injection at 60, 30, or 10 mg/kg of HS9_DSB7_V19A.

FIG. 39 shows total compound, active compound at GLP-1 receptor and free PCSK9 concentrations in rat following a single subcutaneous injection at 60 mg/kg of HS9_DSB7_V19A.

FIGS. 40A-C provides the results from oral glucose tolerance tests (day 0 (A), day 2 (B), day 7 (C), confirming the ability of a single, subcutaneous administration of HS9_DSB7_V19A.

FIG. 41 shows body-weight change over time in the oral glucose tolerance test of FIGS. 40A-C.

FIGS. 42A-C illustrates the results from oral glucose tolerance tests (day 0 (A), day 2 (B), day 7 (C).

FIG. 43 provides body-weight change over time in the oral glucose tolerance test of FIGS. 42A-C.

FIG. 44 shows body weight change in an intraperitoneal glucose tolerance test.

FIG. 45 demonstrates that weekly HS9_DSB7_V19A exhibited a dose dependent reduction in 4 hour fasting blood glucose compared to vehicle control in an intraperitoneal glucose tolerance test.

FIG. 46 shows that weekly dosed HS9_DSB7_V19A exhibited a dose dependent improvement in glucose tolerance as assessed by IPGTT at study day 22.

FIGS. 47A-B shows blood glucose levels in a diet-induced obesity mouse model.

FIGS. 48A-B shows body weight (grams) (A) and % change in body weight (B) in a diet-induced obesity mouse model.

FIG. 49 shows the change in body weight over time in a multiple dose study with HS9_DSB7_V19A.

FIGS. 50A-B show the effect of the GLP-1 component of HS9_DSB7_V19A on glycemic control in a weekly dosing setting, measuring fed glucose (A) and terminal fasting glucose (B).

### DESCRIPTION OF THE SEQUENCES

Table 1 provides a listing of certain sequences referenced in present embodiments.

TABLE 1

| | | |
|---|---|---|
| | | SEQ |
| | | ID |
| Description | Sequence | NO |

GROUP A

| | | |
|---|---|---|
| anti-PCSK9 | QVQLVQSGAEVKKPGASVKVSCKASGYTFTSYYMHWVRQAPGQGLEWMGEISPSGGSTSYNQKFQGRVTMTRDT | 1. |
| antibody | STSTVYMELSSLRSEDTAVYYCARERPLYASDLWGQGTTVTVSS | |
| heavy chain | | |
| (HS9_VH) | | |
| anti-PCSK9 | DIQMTQSPSSLSASVGDRVTITCQASQDVKTAVAWYQQKPGKAPKLLIYSASYRYTGVPSRFSGSGSGTDFTFT | 2. |
| antibody | ISSLQPEDIATYYCQQRYSLWRTFGQGTKLEIK | |
| light chain | | |
| (HS9_Vk) | | |
| GLP-1 | *\hat{ } # | 3. |
| moiety with | HGEGTFTSDLSKQMEEECARLFIEWLKNGGPSSGAPPPGCG | |
| cysteine | With the * designating a cysteine bridge to the linker, | |
| bridge and | the \hat{ } designating a point mutation to reduce potency and match CDTP | |
| double | (maximum efficacy without nausea side effect), and | |
| mutation | the # designating a point mutation to optimize disulfide bonding and | |
| | reduce aggregation | |
| Linker | GGGGSGGGGSGGGGSA | 4. |
| pH | QVQLVQSGAEVKKPGASVKVSCKASGYTFTSYYMHWVRQAPGQGLEWMGEIHPSGGSTSYNQKFQGRVTMTRDT | 5. |
| dependent | STSTVYMELSSLRSEDTAVYYCARERPLYASDLWGQGTTVTVSS | |
| version of | | |
| anti-PCSK9 | | |
| antibody | | |
| heavy chain | | |
| (PC9#2_FG_ | | |
| VH) | | |
| pH | DIQMTQSPSSLSASVGDRVTITCQASQDVHTAVAWYQQKPGKAPKLLIYHASYRYTGVPSRFSGSGSGTDFTFT | 6. |
| dependent | ISSLQPEDIATYYCQQRYSLWRTFGQGTKLEIK | |
| version of | | |
| anti-PCSK9 | | |
| antibody | | |
| light chain | | |
| (PC9#2_FG_ | | |
| Vk) | | |
| GLP-1 | * | 7. |
| moiety with | HGEGTFTSDLSKQMEEECVRLFIEWLKNGGPSSGAPPPSCG | |
| cysteine | | |
| mutation to | | |
| create | | |
| disulfide | | |
| bridge to | | |
| linker | | |
| Antibody A | QVQLVQSGAEVKKPGASVKVSCKASGYTFTSYYMHWVRQAPGQGLEWMGEIHPSGGRTNYNEKFKSRVTMTRDT | 8. |
| pH | STSTVYMELSSLRSEDTAVYYCARERPLYASDLWGQGTTVTVSS | |
| dependent | | |
| heavy chain | | |
| (PC9_2_VH) | | |
| Antibody A | DIQMTQSPSSLSASVGDRVTITCKASQDVHTAVAWYQQKPGKAPKLLIYHASYRYTGVPSRFSGSGSGTDFTFT | 9. |
| pH | ISSLQPEDIATYYCQQRYSLWRTFGQGTKLEIK | |
| dependent | | |
| light chain | | |
| (PC9_2_Vk) | | |
| Antibody B | QVQLVQSGAEVKKPGASVKVSCKASGYTFTSYYMHWVRQAPGQGLEWMGEISPFGGRTNYNEKFKSRVTMTRDT | 10. |
| non pH | STSTVYMELSSLRSEDTAVYYCARERPLYASDLWGQGTTVTVSS | |
| dependent | | |
| heavy chain | | |
| (PC9_1_VH) | | |
| Antibody B | DIQMTQSPSSLSASVGDRVTITCRASQGISSALAWYQQKPGKAPKLLIYSASYRYTGVPSRFSGSGSGTDFTFT | 11. |
| non pH | ISSLQPEDIATYYCQQRYSLWRTFGQGTKLEIK | |
| dependent | | |
| light chain | | |
| (PC9_1_Vk) | | |
| GLP-1 | HGEGTFTSDLSKQMEEEAVRLFIEWLKNGGPSSGAPPPS | 12. |
| moiety | | |
| (exenatide | | |
| (Exe4)) | | |
| GLP-1 | HGEGTFTSDLSKQMEEECVRLFIEWLKNGGPSSGAPPPGCGGGGGSGGGGSGGGGSA | 13. |
| moiety | | |
| DSB#7 with | | |
| linker | | |
| (PC9_HS9_ | | |
| DSB#7) | | |
| anti-PCSK9 | SYYMH | 14. |
| antibody | | |
| heavy chain | | |
| CDR1 | | |
| (PC9_2_HS9) | | |
| anti-PCSK9 | EISPSGGSTSYNQKFQG | 15. |
| antibody | | |
| heavy chain | | |
| CDR2 | | |
| (PC9_2_HS9_ | | |
| VH) | | |
| anti-PCSK9 | EIHPSGGSTSYNQKFQG | 16. |
| antibody | | |
| heavy chain | | |
| CDR2 | | |
| (PC9#2_FG_ | | |
| VH) | | |
| anti-PCSK9 | EIHPSGGRTNYNEKFKS | 17. |
| antibody | | |
| heavy chain | | |
| CDR2 | | |
| (PC9_2_VH) | | |
| anti-PCSK9 | EISPFGGRTNYNEKFKS | 18. |
| antibody | | |
| heavy chain | | |
| CDR2 | | |
| (PC9_1_VH) | | |
| anti-PCSK9 | ERPLYASDL | 19. |
| antibody | | |
| heavy chain | | |
| CDR3 | | |
| anti-PCSK9 | QASQDVKTAVA | 20. |
| antibody | | |
| light chain | | |
| CDR1 | | |
| (PC9_2_HS9_ | | |
| Vk) | | |
| anti-PCSK9 | QASQDVHTAVA | 21. |
| antibody | | |
| light chain | | |
| CDR1 | | |
| (PC9#2_FG_ | | |
| Vk) | | |
| anti-PCSK9 | KASQDVHTAVA | 22. |
| antibody | | |
| light chain | | |
| CDR1 | | |
| (PC9_2_Vk) | | |
| anti-PCSK9 | RASQGISSALA | 23. |
| antibody | | |
| light chain | | |
| CDR1 | | |
| (PC9_1_Vk) | | |
| anti-PCSK9 | SASYRYT | 24. |
| antibody | | |
| light chain | | |
| CDR2 | | |
| (PC9_2_HS9_ | | |
| Vk) | | |
| (PC9_1_Vk) | | |
| anti-PCSK9 | HASYRYT | 25. |
| antibody | | |
| light chain | | |
| CDR2 | | |
| (PC9#2_FG_ | | |
| Vk) | | |
| (PC9_2_Vk) | | |
| anti-PCSK9 | QQRYSLWRT | 26. |
| antibody | | |
| light chain | | |
| CDR3 | | |
| linker repeat | GGGGS | 27. |
| GLP-1 | HGEGTFTSDVSSYLEEQAAKEFIAWLVKGGG | 28. |
| moiety from | | |
| dulaglutide | | |
| (GLP-1 L) | | |
| Human | HAEGTFTSDVSSYLEGQAAKEFIAWLVKGRG | 29. |
| GLP-1 (7- | | |
| 37) | | |
| GLP-1 with | HGEGTFTSCLSKQMEEEAVRLFIEWLKNGGPSSGAPPPSGGGGGGGGGGGCGG | 30. |
| disulfide | | |
| Bridge | | |
| (DSB) #1 | | |
| DSB#1 | | |
| GLP-1 with | HGECTFTSDLSKQMEEEAVRLFIEWLKNGGPSSGAPPPSGGGGGGGGGGGGCG | 31. |
| DSB#2 | | |
| GLP-1 with | HGEGTFTSDLSKQMEEECVRLFIEWLKNGGPSSGAPPGC | 32. |
| DSB#3 | | |
| GLP-1 with | HGEGTFTSDLSKQMEEEAVRCFIEWLKNGGPSSGAGGCS | 33. |
| DSB#4 | | |
| GLP-1 with | HGEGTFTSCLSKQMEEEAVRLFIEWLKNGGPSSGAPPPSGGGGGGGGGCGG | 34. |
| DSB#5 | | |
| GLP-1 with | HGEGTFTSCLSKQMEEEAVRLFIEWLKNGGPSSGAPPPSGGGGGGGGGGCGGG | 35. |
| DSB#6 | | |
| GLP-1 with | HGEGTFTSDLSKQMEEECVRLFIEWLKNGGPSSGAPPPGCG | 36. |
| DSB#7 | | |
| GLP-1 with | HGEGTFTSDLSKQMEEECVRLFIEWLKNGGPSSGAPPGGCG | 37. |
| DSB#8 | | |
| GLP-1 with | HGEGTFTSDLSKQMEEEAVRCFIEWLKNGGPSSGAPPCGG | 38. |
| DSB#9 | | |
| GLP-1 with | HGEGTFTSDLSKQMEEEAVRCFIEWLKNGGPSSGAPPGCG | 39. |
| DSB#10 | | |
| GLP-1 with | HGEGTFTSDLSKQMEEEAVRLFIECLKNGGPSSGACGGS | 40. |
| DSB#11 | | |
| GLP-1 with | HGEGTFTSDLSKQMEEEAVRLFIECLKNGGPSSGAPCPS | 41. |
| DSB#12 | | |
| GLP-1 with | HGEGTFTSDLSKQMEEEAVRLFIECLKNGGPSSGAPPCS | 42. |
| DSB#13 | | |
| PC9#2_GL | HGEGTFTSDVSSYLEEQAAKEFIAWLVKGGGGGGGSGGGGSGGGGSADIQMTQSPSSLSASVGDRVTITCKASQ | 43. |
| P1 VL with | DVHTAVAWYQQKPGKAPKLLIYHASYRYTGVPSRFSGSGSGTDFTFTISSLQPEDIATYYCQQRYSLWRTFGQG | |
| the linker of | TKLEIKR | |
| SEQ ID | | |
| NO: 4 | | |
| underlined | | |
| (also | | |
| referenced as | | |
| PC9_2_GLP1) | | |
| DSB#7 | HVEGTFTSDLSKQMEEECVRLFIEWLKNGGPSSGAPPPGCG | 44. |
| Variant G2V | | |
| DSB#7 | HGEGTFTSDLSKQMAEECVRLFIEWLKNGGPSSGAPPPGCG | 45. |
| Variant | | |
| E15A | | |
| DSB#7 | HGEGTFTSDLSKMEEECVRLFIEWIKNGGPSSGAPPPGCG | 46. |
| Variant L26I | | |
| HS9_DSB7_ | [symbol] [symbol] GGGGSGGGGSGGGGSADIQMTQSPSSLSASVGD | 47. |
| V19A | RVTITCQASQDVKTAVAWYQQKPGKAPKLLIYSASYRYTGVPSRFSGSGSGTDFTFTISSLQPEDIATYYCQQR | |
| VL | YSLWRTFGQGTKLEIK | |
| PC9_2_DSB | [symbol] [symbol] GGGGGSGGGGSGGGGSADIQMT | 48. |
| #1 | QSPSSLSASVGDRVTITCKASQDVHTAVAWYQQKPGKAPKLLIYHASYRYTGVPSRFSGSGSGTDFTFTISSLQ | |
| VL | PEDIATYYCQQRYSLWRTFGQGTKLEIKR | |
| PC9_2_DSB | [symbol] [symbol] GGGGSGGGGSGGGGSADIQMTQSPSSLSASVGDRV | 49. |
| #3 | TITCKASQDVHTAVAWYQQKPGKAPKLLIYHASYRYTGVPSRFSGSGSGTDFTFTISSLQPEDIATYYCQQRYS | |
| VL | LWRTFGQGTKLEIKR | |
| PC9_2_NGS | [symbol] [symbol] GGGGSGGGGSGGGGSADIQMTQSPSSLSASVGDRVTITCKASQ | 50. |
| #7 | DVHTAVAWYQQKPGKAPKLLIYHASYRYTGVPSRFSGSGSGTDFTFTISSLQPEDIATYYCQQRYSLWRTFGQG | |
| VL | TKLEIKR | |
| PC9_2_DSB | [symbol] [symbol] GGGGSGGGGSGGGGSADIQMTQSPSSLSASVGD | 51. |
| #7 | RVTITCKASQDVHTAVAWYQQKPGKAPKLLIYHASYRYTGVPSRFSGSGSGTDFTFTISSLQPEDIATYYCQQR | |
| VL | YSLWRTFGQGTKLEIKR | |
| HS9_DSB# | [symbol] [symbol] GGGGSGGGGSGGGGSADIQMTQSPSSLSASVGD | 52. |
| 7 | RVTITCQASQDVKTAVAWYQQKPGKAPKLLIYSASYRYTGVPSRFSGSGSGTDFTFTISSLQPEDIATYYCQQR | |
| VL | YSLWRTFGQGTKLEIK | |

GROUP B

| | | |
|---|---|---|
| VH | QVQLVQSGAEVKKPGASVKVSCKASGYTFTSYYMHWVRQAPGQGLEWMGEIHPSGGRTNYNEKFKSRVTMTRDT | 53. |
| AB1 | STSTVYMELSSLRSEDTAVYYCARERPLYASDLWGQGTTVTVSS | |
| (pH-Dep) | | |
| VL | DIQMTQSPSSLSASVGDRVTITCKASQDVHTAVAWYQQKPGKAPKLLIYHASYRYTGVPSRFSGSGSGTDFTFT | |
| AB1 | ISSLQPEDIATYYCQQRYSLWRTFGQGTKLEIK | 54. |
| (pH-Dep) | | |
| VH | QVQLVQSGAEVKKPGASVKVSCKASGYTFTSYYMHWVRQAPGQGLEWMGEISPFGGRTNYNEKFKSRVTMTRDT | 55. |
| AB2 | STSTVYMELSSLRSEDTAVYYCARERPLYASDLWGQGTTVTVSS | |
| L1L3 | | |
| VL | DIQMTQSPSSLSASVGDRVTITCRASQGISSALAWYQQKPGKAPKLLIYSASYRYTGVPSRFSGSGSGTDFTFT | 56. |
| AB2 | ISSLQPEDIATYYCQQRYSLWRTFGQGTKLEIK | |
| L1L3 | | |
| VH | EVQLQQSGPELVKPGASVKISCKASGYTFTDYYMNWVKQSHGKSLEWIGDINPNNGGTTYNQKFKGKATLTVDK | 57. |
| AB3 | SYSTAYMELRSLTSEDSAVYYCARWLLFAYWGQGTLVTVSA | |
| 4A5 | | |
| VL | DIVMTQSQKFMSTSVGDRVSVTCKASQNVGTNVAWYQQKPGQSPKALIYSASYRYSGVPDRFTGSGSGTDFTLT | 58. |
| AB3 | ISNVLSEDLAEYFCQQFYSYPYTFGGGTKLEIKR | |
| 4A5 | | |
| VH | QVQLQQPGAELVKPGASVKLSCKASGYTFTSYWMHWVKQRPGQGLEWIGEINPSNGRTNYNEKFKSKATLTVDK | 59. |
| AB4 | SSSTAYMQLSSLTSEDSAVYYCARERPLYAMDYWGQGTSVTVSS | |
| 5A10 | | |
| VL | DIVMTQSHKFMSTSVGDRVSITCKASQDVSTAVAWYQQKPGQSPKLLIYSASYRYTGVPDRFTGSGSGTDFTFT | 60. |
| AB4 | ISSVQAEDLAVYYCQQRYSTPRTFGGGTKLEIKR | |
| 5A10 | | |
| VH | EVQLQQSGPELVKPGASVKISCKASGYTFTDYYMNWVKQSHGKSLEWIGDINPNNGGTSYNQKFKGKATLTVDK | 61. |
| AB4 | SSSTAYMELRSLTSEDSAVYYCAGGGIYYRYDRNYFDYWGQGTTLTVSS | |
| 6F6 | | |
| VL | DIQMTQTTSSLSASLGDRVTISCSASQGISNYLNWYQQKPDGTVKLLIYYTSSLHSGVPSRFSGSGSGTDYSLT | 62. |
| AB4 | ISNLEPEDIATYYCQQYSKLPFTFGSGTKLEIK | |
| 6F6 | | |
| VH | EVKLVESEGGLVQPGSSMKLSCTASGFTFSDYYMAWVRQVPEKGLEWVANINYDGSNTSYLDSLKSRFIISRDN | 63. |
| AB4 | AKNILYLQMSSLKSEDTATYYCAREKFAAMDYWGQGTSVTVSS | |
| 7D4 | | |
| VL | DIVMTQSHKFMSTSFGDRVSITCKASQDVSNALAWYQQKPGHSPKLLIFSASYRYTGVPDRFTGSGSGTDFTFT | 64. |
| AB4 | ISSVQAEDLAVYYCQQHYSTPWTFGGGTKLEIKR | |
| 7D4 | | |

GROUP C

| | | |
|---|---|---|
| VH | QVQLVESGGGVVQPGRSLRLSCAASGFTFSSYGMHWVRQAPGKGLEWVAVIWYDGSDKYYADSVKGRFTISRDN | 65. |
| A74 | SKNTLYLQMNSLRAEDTAVYYCARETGLPKLYYYGMDVWGQGTTVTVSS | |
| 30A4 | | |
| VH | QVQLQESGPGLVKPSQTLSLTCTVSGGSISSSDYYWSWIRQHPGKGLEWIGYIYYSGSTYYNPSLKSRITISVD | 66. |
| A85 | TSKNLFSLKLSSVTAADTAVYYCARGGVTTYYYAMDVWGQGTTVTVSS | |
| 3C4 | | |
| VH | EVQLLESGGGLVQPGGSLRLSCAASGFTFSSYAMNWVRQAPGKGLEWVSTISGSGDNTYYADSVKGRFTISRDN | 67. |
| A71 | SKNTLYLQMNSLRAEDTAVYYCAKKFVLMVYAMLDYWGQGTLVTVSS | |
| 23B5 | | |
| VH | EVQLLESGGGLVQPGGSLRLSCAASGFTFSSYAMNWVRQAPGKGLEWVSTISGSGGNTYYADSVKGRFTISRDN | 68. |
| A72 | SKNTLYLQMNSLRAEDTAVYYCAKKFVLMVYAMLDYWGQGTLVTVSS | |
| 25G4 | | |
| VH | EVQLVESGGGLVKPGGSLRLSCAASGFTFSSYSMNWVRQAPGKGLEWVSSISSSSSYISYADSVKGRFTISRD | 69. |
| A67 | NAKNSLYLQMNSLRAEDTAVYFCARDYDFWSAYYDAFDVWGQGTMVTVSS | |
| 31H4 | | |
| VH | QVQLQESGPGLVKPSQTLSLTCTVSGGSISSGGYYWSWIRQHPGKGLEWIGYIYNSGSTYYNPSLKSRVTISVD | 70. |
| A87 | TSKNQFSLKLSSVTAADTAVYYCAREDTAMVPYFDYWGQGTLVTVSS | |
| 27B2 | | |
| VH | QVQLVQSGAEVKKPGASVKVSCKASGYTFPSYGISWVRQAPGQGLEWMGWISAYNGNTNYAEKLQGRVTMTTDT | 71. |
| A58 | STSTAYMEVRSLRSDDTAVFYCARGYVMDVWGQGTTVTVSS | |
| 25A7 | | |
| VH | QVQLVQSGAEVKRPGASVKVSCKASGYTLTSYGISWVRQAPGQGLEWMGWISVYNGNTNYAQKVQGRVTMTTDT | 72. |
| A52 | STSTVYMELRSLSSDDTAVYYCARGYGMDVWGQGTTVTVSS | |
| 27H5 | | |
| VH | QVQLVQSGAEVKKPGASVKVSCKASGYTLTSYGISWVRQAPGQGLEWMGWISFYNGNTNYAQKVQGRVTMTTDT | 73. |
| A51 | STSTVYMELRSLRSDDTAVYYCARGYGMDVWGQGTTVTVSS | |
| 26H5 | | |
| VH | QIQLVQSGAEVKKPGASVKVSCKASGYTLTSYGISWVRQAPGQGLEWMGWISFYNGNTNYAQKVQGRVTMTTDT | 74. |
| A53 | STSTVYMELRSLRSDDTAVYFCARGYGMDVWGQGTTVTVSS | |
| 31D1 | | |
| VH | QIQLVQSGAEVKKPGASVKVSCKASGYPLTSYGISWVRQAPGQGLEWMGWISAYNGNTNYAQKVQGSVTMTTDT | 75. |
| A48 | STSTVYMELRSLRSDDTAVYYCARGYGMDVWGQGTTVTVSS | |
| 20D10 | | |
| VH | QVQLVQSGAEVKKPGASLKVSCKASGYSLTSYGISWVRQAPGQGLEWMGWISAYNGNTNYAQKVQGRVTMTTDT | 76. |
| A54 | STSTVYMEVRSLRSDDTAVYYCARGYGMDVWGQGTTVTVSS | |
| 27E7 | | |
| VH | QVQLVQSGAEVKKPGASVKVSCKASGYPLTSYGISWVRQAPGQGLEWMGWISAYNGNTNYAQKVQGRVTMTTDT | 77. |
| A55 | STSTVYMELRSLRSDDTAVYYCARGYGMDVWGQGTTVTVSS | |
| 30B9 | | |
| VH | QVQLVQSGAEVKKPGASVKVSCKASGYALTSYGISWVRQAPGQGLEWMGWISAYNGNTNYAQKVQGRVTMTTDT | 78. |
| A56 | STSTVYMELRSLRSDDTAVYYCARGYGMDVWGQGTTVTVSS | |
| 19H9 | | |
| VH | QVQLVQSGAEVKKPGASVKVSCKASGYTLTSYGISWVRQAPGQGLEWMGWVSFYNGNTNYAQKLQGRGTMTTDP | 79. |
| A49 | STSTAYMELRSLRSDDTAVYYCARGYGMDVWGQGTTVTVSS | |
| 21B12 | | |
| VH | QVQLVQSGAEVKKPGASVKVSCKASGYSFTSYGISWVRQAPGQGLEWMGWVSAYNGNTNYAQKFQGRVTMTTDT | 80. |
| A57 | STSTAYMELRSLRSDDTAVYYCARGYVMDVWGQGTTVTVSS | |
| 17C2 | | |
| VH | QVQLVQSGAEVKKPGASVKVSCKASGYTLTSYGISWVRQAPGQGLEWMGWVSFYNGNTNYAQKLQGRGTMTTDP | 81. |
| A50 | STSTAYMELRSLRSDDTAVYYCARGYGMDVWGQGTTVTVSS | |
| 23G1 | | |
| VH | QVQLQQSGPGLVKPSQTLSLTCAISGDSVSSNSAAWNWIRQSPSRGLEWLGRTYYRSKWYKNYSVSVKSRITIN | 82. |
| A91 | PDTSKNQFSLQLNSVTPGDTAVYYCARGGPTAAFDYWGQGTLVTVSS | |
| 13H1 | | |
| VH | EVQLVESGGGLVQPGGSLRLSCVVSGFTFSSYWMSWVRQAPGKGLEWVANIKQDGSEKYYVDSVKGRFTISRDN | 83. |
| A64 | AKNSLYLQMNSLRAEDTAVYYCARESNWGFAFDIWGQGTMVTVSS | |
| 9C9 | | |
| VH | EVQLVESGGGLVQPGGSLRLSCAASGFTFSRYWMSWVRQAPGKGLEWVANIKHDGSEKYYVDSVKGRFTISRDN | 84. |
| A62 | AKNSLYLQMNSLRAEDTAVYYCARESNWGFAFDVWGHGTMVTVSS | |
| 9H6 | | |
| VH | QVQLQQWGAGLLKPSETLSLTCAVYGGSFSAYYWNWIRQPPGKGLEWIGEINHSGRTDYNPSLKSRVTISVDTS | 85. |
| A89 | KKQFSLKLNSVTAADTAVYYCARGQLVPFDYWGQGTLVTVSS | |
| 31A4 | | |
| VH | EVQLVESGGGLVQPGGSLRLSCAASGLTFSNFWMSWVRQAPGKGLEWVANIKQDGSEKYYVDSVKGRFTISRDN | 86. |
| A65 | AKNSLYLQMNSLRAEDTAVYSCTRESNWGFAFDIWGQGTMVTVSS | |
| 1A12 | | |
| VH | QVHLVESGGGVVQPGRSLRLSCAASGFTFNSFGMHWVRQAPGKGLEWVALIWSDGSDEYYADSVKGRFTISRDN | 87. |
| A79 | SKNTLYLQMNSLRAEDTAVYYCARAIAALYYYYGMDVWGQGTTVTVSS | |
| 16F12 | | |
| VH | QVQLVESGGGVVQPGRSLRLSCAASGFTFSSFGMHWVRQAPGKGLEWVALIWNDGSNKYYADSVKGRFTISRDN | 88. |
| A80 | SKNTLYLQMNSLRAEDTAVYYCARAIAALYYYYGMDVWGQGTTVTVSS | |
| 22E2 | | |
| VH | QVHLVESGGGVVQPGRSLRLSCAASGFTFNSFGMHWVRQAPGKGLEWVALIWSDGSDKYYADSVKGRFTISRDN | 89. |
| A76 | SKNTLYLQMNSLRAEDTAVYYCARAIAALYYYYGMDVWGQGTTVTVSS | |
| 27A6 | | |
| VH | QVQLVESGGGVVQPGRSLRLSCAASGFTFSSFGMHWVRQAPGKGLEWVALIWNDGSNKYYADSVKGRFTISRDN | 90. |
| A77 | SKNTLYLQMNSLRAEDTAVYYCARAIAALYYYYGMDVWGHGTTVTVSS | |
| 28B12 | | |
| VH | QVQLVESGGGVVQPGRSLRLSCAASGFTFSSFGMHWVRQAPGKGLEWVALIWNDGSNKYYADSVKGRFTISRDN | 91. |
| A78 | SKNTLYLQMNSLRAEDTAVYYCARAIAALYYYYGMDVWGQGTTVTVSS | |
| 28D6 | | |
| VH | QVQLVESGGGVVQPGRSLRLSCAASGFTFRSYGMHWVRQAPGKGLEWVALIWHDGSNTYYVDSVKGRFTISRDN | 92. |
| A83 | SKNTLYLQMNSLRAEDTAVYYCARGIAVAYYYYGMDVWGQGTTVTVSS | |
| 31G11 | | |
| VH | EVQLLESGGGLVQPGGSLRLSCAASGFTFSSYAMSWVRQAPGKGLEWVSTISGSGGRTYYADSVKGRFTISRDN | 93. |
| A69 | SKNTLYLQMNSLRAEDTAVYYCAKEVGSPFDYWGQGTLVTVSS | |
| 13B5 | | |
| VH | QVQLVESGGGVVQPGRSLRLSCAASGFTFSSYGMHWVRQAPGKGLEWVAIIWYDGSNKYYADSVKGRFTISRDN | 94. |
| A81 | SKNTLYLQMNSLRAEDTAVYYCARRGGLAARPGGMDVWGQGTTVTVSS | |
| 31B12 | | |
| VH | QVQLVQSGAEVKKPGASVKVSCKASGYTFTSYGISWVRQAPGQGLEWMGWISTYNGNTNYAQKVQGRVIMITDT | 95. |
| A60 | STSTAYMELRSLRSDDTAVYYCARGYTRDYWGQGTLVTVSS | |
| 3B6 | | |
| VL | DIVMTQSPLSLSVTPGEPPSISCRSSQSLLHSNGYNFLNWYLQKPGQSPQLLIYLGSHRASGVPDRFSGSGSGT | 96. |
| 5 | DFTLEISRVEAEDVGVYYCMQVLQTPFTFGPGTKVDIK | |
| 30A4 | | |
| VL | DIQMTQSPSSLSASVGDRVTITCRASQRISNYLSWYLQKPGIAPKLLIYAASSLQSGVPSRFSGSGSGTDFTLT | 97. |
| 7 | ISSLQSEDFATYYCQQSYSTPLIFGGGTKVEIK | |
| 3C4 | | |
| VL | DILMTQSPSSLSASVGDRVTITCRASQSISSYLNWYQQKPGKAPKVLIYAASSLQSGVPSRFSGSGSGTDFTLT | 98. |
| 9 | INSLQPEDFATYYCQQSYSSPITFGQGTRLEIK | |
| 23B5 | | |
| VL | DIQMTQSPSSLSASVGDRVTITCRASQSISIYLNWYQQKPGKAPYLLIYAAASLQSGVPSRFSGSGSGTDFTLT | 99. |
| 10 | ISSLQPEDFATYYCQQSYSAPITFGQGTRLEIK | |
| 25G4 | | |
| VL | QSVLTQPPSVSGAPGQRVTISCTGSSSNIGAGYDVHWYQQLPGTAPKLLISGNSNRPSGVPDRFSGSKSGTSAS | 100. |
| 12 | LAITGLQAEDEADYYCQSYDSSLSGSVFGGGTKLTVL | |
| 31H4 | | |
| VL | QSVLTQPPSVSGAPGQRVTISCTGSSSNIGAHYDVHWYQQVPGTAPKLLIYGNTYRPSGVPDRFSGSKSGTSAS | 101. |
| 13 | LAITGLQAEDEADYYCQSYDNSLSGVVFGGGTKLTVL | |
| 27B2 | | |
| VL | QSALTQPASVSGSPGQSITISCTGTSSDVGRYNSVSWYQHHPGKAPKVMIYEVSNRPSGVSTRFSGSKSGNTAS | 102. |
| 15 | LTISGLQAEDEADYYCSSYTSSSVVFGGGTKLTVL | |
| 25A7 | | |
| VL | QSALTQPASVSGSPGQSITISCTGTSSDVGGYNSVSWYQQHPGKPPKLMIYEVSNRPSGVSIRFSGSKSGNTAS | 103. |
| 16 | LTISGLQAEDEADYFCSSYTSTSMVFGGGTKLTVL | |
| 27H5 | | |
| VL | QSALTQPASVSGSPGQSITISCTGTSSDVGGYNSVSWYQQHPGKPPKLMIYEVSNRPSGVSIRFSGSKSGNTAS | 104. |
| 17 | LTISGLQAEDEADYFCSSYTSTSMVFGGGTKLTVL | |
| 26H5 | | |
| VL | QSALTQPASVSGSPGQSITISCIGTSSDVGGYNSVSWYQQHPGKPPKLMIYEVSNRPSGVSNRFSGSKSGNTAS | 105. |
| 18 | LTISGLQAEDEADYFCSSYTSTSMVFGGGTKLAVL | |
| 31D1 | | |
| VL | QSALTQPASVSGSPGQSITISCTGTSSDVGGYNSVSWYQQYPGKPPKLKIYEVSNRPSGVSNRFSGSKSGNTAS | 106. |
| 19 | LTISGLQAEDEADYFCSSYTSTSMVFGGGTKLTVL | |
| 20D10 | | |
| VL | QSALTQPASVSGSPGQSITISCTGTSSDVGGYNSVSWYQQHPGKPPKLMIYEVSNRPSGVSNRFSGSKSGNTAS | 107. |
| 20 | LTISGLQAEDEADYFCSSYTSTSMVFGGGTKLTVL | |
| 27E7 | | |
| VL | QSALTQPASVSGSPGQSITISCTGTSSDVGGYNSVSWYQQHPGKPPKLMIYEVSNRPSGVSNRFSGSKSGNTAS | 108. |
| 21 | LTISGLQAEDEADYFCSSYTSTSMVFGGGTKLTVL | |
| 30B9 | | |
| VL | QSALTQPASVSGSPGQSITISCTGTNSDVGGYNSVSWYQQHPGKPPKLMIYEVSNRPSGISNRFSGSKSGNTAS | 109. |
| 22 | LTISGLQAEDEADYFCSSYTSTSMVFGGGTKLTVL | |
| 19H9 | | |
| VL | QSALTQPASVSGSPGQSITISCTGTSSDVGGYNSVSWYQQHPGKAPKLMIYEVSNRPSGVSNRFSGSKSGNTAS | 110. |
| 23 | LTISGLQAEDEADYYCNSYTSTSMVFGGGTKLTVL | |
| 21B12 | | |
| VL | QSALTQPASVSGSPGQSITISCTGTSSDVGAYNSVSWYQQHPGKAPKRMIYEVSNRPSGVSNRFSGSKSGNTAS | 111. |
| 24 | LTISGLQAEDEADYYCSSYTSTNMVFGGGTKLTVL | |
| 17C2 | | |
| VL | QSALTQPASVSGSPGQSITISCTGTSSDVGGYNSVSWYQQHPGKAPKLMIYEVTNRPSGVSNRFSGSKSGNTAS | 112. |
| 26 | LTISGLQAEDEADYYCNSYTSTSMVFGGGTKLTVL | |
| 23G1 | | |
| VL | LSALTQPASVSGSPGQSITISCTGTSSDVGNYNLVSWYQQYSGKAPKLMIYEVSKRPSGVSNRFSGSKSGNTAS | 113. |
| 28 | LTISGLQAEDEADYYCCSYAGSSTLVFGGGTKLTVL | |
| 13H1 | | |
| VL | QSVLTQPPSASGTPGQRVTISCSGSSSNIGSKTVNWYQQVPGTAPKLLIYRNNQRPLGVPDRFSGSKSGTSASL | 114. |
| 30 | AISGLQSEDEADYYCAAWDDSLNWVFGGGTKLTVL | |
| 9C9 | | |
| VL | QSVLTQPPSASGPPGQRVTISCSGSSSNIGSNTVNWYQQLPGTAPKLLIYSNNRRPSGVPDRFSGSKSGTSASL | 115. |
| 31 | AISGLQSEDEADYYCAAWDDSLNWVFGGGTKLTVL | |
| 9H6 | | |
| VL | QSVLTQPPSASGTPGQRVTISCSGSSSNIGSNTVNWYQQLPGTAPKLLIYSNNQRPSGVPDRFSGSKSGTSASL | 116. |
| 32 | AISGLQSEDEADYYCAVWDDSLNGWVFGGGTKLTVL | |
| 31A4 | | |
| VL | QSVLTQPPSASGTPGQRVTISGSGSSSNIGSKTVNWYQQFPGTAPKLLIYSNNRRPSGVPDRFSGSKSGTSASL | 117. |
| 33 | AISGLQSEDEADYYCAAWDDSLNWVFGAGTKLTVL | |
| 1A12 | | |
| VL | QSVLTQPPSVSAAPGQKVTISGSGSSSNIGNNFVSWYQQLPGTAPKLLIYDYNKRPSGIPDRFSGSKSGTSATL | 118. |
| 35 | GITGLQTGDEADYYCGTWDSSLSAYVFGTGTRVIVL | |
| 16F12 | | |
| VL | QSVLTQPPSVSAAPGQKVTISGSGSSSNIGNNFVSWYQQLPGTAPKLLIYDYNKRPSGIPDRFSGSKSGTSATL | 119. |
| 36 | GITGLQTGDEADYYCGTWDSSLSGYVFGTGTRVTVL | |
| 22E2 | | |
| VL | QSVLTQPPSVSAAPGQKVTISGSGSSSNIGNNFVSWYQQFPGTAPKLLIYDYNKRPSGIPDRFSGSKSGTSATL | 120. |
| 37 | GITGLQTGDEADYYCGTWDSSLSSYVFGTGTRVTVL | |
| 27A6 | | |
| VL | QSVLTQPPSVSAAPGQKVTISGSGSSSNIGNNFVSWYQQLPGTAPKLLIYDYNKRPSGIPDRFSGSKSGTSATL | 121. |
| 38 | GITGLQTGDEADYYCGTWDSSLSGYVFGTGTRVTVL | |
| 28B12 | | |
| VL | QSVLTQPPTVSAAPGQKVTISGSGSSSNIGNNFVSWYQQLPGTAPKLLIYDYNKRPSGIPDRFSGSKSGTSATL | 122. |
| 39 | GITGLQTGDEADYYCGTWDSSLSGYVFGTGTRVTVL | |
| 28D6 | | |
| VL | QSVLTQPPSVSAAPGQKVTISGSGSSSNIGNNFVSWYQQLPGTAPKLLIYDSNKRPSGIPDRFSGSKSGTSATL | 123. |
| 40 | DITGLQTGDEADYYCGTWDSSLSAYVFGTGTKVTVL | |
| 31G11 | | |
| VL | QSVLTQPPSVSAAPGQKVTISGSGSNSNIGNNYVSWYQQLPGTAPKLLIYDNNKRPSGIPDRFSGSNSGTSATL | 124. |
| 42 | GITGLQTGDEADYYCGTWDSSLSAVVFGGGTKLTVL | |
| 13B5 | | |
| VL | SYELTQPPSVSVSPGQTARITCSGDKLGDKYACWYQQKPGQSPVLVIYQNTKWPLGIPERFSGSKSGNTVTLTI | 125. |
| 44 | SGTQAMDEADYYCQAWDSSTVVFGGGTKLTVL | |
| 31B12 | | |
| VL | QPVLTQPLFASASLGASVTLTCTLSSGYSSYEVDWYQQRPGKGPRFVMRVDTGGIVGSKGEGIPDRFSVLGSGL | 126. |
| 46 | NRYLTIKNIQEEDESDYHCGADHGSGTNFVVVFGGGTKLTVL | |
| 3B6 | | |

GROUP D

| | | |
|---|---|---|
| VH | QVQLVESGGGVVQPGRSLRLSCAASGFTFSSYGMHWVRQAPGKGLEWVAFIGFDGSNIYYGDSVRGRIIISRDN | 127. |
| 66 | SENTLYLEMNSLRAEDTAVYYCAREKGLDWGQGTLVTVSS | |
| VH | EVQLVESGGGLVQPGGSLRLSCAASGFTFNNYAMNWVRQAPGKGLDWVSTISGSGGTTNYADSVKGRFIISRDS | 128. |
| 90 | SKHTLYLQMNSLRAEDTAVYYCAKDSNWGNFDLWGRGTLVTVSS | |
| H1H316P | | |
| VH | QVQLQESGPGLVKPSETLSLTCTVSGDSINTYYWSWFRQPPGKGLEWIGYIYYSGTTNYNPSLKSRVTISIDTP | 129. |
| 138 | RNQFSLKLISVTAADTAVYYCARERITMIRGVTLYYYSYGMDVWGQGTTVTVSS | |
| VH | EMQLVESGGGLVQPGGSLRLSCAASGFTFSSHWMKWVRQAPGKGLEWVANINQDGSEKYYVDSVKGRFTISRDN | 130. |
| 218 | AKNSLFLQMNSLRAEDTAVYYCARDIVLMVYDMDYYYYGMDVWGQGTTVTVSS | |
| H1M300N | | |
| VH | QVQLVQSGGGLVQPGGSLRLSCAASGFTLSSYDMHWVRQSTGKGLEWVSAIGSTGDTYYPGSVKGRFTITREKA | 131. |
| 2 | KNSVYLQMNSLRAGDTAVYYCVREGWEVPFDYWGQGTLVTVSS | |
| VH | EVQLVESGGGLVQPGGSLRLSCAASGFTLSSYDMHWVRQSTGKGLEWVSAIGSTGDTYYPGSVKGRFTITREKA | 132. |
| 18 | KNSVYLQMNSLRAGDTAVYYCVREGWEVPFDYWGQGTLVTVSS | |
| VH | EVQLVESGGGLVQPGGSLRLSCAASGFTLSSYDMHWVRQATGKGLEWVSAIGSTGDTYYPGSVKGRFTISRENA | 133. |
| 22 | KNSLYLQMNSLRAGDTAVYYCVREGWEVPFDYWGQGTLVTVSS | |
| VH | QVQLVQSGGGVVQPGRSLRLSCAASGFTFSSYGMHWVRQAPGKGLEWVAFIGFDGSNIHYGDSVRGRIIISRDN | 134. |
| 26 | SENTLYLEMNSLRAEDTAMYYCAREKGLDWGQGTTVIVSS | |
| VH | QVQLVESGGGVVQPGRSLRLSCAASGFTFSSYGMHWVRQAPGKGLEWVAFIGFDGSNIHYGDSVRGRIIISRDN | 135. |
| 42 | SENTLYLEMNSLRAEDTAMYYCAREKGLDWGQGTLVTVSS | |
| VH | QVQLVESGGGVVQPGRSLRLSCAASGFTFSSYGMHWVRQAPGKGLEWVAVIGFDGSNIYYADSVKGRFTISRDN | 136. |
| 46 | SKNTLYLQMNSLRAEDTAVYYCAREKGLDWGQGTLVTVSS | |
| VH | QVQLQESGGGVVQPGRSLRLSCAASGFTFSSYGMHWVRQAPGKGLEWVAFIGFDGSNIYYGDSVRGRIIISRDN | 137. |
| 50 | SENTLYLEMNSLRAEDTAVYYCAREKGLDWGQGTLVTVSS | |
| VH | QVQLVESGGGVVQPGRSLRLSCAASGFTFSSYGMHWVRQAPGKGLEWVAVIGFDGSNIYYADSVKGRFTISRDN | 138. |
| 70 | SKNTLYLQMNSLRAEDTAVYYCAREKGLDWGQGTLVTVSS | |
| VH | EVQLVESGGGLVQPGGSLRLSCAASGFTFNNYAMSWVRQAPGKGLEWVSAISGSGGTTYYADSVKGRFTISRDN | 139. |
| 94 | SKNTLYLQMNSLRAEDTAVYYCAKDSNWGNFDLWGRGTLVTVSS | |
| VH | QVQLVQSGGGLVQPGGSLRLSCAVSGFTLSSYDMHWVRQPTGKGLEWVSAIGSTGDTYYPGSVKGRFTISRENA | 140. |
| 98 | KNSLYLQMNSLRAGDTAVYYCAREGWDVPFDFWGQGTLVTVSS | |
| VH | EVQLVESGGGLVQPGGSLRLSCAVSGFTLSSYDMHWVRQPTGKGLEWVSAIGSTGDTYYPGSVKGRFTISRENA | 141. |
| 114 | KNSLYLQMNSLRAGDTAVYYCAREGWDVPFDFWGQGTLVTVSS | |
| VH | EVQLVESGGGLVQPGGSLRLSCAASGFTLSSYDMHWVRQATGKGLEWVSAIGSTGDTYYPGSVKGRFTISRENA | 142. |
| 118 | KNSLYLQMNSLRAGDTAVYYCAREGWDVPFDFWGQGTLVTVSS | |
| VH | QVQLQESGPGLVKPSETLSLTCTVSGDSINTYYWSWFRQPPGKGLEWIGYIYYSGTTNYNPSLKSRVTISIDTP | 143. |
| 122 | RNQFSLKLISVTAADTAVYYCARERITMIRGVTLYYYSYGMDVWGQGTTVTVSS | |
| VH | QVQLQESGPGLVKPSETLSLTCTVSGDSINTYYWSWIRQPPGKGLEWIGYIYYSGTTNYNPSLKSRVTISVDTS | 144. |
| 142 | KNQFSLKLSSVTAADTAVYYCARERITMIRGVTLYYYSYGMDVWGQGTTVTVSS | |
| VH | QVQLVQSGAEVKKPGASVKVSCKASGYTFTNYGISWVRQAPGQGLELMGWISGYNGNTNYAQELQARVTMTTDT | 145. |
| 146 | STSTAYMELRNLRSDDTAVYYCARDRVVVAAANYYFYSMDVWGQGTTVTVSS | |
| VH | QVQLVQSGAEVKKPGASVKVSCKASGYTFTNYGISWVRQAPGQGLELMGWISGYNGNTNYAQELQARVTMTTDT | 146. |
| 162 | STSTAYMELRNLRSDDTAVYYCARDRVVVAAANYYFYSMDVWGQGTTVTVSS | |
| VH | QVQLVQSGAEVKKPGASVKVSCKASGYTFTNYGISWVRQAPGQGLEWMGWISGYNGNTNYAQKLQGRVTMTTDT | 147. |
| 166 | STSTAYMELRSLRSDDTAVYYCARDRVVVAAANYYFYSMDVWGQGTTVTVSS | |
| VH | QVHLKESGPTLVKPTQTLTLTCTFSGFSLITSGVGVGWIRQPPGKALEWLALIYWNGDKRYSPSLKSRLTITKD | 148. |
| 170 | TSKNQVVLTMTNMDPVDTATYYCAHRITETSYYFYYGMDVWGQGTTVTVSS | |
| VH | QITLKESGPTLVKPTQTLTLTCTFSGFSLITSGVGVGWIRQPPGKALEWLALIYWNGDKRYSPSLKSRLTITKD | 149. |
| 186 | TSKNQVVLTMTNMDPVDTATYYCAHRITETSYYFYYGMDVWGQGTTVTVSS | |
| VH | QITLKESGPTLVKPTQTLTLTCTFSGFSLITSGVGVGWIRQPPGKALEWLALIYWNGDKRYSPSLKSRLTITKD | 150. |
| 190 | TSKNQVVLTMTNMDPVDTATYYCAHRITETSYYFYYGMDVWGQGTTVTVSS | |
| VH | QITLKESGPTLVKPSQTLTLTCTFSGFSLSTSGVGVGWIRQPPGKALEWLALIYWNSDKRYSPSLKSRLTITKD | 151. |
| 194 | TSKNQVVLTMTNMDPVDTATYYCAHRHDSSSYYFYYGMDVWGQGITVTVSS | |
| VH | QITLKESGPTLVKPSQTLTLTCTFSGFSLSTSGVGVGWIRQPPGKALEWLALIYWNSDKRYSPSLKSRLTITKD | 152. |
| 210 | TSKNQVVLTMTNMDPVDTATYYCAHRHDSSSYYFYYGMDVWGQGTTVTVSS | |
| VH | QITLKESGPTLVKPTQTLTLTCTFSGFSLSTSGVGVGWIRQPPGKALEWLALIYWNSDKRYSPSLKSRLTITKD | 153. |
| 214 | TSKNQVVLTMTNMDPVDTATYYCAHRHDSSSYYFYYGMDVWGQGTTVTVSS | |
| VH | EVQLVESGGGLVQPGGSLRLSCAASGFTFSSHWMKWVRQAPGKGLEWVANINQDGSEKYYVDSVKGRFTISRDN | 154. |
| 234 | AKNSLFLQMNSLRAEDTAVYYCARDIVLMVYDMDYYYYGMDVWGQGTTVTVSS | |
| VH | EVQLVESGGGLVQPGGSLRLSCAASGFTFSSHWMSWVRQAPGKGLEWVANINQDGSEKYYVDSVKGRFTISRDN | 155. |
| 238 | AKNSLYLQMNSLRAEDTAVYYCARDIVLMVYDMDYYYYGMDVWGQGTTVTVSS | |
| VH | QVQLVESGGGVVQPGRSLRLSCAVSGFTFSSYGMHWVRQAPGKGLEWVAAISYDGSNKYYVDSVKGRFTISRDN | 156. |
| 242 | SKKTLYLQMNSLRAEDTAVYNCAKNIVLVMYDIDYHYYGMDVWGQGTTVTVSS | |
| VH | QVQLVESGGGVVQPGRSLRLSCAVSGFTFSSYGMHWVRQAPGKGLEWVAAISYDGSNKYYVDSVKGRFTISRDN | 157. |
| 258 | SKKTLYLQMNSLRAEDTAVYNCAKNIVLVMYDIDYHYYGMDVWGQGTTVTVSS | |
| VH | QVQLVESGGGVVQPGRSLRLSCAASGFTFSSYGMHWVRQAPGKGLEWVAVISYDGSNKYYADSVKGRFTISRDN | 158. |
| 262 | SKNTLYLQMNSLRAEDTAVYYCAKNIVLVMYDIDYHYYGMDVWGQGTTVTVSS | |
| VH | QVQLVESGGGVVQPGRSLRLSCAVSGFTFSSYGMHWVRQAPGKGLEWVAAISYDGSNKYYVDSVKGRFTISRDN | 159. |
| 266 | SKKTLYLQMNSLRAEDTAVYNCAKNIVLVMYDIDYHYYGMDVWGQGTTVTVSS | |
| VH | QVQLVESGGGVVQPGRSLRLSCAVSGFTFSSYGMHWVRQAPGKGLEWVAAISYDGSNKYYVDSVKGRFTISRDN | 160. |
| 282 | SKKTLYLQMNSLRAEDTAVYNCAKNIVLVMYDIDYHYYGMDVWGQGTTVTVSS | |
| VH | QVQLVESGGGVVQPGRSLRLSCAASGFTFSSYGMHWVRQAPGKGLEWVAVISYDGSNKYYADSVKGRFTISRDN | 161. |
| 286 | SKNTLYLQMNSLRAEDTAVYYCAKNIVLVMYDIDYHYYGMDVWGQGTTVTVSS | |
| VH | QITLKESGPTLVKPTQTLTLTCTFSGFSLSASGVGVGWFRQPPGKALEWLALIYWNDDKRYSPSLKNSLTITKD | 162. |
| 290 | TSKNQVVLTMTNMDPVDTATYYCAHRIHLWSYFYYGMDVWGQGTTVTVSS | |
| VH | QITLKESGPTLVKPTQTLTLTCTFSGFSLSASGVGVGWFRQPPGKALEWLALIYWNDDKRYSPSLKNSLTITKD | 163. |
| 306 | TSKNQVVLTMTNMDPVDTATYYCAHRIHLWSYFYYGMDVWGQGTTVTVSS | |
| VH | QITLKESGPTLVKPTQTLTLTCTFSGFSLSASGVGVGWIRQPPGKALEWLALIYWNDDKRYSPSLKSRLTITKD | 164. |
| 310 | TSKNQVVLTMTNMDPVDTATYYCAHRIHLWSYFYYGMDVWGQGTTVTVSS | |
| VH | QVQLVQSGPEVKNPGASVKVSCKASGYTFTTYGISWVRQAPGQGLEWMGWISGYNGKTNDAQKFQDRVAMTTDT | 165. |
| 314 | STSTAYMELRSLRSDDTAIYYCSRDRLVVPPALNYSYYVMDVWGQGTTVTVSS | |
| VH | QVQLVQSGPEVKNPGASVKVSCKASGYTFTTYGISWVRQAPGQGLEWMGWISGYNGKTNDAQKFQDRVAMTTDT | 166. |
| 330 | STSTAYMELRSLRSDDTAIYYCSRDRLVVPPALNYSYYVMDVWGQGTTVTVSS | |
| VH | QVQLVQSGAEVKKPGASVKVSCKASGYTFTTYGISWVRQAPGQGLEWMGWISGYNGKTNYAQKLQGRVTMTTDT | 167. |
| 334 | STSTAYMELRSLRSDDTAVYYCSRDRLVVPPALNYSYYVMDVWGQGTTVTVSS | |
| VH | EVQLVESGGGLVKPGGSLRLSCAASGFTFSSYSMDWVRQAPGKGLEWVSSISSSSSYIYYADSVKGRFTISRDT | 168. |
| 338 | AKNSLYLQMNSLRDEDTAVYYCAREGSSRLFDYWGQGTLVTVSS | |
| VH | EVQLVESGGGLVKPGGSLRLSCAASGFTFSSYSMDWVRQAPGKGLEWVSSISSSSSYIYYADSVKGRFTISRDT | 169. |
| 354 | AKNSLYLQMNSLRDEDTAVYYCAREGSSRLFDYWGQGTLVTVSS | |
| VH | EVQLVESGGGLVKPGGSLRLSCAASGFTFSSYSMNWVRQAPGKGLEWVSSISSSSSYIYYADSVKGRFTISRDN | 170. |
| 358 | AKNSLYLQMNSLRAEDTAVYYCAREGSSRLFDYWGQGTLVTVSS | |
| VH | QVHLVESGGGLVKPGGSLRLSCAASGFTFSDHYMSWIRQAPGKGLEWISYISNDGGTKYYVDSVEGRFIISRDN | 171. |
| 362 | AKNSLYLHMNSLRADDTAVYYCARDQGYIGYDSYYYYSYGMDVWGQGTTVTVAS | |
| VH | QVQLVESGGGLVKPGGSLRLSCAASGFTFSDHYMSWIRQAPGKGLEWISYISNDGGTKYYVDSVEGRFIISRDN | 172. |
| 378 | AKNSLYLHMNSLRADDTAVYYCARDQGYIGYDSYYYYSYGMDVWGQGTTVTVSS | |
| VH | QVQLVESGGGLVKPGGSLRLSCAASGFTFSDHYMSWIRQAPGKGLEWVSYISNDGGTKYYADSVKGRFTISRDN | 173. |
| 382 | AKNSLYLQMNSLRAEDTAVYYCARDQGYIGYDSYYYYSYGMDVWGQGTTVTVSS | |
| VH | EVQKVESGGGLVKPGGSLRLSCTASGFTFSTYNMNWVRQAPGKGLEWVSSIRSSSNYIYYADSVKGRFTISRDN | 174. |
| 386 | AKNSLYLQMNSLRADDTAVYYCARDGSSWYDYSDYWGQGTLVTVSS | |
| VH | EVQLVESGGGLVKPGGSLRLSCTASGFTFSTYNMNWVRQAPGKGLEWVSSIRSSSNYIYYADSVKGRFTISRDN | 175. |
| 402 | AKNSLYLQMNSLRADDTAVYYCARDGSSWYDYSDYWGQGTLVTVSS | |
| VH | EVQLVESGGGLVKPGGSLRLSCAASGFTFSTYNMNWVRQAPGKGLEWVSSIRSSSNYIYYADSVKGRFTISRDN | 176. |
| 406 | AKNSLYLQMNSLRAEDTAVYYCARDGSSWYDYSDYWGQGTLVTVSS | |
| VH | EVQLVESGGGLVKPGGSLRLSCTASGFTFSTYNMNWVRQAPGKGLEWVSSIRSSSNYIYYADSVKGRFTISRDN | 177. |
| 410 | AKNSLYLQMNSLRADDTAVYYCARDGSSWYDYSDYWGQGTLVTVSS | |
| VH | EVQLVESGGGLVKPGGSLRLSCTASGFTFSTYNMNWVRQAPGKGLEWVSSIRSSSNYIYYADSVKGRFTISRDN | 178. |
| 426 | AKNSLYLQMNSLRADDTAVYYCARDGSSWYDYSDYWGQGTLVTVSS | |
| VH | EVQLVESGGGLVKPGGSLRLSCAASGFTFSTYNMNWVRQAPGKGLEWVSSIRSSSNYIYYADSVKGRFTISRDN | 179. |
| 430 | AKNSLYLQMNSLRAEDTAVYYCARDGSSWYDYSDYWGQGTLVTVSS | |
| VH | EVQLVESGGGLVKPGGSLRLSCTASGFTFSTYNMNWVRQAPGKGLEWVSSIRSSSNYIYYADSVKGRFTISRDN | 180. |
| 434 | AKSSLYLQMNSLRAEDTAVYYCARDGSSWYDYSDYWGQGTLVTVSS | |
| VH | EVQLVESGGGLVKPGGSLRLSCTASGFTFSTYNMNWVRQAPGKGLEWVSSIRSSSNYIYYADSVKGRFTISRDN | 181. |
| 450 | AKSSLYLQMNSLRAEDTAVYYCARDGSSWYDYSDYWGQGTLVTVSS | |
| VH | EVQLVESGGGLVKPGGSLRLSCAASGFTFSTYNMNWVRQAPGKGLEWVSSIRSSSNYIYYADSVKGRFTISRDN | 182. |
| 454 | AKNSLYLQMNSLRAEDTAVYYCARDGSSWYDYSDYWGQGTLVTVSS | |
| VH | EVQLVESGGGLVKPGGSLRLSCTASGFTFSTYNMNWVRQAPGKGLEWVSSIRSSSNYIYYADSVKGRFTISRDN | 183. |
| 458 | AKNSLYLQMNSLRADDTAVYYCARDGSSWYDYSDYWGQGTLVTVSS | |
| VH | EVQLVESGGGLVKPGGSLRLSCTASGFTFSTYNMNWVRQAPGKGLEWVSSIRSSSNYIYYADSVKGRFTISRDN | 184. |
| 474 | AKNSLYLQMNSLRADDTAVYYCARDGSSWYDYSDYWGQGTLVTVSS | |
| VH | EVQLVESGGGLVKPGGSLRLSCAASGFTFSTYNMNWVRQAPGKGLEWVSSIRSSSNYIYYADSVKGRFTISRDN | 185. |
| 478 | AKNSLYLQMNSLRAEDTAVYYCARDGSSWYDYSDYWGQGTLVTVSS | |
| VH | EVQLVESGGGLVQPGGSLRLSCVVSGFTFGDYDMHWVRQATGRGLEWVSGIAPAGDTSYTGSVKGRFTISRENA | 186. |
| 482 | KNSLHLQMNSLTTGDTAIYYCAREDIAVPGFDYWGQGTLVTVSS | |
| VH | EVQLVESGGGLVQPGGSLRLSCVVSGFTFGDYDMHWVRQATGRGLEWVSGIAPAGDTSYTGSVKGRFTISRENA | 187. |
| 498 | KNSLHLQMNSLTTGDTAIYYCAREDIAVPGFDYWGQGTLVTVSS | |
| VH | EVQLVESGGGLVQPGGSLRLSCAASGFTFGDYDMHWVRQATGKGLEWVSAIAPAGDTYYPGSVKGRFTISRENA | 188. |
| 502 | KNSLYLQMNSLRAGDTAVYYCAREDIAVPGFDYWGQGTLVTVSS | |
| VH | QILLVQSGPEVKEPGASVKVSCKASGYTFTNYAISWVRQVPGQGLEWMGWVSAYNGHTNYAHEVQGRVTMTTDT | 189. |
| 506 | STTTAYMELRSLRSDDTAMYYCARGGVVVPVAPHFYNGMDVWGQGTTVTVSS | |
| VH | QVQLVQSGPEVKEPGASVKVSCKASGYTFTNYAISWVRQVPGQGLEWMGWVSAYNGHTNYAHEVQGRVTMTTDT | 190. |
| 522 | STTTAYMELRSLRSDDTAMYYCARGGVVVPVAPHFYNGMDVWGQGTTVTVSS | |
| VH | QVQLVQSGAEVKKPGASVKVSCKASGYTFTNYAISWVRQAPGQGLEWMGWVSAYNGHTNYAQKLQGRVTMTTDT | 191. |
| 526 | STSTAYMELRSLRSDDTAVYYCARGGVVVPVAPHFYNGMDVWGQGTTVTVSS | |
| VH | EVQLVESGGGLVQPGGSLRLSCAASGFTLSSYDMHWVRQATGKGLEWVSAIGSTGDTYYTGSVMGRFTISRDAA | 192. |
| 530 | KNSFYLEMNSLRVGDTAVYYCAREGIRTPYDYWGQGARVTVSS | |
| VH | EVQLVESGGGLVQPGGSLRLSCAASGFTLSSYDMHWVRQATGKGLEWVSAIGSTGDTYYTGSVMGRFTISRDAA | 193. |
| 546 | KNSFYLEMNSLRVGDTAVYYCAREGIRTPYDYWGQGTLVTVSS | |
| VH | EVQLVESGGGLVQPGGSLRLSCAASGFTLSSYDMHWVRQATGKGLEWVSAIGSTGDTYYPGSVKGRFTISRENA | 194. |
| 550 | KNSLYLQMNSLRAGDTAVYYCAREGIRTPYDYWGQGTLVTVSS | |
| VH | EVQLVESGGGLVQPGGSLRLSCAASGFTLSSYDMHWVRQATGKGLEWVSAIGSTGDTYYTGSVMGRFTISRDAA | 195. |
| 554 | KNSFYLEMNSLRVGDTAVYYCAREGIRTPYDYWGQGARVTVSS | |
| VH | EVQLVESGGGLVQPGGSLRLSCAASGFTLSSYDMHWVRQATGKGLEWVSAIGSTGDTYYTGSVMGRFTISRDAA | 196. |
| 570 | KNSFYLEMNSLRVGDTAVYYCAREGIRTPYDYWGQGTLVTVSS | |
| VH | EVQLVESGGGLVQPGGSLRLSCAASGFTLSSYDMHWVRQATGKGLEWVSAIGSTGDTYYPGSVKGRFTISRENA | 197. |
| 574 | KNSLYLQMNSLRAGDTAVYYCAREGIRTPYDYWGQGTLVTVSS | |
| VH | EVQLVESGGGLVQPGRSLRLSCAASGFTFDDYAMHWVRQAPGKGLEWVSGINWNSGSIGYADSVKGRFTISRDN | 198. |
| 578 | AKHSLYLQMNSLRPEDTALYYCVKEVTTGYYYGMDVWGQGTTVTVSS | |
| VH | EVQLVESGGGLVQPGRSLRLSCAASGFTFDDYAMHWVRQAPGKGLEWVSGINWNSGSIGYADSVKGRFTISRDN | 199. |
| 594 | AKHSLYLQMNSLRPEDTALYYCVKEVTTGYYYGMDVWGQGTTVTVSS | |
| VH | EVQLVESGGGLVQPGRSLRLSCAASGFTFDDYAMHWVRQAPGKGLEWVSGINWNSGSIGYADSVKGRFTISRDN | 200. |
| 598 | AKNSLYLQMNSLRAEDTALYYCVKEVTTGYYYGMDVWGQGTTVTVSS | |
| VH | EVQLLESGGGLVQPGGSLRLSCAASGFTFSSYAMNWVRQAPGKGLDWVSGISGNGGSTYYADSVKGRFTISRDI | 201. |
| 602 | SKNTLYVQMHSLRVEDTAVYYCAKARYYDFWGGNFDLWGRGTQVTVSS | |
| VH | EVQLLESGGGLVQPGGSLRLSCAASGFTFSSYAMNWVRQAPGKGLDWVSGISGNGGSTYYADSVKGRFTISRDI | 202. |
| 618 | SKNTLYVQMHSLRVEDTAVYYCAKARYYDFWGGNFDLWGRGTLVTVSS | |
| VH | EVQLLESGGGLVQPGGSLRLSCAASGFTFSSYAMSWVRQAPGKGLEWVSAISGNGGSTYYADSVKGRFTISRDN | 203. |
| 622 | SKNTLYLQMNSLRAEDTAVYYCAKARYYDFWGGNFDLWGRGTLVTVSS | |
| VH | QVQLVQSGPEVKNPGASVKVSCKASGYTFTTYGISWVRQAPGQGLEWMGWISGYNGKTNDAQKFQDRVAMTTDT | 204. |
| 626 | STSTAYMELRSLRSDDTAIYYCSRDRLVVPPALYYSYYVMDVWGQGTTVIVSSS | |
| VH | QVQLVQSGPEVKNPGASVKVSCKASGYTFTTYGISWVRQAPGQGLEWMGWISGYNGKTNDAQKFQDRVAMTTDT | 205. |
| 642 | STSTAYMELRSLRSDDTAIYYCSRDRLVVPPALYYSYYVMDVWGQGTTVIVSS | |
| VH | QVQLVQSGAEVKKPGASVKVSCKASGYTFTTYGISWVRQAPGQGLEWMGWISGYNGKTNYAQKLQGRVTMTTDT | 206. |
| 646 | STSTAYMELRSLRSDDTAVYYCSRDRLVVPPALYYSYYVMDVWGQGTTVTVSS | |
| VH | QVQLVQSGPEVKNPGASVKVSCKASGYTFTTYGISWVRQAPGQGLEWMGWISGYNGKTNDAQKFQDRVAMTTDT | 207. |
| 650 | STSTAYMELRSLRSDDTAIYYCSRDRLVVPPALNYYYYVMDVWGQGTTVIVSS | |
| VH | QVQLVQSGPEVKNPGASVKVSCKASGYTFTTYGISWVRQAPGQGLEWMGWISGYNGKTNDAQKFQDRVAMTTDT | 208. |
| 666 | STSTAYMELRSLRSDDTAIYYCSRDRLVVPPALNYYYYVMDVWGQGTTVIVSS | |
| VH | QVQLVQSGAEVKKPGASVKVSCKASGYTFTTYGISWVRQAPGQGLEWMGWISGYNGKTNYAQKLQGRVTMTTDT | 209. |
| 670 | STSTAYMELRSLRSDDTAVYYCSRDRLVVPPALNYYYYVMDVWGQGTTVTVSS | |
| VH | QVQLVQSGPEVKNPGASVKVSCKASGYTFTTYGISWVRQAPGQGLEWMGWISGYNGKTNDAQKFQDRVAMTTDT | 210. |
| 674 | STSTAYMELRSLRSDDTAIYYCSRDRLVVPPALYYYYYVMDVWGQGTTVTVSS | |
| VH | QVQLVQSGPEVKNPGASVKVSCKASGYTFTTYGISWVRQAPGQGLEWMGWISGYNGKTNDAQKFQDRVAMTTDT | 211. |
| 690 | STSTAYMELRSLRSDDTAIYYCSRDRLVVPPALYYYYYVMDVWGQGTTVTVSS | |
| VH | QVQLVQSGAEVKKPGASVKVSCKASGYTFTTYGISWVRQAPGQGLEWMGWISGYNGKTNYAQKLQGRVTMTTDT | 212. |
| 694 | STSTAYMELRSLRSDDTAVYYCSRDRLVVPPALYYYYYVMDVWGQGTTVTVSS | |
| VH | QVHLVESGGGLVKPGGSLRLSCAASGFTFSDHYMSWIRQAPGKGLEWISYISNDGGTKYYVDSVEGRFIISRDN | 213. |
| 698 | AKNSLYLHMNSLRADDTAVYYCARDQGYIGYDSYYYYSYGMDVWGQGTTVTVAS | |
| VH | QVQLVESGGGLVKPGGSLRLSCAASGFTFSDHYMSWIRQAPGKGLEWISYISNDGGTKYYVDSVEGRFIISRDN | 214. |
| 714 | AKNSLYLHMNSLRADDTAVYYCARDQGYIGYDSYYYYSYGMDVWGQGTTVTVSS | |
| VH | QVQLVESGGGLVKPGGSLRLSCAASGFTFSDHYMSWIRQAPGKGLEWVSYISNDGGTKYYADSVKGRFTISRDN | 215. |
| 718 | AKNSLYLQMNSLRAEDTAVYYCARDQGYIGYDSYYYYSYGMDVWGQGTTVTVSS | |
| VH | QILLVQSGPEVKEPGASVKVSCKASGYTFTNYAISWVRQVPGQGLEWMGWVSAYNGHTNYAHEVQGRVTMTTDT | 216. |
| 722 | STTTAYMELRSLRSDDTAMYYCARGGVVVPVAPHFYNGMDVWGQGTTVTVSS | |
| VH | QVQLVQSGPEVKEPGASVKVSCKASGYTFTNYAISWVRQVPGQGLEWMGWVSAYNGHTNYAHEVQGRVTMTTDT | 217. |
| 738 | STTTAYMELRSLRSDDTAMYYCARGGVVVPVAPHFYNGMDVWGQGTTVTVSS | |
| VH | QVQLVQSGAEVKKPGASVKVSCKASGYTFTNYAISWVRQAPGQGLEWMGWVSAYNGHTNYAQKLQGRVTMTTDT | 218. |
| 742 | STSTAYMELRSLRSDDTAVYYCARGGVVVPVAPHFYNGMDVWGQGTTVTVSS | |
| VL | DIVMTQSPDSLAVSLGERATINCKSSQSVFHTSNNKNYLVWYQQKPGQPPKLLIYWASTRESGVPDRFSGSGSG | 219. |
| 68 | TDFTLTISSLQAEDVANYYCHQYYSIPWTFGQGTKVEIK | |
| VL | DIVMTQSPDSLAVSLGERATINCKSSQSVLYRSNNRNFLGWYQQKPGQPPNLLIYWASTRESGVPDRFSGSGSG | 220. |
| 92 | TDFTLTISSLQAEDVAVYYCQQYYTTPYTFGQGTKLEIK | |
| H1H316P | | |
| VL | DIQMTQSPSFLSASVGDRVTITCWASQDISSYLAWYQQKPGIAPKLLIYAASTLQSGVPSRFGGSGSGTEFTLT | 221. |
| 140 | ISSLQPEDFATYYCQQLNSYPRTFGQGTKVEIK | |
| VL | DIVMTQSPLSLPVTPGEPASISCRSSQSLLHSNGNNYLDWYLQKPGQSPQLLIYLGSNRASGVPDRFSGSGSGT | 222. |
| 226 | DFTLKISRVEAEDVGVYYCMQTLQTPLTFGGGTKVEIK | |
| H1M300N | | |
| VL | DIQMTQSPATLSVSPGERAALSCRASQSVSSNLAWYHQKPGQAPRLLIYGASTRATGIPARFSGIGSGTEFTLI | 223. |
| 10 | ISSLQSEDFAFYFCQQYNNWPPFTFGPGTKVEIKR | |
| VL | EIVMTQSPATLSVSPGERAALSCRASQSVSSNLAWYHQKPGQAPRLLIYGASTRATGIPARFSGIGSGTEFTLI | 224. |
| 20 | ISSLQSEDFAFYFCQQYNNWPPFTFGPGTKVDIK | |
| VL | EIVMTQSPATLSVSPGERATLSCRASQSVSSNLAWYQQKPGQAPRLLIYGASTRATGIPARFSGSGSGTEFTLT | 225. |
| 24 | IsSLQSEDFAVYYCQQYNNWPPFTFGPGTKVDIK | |
| VL | AIQMTQSPSTLSASVGDRVTITCRASQSISSWLAWYQQKPGKAPKLLIYKASSLESGVPSRFSGSGSGTEFTLT | 226. |
| 34 | ISSLQPDDFATYYCQQYNSYYTFGQGTKVEIKR | |
| VL | DIQMTQSPSTLSASVGDRVTITCRASQSISSWLAWYQQKPGKAPKLLIYKASSLESGVPSRFSGSGSGTEFTLT | 227. |
| 44 | ISSLQPDDFATYYCQQYNSYYTFGQGTKLEIK | |
| VL | DIQMTQSPSTLSASVGDRVTITCRASQSISSWLAWYQQKPGKAPKLLIYKASSLESGVPSRFSGSGSGTEFTLT | 228. |
| 48 | ISSLQPDDFATYYCQQYNSYYTFGQGTKLEIK | |
| VL | AIQMTQSPDSLAVSLGERATINCKSSQSVFHTSNNKNYLVWYQQKPGQPPKLLIYWASTRESGVPDRFSGSGSG | 229. |
| 58 | TDFTLTISSLQAEDVANYYCHQYYSIPWTFGQGTKVEIKR | |
| VL | DIVMTQSPDSLAVSLGERATINCKSSQSVFHTSNNKNYLAWYQQKPGQPPKLLIYWASTRESGVPDRFSGSGSG | 230. |
| 72 | TDFTLTISSLQAEDVAVYYCHQYYSIPWTFGQGTKVEIK | |
| VL | DIQMTQSPDSLAVSLGERATINCKSSQSVLYRSNNRNFLGWYQQKPGQPPNLLIYWASTRESGVPDRFSGSGSG | 231. |
| 82 | TDFTLTISSLQAEDVAVYYCQQYYTTPYTFGQGTKVEIKR | |
| VL | DIVMTQSPDSLAVSLGERATINCKSSQSVLYRSNNRNFLAWYQQKPGQPPKLLIYWASTRESGVPDRFSGSGSG | 232. |
| 96 | TDFTLTISSLQAEDVAVYYCQQYYTTPYTFGQGTKLEIK | |
| VL | AIQLTQSPSSLSASVGDRVTITCRASQDIRNDLGWYQQKPGKAPKLLIYAASSLQSGVPSRFSGSGSGTDFTLT | 233. |
| 106 | ISSLQPEDFATYYCLQDYNYPWTFGQGTKVEIKR | |
| VL | AIQMTQSPSSLSASVGDRVTITCRASQDIRNDLGWYQQKPGKAPKLLIYAASSLQSGVPSRFSGSGSGTDFTLT | 234. |
| 116 | ISSLQPEDFATYYCLQDYNYPWTFGQGTKVEIK | |
| VL | AIQMTQSPSSLSASVGDRVTITCRASQDIRNDLGWYQQKPGKAPKLLIYAASSLQSGVPSRFSGSGSGTDFTLT | 235. |
| 120 | ISSLQPEDFATYYCLQDYNYPWTFGQGTKVEIK | |
| VL | DIQMTQSPSFLSASVGDRVTITCWASQDISSYLAWYQQKPGIAPKLLIYAASTLQSGVPSRFGGSGSGTEFTLT | 236. |
| 130 | ISSLQPEDFATYYCQQLNSYPRTFGQGTKVEIKR | |
| VL | DIQMTQSPSSLSASVGDRVTITCRASQDISSYLGWYQQKPGKAPKRLIYAASSLQSGVPSRFSGSGSGTEFTLT | 237. |
| 144 | ISSLQPEDFATYYCQQLNSYPRTFGQGTKVEIK | |
| VL | AIQMTQSPLSLSVTLGQPASISCRSSQSLVYSDGDTYLNWFQQRPGQSPRRLIYKVSNRDSGVPDRFSGSGSGT | 238. |
| 154 | AFTLKISGVEAEDVGVYYCMQATHWPRTFGQGTKVEIKR | |
| VL | DVVMTQSPLSLSVTLGQPASISCRSSQSLVYSDGDTYLNWFQQRPGQSPRRLIYKVSNRDSGVPDRFSGSGSGT | 239. |
| 164 | AFTLKISGVEAEDVGVYYCMQATHWPRTFGQGTKVEIK | |
| VL | DVVMTQSPLSLPVTLGQPASISCRSSQSLVYSDGDTYLNWFQQRPGQSPRRLIYKVSNRDSGVPDRFSGSGSGT | 240. |
| 168 | DFTLKISRVEAEDVGVYYCMQATHWPRTFGQGTKVEIK | |
| VL | DIQMTQSPLSLPVTPGEPASISCRSSQSLLHSHGYDYLDWYLQKPGQSPQLLIYLGSNRASGVPDRFSGSGSGT | 241. |
| 178 | DFTLKISRVEAEDVGVYYCMQALQTPLTFGGGTKVEIKR | |
| VL | DIVMTQSPLSLPVTPGEPASISCRSSQSLLHSHGYDYLDWYLQKPGQSPQLLIYLGSNRASGVPDRFSGSGSGT | 242. |
| 188 | DFTLKISRVEAEDVGVYYCMQALQTPLTFGGGTKVEIK | |
| VL | DIVMTQSPLSLPVTPGEPASISCRSSQSLLHSHGYDYLDWYLQKPGQSPQLLIYLGSNRASGVPDRFSGSGSGT | 243. |
| 192 | DFTLKISRVEAEDVGVYYCMQALQTPLTFGGGTKVEIK | |
| VL | DIQMTQSPLSLPVTPGEPASISCRSSQSLLHSHGYNYLDWYLQKPGQSPQLLIYLGSNRASGVPDRFSGGGSGT | 244. |
| 202 | DFTLKISRVEAEDVGIYYCMQALQTPLTFGGGTKVEIKR | |
| VL | DIVMTQSPLSLPVTPGEPASISCRSSQSLLHSHGYNYLDWYLQKPGQSPQLLIYLGSNRASGVPDRFSGGGSGT | 245. |
| 212 | DFTLKISRVEAEDVGIYYCMQALQTPLTFGGGTKVEIK | |
| VL | DIVMTQSPLSLPVTPGEPASISCRSSQSLLHSHGYNYLDWYLQKPGQSPQLLIYLGSNRASGVPDRFSGSGSGT | 246. |
| 216 | DFTLKISRVEAEDVGVYYCMQALQTPLTFGGGTKVEIK | |
| VL | DIVMTQSPLSLPVTPGEPASISCRSSQSLLHSNGNNYLDWYLQKPGQSPQLLIYLGSNRASGVPDRFSGSGSGT | 247. |
| 236 | DFTLKISRVEAEDVGVYYCMQTLQTPLTFGGGTKVEIK | |
| VL | DIVMTQSPLSLPVTPGEPASISCRSSQSLLHSNGNNYLDWYLQKPGQSPQLLIYLGSNRASGVPDRFSGSGSGT | 248. |
| 240 | DFTLKISRVEAEDVGVYYCMQTLQTPLTFGGGTKVEIK | |
| VL | DIVMTQSPLSLPVTPGEPASISCRSSQSLLHSNGYNYLDWYLQKPGQSPQLLIYLGFNRASGVPDRFSGSGSGT | 249. |
| 250 | DFTLKISRVEAEDVGVYYCMQALQTPLTFGGGTKVEIR | |
| VL | DIVMTQSPLSLPVTPGEPASISCRSSQSLLHSNGYNYLDWYLQKPGQSPQLLIYLGFNRASGVPDRFSGSGSGT | 250. |
| 260 | DFTLKISRVEAEDVGVYYCMQALQTPLTFGGGTKVEIK | |
| VL | DIVMTQSPLSLPVTPGEPASISCRSSQSLLHSNGYNYLDWYLQKPGQSPQLLIYLGFNRASGVPDRFSGSGSGT | 251. |
| 264 | DFTLKISRVEAEDVGVYYCMQALQTPLTFGGGTKVEIK | |
| VL | DIVMTQSPLSLPVTPGEPASISCRSSQSLLHSNGYNYLDWYLQKPGQSPQLLIYLGFNRASGVPDRFSGSGSGT | 252. |
| 274 | DFTLKISRVEAEDVGVYYCMQALQTPLTFGGGTKVEIR | |
| VL | DIVMTQSPLSLPVTPGEPASISCRSSQSLLHSNGYNYLDWYLQKPGQSPQLLIYLGFNRASGVPDRFSGSGSGT | 253. |
| 284 | DFTLKISRVEAEDVGVYYCMQALQTPLTFGGGTKVEIK | |
| VL | DIVMTQSPLSLPVTPGEPASISCRSSQSLLHSNGYNYLDWYLQKPGQSPQLLIYLGFNRASGVPDRFSGSGSGT | 254. |
| 288 | DFTLKISRVEAEDVGVYYCMQALQTPLTFGGGTKVEIK | |
| VL | DIVMTQSPLSLPVTPGEPASISCRSSQTLLHSNGYNYFDWYLQKPGQSPQLLIYLGSNRASGVPDRFSGSGSGT | 255. |
| 298 | DFTLKISRVEAEDVGIYYCMQALQTPLTFGGGTKVEIR | |
| VL | DIVMTQSPLSLPVTPGEPASISCRSSQTLLHSNGYNYFDWYLQKPGQSPQLLIYLGSNRASGVPDRFSGSGSGT | 256. |
| 308 | DFTLKISRVEAEDVGIYYCMQALQTPLTFGGGTKVEIK | |
| VL | DIVMTQSPLSLPVTPGEPASISCRSSQTLLHSNGYNYLDWYLQKPGQSPQLLIYLGSNRASGVPDRFSGSGSGT | 257. |
| 312 | DFTLKISRVEAEDVGVYYCMQALQTPLTFGGGTKVEIK | |
| VL | DVVMTQSPLSLPVTLGQPASISCRSSQSLVYSDGNTYLNWSQQRPGQSPRRLIYKVSNRDSGVPDRFSGSGSGT | 258. |
| 322 | DFTLKISRVEAEDVGVYYCMQGTHWPYTFGQGTKLEIK | |
| VL | DVVMTQSPLSLPVTLGQPASISCRSSQSLVYSDGNTYLNWSQQRPGQSPRRLIYKVSNRDSGVPDRFSGSGSGT | 259. |
| 332 | DFTLKISRVEAEDVGVYYCMQGTHWPYTFGQGTKLEIK | |
| VL | DVVMTQSPLSLPVTLGQPASISCRSSQSLVYSDGNTYLNWFQQRPGQSPRRLIYKVSNRDSGVPDRFSGSGSGT | 260. |
| 336 | DFTLKISRVEAEDVGVYYCMQGTHWPYTFGQGTKLEIK | |
| VL | DIQMTQSPSTLSASVGDRVTITCRASQSISSWLAWYQQRPGKAPKLLIYKASSLEGGVPSRFSGSGSGTEFTLT | 261. |
| 346 | ISSLQPEDFATYYCQQYNSYWYTFGQGTKLEIK | |
| VL | DIQMTQSPSTLSASVGDRVTITCRASQSISSWLAWYQQRPGKAPKLLIYKASSLEGGVPSRFSGSGSGTEFTLT | 262. |
| 356 | IsSLQPEDFATYYCQQYNSYWYTFGQGTKLEIK | |
| VL | DIQMTQSPSTLSASVGDRVTITCRASQSISSWLAWYQQKPGKAPKLLIYKASSLESGVPSRFSGSGSGTEFTLT | 263. |
| 360 | 15SLQPDDFATYYCQQYNSYWYTFGQGTKLEIK | |
| VL | KIVLIQSPGILPLFPGERATLSCRASQSVNNKFLAWYQQKSGQAPRLLIYGASSRATGIPDRFSGSGSGTDFTL | 264. |
| 370 | IISGLEPEDFEVYYCQVYGNSLTLGGGTKVEIK | |
| VL | EIVLIQSPGILPLFPGERATLSCRASQSVNNKFLAWYQQKSGQAPRLLIYGASSRATGIPDRFSGSGSGTDFTL | 265. |
| 380 | IISGLEPEDFEVYYCQVYGNSLTLGGGTKVEIK | |
| VL | EIVLIQSPGILSLSPGERATLSCRASQSVNNKFLAWYQQKPGQAPRLLIYGASSRATGIPDRFSGSGSGTDFTL | 266. |
| 384 | IISRLEPEDFAVYYCQVYGNSLTFGGGTKVEIK | |
| VL | DIQMTQSPSTLSASVGDRVTITCRASQSISSWLAWYQQIPGKAPKLLIYKASSLENGVPSRFSGSGSGTEFTLI | 267. |
| 394 | ISSLQPDDFATYYCQQYISYSRTFGQGTKVEIK | |
| VL | DIQMTQSPSTLSASVGDRVTITCRASQSISSWLAWYQQIPGKAPKLLIYKASSLENGVPSRFSGSGSGTEFTLI | 268. |
| 404 | ISSLQPDDFATYYCQQYISYSRTFGQGTKVEIK | |
| VL | DIQMTQSPSTLSASVGDRVTITCRASQSISSWLAWYQQKPGKAPKLLIYKASSLESGVPSRFSGSGSGTEFTLT | 269. |
| 408 | ISSLQPDDFATYYCQQYISYSRTFGQGTKVEIK | |
| VL | DIQMTQSPSTLSASVGDRVTITCRASQSISSWLAWYQQIPGKAPKLLIYKASSLENGVPSRFSGSGSGTEFTLI | 270. |
| 418 | ISSLQPDDFATYYCQQYISYSRTFGQGTKVEIK | |
| VL | DIQMTQSPSTLSASVGDRVTITCRASQSISSWLAWYQQIPGKAPKLLIYKASSLENGVPSRFSGSGSGTEFTLI | 271. |
| 428 | ISSLQPDDFATYYCQQYISYSRTFGQGTKVEIK | |
| VL | DIQMTQSPSTLSASVGDRVTITCRASQSISSWLAWYQQKPGKAPKLLIYKASSLESGVPSRFSGSGSGTEFTLT | 272. |
| 432 | ISSLQPDDFATYYCQQYISYSRTFGQGTKVEIK | |
| VL | DIQMTQSPSTLSASVGDRVTITCRASQSISSWLAWYQQVPGKAPKLLIYKASSLENGVPSRFSGSGSGTEFTLI | 273. |
| 442 | ISSLQPDDFATYYCQQYISYSRTFGQGTKVEIK | |
| VL | DIQMTQSPSTLSASVGDRVTITCRASQSISSWLAWYQQVPGKAPKLLIYKASSLENGVPSRFSGSGSGTEFTLI | 274. |
| 452 | IsSLQPDDFATYYCQQYISYSRTFGQGTKVEIK | |
| VL | DIQMTQSPSTLSASVGDRVTITCRASQSISSWLAWYQQKPGKAPKLLIYKASSLESGVPSRFSGSGSGTEFTLT | 275. |
| 456 | ISSLQPDDFATYYCQQYISYSRTFGQGTKVEIK | |
| VL | DIQMTQSPSTLSASVGDRVTITCRASQSISSWLAWYQQIPGKAPKLLIYKASSLENGVPSRFSGSGSGTEFTLI | 276. |
| 466 | ISSLQPDDFATYYCQQYISYSRTFGQGTKVEIK | |
| VL | DIQMTQSPSTLSASVGDRVTITCRASQSISSWLAWYQQIPGKAPKLLIYKASSLENGVPSRFSGSGSGTEFTLI | 277. |
| 476 | ISSLQPDDFATYYCQQYISYSRTFGQGTKVEIK | |
| VL | DIQMTQSPSTLSASVGDRVTITCRASQSISSWLAWYQQKPGKAPKLLIYKASSLESGVPSRFSGSGSGTEFTLT | 278. |
| 480 | ISSLQPDDFATYYCQQYISYSRTFGQGTKVEIK | |
| VL | EIVMTQSPATLSVSPGERGTLSCRASQSVSSNLAWYQQKPGQAPRLLIYGASTRATGFPARFSGSGSGTEFTLT | 279. |
| 490 | ISSLQSEDFAVYYCQQYNKWPPFTFGPGTKVDFK | |
| VL | EIVMTQSPATLSVSPGERGTLSCRASQSVSSNLAWYQQKPGQAPRLLIYGASTRATGFPARFSGSGSGTEFTLT | 280. |
| 500 | ISSLQSEDFAVYYCQQYNKWPPFTFGPGTKVDIK | |
| VL | EIVMTQSPATLSVSPGERATLSCRASQSVSSNLAWYQQKPGQAPRLLIYGASTRATGIPARFSGSGSGTEFTLT | 281. |
| 504 | ISSLQSEDFAVYYCQQYNKWPPFTFGPGTKVDIK | |
| VL | DIVMTQFPLSLPVTPGEPASISCRSSQSLLHINEYNYLDWYLKKPGQSPQLLIYLGFNRASGVPDRFSGSGSGT | 282. |
| 514 | DFTLKISRVEAEDVGVYYCMQALQTPWTLGQGTKVEIK | |
| VL | DIVMTQSPLSLPVTPGEPASISCRSSQSLLHINEYNYLDWYLKKPGQSPQLLIYLGFNRASGVPDRFSGSGSGT | 283. |
| 524 | DFTLKISRVEAEDVGVYYCMQALQTPWTLGQGTKVEIK | |
| VL | DIVMTQSPLSLPVTPGEPASISCRSSQSLLHINEYNYLDWYLQKPGQSPQLLIYLGSNRASGVPDRFSGSGSGT | 284. |
| 528 | DFTLKISRVEAEDVGVYYCMQALQTPWTFGQGTKVEIK | |
| VL | EIVMTQSPATLSVSPGERATLSCRASQSVSSNVAWYQQKPGQAPRLLIYGASTRATGIPARFSGSGSGTEFTLT | 285. |
| 538 | ISSLQSEDFAVYYCQQYNNWPPFTFGPGTKVDIK | |
| VL | EIVMTQSPATLSVSPGERATLSCRASQSVSSNVAWYQQKPGQAPRLLIYGASTRATGIPARFSGSGSGTEFTLT | 286. |
| 548 | ISSLQSEDFAVYYCQQYNNWPPFTFGPGTKVDIK | |
| VL | EIVMTQSPATLSVSPGERATLSCRASQSVSSNLAWYQQKPGQAPRLLIYGASTRATGIPARFSGSGSGTEFTLT | 287. |
| 552 | ISSLQSEDFAVYYCQQYNNWPPFTFGPGTKVDIK | |
| VL | EIVMTQSPATLSVSPGERATLSCRASQSVSSNVAWYQQKPGQAPRLLIYGASTRATGIPARFSGSGSGTEFTLT | 288. |
| 562 | ISSLQSEDFAVYYCQQYNNWPPFTFGPGTKVDIK | |
| VL | EIVMTQSPATLSVSPGERATLSCRASQSVSSNVAWYQQKPGQAPRLLIYGASTRATGIPARFSGSGSGTEFTLT | 289. |
| 572 | ISSLQSEDFAVYYCQQYNNWPPFTFGPGTKVDIK | |
| VL | EIVMTQSPATLSVSPGERATLSCRASQSVSSNLAWYQQKPGQAPRLLIYGASTRATGIPARFSGSGSGTEFTLT | 290. |
| 576 | ISSLQSEDFAVYYCQQYNNWPPFTFGPGTKVDIK | |
| VL | DIQLTQSPSFLSASVGDRVTITCWASQGISSYLAWYQKKPGKAPNLLIYDASTLQSGVPSRFSGSGSGTEFTLT | 291. |
| 586 | LSSLQPEDFATYYCQQLNIYPFTFGPGTKVDIK | |
| VL | DIQLTQSPSFLSASVGDRVTITCWASQGISSYLAWYQKKPGKAPNLLIYDASTLQSGVPSRFSGSGSGTEFTLT | 292. |
| 596 | LSSLQPEDFATYYCQQLNIYPFTFGPGTKVDIK | |
| VL | DIQLTQSPSFLSASVGDRVTITCRASQGISSYLAWYQQKPGKAPKLLIYDASTLQSGVPSRFSGSGSGTEFTLT | 293. |
| 600 | ISSLQPEDFATYYCQQLNIYPFTFGPGTKVDIK | |
| VL | EIVLTQSPGTLSLSPGERATLSCRASQSVSIRYLAWYQQKPGQAPRLLIYGASSRATGIPDRFSVSVSGTDFTL | 294. |
| 610 | TITRLEPEDFAVYYCQQYGSSPLTFGGGTKVEIK | |
| VL | EIVLTQSPGTLSLSPGERATLSCRASQSVSIRYLAWYQQKPGQAPRLLIYGASSRATGIPDRFSVSVSGTDFTL | 295. |
| 620 | TITRLEPEDFAVYYCQQYGSSPLTFGGGTKVEIK | |
| VL | EIVLTQSPGTLSLSPGERATLSCRASQSVSIRYLAWYQQKPGQAPRLLIYGASSRATGIPDRFSGSGSGTDFTL | 296. |
| 624 | TISRLEPEDFAVYYCQQYGSSPLTFGGGTKVEIK | |
| VL | DVVMTQSPLSLPVTLGQPASISCRSSQSLVYSDGNTYLNWFQQRPGQSPRRLIYKVSNRDSGVPDRFSGSGSGT | 297. |
| 634 | DFTLKISRVEAEDVGVYYCMQGTHWPYTFGQGTKLEIK | |
| VL | DVVMTQSPLSLPVTLGQPASISCRSSQSLVYSDGNTYLNWFQQRPGQSPRRLIYKVSNRDSGVPDRFSGSGSGT | 298. |
| 644 | DFTLKISRVEAEDVGVYYCMQGTHWPYTFGQGTKLEIK | |
| VL | DVVMTQSPLSLPVTLGQPASISCRSSQSLVYSDGNTYLNWFQQRPGQSPRRLIYKVSNRDSGVPDRFSGSGSGT | 299. |
| 648 | DFTLKISRVEAEDVGVYYCMQGTHWPYTFGQGTKLEIK | |
| VL | DVVMTQSPLSLPVTLGQPASISCRSSQSLVYSDGNTYLNWFQQRPGQSPRRLIYKVSNRDSGVPDRFSGSGSGT | 300. |
| 658 | DFTLKISRVEAEDVGVYYCMQGTHWPYTFGQGTKLEIK | |
| VL | DVVMTQSPLSLPVTLGQPASISCRSSQSLVYSDGNTYLNWFQQRPGQSPRRLIYKVSNRDSGVPDRFSGSGSGT | 301. |
| 668 | DFTLKISRVEAEDVGVYYCMQGTHWPYTFGQGTKLEIK | |
| VL | DVVMTQSPLSLPVTLGQPASISCRSSQSLVYSDGNTYLNWFQQRPGQSPRRLIYKVSNRDSGVPDRFSGSGSGT | 302. |
| 672 | DFTLKISRVEAEDVGVYYCMQGTHWPYTFGQGTKLEIK | |
| VL | DVVMTQSPLSLPVTLGQPASISCRSSQSLVYSDGNTYLNWFQQRPGQSPRRLIYKVSNRDSGVPDRFSGSGSGT | 303. |
| 682 | DFTLKISRVEAEDVGVYYCMQGTHWPYTFGQGTKLEIK | |
| VL | DVVMTQSPLSLPVTLGQPASISCRSSQSLVYSDGNTYLNWFQQRPGQSPRRLIYKVSNRDSGVPDRFSGSGSGT | 304. |
| 692 | DFTLKISRVEAEDVGVYYCMQGTHWPYTFGQGTKLEIK | |
| VL | DVVMTQSPLSLPVTLGQPASISCRSSQSLVYSDGNTYLNWFQQRPGQSPRRLIYKVSNRDSGVPDRFSGSGSGT | 305. |
| 696 | DFTLKISRVEAEDVGVYYCMQGTHWPYTFGQGTKLEIK | |
| VL | KIVLIQSPGILPLFPGERATLSCRASQSVNNKFLAWYQQKSGQAPRLLIYGASSRATGIPDRFSGSGSGTDFTL | 306. |
| 706 | TISGLEPEDFEVYYCQVYGNSLTFGGGTKVEIK | |
| VL | EIVLIQSPGILPLFPGERATLSCRASQSVNNKFLAWYQQKSGQAPRLLIYGASSRATGIPDRFSGSGSGTDFTL | 307. |
| 716 | TISGLEPEDFEVYYCQVYGNSLTFGGGTKVEIK | |
| VL | EIVLIQSPGILSLSPGERATLSCRASQSVNNKFLAWYQQKPGQAPRLLIYGASSRATGIPDRFSGSGSGTDFTL | 308. |
| 720 | TISRLEPEDFAVYYCQVYGNSLTFGGGTKVEIK | |
| VL | DIVMTQFPLSLPVTPGEPASISCRSSQSLLHINEYNYLDWYLKKPGQSPQLLIYLGFNRASGVPDRFSGSGSGT | 309. |
| 730 | DFTLKISRVEAEDVGVYYCMQALQTPWTFGQGTKVEIK | |
| VL | DIVMTQSPLSLPVTPGEPASISCRSSQSLLHINEYNYLDWYLKKPGQSPQLLIYLGFNRASGVPDRFSGSGSGT | 310. |
| 740 | DFTLKISRVEAEDVGVYYCMQALQTPWTFGQGTKVEIK | |
| VL | DIVMTQSPLSLPVTPGEPASISCRSSQSLLHINEYNYLDWYLQKPGQSPQLLIYLGSNRASGVPDRFSGSGSGT | 311. |
| 744 | DFTLKISRVEAEDVGVYYCMQALQTPWTFGQGTKVEIK | |

GROUP E

| | | |
|---|---|---|
| VH | EVQLVESGGGLVQPGGSLRLSCAASGFTFTGYAIHWVRQAPGKGLEWVGRISPANGNTNYADSVKGRFTISADT | 312. |
| 508.20 | SKNTAYLQMNSLRAEDTAVYYCARWIGSRELYIMDYWGQGTLVTVSS | |
| VH | EVQLVESGGGLVQPGGSLRLSCAASGFTFTGYAIHWVRQAPGKGLEWVGRISPANGNTNYADSVKGRFTISADT | 313. |
| 508.04 | SKNTAYLQMNSLRAEDTAVYYCARWIGSRELYIMDYWGQGTLVTVSS | |
| VH | EVQLVESGGGLVQPGGSLRLSCAASGFTFTGYAIHWVRQAPGKGLEWVGRISPANGNTNYADSVKGRFTISADT | 314. |
| 508.06 | SKNTAYLQMNSLRAEDTAVYYCARWIGSRELYIMDYWGQGTLVTVSS | |
| VH | EVQLVESGGGLVQPGGSLRLSCAASGFTFTRHTIHWVRQAPGKGLEWVGRISPANGNTNYADSVKGRFTISADT | 315. |
| 508.28 | SKNTAYLQMNSLRAEDTAVYYCARWIGSRELYIMDYWGQGTLVTVSS | |
| VH | EVQLVESGGGLVQPGGSLRLSCAASGFTFSSTAIHWVRQAPGKGLEWVGRISPANGNTNYADSVKGRFTISADT | 316. |
| 508.33 | SKNTAYLQMNSLRAEDTAVYYCARWIGSRELYIMDYWGQGTLVTVSS | |
| VH | EVQLVESGGGLVQPGGSLRLSCAASGFTFTGYAIHWVRQAPGKGLEWVGRISPANGNTNYADSVKGRFTISADT | 317. |
| 508.84 | SKNTAYLQMNSLRAEDTAVYYCARWIGSRELYIMDYWGQGTLVTVSS | |
| VL | DIQMTQSPSSLSASVGDRVTITCRASQDVSSAVAWYQQKPGKAPKLLIYSASSLYSGVPSRFSGSRSGTDFTLT | 318. |
| 508.20 | ISSLQPEDFATYYCQQSYTTPPTFGQGTKVEIKR | |
| VL | DIQMTQSPSSLSASVGDRVTITCRASQDVSSAVAWYQQKPGKAPKLLIYSASSLYSGVPSRFSGSRSGTDFTLT | 319. |
| 508.04 | ISSLQPEDFATYYCQQSYPAPATFGQGTKVEIKR | |
| VL | DIQMTQSPSSLSASVGDRVTITCRASQDVSSAVAWYQQKPGKAPKLLIYSASSLYSGVPSRFSGSRSGTDFTLT | 320. |
| 508.06 | ISSLQPEDFATYYCQQSYPSPATFGQGTKVEIKR | |
| VL | DIQMTQSPSSLSASVGDRVTITCRASQDVSSAVAWYQQKPGKAPKLLIYSASSLYSGVPSRFSGSRSGTDFTLT | 321. |
| 508.28 | ISSLQPEDFATYYCQQSYRIQPTFGQGTKVEIKR | |
| VL | DIQMTQSPSSLSASVGDRVTITCRASQDVSSAVAWYQQKPGKAPKLLIYSASSLYSGVPSRFSGSRSGTDFTLT | 322. |
| 508.33 | ISSLQPEDFATYYCQQSYPALHTFGQGTKVEIKR | |
| VL | DIQMTQSPSSLSASVGDRVTITCRASQDVSSAVAWYQQKPGKAPKLLIYSASSLYSGVPSRFSGSRSGTDFTLT | |
| 508.84 | ISSLQPEDFATYYCQQSYPAPSTFGQGTKVEIKR | 323. |

GROUP F

| | | |
|---|---|---|
| VH | EVQLVESGGGLVKPGGSLRLSCAASGFPFSKLGMVWVRQAPGKGLEWVSTISSGGGYTYYPDSVKGRFTISRDN | 324. |
| | AKNSLYLQMNSLRAEDTAVYYCAREGISFQGGTYTYVMDYWGQGTLVTVSS | |
| VL | DIVMTQSPLSLPVTPGEPASISCRSSKSLIAIRNGITYSYWYLQKPGQSPQLLIYQLSNLASGVPDRFSGSGSGT | |
| | DFTLKISRVEAEDVGVYYCYQNLELPLTFGQGTKVEIK | 325. |

GROUP G

| | | |
|---|---|---|
| VH | QVQLVQSGAEVKKPGESLKISCKGSGYSFTNYWISWVRQMPGKGLEWMGIIYPGDSYTNYSPSFQGQVTISADK | 326. |
| IB20 | SISTAYLQWSSLKASDTAMYYCARDYWYKPLFDIWGQGTLVTVSS | |
| VL | DIVMTQSPDSLAVSLGERATINCRSSQSVLYSSNNKNYLAWYQQKPGQPPKLLIYWASTRESGVPDRFSGSGSG | 327. |
| IB20 | TDFTLTISLQAEDVAVYYCQQYSSFPITFGQGTKVEIKR | |
| IB20 Variant | GYSFTX₁YX₂IX₃ | 328. |
| HC CDR1 | X₁ = N or D | |
| | X₂ = W or Y | |
| IB20 Variant | WMGX₁IYPGDSX₂TX₃YX₄X₅X₆FQG | 329. |
| HC CDR2 | X₁ = I, R, W, L, or M | |
| | X₂ = Y or D | |
| | X₃ = N, R, H, S, K, Q | |
| | X₄ = S or N | |
| | X₅ = P, Q, H | |
| | X₆ = S, K, N, or R | |
| IB20 Variant | DX₁X₂X₃X₄X₅X₆X₇DX₈ | 330. |
| HC CDR3 | X₁ = Y, R, or H | |
| | X₂ = W, Y, G, A, or F | |
| | X₃ = Y or S | |
| | X₄ = K, R, T, G, S, D, E, H, or N | |
| | X₅ = P, S, G, D, A, H, R, or Y | |
| | X₆ = L, Y, F, A, D, H, P, or S | |
| | X₇ = F or S | |
| | X₈ = I, Vr Y, F, or N | |
| IB30 Variant | WMGX₁TYPGDSYTX₂YSX₃SFQG | 331. |
| VH CDR2 | X₁ = I or R | |
| | X₂ = N, R, H, or S | |
| | X₃ = P or Q | |
| IB30 Variant | DYWYX₁X₂X₃FDX₄ | 332. |
| VH CDR3 | X₁ = K, G, R, S, D, E, H, N, or Q | |
| | X₂ = P, G, D, S, A, H, R, or Y | |
| | X₃ = L, A, Y, D, F, H, P, S, or V | |
| | X₄ = I, Y, F, or N | |
| IB20 Variant | X₁ Ser Ser Gln Ser Val X₂ X₃ Ser X₄ X₅ X₆ Lys Asn X₇ Leu X₈ | 333. |
| LC CDR1 | X₁ = Arg, Lys, His, Asn, Gln or Ser | |
| | X₂ = Leu or Phe | |
| | X₃ = Tyr or His | |
| | X₄ = Ser, Arg or Gly | |
| | X₅ = Asn or Thr | |
| | X₆ = Asn, Arg, His or Ser | |
| | X₇ = Tyr or Phe | |
| | X₈ = Ala or Thr | |
| IB20 Variant | LLIYX₁X₂SX₃RX₄X₅ | 334. |
| LC CDR2 | X₁ = Trp, Phe or Leu | |
| | X₂ = Ala or Thr | |
| | X₃ = Thr, Ile, Ala or Val | |
| | X₄ = Glu, Ala or Lys | |
| | X₅ = Ser or Thr | |
| IB20 Variant | QQYX₁X₂X₃PX₄ | 335. |
| LC CDR3 | X₁ = Ser or Tyr | |
| | X₂ = Ser or Thr | |
| | X₃ = Phe, Tyr, Leu, Thr, His, Ile, Asn, Pro or Ser | |
| | X₄ = Ile, Arg, Val, Tyr, Asp, Phe, Gly, His, Leu, Asn or Ser | |
| 1B20 | RSSQSVLYSSNNKNX₁LX₂ | 336. |
| Antibody | X₁ = Tyr or Phe | |
| Variant VL | X₂ = Ala or Thr | |
| CDR1 | | |
| Sequence | | |
| 1B20 | LLIYX₁ASTRX₂X₃ | 337. |
| Antibody | X₁ = Trp, Phe or Leu | |
| Variant VL | X₂ = Glu or Lys | |
| CDR2 | X₃ = Ser or Thr | |
| Sequence | | |
| 1B20 | QQYSSX₁PX₂ | 338. |
| Antibody | X₁ = Phe, Tyr, Thr, Ile, Asn or Ser | |
| Variant VL | X₂ = Ile, Tyr, Arg, Phe, His, Leu, Asn or Ser | |
| CDR3 | | |
| Sequence | | |
| VH; 1B20 | QVQLVQSGAEVKKPGESLKISCKGSGYSFTX₁YX₂IX₃WVRQMPGKGLEWMGX₄IYPGDSX₅TX₆YX₇X₈X₉F | 339. |
| VARIANT | QGQVTISADKSISTAYLQWSSLKASDTAMYYCARDX₁₀X₁₁X₁₂X₁₃X₁₄X₁₅X₁₆DX₁₇WGQGTLVTVSS | |
| SEQUENCE | X₁ = Asn or Asp | |
| | X₂ = Trp or Tyr | |
| | X₃ = Ser, Thr or Ala | |
| | X₄ = Ile, Arg, Trp, Leu or Met | |
| | X₅ = Tyr or Asp | |
| | X₆ = Asn, Arg, His, Ser, Lys or Gln | |
| | X₇ = Ser or Asn | |
| | X₈ = Pro, Gln or His | |
| | X₉ = Ser, Lys, Asn or Arg | |
| | X₁₀ = Tyr, Arg or His | |
| | X₁₁ = Trp, Tyr or Gly | |
| | X₁₂ = Tyr or Ser | |
| | X₁₃ = Lys, Arg, Thr, Gly, Ser, Asp, Glu, His, Asn or Gln | |
| | X₁₄ = Pro, Ser, Gly, Asp, Ala, His, Arg or Tyr | |
| | X₁₅ = Leu, Tyr, Phe, Ala, Asp, His, Pro, Ser or Val | |
| | X₁₆ = Phe or Ser | |
| | X₁₇ = Ile, Val, Tyr, Phe or Asn | |
| VH; 1B20 | QVQLVQSGAEVKKPGESLKISCKGSGYSFTX₁YX₂IX₃WVRQMPGKGLEWMGX₄IYPGDSYTX₅YSX₆SFQGQ | 340. |
| VARIANT | VTISADKSISTAYLQWSSLKASDTAMYYCARDWYWX₇X₈X₉FDX₁₀WGQGTLVTVSS | |
| SEQUENCE | X₁ = Asn or Asp | |
| | X₂ = Trp or Tyr | |
| | X₃ = Ser, Thr or Ala | |
| | X₄ = Ile or Arg | |
| | X₅ = Asn, Arg, His, Ser, Lys or Gln | |
| | X₆ = Pro or Gln | |
| | X₇ = Lys, Gly, Arg, Ser, Asp, Glu, His, Asn or Gln | |
| | X₈ = Pro, Gly, Asp, Ser, Ala, His, Arg or Tyr | |
| | X₉ = Leu, Ala, Tyr, Asp, Phe, His, Pro, Ser or Val | |
| | X₁₀ = Ile, Tyr, Phe or Asn | |
| VL; 1B20 | DIVMTQSPDSLAVSLGERATINCX₁SSQSVX₂X₃SX₄X₅X₆KNX₇LX₈WYQQKPGQPPKLLIYX₉X₁₀SX₁₁RX₁₂ | 341. |
| Variant | X₁₃GVPDRFSGSGSGTDFTLTISSLQAEDVAVYYCQQYX₁₄X₁₅X₁₆PX₁₇TFGQGTKVEIKR | |
| Sequence | X₁ = Arg, Lys, His, Asn, Gln or Ser | |
| | X₂ = Leu or Phe | |
| | X₃ = Tyr or His | |
| | X₄ = Ser, Arg or Gly | |
| | X₅ = Asn or Thr | |
| | X₆ = Asn, Arg, His or Ser | |
| | X₇ = Tyr or Phe | |
| | X₈ = Ala or Thr | |
| | X₉ = Trp, Phe or Leu | |
| | X₁₀ = Ala or Thr | |
| | X₁₁ = Thr, Ile, Ala or Val | |
| | X₁₂ = Glu, Ala or Lys | |
| | X₁₃ = Ser or Thr | |
| | X₁₄ = Ser or Tyr | |
| | X₁₅ = Ser or Thr | |
| | X₁₆ = Phe, Tyr, Leu, Thr, His, Ile, Asn, Pro or Ser | |
| | X₁₇ = Ile, Arg, Val, Tyr, Asp, Phe, Gly, His, Leu, Asn or Ser | |
| VL; 1B20 | DIVMTQSPDSLAVSLGERATINCRSSQSVLYSSNNKNX₁LX₂WYQQKPGQPPKLLIYX₃ASTRX₄X₅GVPDRFSGS | 342. |
| VARIANT | GSGTDFTLTISSLQAEDVAVYYCQQYSSX₆PX₇TFGQGTKVEIKR | |
| SEQUENCE | X₁ = Y or F | |
| | X₂ = A or T | |
| | X₃ = W, F, or L | |
| | X₄ = E or K | |
| | X₅ = S or T | |
| | X₆ = F, Y, T, I, N, or S | |
| | X₇ = I, Y, R, F, H, L, N, or S | |
| VH; 1B20 | QVQLVQSGAEVKKPGESLKISCKGSGYSFTNYYISWVRQMPGKGLEWMGLIYPGDSYTRY | 343. |
| Variant | NPSFQGQVTISADKSISTAYLQWSSLKASDTAMYYCARDYYSRPFSDIWGQGTLVTVSS | |
| Sequence | | |
| F120 | | |
| VH; 1B20 | QVQLVQSGAEVKKPGESLKISCKGSGYSFTNYYISWVRQMPGKGLEWMGLIYPGDSYTRY | 344. |
| Variant | NPKFQGQVTISADKSISTAYLQWSSLKASDTAMYYCARDYYSRPYSDVWGQGTLVTVSS | |
| Sequence | | |
| F116 | | |
| VH; 1B20 | QVQLVQSGAEVKKPGESLKISCKGSGYSFTDYYISWVRQMPGKGLEWMGLIYPGDSYTRY | 345. |
| Variant | SPNFQGQVTISADKSISTAYLQWSSLKASDTAMYYCARDHGYRPYSDIWGQGTLVTVSS | |
| Sequence | | |
| F119 | | |
| VH; 1B20 | QVQLVQSGAEVKKPGESLKISCKGSGYSFTNYYISWVRQMPGKGLEWMGLIYPGDSYTNY | 346. |
| Variant | NPNFQGQVTISADKSISTAYLQWSSLKASDTAMYYCARDHYSTPFFDVWGQGTLVTVSS | |
| Sequence | | |
| F113 | | |
| VH; 1B20 | QVQLVQSGAEVKKPGESLKISCKGSGYSFTNYYISWVRQMPGKGLEWMGLIYPGDSYTRY | 347. |
| Variant | NRKFQGQVTISADKSISTAYLQWSSLKASDTAMYYCARDYYSRPLSDVWGQGTLVTVSS | |
| Sequence E2 | | |
| VH; 1B20 | QVQLVQSGAEVKKPGESLKISCKGSGYSFTNYYISWVRQMPGKGLEWMGLIYPGDSYTRY | 348. |
| Variant | SPRFQGQVTISADKSISTAYLQWSSLKASDTAMYYCARDHGYKPYSDIWGQGTLVTVSS | |
| Sequence G4 | | |
| VH; 1B20 | QVQLVQSGAEVKKPGESLKISCKGSGYSFTDYYISWVRQMPGKGLEWMGLIYPGDSYTRY | 349. |
| Variant | SPSFQGQVTISADKSISTAYLQWSSLKASDTAMYYCARDYYSKPLFDVWGQGTLVTVSS | |
| Sequence F4 | | |
| VH; 1B20 | QVQLVQSGAEVKKPGESLKISCKGSGYSFTNYYISWVRQMPGKGLEWMGIIYPGDSYIHY | 350. |
| Variant | NQNFQGQVTISADKSISTAYLQWSSLKASDTAMYYCARDYYSRPFSDIWGQGTLVTVSS | |
| Sequence B9 | | |
| VH; 1B20 | QVKLVQSGAEVKKPGESLKISCKGSGYSFTNYYIAWVRQMPGKGLEWMGIIYPGDSYTHY | 351. |
| Variant | NPKFQGQVTISADKSISTAYLQWSSLKASDTAMYYCARDHGYKPFSDIWGQGTLVTVSS | |
| Sequence C3 | | |
| VH; 1B20 | QVQLVQSGAEVKKPGESLKISCKGSGYSFTNYYIAWVRQMPGKGLEWMGVIYPGDSYTRY | 352. |
| Variant | NPKFQGQVTISADKSISTAYLQWSSLKASDTAMYYCARDYWSRPYFDIWGQGTLVTVSS | |
| Sequence F2 | | |
| VH; 1B20 | QVQLVQSGAEVKKPGESLKISCKGSGYSFTNYYIAWVRQMPGKGLEWMGIIYPGDSYTHY | 353. |
| Variant | NPKFQGQVTISADKSISTAYLQWSSLKASDTAMYYCARDHWSRPFSDVWGQGTLVTVSS | |
| Sequence F7 | | |
| VH; 1B20 | QVQLVQSGAEVKKPGESLKISCKGSGYSFTNYYISWVRQMPGKGLEWMGIIYPGDSYTRY | 354. |
| Variant | NPNFQGQVTISADKSISTAYLQWSSLKASDTAMYYCARDYWSKPLSDVWGQGTLVTVSS | |
| Sequence A7 | | |
| VH; 1B20 | QVQLVQSGAEVKKPGESLKISCKGSGYSFTDYYISWVRQMPGKGLEWMGLIYPGDSYTHY | 355. |
| Variant | NPNFQGQVTISADKSISTAYLQWSSLKASDTAMYYCARDHWSKPYFDVWGQGTLVTVSS | |
| Sequence G8 | | |
| VH; 1B20 | QVQLVQSGAEVKKPGESLKISCKGSGYSFTNYYITWVRQMPGRGLEWMGIIYPGDSYTRY | 356. |
| Variant | NPKFQGQVTISADKSISTAYLQWSSLKASDTAMYYCARDHWSKPFSDVWGQGTLVTVSS | |
| Sequence H4 | | |
| VH; 1B20 | QVQLVQSGAEVKKPGESLKISCKGSGYSFTNYYITWVRQMPGKGLEWMGLIYPGDSYTRY | 357. |
| Variant | SPRFQGQVTISADKSISTAYLQWSSLKASDTAMYYCARDHWSRPYSDVWGQGTLVTVSS | |
| Sequence D5 | | |
| VH; 1B20 | QVQLVQSGAEVKKPGESLKISCKGSGYSFTDYYIAWVRQMPGKGLEWMGIIYPGDSYTRY | 358. |
| Variant | NPKFQGQVTISADKSISTAYLQWSSLKASDTAMYYCARDYWSRPFSDVWGQGTLVTVSS | |
| Sequence D4 | | |
| VH; 1B20 | QVQLVQSGAEVKKPGESLKISCKGSGYSFTDYYISWVRQMPGKGLEWMGLIYPGDSYTHY | 359. |
| Variant | NPNFQGQVTISADKSISTAYLQWSSLKASDTAMYYCARDYWSKPLFDVWGQGTLVTVSS | |
| Sequence B4 | | |
| VH; 1B20 | QVQLVQSGAEVKKPGESLKISCKGSGYSFTDYYIAWVRQMPGKGLEWMGIIYPGDSYTRY | 360. |
| Variant | NPRFQGQVTISADKSISTAYLQWSSLKASDTAMYYCARDYWSKPLSDIWGQGTLVTVSS | |
| Sequence H1 | | |
| VH; 1B20 | QVKLVQSGAEVKKPGESLKISCKGSGYSFTDYYISWVRQMPGKGLEWMGIIYPGDSYTHY | 361. |
| Variant | NPKFQGQVTISADKSISTAYLQWSSLKASDTAMYYCARDRWSKPLFDVWGQGTLVTVSS | |
| Sequence G2 | | |
| VH; 1B20 | QVQLVQSGAEVKKPGESLKISCKGSGYSFTDYYISWVRQMPGKGLEWMGLIYPGDSYTRY | 362. |
| Variant | NPSFQGQVTISADKSISTAYLQWSSLKASDTAMYYCARDYWSKPLSDIWGQGTLVTVSS | |
| Sequence A1 | | |
| VH; 1B20 | QVQLVQSGAEVKKPGESLKISCKGSGYSFTDYYISWVRQMPGKGLEWMGSIYPGDSYTHY | 363. |
| Variant | SHKFQGQVTISADKSISTAYLQWSSLKASDTAIYYCARDHWSRPFFDVWGQGTLVTVSS | |
| Sequence A4 | | |
| VH; 1B20 | QVQLVQSGAEVKKPGESLKISCKGSGYSFTDYYIAWVRQMPGKGLEWMGLIYPGDSYTSY | 364. |
| Variant | NPRFQGQVTISADKSISTAYLQWSSLKASDTAMYYCARDYWSTPYFDIWGQGTLVTVSS | |
| Sequence C2 | | |
| VH; 1B20 | QVQLVQSGAEVKKPGESLKISCKGSGYSFTDYYISWVRQMPGKGLEWMGIIYPGDSYTSY | 365. |
| Variant | SPRFQGQVTISADKSISTAYLQWSSLKASDTAMYYCARDYWSRPLFDVWGQGTLVTVSS | |
| Sequence H5 | | |
| VH; 1B20 | QVQLVQSGAEVKKPGESLKISCKGSGYSFTNYYISWVRQMPGKGLEWMGLIYPGDSYTHY | 366. |
| Variant | NPSFQGQVTISADKSISTAYLQWSSLKASDTAMYYCARDHWYKPFSDIWGQGTLVTVSS | |
| Sequence F6 | | |
| VH; 1B20 | QVQLVQSGAEVKKPGESLKISCKGSGYSFTDYYISWVRQMPGKGLEWMGIIYPGDSYTNY | 367. |
| Variant | NPSFQGQVTISADKSISTAYLQWSSLKASDTAMYYCARDYWYRPFSDIWGQGTLVTVSS | |
| Sequence B6 | | |
| VH; 1B20 | QVQLVQSGAEVKKPGESLKISCKGSGYSFTDYYISWVRQMPGKGLEWMGIIYPGDSYTHY | 368. |
| Variant | SPNFQGQVTISADKSISTAYLQWSSLKASDTAMYYCARDYWYTPFFDVWGQGTLVTVSS | |
| Sequence B1 | | |
| VH; 1B20 | QVQLVQSGAEVKKPGESLKISCKGSGYSFTDYYISWVRQMPGKGLEWMGLIYPGDSYTNY | 369. |
| Variant | SPKFQGQVTISADKSISTAYLQWSSLKASDTAMYYCARDHWSKPLSDVWGQGTLVTVSS | |
| Sequence F1 | | |
| VH; 1B20 | QVQLVQSGAEVKKPGESLKISCKGSGYSFTNYYISWVRQMPGKGLEWMGMIYPGDSYTHY | 370. |
| Variant | SPNFQGQVTISADKSISTAYLQWSSLKASDTAMYYCARDYWSKPYFDVWGQGTLVTVSS | |
| Sequence A8 | | |
| VH; 1B20 | QVQLVQSGAEVKKPGESLKISCKGSGYSFTNYYISWVRQMPGKGLEWMGMIYPGDSYTSY | 371. |
| Variant | NPKFQGQVTISADKSISTAYLQWSSLKASDTAMYYCARDHWSKPYFDIWGQGTLVTVSS | |
| Sequence B3 | | |
| VH; 1B20 | QVQLVQSGAEVKKPGESLKISCKGSGYSFTNYYISWVRQMPGKGLEWMGMIYPGDSYTNY | 372. |
| Variant | NQKFQGQVTISADKSISTAYLQWSSLKASDTAMYYCARDYWSRPYSDIWGQGTLVTVSS | |
| Sequence F8 | | |
| VH; 1B20 | QVQLVQSGAEVKKPGESLKISCKGSGYSFTDYYISWVRQMPGKGLEWMGMIYPGDSYTHY | 373. |
| Variant | SQNFQGQVTISADKSISTAYLQWSSLKASDTAMYYCARDHWSRPFFDVWGQGTLVTVSS | |
| Sequence H8 | | |
| VH; 1B20 | QVQLVQSGAEVKKPGESLKISCKGSGYSFTDYYISWVRQMPGKGLEWMGMIYPGDSYTRY | 374. |
| Variant | SPSFQGQVTISADKSISTAYLQSSSLKASDTAMYYCARDYWYKPFSDVWGQGTLVTVSS | |
| Sequence B5 | | |
| VH; 1B20 | QVQLVQSGAEVKKPGESLKISCKGSGYSFTNYYISWVRQMPGKGLEWMGMIYPGDSYTSY | 375. |
| Variant | NPNFQGQVTISADKSISTAYLQWSSLKASDTAMYYCARDHWSKPFFDIWGQGTLVTVSS | |
| Sequence E1 | | |
| VH; 1B20 | QVQLVQSGAEVKKPGESLKISCKGSGYSFTNYYITWVRQMPGKGLEWMGMIYPGDSYTRY | 376. |
| Variant | SPKFQGQVTISADKSISTAYLQWSSLKASDTAMYYCARDHWSKPLSDVWGQGTLVTVSS | |
| Sequence E8 | | |
| VH; 1B20 | QVQLVQSGAEVKKPGESLKISCKGSGYSFTNYYISWVRQMPGKGLEWMGMIYPGDSYTRY | 377. |
| Variant | SPKFQGQVTISADKSISTAYLQWSSLKASDTAMYYCARDHWYRPYSDIWGQGTLVTVSS | |
| Sequence C1 | | |
| VH; 1B20 | QVQLVQSGAEVKKPGESLKISCKGSGYSFTNYYITWVRQMPGKGLEWMGMIYPGDSYTHY | 378. |
| Variant | SQRFQGQVTISADKSISTAYLQWSSLKASDTAIYYCARDHWSRPLFDVWGQGTLVTVSS | |
| Sequence H3 | | |
| VH; 1B20 | QVQLVQSGAEVKKPGESLKISCKGSGYSFTNYYISWVRQMPGKGLEWMGMIYPGDSYTRY | 379. |
| Variant | SPKFQGQVTISADKSISTAYLQWSSLKASDTAMYYCARDHWYRPYSDIWGQGTLVTVSS | |
| Sequence A9 | | |
| VH; 1B20 | QVQLVQSGAEVKKPGESLKISCKGSGYSFTDYYISWVRQMPGKGLEWMGWIYPGDSYTHY | 380. |
| Variant | SPSFQGQVTISADKSISTAYLQWSSLKASDTAMYYCARDHWSKPYFDVWGQGTLVTVSS | |
| Sequence G7 | | |
| VH; 1B20 | QVQLVQSGAEVKKPGESLKISCKGSGYSFTNYYITWVRQMPGKGLEWMGMIYPGDSYTHY | 381. |
| Variant | NPSFQGQVTISADKSISTAYLQWSSLKASDTAMYYCARDHWYKPLFDIWGQGTLVTVSS | |
| Sequence C6 | | |
| VH; 1B20 | QVQLVQSGAEVKKPGESLKISCKGSGYSFTDYYINWVRQMPGKGLEWMGMIYPGDSYTNY | 382. |
| Variant | NPKFQGQVTISADKSISTAYLQWSSLKASDTAIYYCARDHWSRPFSDVWGQGTLVTVSS | |
| Sequence G6 | | |
| VH; 1B20 | QVQLVQSGAEVKKPGESLKISCKGSGYSFTNYYISWVRQMPGKGLEWMGWIYPGDSYTSY | 383. |
| Variant | NQKFQGQVTISADKSISTAYLQWSSLKASDTAMYYCARDYWSKPYSDVWGQGTLVTVSS | |
| Sequence E4 | | |
| VH; 1B20 | QVQLVQSGAEVKKPGESLKISCKGSGYSFTDYYITWVRQMPGKGLEWMGMIYPGDSYTNY | 384. |
| Variant | RHNFQGQVTISADKSISTAYLQWSSLKASDTAMYYCARDYWSRPYFDIWGQGTLVTVSS | |
| Sequence F5 | | |
| VH; 1B20 | QVQLVQSGAEVKKPGESLKISCKGSGYSFTDYYISWVRQMPGKGLEWMGWIYPGDSYTHY | 385. |
| Variant | SHNFQGQVTISADKSISTAYLQWSSLKASDTAMYYCARDHWSRPFSDVWGQGTLVTVSS | |
| Sequence C7 | | |
| VH; 1B20 | QVQLVQSGAEVKKPGESLKISCKGSGYSFTDYYISWVRQMPGKGLEWMGMIYPGDSYTSY | 386. |
| Variant | SPRFQGQVTISADKSISTAYLQWSSLKASDTAMYYCARDYWYRPFSDIWGQGTLVTVSS | |
| Sequence E3 | | |
| VH; 1B20 | QVQLVQSGAEVKKPGESLKISCKGSGYSFTDYYITWVRQMPGKGLEWMGMIYPGDSYTRY | 387. |
| Variant | SPKFQGQVTISADKSISTAYLQWSSLKASDTAMYYCARDYWSKPFFDVWGQGTLVTVSS | |
| Sequence D3 | | |
| VH; 1B20 | QVQLVQSGAEVKKPGESLKISCKGSGYSFTDYYISWVRQMPGKGLEWMGMIYPGDSYTHY | 388. |
| Variant | SQSFQGQVTISADKSISTAYLQWSSLKASDTAMYYCARDHWYRPLFDIWGQGTLVTVSS | |
| Sequence D8 | | |
| VH; 1B20 | QVQLVQSGAEVKKPGESLKISCKGSGYSFTNYYIAWVRQMPGKGLEWMGMIYPGDSYTSY | 389. |
| Variant | SHRFQGQVTISADKSISTAYLQWSSLKASDTAMYYCARDHWYRPYFDIWGQGTLVTVSS | |
| Sequence C8 | | |
| VH; 1B20 | QVQLVQSGAEVKKPGESLKISCKGSGYSFTDYYISWVRQMPGKGLEWMGWIYPGDSYTHY | 390. |
| Variant | NPNFQGQVTISADKSISTAYLQWSSLKASDTAMYYCARDRWSTPYFDVWGQGTLVTVSS | |
| Sequence E5 | | |
| VH; 1B20 | QVQLVQSGAEVKKPGESLKISCKGSGYSFTNYYISWVRQMPGKGLEWMGMIYPGDSYTHY | 391. |
| Variant | SPNFQGQVTISADKSISTAYLQWSSLKASDTAMYYCARDYWSKPYFDVWGQGTLVTVSS | |
| Sequence B8 | | |
| VH; 1B20 | QVQLVQSGAEVKKPGESLKISCKGSGYSFTDYYISWVRQMPGKGPEWMGMIYPGDSYTRY | 392. |
| Variant | SQKFQGQVTISADKSISTAYLQWSSLKASDTAIYYCARDHWSRPLSDIWGQGTLVTVSS | |
| Sequence H7 | | |
| VH; 1B20 | QVQLVQSGAEVKKPGESLKISCKGSGYSFTDYYISWVRQMPGKGLEWMGWIYPGDSYTHY | 393. |
| Variant | NPMFQGQVTISADKSISTAYLQWSSLKASDTAMYYCARDRWSRPYFDIWGQGTLVTVSS | |
| Sequence A5 | | |
| VH; 1B20 | QVQLVQSGAEVKKPGESLKISCKGSGYSFTDYYISWVRQMPGKGLEWMGMIYPGDSYTRY | 394. |
| Variant | SPNFQGQVTISADKSISTAYLQWSSLKASDTAMYYCARDHWYRPLSDIWGQGTLVTVSS | |
| Sequence A3 | | |
| 1B20 | QVQLVQSGAEVKKPGESLKISCKGSGYSFTNYWISWVRQMPGKGLEWMGIIYPGDSYTKY | 395. |
| Antibody | SPSFQGQVTISADKSISTAYLQWSSLKASDTAMYYCARDYWYKPLFDIWGQGTLVTVSS | |
| Variant VH | | |
| Sequence | | |
| N59K | | |
| 1B20 | QVQLVQSGAEVKKPGESLKISCKGSGYSFTNYWISWVRQMPGKGLEWMGIIYPGDSYTQY | 396. |
| Antibody | SPSFQGQVTISADKSISTAYLQWSSLKASDTAMYYCARDYWYKPLFDIWGQGTLVTVSS | |
| Variant VH | | |
| Sequence | | |
| N59Q | | |
| 1B20 | QVQLVQSGAEVKKPGESLKISCKGSGYSFTNYWISWVRQMPGKGLEWMGIIYPGDSYTRY | 397. |
| Antibody | SPSFQGQVTISADKSISTAYLQWSSLKASDTAMYYCARDYWYKPLFDIWGQGTLVTVSS | |
| Variant VH | | |
| Sequence | | |
| N59R | | |
| 1B20 | QVQLVQSGAEVKKPGESLKISCKGSGYSFTNYWISWVRQMPGKGLEWMGIIYPGDSYTNY | 398. |
| Antibody | SPSFQGQVTISADKSISTAYLQWSSLKASDTAMYYCARDYAYKPLFDIWGQGTLVTVSS | |
| Variant VH | | |
| Sequence | | |
| W101A | | |
| 1B20 | QVQLVQSGAEVKKPGESLKISCKGSGYSFTNYWISWVRQMPGKGLEWMGIIYPGDSYTNY | 399. |
| Antibody | SPSFQGQVTISADKSISTAYLQWSSLKASDTAMYYCARDYFYKPLFDIWGQGTLVTVSS | |
| Variant VH | | |
| Sequence | | |
| W101F | | |
| 1B20 | QVQLVQSGAEVKKPGESLKISCKGSGYSFTNYWISWVRQMPGKGLEWMGIIYPGDSYTNY | 400. |
| Antibody | SPSFQGQVTISADKSISTAYLQWSSLKASDTAMYYCARDYYYKPLFDIWGQGTLVTVSS | |
| Variant VH | | |
| Sequence | | |
| W101Y | | |

GROUP H

| | | |
|---|---|---|
| Linker | GGGGGGGGGGGCGG | 401. |
| Sequence | | |
| (used in | | |
| DSB#1) | | |
| Linker | GGGGGGGGGGGGCG | 402. |
| Seqeunce | | |
| (used in | | |
| DSB#2) | | |
| Linker | GGGGSGGGGS | 403. |

Group I

| | | |
|---|---|---|
| PC9#3 | EVQLVESGGGLVQPGGSLRLSCAASGFTFNNYAMNWVRQAPGKGLDWVSTISGSGGTTNYADSVKGRFIISRDS | 404. |
| variable heavy | SKHTLYLQMNSLRAEDTAVYYCAKDSNWGNFDLWGRGTLVTVSS | |
| chain | | |
| PC9#3 | DIVMTQSPDSLAVSLGERATINCKSSQSVLYRSNNRNFLGWYQQKPGQPPNLLIYWASTRESGVPDRFSGSGSG | 405. |
| variable light | TDFTLTISSLQAEDVAVYYCQQYYTTPYTFGQGTKLEIK | |
| chain | | |
| PC9#4 | EVQLVESGGGLVKPGGSLRLSCAASGFTFSSYSMNWVRQAPGKGLEWVSSISSSSSYISYADSVKGRFTISRDN | 406. |
| variable heavy | AKNSLYLQMNSLRAEDTAVYFCARDYDFWSAYYDAFDVWGQGTMVTVSS | |
| chain | | |
| PC9#4 | QSVLTQPPSVSGAPGQRVTISCTGSSSNIGAGYDVHWYQQLPGTAPKLLISGNSNRPSGVPDRFSGSKSGTSAS | 407. |
| variable light | LAITGLQAEDEADYYCQSYDSSLSGSVFGGGTKLTVL | |
| chain | | |
| PC9#5 | EMQLVESGGGLVQPGGSLRLSCAASGFTFSSHWMKWVRQAPGKGLEWVANINQDGSEKYYVDSVKGRFTISRDN | 408. |
| variable heavy | AKNSLFLQMNSLRAEDTAVYYCARDIVLMVYDMDYYYYGMDVWGQGTTVTVSS | |
| chain | | |
| PC9#5 | DIVMTQSPLSLPVTPGEPASISCRSSQSLLHSNGNNYLDWYLQKPGQSPQLLIYLGSNRASGVPDRFSGSGSGT | 409. |
| variable light | DFTLKISRVEAEDVGVYYCMQTLQTPLTFGGGTKVEIK | |
| chain | | |
| PC9#6 | QVQLVQSGAEVKKPGSSVKVSCKASGGTFNSHAISWVRQAPGQGLEWMGGINPILGIANYAQKFQGRVTITADE | 410. |
| variable heavy | STSTAYMELSSLRSEDTAVYYCARHYEIQIGRYGMNVYYLMYRFASWGQGTLVTVSS | |
| chain | | |
| PC9#6 | DIQMTQSPSSLSASVGDRVTITCRASQGIRSALNWYQQKPGKAPKLLIYNGSTLQSGVPSRFSGSGSGTDFTLT | 411. |
| variable light | ISSLQPEDFAVYYCQQFDGDPTFGQGTKVEIK | |
| chain | | |
| PC9#7 | QVQLVQSGAEVKKPGESLKISCKGSGYSFTNYWISWVRQMPGKGLEWMGIIYPGDSYTNYSPSFQGQVTISADK | 412. |
| variable heavy | SISTAYLQWSSLKASDTAMYYCARDYWYKPLFDIWGQGTLVTVSS | |
| chain | | |
| PC9#7 | DIVMTQSPDSLAVSLGERATINCRSSQSVLYSSNNKNYLAWYQQKPGQPPKLLIYWASTRESGVPDRFSGSGSG | 413. |
| variable light | TDFTLTISSLQAEDVAVYYCQQYSSFPITFGQGTKVEIK | |
| chain | | |
| NIP228 | QVNLRESGGGVVQPGRSLRLSCAASGFTFSSYGMHWVRQAPGEGLEWVSAISGSGGSTYYADSVKGRFTISRDN | 414. |
| variable heavy | SKNTLYLQMNSLRAEDTAVYYCAKRFGEFAFDIWGRGTTVTVSS | |
| chain | | |
| NIP228 | AIRMTQSPSSLSASVGDRVTITCRASQSISSYLNWYQQKPGKAPKLLIYAASSLQSGVPSRFSGSGSGTDFTLT | 415. |
| variable light | ISSLQPEDFATYYCQQSYSTPLTFGGGTKVEIK | |
| chain | | |
| Human IgG4 | ESKYGPPCPPCPAPEAAGGPSVFLFPPKPKDTLMISRTPEVTCVVVDVSQEDPEVQFNWYVDGVEVHNAKTKPR | 416. |
| Fc fragment | EEQFNSTYRVVSVLTVLHQDWLNGKEYKCKVSNKGLPSSIEKTISKAKGQPREPQVYTLPPSQEEMTKNQVSLT | |
| | CLVKGFYPSDIAVEWESNGQPENNYKTTPPVLDSDGSFFLYSRLTVDKSRWQEGNVFSCSVMHEALHNHYTQKS | |
| | LSLSLG | |
| Human | QVQLVQSGAEVKKPGASVKVSCKASGYTFTSYYMHWVRQAPGQGLEWMGIINPSGGSTSYAQKFQGRVTMTRDT | 417. |
| germline 1-46 | STSTVYMELSSLRSEDTAVYYCARD | |
| Human | DIQMTQSPSSLSASVGDRVTITCQASQDISNYLNWYQQKPGKAPKLLIYDASNLETGVPSRFSGSGSGTDFTFT | 418. |
| germline VK1 | ISSLQPEDIATYYCQQ | |
| O18 O8 | | |
| (DPK1) | | |
| HS9_DSB7_ | HGEGTFTSDLSKQMEEECARLFIEWLKNGGPSSGAPPPGCGGGGGSGGGGSADIQMTQSPSSLSASVGDRVTIT | 419. |
| V19A_L2 | CQASQDVKTAVAWYQQKPGKAPKLLIYSASYRYTGVPSRFSGSGSGTDFTFTISSLQPEDIATYYCQQRYSLWR | |
| | TFGQGTKLEIKRTVAAPSVFIFPPSDEQLKSGTASVVCLLNNFYPREAKVQWKVDNALQSGNSQESVTEQDSKD | |
| | STYSLSSTLTLSKADYEKHKVYACEVTHQGLSSPVTKSFNRGEC | |
| HS9_DSB7_ | HGEGTFTSDLSKQMEEECARLFIEWLKNGGPSSGAPPPGCGGGGGSADIQMTQSPSSLSASVGDRVTITCQASQ | 420. |
| V19A_L1 | DVKTAVAWYQQKPGKAPKLLIYSASYRYTGVPSRFSGSGSGTDFTFTISSLQPEDIATYYCQQRYSLWRTFGQG | |
| | TKLEIKRTVAAPSVFIFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSFNRGEC | |
| HS9_DSB7_ | HGEGTFTSDLSKQMEEECARLFIEWLKNGGPSSGAPPPGCGADIQMTQSPSSLSASVGDRVTITCQASQDVKTA | 421. |
| V19A_L0 | VAWYQQKPGKAPKLLIYSASYRYTGVPSRFSGSGSGTDFTFTISSLQPEDIATYYCQQRYSLWRTFGQGTKLEI | |
| | KRTVAAPSVFIFPPSDEQLKSGTASVVCLLNNFYPREAKVQWKVDNALQSGNSQESVTEQDSKDSTYSLSSTLT | |
| | LSKADYEKHKVYACEVTHQGLSSPVTKSFNRGEC | |
| 2.7A4_VH | EVQLVESGGGLVKPGGSLRLSCAASGFTFSTYSMNWVRQAPGKGLEWVSSISSSGDYIYYADSVKGRFTISRDN | 422. |
| | AKNSLYLQMNSLRAEDTAVYYCARDLVTSMVAFDYWGQGTLVTVSS | |
| 2.7A4_VL | SYELTQPPSVSVSPGQTARITCSGDALPQKYVFWYQQKSGQAPVLVIYEDSKRPSGIPERFSGSSSGTMATLTI | 423. |
| | SGAQVEDEADYYCYSTDRSGNHRVFGGGTKLTVL | |

GROUP J

| | | |
|---|---|---|
| HS1_VH | QVQLVQSGAEVKKPGASVKVSCKASGYTFTSYYMHWVRQAPGQGLEWMGEIKPSGGSTSYNQKFQGRVTMTRDTSTST | 424. |
| | VYMELSSLRSEDTAVYYCARERPLYASDLWGQGTTVTVSS | |
| HS1_VL | DIQMTQSPSSLSASVGDRVTITCQASQDVYTAVAWYQQKPGKAPKLLIYYASYRYTGVPSRFSGSGSGTDFTFTISSL | 425. |
| | QPEDIATYYCQQRYSLWRTFGQGTKLEIK | |
| HS2_VH | QVQLVQSGAEVKKPGASVKVSCKASGYTFTSYYMHWVRQAPGQGLEWMGEIKPSGGSTSYNQKFQGRVTMTRDTSTST | 426. |
| | VYMELSSLRSEDTAVYYCARERPLYASDLWGQGTTVTVSS | |
| HS2_VL | DIQMTQSPSSLSASVGDRVTITCQASQDVYTAVAWYQQKPGKAPKLLIYSASYRYTGVPSRFSGSGSGTDFTFTISSL | 427. |
| | QPEDIATYYCQQRYSLWRTFGQGTKLEIK | |
| HS3_VH | QVQLVQSGAEVKKPGASVKVSCKASGYTFTSYYMHWVRQAPGQGLEWMGEIKPSGGSTSYNQKFQGRVTMTRDTSTST | 428. |
| | VYMELSSLRSEDTAVYYCARERPLYASDLWGQGTTVTVSS | |
| HS3_VL | DIQMTQSPSSLSASVGDRVTITCQASQDVKTAVAWYQQKPGKAPKLLIYYASYRYTGVPSRFSGSGSGTDFTFTISSL | 429. |
| | QPEDIATYYCQQRYSLWRTFGQGTKLEIK | |
| HS4_VH | QVQLVQSGAEVKKPGASVKVSCKASGYTFTSYYMHWVRQAPGQGLEWMGEIKPSGGSTSYNQKFQGRVTMTRDTSTST | 430. |
| | VYMELSSLRSEDTAVYYCARERPLYASDLWGQGTTVTVSS | |
| HS4_VL | DIQMTQSPSSLSASVGDRVTITCQASQDVKTAVAWYQQKPGKAPKLLIYSASYRYTGVPSRFSGSGSGTDFTFTISSL | 431. |
| | QPEDIATYYCQQRYSLWRTFGQGTKLEIK | |
| HS5_VH | QVQLVQSGAEVKKPGASVKVSCKASGYTFTSYYMHWVRQAPGQGLEWMGEIKPSGGSTSYNQKFQGRVTMTRDTSTST | 432. |
| | VYMELSSLRSEDTAVYYCARERPLYASDLWGQGTTVTVSS | |
| HS5_VL | DIQMTQSPSSLSASVGDRVTITCQASQDVSTAVAWYQQKPGKAPKLLIYSASYRYTGVPSRFSGSGSGTDFTFTISSL | 433. |
| | QPEDIATYYCQQRYSLWRTFGQGTKLEIK | |
| HS6_VH | QVQLVQSGAEVKKPGASVKVSCKASGYTFTSYYMHWVRQAPGQGLEWMGEISPSGGSTSYNQKFQGRVTMTRDTSTST | 434. |
| | VYMELSSLRSEDTAVYYCARERPLYASDLWGQGTTVTVSS | |
| HS6_VL | DIQMTQSPSSLSASVGDRVTITCQASQDVYTAVAWYQQKPGKAPKLLIYYASYRYTGVPSRFSGSGSGTDFTFTISSL | 435. |
| | QPEDIATYYCQQRYSLWRTFGQGTKLEIK | |
| HS7_VH | QVQLVQSGAEVKKPGASVKVSCKASGYTFTSYYMHWVRQAPGQGLEWMGEISPSGGSTSYNQKFQGRVTMTRDTSTST | 436. |
| | VYMELSSLRSEDTAVYYCARERPLYASDLWGQGTTVTVSS | |
| HS7_VL | DIQMTQSPSSLSASVGDRVTITCQASQDVYTAVAWYQQKPGKAPKLLIYSASYRYTGVPSRFSGSGSGTDFTFTISSL | 437. |
| | QPEDIATYYCQQRYSLWRTFGQGTKLEIK | |
| HS8_VH | QVQLVQSGAEVKKPGASVKVSCKASGYTFTSYYMHWVRQAPGQGLEWMGEISPSGGSTSYNQKFQGRVTMTRDTSTST | 438. |
| | VYMELSSLRSEDTAVYYCARERPLYASDLWGQGTTVTVSS | |
| HS8_VL | DIQMTQSPSSLSASVGDRVTITCQASQDVKTAVAWYQQKPGKAPKLLIYYASYRYTGVPSRFSGSGSGTDFTFTISSL | 439. |
| | QPEDIATYYCQQRYSLWRTFGQGTKLEIK | |
| HS9_VH | QVQLVQSGAEVKKPGASVKVSCKASGYTFTSYYMHWVRQAPGQGLEWMGEISPSGGSTSYNQKFQGRVTMTRDTSTST | 440. |
| | VYMELSSLRSEDTAVYYCARERPLYASDLWGQGTTVTVSS | |
| HS9_VL | DIQMTQSPSSLSASVGDRVTITCQASQDVKTAVAWYQQKPGKAPKLLIYSASYRYTGVPSRFSGSGSGTDFTFTISSL | 441. |
| | QPEDIATYYCQQRYSLWRTFGQGTKLEIK | |
| HS10_VH | QVQLVQSGAEVKKPGASVKVSCKASGYTFTSYYMHWVRQAPGQGLEWMGEISPSGGSTSYNQKFQGRVTMTRDTSTST | 442. |
| | VYMELSSLRSEDTAVYYCARERPLYASDLWGQGTTVTVSS | |
| HS10_VL | DIQMTQSPSSLSASVGDRVTITCQASQDVSTAVAWYQQKPGKAPKLLIYSASYRYTGVPSRFSGSGSGTDFTFTISSL | 443. |
| | QPEDIATYYCQQRYSLWRTFGQGTKLEIK | |
| HS9_DSB7_ | HYEGTFTSDLSKQMEEECVRLFIEWLKNGGPSSGAPPPGCGGGGGSGGGGSGGGGSADIQMTQSPSSLSASVGDRVT | 444. |
| G2Y | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HVEGTFTSDLSKQMEEECVRLFIEWLKNGGPSSGAPPPGCGGGGGSGGGGSGGGGSADIQMTQSPSSLSASVGDRVT | 445. |
| G2V | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HTEGTFTSDLSKQMEEECVRLFIEWLKNGGPSSGAPPPGCGGGGGSGGGGSGGGGSADIQMTQSPSSLSASVGDRVT | 446. |
| G2T | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HQEGTFTSDLSKQMEEECVRLFIEWLKNGGPSSGAPPPGCGGGGGSGGGGSGGGGSADIQMTQSPSSLSASVGDRVT | 447. |
| G2Q | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HNEGTFTSDLSKQMEEECVRLFIEWLKNGGPSSGAPPPGCGGGGGSGGGGSGGGGSADIQMTQSPSSLSASVGDRVT | 448. |
| G2N | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HIEGTFTSDLSKQMEEECVRLFIEWLKNGGPSSGAPPPGCGGGGGSGGGGSGGGGSADIQMTQSPSSLSASVGDRVT | 449. |
| G2I | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HFEGTFTSDLSKQMEEECVRLFIEWLKNGGPSSGAPPPGCGGGGGSGGGGSGGGGSADIQMTQSPSSLSASVGDRVT | 450. |
| G2F | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMGEECVRLFIEWLKNGGPSSGAPPPGCGGGGGSGGGGSGGGGSADIQMTQSPSSLSASVGDRVT | 451. |
| E15G | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWLKNGGPSSGAPPPGCGGGGGSGGGGSGGGGSADIQMTQSPSSLSASVGDRVT | 452. |
| E15A | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECTRLFIEWLKNGGPSSGAPPPGCGGGGGSGGGGSGGGGSADIQMTQSPSSLSASVGDRVT | 453. |
| V19T | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECSRLFIEWLKNGGPSSGAPPPGCGGGGGSGGGGSGGGGSADIQMTQSPSSLSASVGDRVT | 454. |
| V19S | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECGRLFIEWLKNGGPSSGAPPPGCGGGGGSGGGGSGGGGSADIQMTQSPSSLSASVGDRVT | 455. |
| V19G | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECARLFIEWLKNGGPSSGAPPPGCGGGGGSGGGGSGGGGSADIQMTQSPSSLSASVGDRVT | 456. |
| V19A | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFTEWLKNGGPSSGAPPPGCGGGGGSGGGGSGGGGSADIQMTQSPSSLSASVGDRVT | 457. |
| I23T | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFSEWLKNGGPSSGAPPPGCGGGGGSGGGGSGGGGSADIQMTQSPSSLSASVGDRVT | 458. |
| I23S | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFGEWLKNGGPSSGAPPPGCGGGGGSGGGGSGGGGSADIQMTQSPSSLSASVGDRVT | 459. |
| I23G | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFAEWLKNGGPSSGAPPPGCGGGGGSGGGGSGGGGSADIQMTQSPSSLSASVGDRVT | 460. |
| I23A | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWTKNGGPSSGAPPPGCGGGGGSGGGGSGGGGSADIQMTQSPSSLSASVGDRVT | 461. |
| L26T | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWSKNGGPSSGAPPPGCGGGGGSGGGGSGGGGSADIQMTQSPSSLSASVGDRVT | 462. |
| L26S | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWPKNGGPSSGAPPPGCGGGGGSGGGGSGGGGSADIQMTQSPSSLSASVGDRVT | 463. |
| L26P | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWNKNGGPSSGAPPPGCGGGGGSGGGGSGGGGSADIQMTQSPSSLSASVGDRVT | 464. |
| L26N | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWQKNGGPSSGAPPPGCGGGGGSGGGGSGGGGSADIQMTQSPSSLSASVGDRVT | 465. |
| L26Q | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWMKNGGPSSGAPPPGCGGGGGSGGGGSGGGGSADIQMTQSPSSLSASVGDRVT | 466. |
| L26M | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWIKNGGPSSGAPPPGCGGGGGSGGGGSGGGGSADIQMTQSPSSLSASVGDRVT | 467. |
| L26I | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWHKNGGPSSGAPPPGCGGGGGSGGGGSGGGGSADIQMTQSPSSLSASVGDRVT | 468. |
| L26H | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWGKNGGPSSGAPPPGCGGGGGSGGGGSGGGGSADIQMTQSPSSLSASVGDRVT | 469. |
| L26G | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWEKNGGPSSGAPPPGCGGGGGSGGGGSGGGGSADIQMTQSPSSLSASVGDRVT | 470. |
| L26E | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7_ | HGEGTFTSDLSKQMAEECVRLFIEWDKNGGPSSGAPPPGCGGGGGSGGGGSGGGGSADIQMTQSPSSLSASVGDRVT | 471. |
| L26D | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| HS9_DSB7 | HGEGTFTSDLSKQMAEECVRLFIEWPKNGGPSSGAPPPGCGGGGGSGGGGSGGGGSADIQMTQSPSSLSASVGDRVT | 472. |
| | ITCQASQDVKTAVAWYQQKPGKAPKLLIYSASYRYTGVPSRFSGSGSGTDFTFTISSLQPEDIATYYCQQRYSLWRT | |
| | FGQGTKLEIK | |
| GLP-1 | HGEGTFTSDVSSYLEEQAAKEFIAWLVKGG | 473. |
| analogue | | |
| NGS#1 | HGEGTFTSDVSSYLEEQNASEFIAWLVKGG | 474. |
| NGS#2 | HGEGTFTSDVSSYLEEQAAKEFIAWLVNGS | 475. |
| NGS#3 | HGEGTFTSDVSSYLEEQAAKEFIAWLVKNG | 476. |
| NGS#4 | HGEGTFTSDVSSYLEEQAAKEFIAWLVKGN | 477. |
| NGS#5 | HGEGTFTSDVSSYLEEQAAKEFIAWLVKGG | 478. |
| NGS#6 | HGEGTFTSDVSSYLEEQAAKEFIAWLVKGG | 479. |
| NGS#7 | HGEGTFTSDVSSYLEEQAAKEFIANLSKGG | 480. |
| NGS#8 | HGEGTFTSDVSSYLEEQAAKEFIANLTKGG | 481. |
| Linker | GGGGGSGGGGSGGGGSA | 482. |
| Linker | SGGGGSGGGGSGGGGSA | 483. |
| Linker | GSGGGSGGGGSGGGGSA | 484. |
| Linker | NGSGGSGGGGSGGGGSA | 485. |
| Linker | GNGSGSGGGGSGGGGSA | 486. |
| PC9_2_ | [symbol] [symbol] GGGGSGGGGSGGGGSADIQMTQSP | 487. |
| DSB#1 | SSLSASVGDRVTITCKASQDVHTAVAWYQQKPGKAPKLLIYHASYRYTGVPSRFSGSGSGTDFTFTISSLQPEDIAT | |
| VL | YYCQQRYSLWRTFGQGTKLEIK | |
| (Version | | |
| B)(with | | |
| Kat end | | |
| of | | |
| variable | | |
| light | | |
| chain) | | |
| PC9_2_ | [symbol] [symbol] GGGGSGGGGSGGGGSADIQMTQSPSSLSASVGDRVTIT | 488. |
| DSB#3 | CKASQDVHTAVAWYQQKPGKAPKLLIYHASYRYTGVPSRFSGSGSGTDFTFTISSLQPEDIATYYCQQRYSLWRTFG | |
| VL | QGTKLEIK | |
| (Version | | |
| B)(with | | |
| Kat end | | |
| of | | |
| variable | | |
| light | | |
| chain) | | |
| PC9_2_ | [symbol] [symbol] GGGGSGGGGSGGGGSADIQMTQSPSSLSASVGDRVTITCKASQDVH | 489. |
| NGS#7 | TAVAWYQQKPGKAPKLLIYHASYRYTGVPSRFSGSGSGTDFTFTISSLQPEDIATYYCQQRYSLWRTFGQGTKLEIK | |
| VL | | |
| (Version | | |
| B)(with | | |
| Kat end | | |
| of | | |
| variable | | |
| light | | |
| chain) | | |
| PC9_2_ | [symbol] [symbol] GGGGSGGGGSGGGGSADIQMTQSPSSLSASVGDRVT | 490. |
| DSB#7 | ITCKASQDVHTAVAWYQQKPGKAPKLLIYHASYRYTGVPSRFSGSGSGTDFTFTISSLQPEDIATYYCQQRYSLWRT | |
| VL | FGQGTKLEIK | |
| (Version | | |
| B)(with | | |
| Kat end | | |
| of | | |
| variable | | |
| light | | |
| chain) | | |
| Engineered | | 491. |
| PCSK9 | EVQLVESGGGLVKPGGSLRLSCAASGFPFSKLGMVWVRQAPGKGLEWVSTISSGGGYTYYPDSVKGRFTISRDNAKN | |
| antibody | SLY | |
| VH | LQMNSLRAEDTAVYYCAREGISFQGGTYTYVMDYWGQGTLVTVSS | |
| Engineered | DIVMTQSPLSLPVTPGEPASISCRSSKSLLHRNGITYSYWYLQKPGQSPQLLIYQLSNLASGVPDRFSGSGSGTDFT | 492. |
| PCSK9 | LKI | |
| antibody | SRVEAEDVGVYYCYQNLELPLTFGQGTKVEIK | |
| VL | | |
| Engineered | GFPFSKLGMV | 493. |
| PCSK9 | | |
| antibody | | |
| VH | | |
| CDR1 | | |
| Engineered | TISSGGGYTYYPDSVK | 494. |
| PCSK9 | | |
| antibody | | |
| VH | | |
| CDR2 | | |
| Engineered | EGISFQGGTYTYVMDY | 495. |
| PCSK9 | | |
| antibody | | |
| VH | | |
| CDR3 | | |
| Engineered | RSSKSLLHRNGITYSY | 496. |
| PCSK9 | | |
| antibody | | |
| VL | | |
| CDR1 | | |
| Engineered | QLSNLAS | 497. |
| PCSK9 | | |
| antibody | | |
| VL | | |
| CDR2 | | |
| Engineered | YQNLELPLT | 498. |
| PCSK9 | | |
| antibody | | |
| VL | | |
| CDR3 | | |

### DESCRIPTION OF THE EMBODIMENTS

### I. Anti-PCSK9-GLP-1 Fusion Molecules

The present disclosure is directed to fusions of antibodies (e.g., anti-PCSK9 antibodies or antigen-binding fragments thereof) with a GLP-1 moiety.

In one embodiment the fusion is constructed as:

GLP-1 moiety-Linker-Antibody Light Chain.

or

GLP-1 moiety-Linker-Antibody Heavy Chain

The fusion protein can be constructed as a genetic fusion. Alternatively, the fusion protein may be constructed as a chemical conjugate, such as through a cysteine:cysteine disulfide bond.

Other arrangements of the GLP-1 moiety and antibody portion are also within the scope herein.

A. Antibodies and Antigen-Binding Fragments Thereof

1. Anti-PCSK9 Portion

The antibody portion may be an anti-PCSK9 antibody or an antigen-binding fragment thereof. In one embodiment, the anti-PCSK9 portion provides an LDLc (bad cholesterol) lowering effect.

In one embodiment, the anti-PCSK9 VL portion may be SEQ ID NO: 2 (PC9_2_HS9). In another embodiment, the anti-PCSK9 VL portion may be an antigen binding portion of SEQ ID NO: 2. In one embodiment, the anti-PCSK9 VL portion may comprise all six CDRs of SEQ ID NO: 2. In one embodiment, the anti-PCSK9 VH portion may be a pH dependent version of the antibody, as shown in SEQ ID NO: 5.

In one embodiment, the antibody or antigen-binding fragment thereof may be pH dependent, such that the antibody binding to the antigen is pH dependent. This can be used to modify half-life of the antibody and/or antigen. By modifying the antibody half-life, in one embodiment, we mean lengthening the half-life. By modifying the antibody half-life, in one embodiment, we mean shortening the half-life. In one embodiment, the antibody half-life may be modified (lengthened or shortened) to maximize the stability of it fusion partner (i.e., GLP-1). By modifying the antigen half-life we may also mean that the antigen-antibody complex can change the antigen's half-life, such as through antibody-mediated degradation (shortening the half-life) or by protecting the antigen from the typical degradation process (lengthening the half-life). In one instance, antibodies may have a higher affinity for the antigen at pH 7.4 as compared to endosomal pH (i.e., pH 5.5-6.0), such that the K_{D} ratio at pH 5/5/pH 7.4 or at pH 6.0/pH 7.2 is 2 or more. Methods of engineering pH dependent antibodies are described in US 2011/0229489 and 2014/0044730, which are incorporated by reference herein.

In one embodiment the anti-PCSK9 portion provides sustained suppression of free PCSK9. In one embodiment, is provides at least 70%, 75%, 80%, 85%, 90%, 95%, or 99% suppression.

In one embodiment, an anti-PCSK9 antibody or antigen binding fragment thereof capable of specifically binding PCSK9 comprises:

a. a heavy chain variable region comprising a sequence which is at least 70%, 75%, 80%, 85%, 90%, 95%, 96%, 97%, 98%, or 99% A identical to SEQ ID NO: 1, 5, 8, or 10; and b. a light chain variable region comprising a sequence which is at least 70%, 75%, 80%, 85%, 90%, 95%, 96%, 97%, 98%, or 99% A identical to SEQ ID NO: 2, 6, 9 or 11.

In one embodiment, an anti-PCSK9 antibody or antigen binding fragment thereof capable of specifically binding PCSK9 comprises:

a. a heavy chain variable region CDR1 sequence comprising a sequence which has one mutation compared to SEQ ID NO: 14;

b. a heavy chain variable region CDR2 sequence comprising a sequence which has one or two mutations compared to SEQ ID NO: 15, 16, 17, or 18;

c. a heavy chain variable region CDR3 sequence comprising a sequence which is one mutation compared to SEQ ID NO: 19.

d. a light chain variable region CDR1 sequence comprising a sequence which has one or two mutations compared to SEQ ID NO: 20, 21, 22, or 23;

e. a light chain variable region CDR2 sequence comprising a sequence which has one mutation compared to SEQ ID NO: 24 or 25; and

f. a light chain variable region CDR3 sequence comprising a sequence which has one mutation compared to SEQ ID NO: 26.

In one embodiment, an anti-PCSK9 antibody or antigen binding fragment thereof capable of specifically binding PCSK9 comprises:

a. a heavy chain variable region CDR1 sequence comprising SEQ ID NO: 14;

b. a heavy chain variable region CDR2 sequence comprising SEQ ID NO: 15, 16, 17, or 18;

c. a heavy chain variable region CDR3 sequence comprising SEQ ID NO: 19.

d. a light chain variable region CDR1 sequence comprising SEQ ID NO: 20, 21, 22, or 23;

e. a light chain variable region CDR2 sequence comprising SEQ ID NO: 24 or 25; and

f. a light chain variable region CDR3 sequence comprising SEQ ID NO: 26.

In another embodiment, the anti-PCSK9 portion may comprise an anti-PCSK9 antibody or antigen-binding fragment as described in any of U.S. Pat. Nos. 8,030,457, 8,062,640, 8,357,371, 8,168,762, 8,563,698, 8,829,165, 8,859,741, 8,188,233, WO 2012/088313, US 2012/0195910, U.S. Pat. No. 8,530,414, US 2013/0189278, U.S. Pat. No. 8,344,144, US 2011/0033465, U.S. Pat. Nos. 8,188,234, 8,080,243, US 2011/0229489, US 2010/0233177, US 2013/315927 and US 2013/0071405. Each of these references is incorporated reference for the sequence and description of anti-PSCK9 antibodies and antigen-binding fragments thereof.

In one embodiment, the anti-PCSK9 portion may comprise a heavy and light chain variable region chosen from the sequences in Group B of Table 1 above. Alternatively, the anti-PCSK9 portion may comprise a heavy and light chain variable region with CDRs identical to a heavy and light chain variable region from Group B of Table 1 above. Additionally, the anti-PCSK9 portion may comprise a heavy chain variable region chosen from SEQ ID NOS: 53, or 63 and a light chain variable region chosen from SEQ ID NOS: 54, 56, 58, 60, 62, or 64, Alternatively, the anti-PCSK9 portion may comprise heavy chain CDR1, CDR2, and CDR3 from any one of SEQ ID NOS: 53, 55, 57, 59, 61, or 63 and light chain CDR1, CDR2, and CDR3 from any one of SEQ ID NOS: 54, 56, 58, 60, 62, or 64.

In one embodiment, the anti-PCSK9 portion may comprise a heavy and light chain variable region chosen from the sequences in Group C of Table 1 above. Alternatively, the anti-PCSK9 portion may comprise a heavy and light chain variable region with CDRs identical to a heavy and light chain variable region from Group C of Table 1 above. Additionally, the and-PCSK9 portion may comprise a heavy chain variable region chosen from SE(ID NOS: 65-95 and a light chain variable region chosen from SEQ ID NOS: 96-126. Alternatively, the and-PCSK9 portion may comprise heavy chain CDR1, CDR2, and CDR3 from any one of SEQ ID NOS: 65-95 and light chain CDR1, CDR2, and CDR3 from any one of SEQ ID NOS: 96-126.

In one embodiment, the anti-PCSK9 portion may comprise a heavy and light chain variable region chosen from the sequences in Group D of Table 1 above. Alternatively, the anti-PCSK9 portion may comprise a heavy and light chain variable region with CDRs identical to a heavy and light chain variable region from Group D of Table 1 above. Additionally, the anti-PCSK9 portion may comprise a heavy chain variable region chosen from. SECS ID NOS: 1.27-218 and a light chain variable region chosen from SEQ ID NOS: 219-311. Alternatively, the anti-PCSK9 portion may comprise heavy chain CDR1, CDR2, and CDR3 from any one of SEQ ID NOS: 127-218 and light chain CDR1, CDR2, and CDR3 from any one of SEQ ID NOS: 219-311.

In one embodiment, the anti-PCSK9 portion may comprise a heavy and light chain variable region chosen from the sequences in Group E of Table 1 above. Alternatively, the anti-PCSK9 portion may comprise a heavy and light chain variable region with CDRs identical to a heavy and light chain variable region from Group E of Table 1 above. Additionally, the anti-PCSK9 portion may comprise a heavy chain variable region chosen from SEQ ID NOS: 312-317 and a light chain variable region chosen from SEQ ID NOS: 318-323. Alternatively, the anti-PCSK9 portion may comprise heavy chain CDR1, CDR2, and CDR3 from any one of SEQ ID NOS: 312-317 and light chain CDR1, CDR2, and CDR3 from any one of SEQ ID NOS: 318-323.

In one embodiment, the anti-PCSK9 portion may comprise a heavy and light chain variable region chosen from the sequences in Group F of Table 1 above. Alternatively,the anti-PC portion may comprise a heavy and light chain variable region with CDRs identical to a heavy and light chain variable region from Group F of Table 1 above. Additionally, the anti-PCSK9 portion may comprise a heavy chain variable region chosen from SECS ID NO: 324 and a light chain variable region chosen from SEQ ID NO: 325. Alternatively, the anti-PCSK9 Portion may comprise heavy chain CDR1, CDR2, and CDR3 from any one of SEQ ID NOS: 324 and light chain CDR1, CDR2, and CDR3 from any one of SEQ ID NOS: 325.

In one embodiment, the anti-PCSK9 portion may comprise a heavy and light chain variable region chosen from the sequences in Group G of Table 1 above. Alternatively, the anti-PCSK9 portion may comprise a heavy and light chain variable region with CDRs identical to a heavy and light chain variable region from Group G of Table 1 above. Additionally, the anti-PCSK9 portion may comprise a heavy chain variable region chosen from. SEQ ID NOS: 326, 339, 340, or 343-400 and a light chain variable region chosen from SEQ ID NOS: 327, 341, or 342. Alternatively, the anti-PCSK9 portion may comprise heavy chain CDR1, CDR2, and CDR3 from any one of SEQ ID NOS: 326, 339, 340, or 343-400 and light chain CDR1, CDR2, and CDR3 from any one of SEQ ID NOS: 327, 341, or 342. Further, the anti-PCSK9 portion may comprise heavy chain CDR1, CDR2, and CDR3 from SEQ ID NOS: 328 (HC CDR1) 329 (HC CDR2), 331 (HC CDR2), 330 (HC CDR3), 332 (HC CDR3) and light chain CDR1 CDR2, and CDR3 from SEQ ID NOS: 333 (LC CDR1), 334 (LC CDR2), 333 (LC CDR3), 336 (LC CDR1), (LC CDR2), and 338 (LC CDR3). In one embodiment, the anti-PCSK9 portion may comprise a heavy variable region comprising the amino acid sequence of SEQ ID NO: 491 and light chain variable region comprising the amino acid sequence of SEQ ID NO: 492. Alternatively, the anti-PCSK9 portion may comprise heavy chain CDR1, CDR2, and CDR3 from SEQ ID NOS: 493-495 and light chain CDR1 CDR2, and CDR3 from SEQ ID NOS: 496-498.

In other embodiments, the antibody portion may comprise antibodies other than an anti-PCSK9 antibody (e.g., an anti-B7-H1 antibody). In one embodiment, the anti-B7-H1 antibody may comprise a heavy variable region comprising the amino acid sequence of SEQ ID NO: 422 and light chain variable region comprising the amino acid sequence of SEQ ID NO: 423.

2. Antibody or Antigen-Binding Fragments

As used herein, the term antibody or antigen-binding fragment thereof is used in the broadest sense. It may be man-made such as monoclonal antibodies (mAbs) produced by conventional hybridoma technology, recombinant technology and/or a functional fragment thereof. It may include both intact immunoglobulin molecules for example a polyclonal antibody, a monoclonal antibody (mAb), a monospecific antibody, a bispecific antibody, a polyspecific antibody, a human antibody, a humanized antibody, an animal antibody (e.g. camelid antibody), chimeric antibodies, as well as portions, fragments, regions, peptides and derivatives thereof (provided by any known technique, such as, but not limited to, enzymatic cleavage, peptide synthesis, or recombinant techniques), such as, for example, immunoglobulin devoid of light chains, Fab, Fab′, F (ab′)₂, Fv, scFv, antibody fragment, diabody, Fd, CDR regions, or any portion or peptide sequence of the antibody that is capable of binding antigen or epitope. In one embodiment, the functional part is a single chain antibody, a single chain variable fragment (scFv), a Fab fragment, or a F(ab′)₂ fragment.

An antibody or functional part is said to be “capable of binding” a molecule if it is capable of specifically reacting with the molecule to thereby bind the molecule to the antibody. Antibody fragments or portions may lack the Fc fragment of intact antibody, clear more rapidly from the circulation, and may have less non-specific tissue binding than an intact antibody. Examples of antibody may be produced from intact antibodies using methods well known in the art, for example by proteolytic cleavage with enzymes such as papain (to produce Fab fragments) or pepsin (to produce F(ab′)₂ fragments). Portions of antibodies may be made by any of the above methods, or may be made by expressing a portion of the recombinant molecule. For example, the CDR region(s) of a recombinant antibody may be isolated and subcloned into an appropriate expression vector.

In one embodiment, an antibody or functional part is a human antibody. The use of human antibodies for human therapy may diminish the chance of side effects due to an immunological reaction in a human individual against nonhuman sequences. In another embodiment, the antibody or functional part is humanized. In another embodiment, an antibody or functional part is a chimeric antibody. This way, sequences of interest, such as for instance a binding site of interest, can be included into an antibody or functional part.

In one embodiment, the antibody may have an IgG, IgA, IgM, or IgE isotype. In one embodiment, the antibody is an IgG. In one aspect, the anti-PCSK9 antibody or antigen-binding fragment thereof may be an IgG1.

3. Modifications to the Constant Domain

In one embodiment, the anti-PCSK9 antibody or antigen binding fragment comprises an Fc region. It will be understood that Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein.

in one embodiment, the anti-PCSK9 antibody or antigen-binding portion thereof has a variant Fc region having reduced effector function (e.g., reduced ADCC and/or CDC). In one embodiment, the Fc region has no detectable effector function. In one embodiment, the Fc region comprises at least one non-native amino acid at one or more positions chosen from 234, 235, and 331, as numbered by the EU index as set forth in Kabat. In a specific embodiment, the present invention provides an Fc variant, wherein the Fc region comprises at least one non-native amino acid chosen from 234F, 235F, 235Y, and 331S, as numbered by the EU index as set forth in Kabat. In a further specific embodiment, an Fc variant of the invention comprises the 234F, 235F, and 331S amino acid residues, as numbered by the EU index as set forth in Kabat. In another specific embodiment, an Fc variant of the invention comprises the 234F, 235Y, and 331S amino acid residues, as numbered by the EU index as set forth in Kabat. In a particular embodiment, the anti-PCSK9 antibody or antigen-binding portion thereof has a variant Fc region, wherein the variant comprises a phenylalanine (F) residue at position 234, a phenylalanine (F) residue or a glutamic acid (E) residue at position 235 and a serine (S) residue at position 331, as numbered by the EU index as set forth in Kabat. Such mutation combinations are hereinafter referred to as the triple mutant (TM).

The serine228proline mutation (S228P), as numbered by the EU index as set forth in Kabat, hereinafter referred to as the P mutation, has been reported to increase the stability of a particular IgG4 molecule (Lu et al., J Pharmaceutical Sciences 97(2):960-969, 2008). Note: In Lu et al. it is referred to as position 241 because therein they use the Kabat numbering system, not the “EU index” as set forth in Kabat.

This P mutation may be combined with L235E to further knock out ADCC. This combination of mutations is hereinafter referred to as the double mutation (DM).

B. GLP-1 Moiety

The fusion molecule contains a GLP-1 moiety. GLP-1 may also be referenced by the synonym glucagon-like peptide-1. By GLP-1 we also reference Exendin-4, which is a GLP-1 analog. In one embodiment, the GLP-1 moiety provides glucose control and/or weight loss benefits.

In one embodiment, the full length GLP-1 molecule may be used in the fusion protein. In another embodiment, a fragment of GLP-1 may be used as a GLP-1 moiety in the fusion protein.

In one embodiment, the GLP-1 moiety has a pair of cysteine residues that allows for a disulphide bridge. In one embodiment, the cysteine is an engineered cysteine compared to the parental sequence. In one embodiment, the cysteine is an El8C mutation.

In one embodiment, the GLP-1 potency is reduced at the human GLP-1 receptor compared to wild type GLP-1 (e.g., SEQ ID NO: 29) or a GLP-1 analog (e.g., SEQ ID NO: 12). In another embodiment, the GLP-1 potency at the human GLP-1 receptor is at least about 10×, 20×, 30×, 40×, 50×, 60×, 100×, 125×, 150×, 175×, 200×, or 225× lower than wildtype GLP-1 or a GLP-1 analog. In another embodiment, the potency is reduced by similar amounts as compared to dulaglutide. The GLP-1 potency may be reduced to allow for saturation of PCSK9 while reducing side effects. In one embodiment, the GLP-1 moiety may have at least one mutation that reduces the potency of the GLP-1 moiety. In one embodiment, this offers benefits of reducing side effects, including, but not limited to nausea. In one embodiment, the mutation is a point mutation. In one embodiment, the point mutation is chosen from V19A, G2V, E15A, or L26I with respect to Exendin-4.

In one embodiment, the GLP-1 moiety is comprises any one of SEQ ID NOS: 3, 7, 12, 13, or 28-42. In another embodiment, the GLP-1 moiety is a fragment of any one of SEQ ID NOS: 3, 7, 12, 13, or 28-42 comprising at least 10, 15, 20, or 25 amino acids. In a further embodiment, the GLP-1 moiety comprises a sequence which is at least at least 70%, 75%, 80%, 85%, 90%, 95%, 96%, 97%, 98%, or 99% A identical to any one of SEQ ID NOS: 3, 7, 12, 13, or 28-42. In a further embodiment, the GLP-1 moiety comprises a sequence which has 1, 2, 3, 4, 5, or 6 mutations as compared to any one of SEQ ID NOS: 3, 7, 12, 13, or 28-42.

C. Fusion and Linkers

In one embodiment, the GLP-1 moiety is fused directly or indirectly to the light chain of the anti-PCSK9 antibody or antigen binding fragment. In another embodiment, the GLP-1 moiety is fused directly or indirectly to the heavy chain of the anti-PCSK9 antibody or antigen binding fragment.

In one embodiment, a linker may be used to construct a fusion between the anti-PCSK9 antibody or antigen-binding fragment and the GLP-1 moiety. In another embodiment, the anti-PCSK9 antibody or antigen-binding fragment may be directly conjugated to the GLP-1 moiety.

If a linker is used, the linker may be chosen from any suitable linker for fusion proteins. In one embodiment, the linker may comprise a GGGGS (SEQ ID NO: 27) repeat, either alone or in combination with other amino acids, either as one, two, three, or four sets of repeats. In one embodiment, the linker may comprise other combinations of G and S, either alone or in combination with other amino acids. In some embodiments, the linker has a C-terminal Alanine (A).

In one embodiment, a specific linker may be chosen from GGGGSGGGGSGGGGSA (SEQ ID NO: 4). In one embodiment, the linker allows for a disulfide bridge to form between the C terminus of the GLP-1 moiety and another portion of the GLP-1 molecule, but not with the antibody portion. In such an instance, a linker with limited flexibility may prevent undesired disulfide bridging to the antibody portion.

In one embodiment, the linker is at least 70%, 75%, 80%, 85%, 90%, 95%, 96%, 97%, 98%, or 99% identical to SEQ ID NO: 4.

In one embodiment, the cysteine:cysteine disulfide bridge is formed by making a cysteine substitution mutation in the GLP-1 moiety. In one embodiment, the mutation is E18C.

### II. Nucleic Acids Encoding Fusion Molecules

The present embodiments further provides an isolated, synthetic, or recombinant nucleic acid sequence encoding any of the fusion molecules described in section I above. Such nucleic acids encode the heavy and light chain sequences set forth herein. Alternatively, such nucleic acids include the anti-PCSK9 antibody or antigen-binding portion fused to the GLP-1 moiety portion. Due to the degeneracy of the nucleic acid code, multiple nucleic acids will encode the same amino acid and all are encompassed herein.

### III. Methods of Making Fusion Molecules, Formulation, and Pharmaceutical Compositions

One embodiment includes a method of producing the fusion molecule by culturing host cells under conditions wherein a nucleic acid is expressed to produce the fusion molecule, followed by recovering the fusion molecule. A variety of cell lines may be used for expressing the fusion molecule, including, but not limited to, mammalian cell lines. In one embodiment, the cell lines may be human. In another embodiment, bacterial or insect cell lines may be used. In one embodiment, the cell lines include Chinese hamster ovary (CHO) cells, variants of CHO cells (for example DG44), 293 cells, and NS0 cells. In another embodiment, cell lines include VERY, BHK, Hela, COS, MDCK, 293F, 293T, 3T3, W138, BT483, Hs578T, Sp2/0, HTB2, BT2O, T47D, CRL7O3O, and HsS78Bst cells.

Recombinant expression utilizes construction of an expression vector containing a polynucleotide that encodes the fusion molecule. Once a polynucleotide has been obtained, a vector for the production of the fusion molecule may be produced by recombinant DNA technology well known in the art. Expression vectors may include appropriate transcriptional and translational control signals. This may be accomplished using in vitro recombinant DNA techniques, synthetic techniques, and in vivo genetic recombination. In one embodiment, a replicable vector comprises a nucleic acid sequence encoding an antibody or functional part operably linked to a heterologous promoter.

A variety of host-expression vector systems may be utilized to express the fusion molecule as described in U.S. Pat. No. 5,807,715. For example, mammalian cells such as Chinese hamster ovary cells (CHO), in conjunction with a vector such as the major intermediate early gene promoter element from human cytomegalovirus, are an effective expression system for antibodies (Foecking et al., Gene, 45:101 (1986); and Cockett et al., Bio/Technology, 8:2 (1990)). In addition, a host cell strain may be chosen which modulates the expression of inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the protein of the invention. To this end, eukaryotic host cells which possess the cellular machinery for proper processing of the primary transcript, glycosylation, and phosphorylation of the gene product may be used.

In bacterial systems, a number of expression vectors may be selected depending upon the use intended for the fusion molecule being expressed. For example, when a large quantity of such fusion molecule is to be produced, for the generation of pharmaceutical compositions comprising the fusion molecule, vectors which direct the expression of high levels of fusion protein products that are readily purified may be desirable. Such vectors include, but are not limited to, the E. coli expression vector pUR278 (Ruther et al., EMBO, 12:1791 (1983)), in which the coding sequence may be ligated individually into the vector in frame with the lac Z coding region so that a fusion protein is produced; pIN vectors (Inouye & Inouye, 1985, Nucleic Acids Res. 13:3101-3109 (1985); Van Heeke & Schuster, 1989, J. Biol. Chem., 24:5503-5509 (1989)); and the like. pGEX vectors may also be used to express foreign polypeptides as fusion proteins with glutathione-S-transferase (GST). In general, such fusion proteins are soluble and can easily be purified from lysed cells by adsorption and binding to glutathione-agarose affinity matrix followed by elution in the presence of free glutathione. The pGEX vectors are designed to introduce a thrombin and/or factor Xa protease cleavage sites into the expressed polypeptide so that the cloned target gene product can be released from the GST moiety.

In an insect system, Autographa californica nuclear polyhedrosis virus (AcNPV) is used as a vector to express foreign genes. The virus grows in Spodoptera frugiperda cells. The protein coding sequence may be cloned individually into non-essential regions (for example, the polyhedrin gene) of the virus and placed under control of an AcNPV promoter (for example, the polyhedrin promoter).

In mammalian host cells, a number of virus based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion into a non-essential region of the viral genome (e.g., region E1 or E3) will result in a recombinant virus that is viable and capable of expressing the antibody or functional part in infected hosts (e.g., see, Logan & Shenk, Proc. Natl. Acad. Sci. USA, 81:355-359 (1984)). Specific initiation signals may also be required for efficient translation of inserted antibody or functional part coding sequences. These signals include the ATG initiation codon and adjacent sequences. Furthermore, the initiation codon should generally be in frame with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol., 153:51-544(1987)).

Stable expression can be used for long-term, high-yield production of recombinant proteins. For example, cell lines which stably express the fusion molecule may be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells may be allowed to grow for 1-2 days in an enriched media, and then are switched to a selective media. The selectable marker in the recombinant plasmid confers resistance to the selection and allows cells that stably integrated the plasmid into their chromosomes to grow and form foci which in turn can be cloned and expanded into cell lines. Plasmids that encode the fusion molecule can be used to introduce the gene/cDNA into any cell line suitable for production in culture.

A number of selection systems may be used, including, but not limited to, the herpes simplex virus thymidine kinase (Wigler et al., Cell, 11:223 (1977)), hypoxanthineguanine phosphoribosyltransferase (Szybalska & Szybalski, Proc. Natl. Acad. Sci. USA, 48:202 (1992)), and adenine phosphoribosyltransferase (Lowy et al., Cell, 22:8-17 (1980)) genes can be employed in tk-, hgprt- or aprT-cells, respectively. Also, antimetabolite resistance can be used as the basis of selection for the following genes: dhfr, which confers resistance to methotrexate (Wigler et al., Natl. Acad. Sci. USA, 77:357 (1980); O'Hare et al., Proc. Natl. Acad. Sci. USA, 78:1527 (1981)); gpt, which confers resistance to mycophenolic acid (Mulligan & Berg, Proc. Natl. Acad. Sci. USA, 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11(5):155-215 (1993)); and hygro, which confers resistance to hygromycin (Santerre et al., Gene, 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol., 150:1.

Once the fusion molecule has been produced by recombinant expression, it may be purified by any method known in the art for purification, for example, by chromatography (e.g., ion exchange, affinity, particularly by affinity for the specific antigens Protein A or Protein G, and sizing column chromatography), centrifugation, differential solubility, or by any other standard technique for the purification of proteins.

Further provided are compositions, e.g., pharmaceutical compositions, that contain an effective amount of a dual active fusion molecule as provided herein, formulated for the treatment of metabolic diseases, e.g., Type 2 diabetes.

Compositions of the disclosure can be formulated according to known methods. Suitable preparation methods are described, for example, in Remington's Pharmaceutical Sciences, 19th Edition, A. R. Gennaro, ed., Mack Publishing Co., Easton, Pa. (1995), which is incorporated herein by reference in its entirety. Composition can be in a variety of forms, including, but not limited to an aqueous solution, an emulsion, a gel, a suspension, lyophilized form, or any other form known in the art. In addition, the composition can contain pharmaceutically acceptable additives including, for example, diluents, binders, stabilizers, and preservatives. Once formulated, compositions of the disclosure can be administered directly to the subject.

Carriers that can be used with compositions of the disclosure are well known in the art, and include, without limitation, e.g., thyroglobulin, albumins such as human serum albumin, tetanus toxoid, and polyamino acids such as poly L-lysine, poly L-glutamic acid, influenza, hepatitis B virus core protein, and the like. A variety of aqueous carriers can be used, e.g., water, buffered water, 0.8% saline, 0.3% glycine, hyaluronic acid and the like. Compositions can be sterilized by conventional, well known sterilization techniques, or can be sterile filtered. A resulting composition can be packaged for use as is, or lyophilized, the lyophilized preparation being combined with a sterile solution prior to administration. Compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamineoleate, etc.

### IV. Methods for Use of the Anti-PCSK9˜GLP-1 Fusion Molecule and Kits

The anti-PCSK9˜GLP-1 fusion may be used to treat diabetes or TType 2 diabetes. In one embodiment, the patient has TType 2 diabetes. In one embodiment, the patient has a high cardiovascular risk profile. In another embodiment, the patient has both TType 2 diabetes and a high cardiovascular risk profile. By high cardiovascular risk profile, it means that the patient is at higher risk for a cardiovascular event due to one or more factors: high cholesterol, high LDL cholesterol, low HDL cholesterol, high blood pressure, atherosclerosis, obesity, prior cardiovascular event (including angina, heart attack, transient ischemic attack, stroke, etc.), family history of cardiovascular event, smoking, high triglycerides, lack of physical activity, poorly controlled blood sugars, and the like.

In other embodiments, the anti-PCSK9˜GLP-1 fusion may be used to treat other disease including but not limited to NASH, obesity, hypercholesterolemia, and major adverse cardiovascular events (MACE) including but not limited to acute coronary syndrome (ACS), stroke, heart failure, and malignant dysrhythmnia.

In one embodiment, the fusion molecule has increased stability upon administration. In one embodiment, the increased stability is demonstrated by comparing it to a benchmark control compound dulaglutide, a GLP-1 analog fused with an Fc fragment, in an in vivo administration to mice.

In one embodiment, the fusion molecule as increased potency at the GLP-1 receptor. In one embodiment, the decreased potency is demonstrated at the human GLP-1 receptor over the benchmark control compound dulaglutide and/or wild type GLP-1.

In some embodiments, the fusion molecule promotes weight loss in a subject.

In one embodiment, the fusion molecule is administered by injection.

In other embodiments, the present disclosure provides kits comprising dual active fusion molecules, which can be used to perform the methods described herein. In certain aspects, a kit comprises a dual active fusion molecule disclosed herein in one or more containers. A kit as provided herein can contain additional compositions for combination therapies. One skilled in the art will readily recognize that the disclosed dual active fusion molecules can be readily incorporated into one of the established kit formats that are well known in the art.

Reference will now be made in detail to the present exemplary embodiments, examples of which are illustrated in the accompanying drawings. Wherever possible, the same reference numbers will be used throughout the drawings to refer to the same or like parts. Other embodiments will be apparent to those skilled in the art from consideration of the specification and practice disclosed herein. The embodiments are further explained in the following examples. These examples do not limit the scope of the claims, but merely serve to clarify certain embodiments. It is intended that the specification and examples be considered as exemplary only, with a true scope and spirit being indicated by the following claims.

### EXAMPLES

### Example 1

### Antibody Optimisation

The anti-PCSK9 antibody PC9#2 (SEQ ID NOs. 8 and 9 for the variable heavy and light chains respectively) has been optimised to:

1_Reduce the immunogenicity risk by reverting amino acids to the ones corresponding to the closest human germline sequence without significantly impacting the binding to PCSK9 antigen.

2_Remove pH-dependent binding to PCSK9 by mutating histidine residues VH_52, VL_30 and VL_50 (Kabat numbering).

3_Improve affinity for human PCSK9 at physiological pH in order to efficiently engage with the target and achieve sufficient free PCSK9 suppression following administration of PCSK9/GLP-1 peptide antibody fusion molecules.

A) Germlining

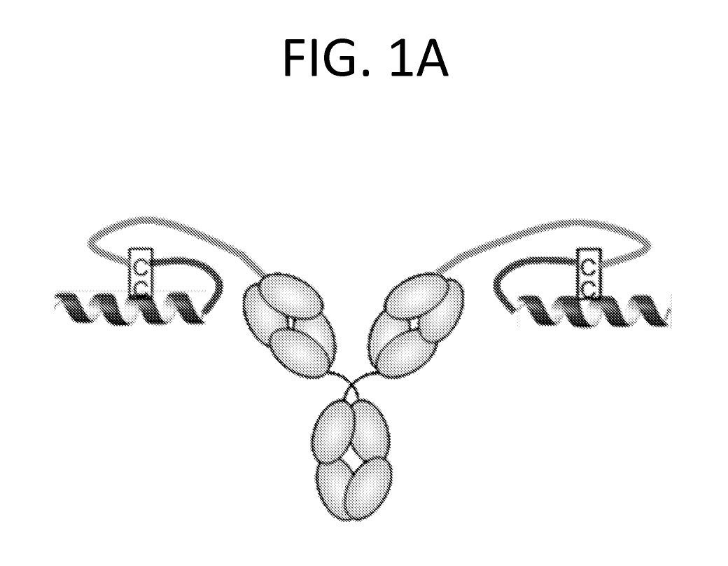

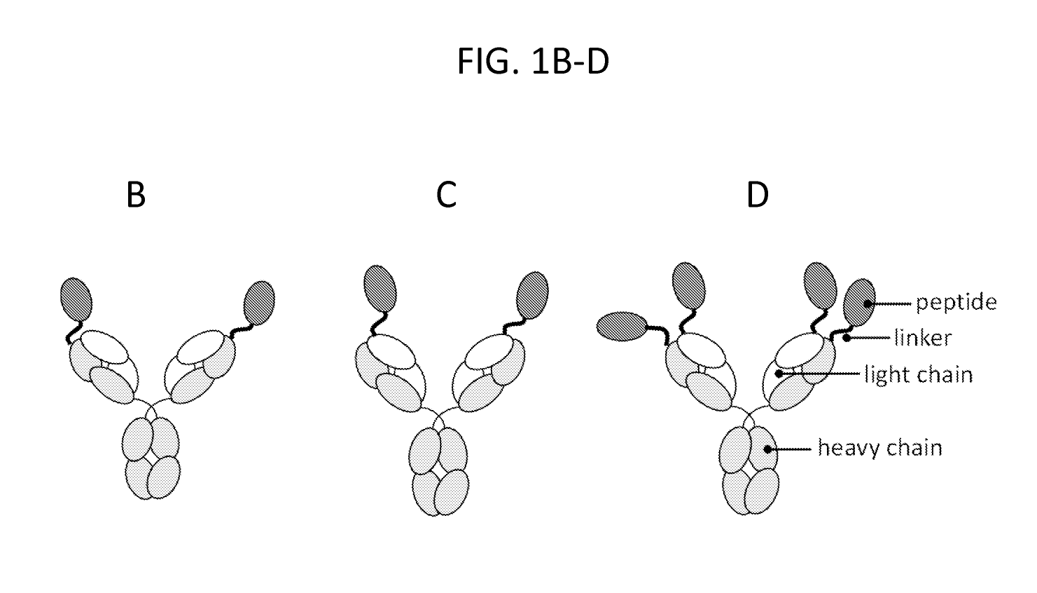

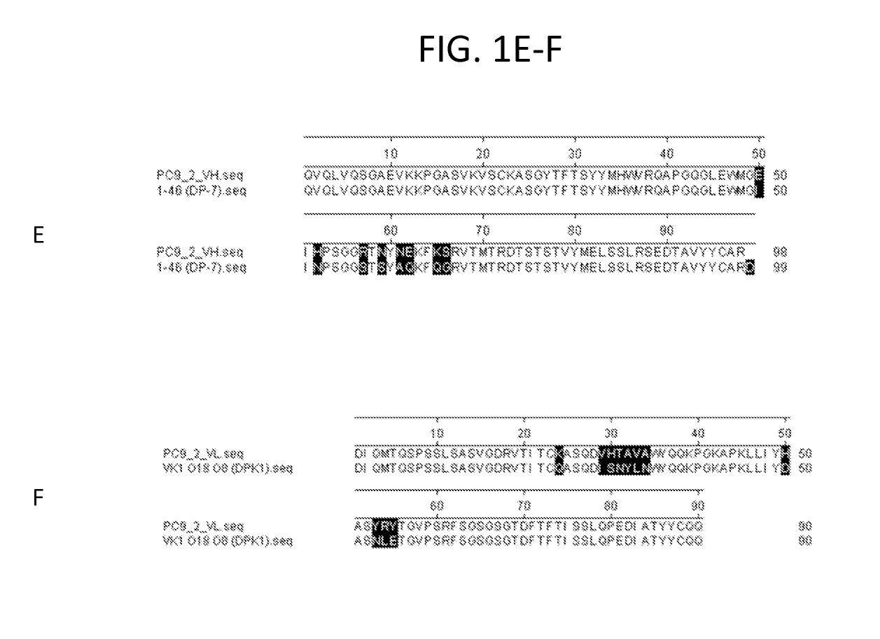

The amino acid sequences of the VH and VL domains of the anti-PCSK9 antibody PC9#2 were aligned to the known human germline sequences in the VBASE database (Tomlinson, 1997; http://vbase.mrc-cpe.cam.ac.uk/), and the closest human germline was identified by sequence similarity to be 1-46 (DP-7) (SEQ ID 417) and VK1 O18 O8 (DPK1) (SEQ ID 418) for the variable heavy and light chains respectively. FIGS. 1E and 1F are showing an alignment of PC9#2 variable domains with those germline sequences.

A structure model of the anti-PCSK9 PC9#2 antibody in complex with human PCSK9 antigen has been generated using the primary amino acid sequence of PC9#2 variable domains and previously described crystal structure of human PCSK9 in complex with another anti-PCSK9 antibody deposited in the Protein Data Bank using PBD ID code 3SQO (Liang et al., 2012, J. Pharm. Exp. Ther., Vol. 340, p228-236).

Using that structure model, the following residues were identified as non-contacting with PCSK9 antigen but nonetheless solvent-exposed: Arg56, Asn58, Glu61, Lys64 and Ser65 in the variable heavy chain as well as Arg24 in the variable light chain (Kabat numbering). Because those residues are different from the closest germline sequences and might be solvent-exposed, they can present some immunogenicity risks. Without being bound by theory, mutating those residues should not significantly impact the ability of the antibody to strongly bind PCSK9 as they should not contribute to the interaction network between the antibody and its antigen.

Mutations R56S, N58S, E61Q, K64Q and S65G were then introduced in PC9#2 heavy chain sequence as well as K24Q in the light chain using standard molecular biology techniques to generate the antibody PC9#2_FG of SEQ ID 5 and 6 for the variable heavy and light chains respectively.

Anti-PCSK9 PC9#2 and PC9#2_FG were produced as human IgG1-TM antibodies as described in Example 2 and characterised for binding to human PCSK9 using Biacore as described in Example 17. Kinetic parameters at pH 7.4 for those compounds are summarised in Table 2. Both antibodies exhibit similar on-rate, off-rate and affinity for human PCSK9 demonstrating that the germlining mutagenesis had no impact on antigen binding.

TABLE 2
Kinetic parameters for human PCSK9 at physiological pH
of germlined and non-germlined version of PC9#2antibody

| Compound | ka (1/Ms) | kd (1/s) | KD (M) |
|---|---|---|---|
| PC9#2 | 2.70E+05 | 5.10E−04 | 1.89E−09 |
| PC9#2_FG | 3.03E+05 | 5.40E−04 | 1.78E−09 |

B) Removing pH Dependent Binding and Improving Affinity for Human PCSK9

Anti-PCSK9 antibodies PC9#2 and PC9#2_FG exhibit pH-dependent binding properties as they bind strongly to human PCSK9 antigen at physiological pH but rapidly dissociate from their target at acidic pH. That feature should enable the antibodies to dissociate from PCSK9 in the acidic compartment of endosome in order to be recycled at cell surface rather than being sent to lysosome for degradation. This should ultimately translate into a longer antibody in vivo half-life.

Table 3 are comparing off-rate (kd) for those two compound with human PCSK9 at pH7.4 (physiological) and 6.0 (acidic) determined by Biacore as described in Example 17 but with the following modifications. After antibody capture onto the CM5 chip, human PCSK9 antigen diluted to concentrations ranging from 1 nM to 200 nM in running buffer pH7.4 (10 mM sodium phosphate pH 7.4, 150 mM sodium chloride, 1 mg/mL BSA, 0.05% Tween20) were injected for 10 minutes. After the association phase, running buffer at pH7.4 or pH6.0 were injected for 10 minutes dissociation phase. Global dissociation rates were calculated using a 1:1 binding kinetics model.

TABLE 3
Dissociation constant (kd) at physiological and acidic pH
of germlined and non-germlined version of PC9#2antibody.

| | kd (1/s) | kd (1/s) | kd |
|---|---|---|---|
| Compound | pH 7.4 | pH 6.0 | ratio |
| PC9#2 | 5.10E−04 | 3.24E−03 | 6.4 |
| PC9#2_FG | 5.40E−04 | 6.47E−03 | 12 |

Long antibody in vivo half-life is not desirable for PCSK9/GLP-1 fusion molecules as it might lead to the accumulation of drug metabolites able to bind PCSK9 but degraded in the GLP-1 analogue peptide and thus unable to activate the GLP-1 receptor.

In addition, genetic fusion of a GLP-1 analogue peptide in front of the light chain of the anti-PCSK9 PC9#2 was slightly impacting the affinity of the compound for human PCSK9 (19 nM) compared to PC9#2 antibody (7 nM) as shown in Example 5, Table 11. Affinity maturation of PC9#2_FG was then required in order to counterbalance the negative impact of GLP-1 analogue light chain fusion on affinity for human PCSK9 antigen.

To remove pH-dependent binding, histidine residues in position 52 of the heavy chain as well as in position 30 and 50 of the light chain need to be mutated. Based on the structure model described above and subsequent analysis of PC9#2 in complex with human PCSK9, the following mutations were identified as potentially beneficial for antibody binding to PCSK9 and could lead to an affinity improvement: heavy chain H52K or H52S, light chain H30Y, H30K or H30S and light chain H50Y or H50S.

Combination of all those mutations in the sequence of PC9#2_FG were generated using standard molecular biology techniques in order to produce optimised antibodies. Table 4 is summarising the different compounds resulting from the combination experiment.

TABLE 4
PC9#2_FG mutations to generate optimised antibodies

| | | | | Antibody | |
|---|---|---|---|---|---|
| # | VH_H52 | VL_H30 | VL_H50 | name | SEQ ID |
| 1 | K | Y | Y | HS1 | 424 and |
| | | | | | 425 |
| 2 | K | Y | S | HS2 | 426 and |
| | | | | | 427 |
| 3 | K | K | Y | HS3 | 428 and |
| | | | | | 429 |
| 4 | K | K | S | HS4 | 430 and |
| | | | | | 431 |
| 5 | K | S | Y | / | |
| 6 | K | S | S | HS5 | 432 and |
| | | | | | 433 |
| 7 | S | Y | Y | HS6 | 434 and |
| | | | | | 435 |
| 8 | S | Y | S | HS7 | 436 and |
| | | | | | 437 |
| 9 | S | K | Y | HS8 | 438 and |
| | | | | | 439 |
| 10 | S | K | S | HS9 | 1 and 2 |
| 11 | S | S | Y | / | |
| 12 | S | S | S | HS10 | 442 and |
| | | | | | 443 |

Combination of H30S and H50Y mutations in PC9#2_FG light chain failed to deliver and compound #5 and #11 have subsequently not been generated.

Antibodies were produced as human IgG1-TM as described in Example 2 and tested for their ability to block the binding of PC9#2 to human PCSK9 using an epitope competition assay as described in Example 3. Data are summarised in Table 5 and show that HS7, HS9 and HS10 have an IC50 at least 10-fold lower compared to the parent antibody PC9#2_FG suggesting that those antibodies may have a significantly better affinity for PCSK9 than PC9#2_FG.

TABLE 5
Inhibition of PC9#2 binding to human PCSK9
using anti-PCSK9 antibodies as competition reagent.

| | | Ratio over |
|---|---|---|
| Antibody | IC50 (M) | PC9#2_FG |
| HS1 | 5.6E−10 | 1.3 |
| HS2 | 9.3E−11 | 7.8 |
| HS3 | 7.0E−10 | 1.0 |
| HS4 | 1.2E−10 | 6.1 |
| HS5 | 2.2E−10 | 3.3 |
| HS6 | 1.7E−10 | 4.3 |
| HS7 | 6.3E−11 | 11.6 |
| HS8 | 1.6E−10 | 4.6 |
| HS9 | 6.6E−11 | 11.1 |
| HS10 | 7.0E−11 | 10.4 |
| PC9#2_FG | 7.3E−10 | 1.0 |

Antibody HS9 has been further characterised and compared to the parental antibody PC9#2_FG for its binding parameters to human PCSK9 at physiological pH by Biacore as described in Example 17. As shown in Table 6, engineered anti-PCSK9 antibody HS9 displays a 3-fold improvement in affinity for human PCSK9 compared to PC9#2_FG at physiological pH, mainly due to a reduction in off-rate (kd).

TABLE 6
Kinetic parameters for human PCSK9 at physiological pH of
engineered antibody HS9 compared to PC9#2_FG

| Antibody | ka (1/Ms) | kd (1/s) | KD (M) |
|---|---|---|---|
| PC9#2_FG | 3.09E+05 | 6.57E−04 | 2.13E−09 |
| HS9 | 3.81E+05 | 2.57E−04 | 6.75E−10 |

In a separate experiment, pH dependent binding of HS9 anti-PCSK9 antibody has been compared to PC9#2_FG using Biacore as described above. Table 7 is showing the dissociation constant (kd) at physiological and acidic pH for those two compounds. Contrary to PC9#2_FG which dissociates more rapidly at pH6.0 than at pH7.4, HS9 displays a lower kd at acidic than at physiological pH demonstrating that it is dissociating from PCSK9 more slowly at pH6.0 than at pH7.4.

TABLE 7
Dissociation constant (kd) at physiological and acidic pH of
engineered antibody HS9 compared to PC9#2_FG.

| | kd (1/s) | kd (1/s) | ratio |
|---|---|---|---|
| Antibody | pH 7.4 | pH 6.0 | kd |
| PC9#2_FG | 6.50E−04 | 7.40E−03 | 11.4 |
| HS9 | 2.04E−04 | 9.82E−05 | 0.5 |

### Example 2

### Preparation of a Dual Action Fusion Molecule

A dual action fusion molecule was made according with a large-scale structure as shown in FIG. 1A of: GLP-1 moiety-Linker-Antibody Light Chain. SEQ ID NO: 3 was used as the GLP-1 moiety, SEQ ID NO: 4 was used as the linker, and SEQ ID NO: 1 and 2, were used as the heavy and light chain respectively.

In this embodiment, the N-terminal end of GLP-1 analogue peptides was free in order to most efficiently engage and activate the GLP-1 receptor; peptides were fused to the N-terminal of antibody variable domains. A linker sequence was used in many constructs between the end of the peptide and the start of variable domain in order to minimize the impact of the fusion on peptide and/or antibody activities. Peptides were fused either at the heavy chain or the light chain of antibodies in order to obtain two peptide moieties per fusion molecule. Large scale structure of such fusions are shown FIGS. 1B (heavy chain fusion) and 1C (light chain fusion). In some embodiments, peptides may be fused at both the heavy and light chains to display four peptide moieties per fusion molecule (shown prophetically in FIG. 1D).

Genes coding for GLP-1 analogue peptides in fusion with antibody variable domains were built by overlapping PCR using standard methods. Unique restriction sites were incorporated at the 5′ and 3′ end of the DNA fragment to enable cloning in the expression vectors.

The VH domain, with or without a peptide/linker fusion, was cloned into a vector containing the human heavy chain constant domains and regulatory elements to express whole IgG1 triple mutant (IgG1-TM) heavy chain in mammalian cells. IgG1-TM format is similar to human IgG1 but Fc sequence incorporating mutations L234F, L235E and P331S to reduce its ability to trigger antibody-dependent cell-mediated cytotoxicity and complement-dependent cytotoxicity (Oganesyan V. et al., 2008, Acta Cryst., D64: 700-704). The VL domain, with or without peptide/linker fusion, was cloned into a vector for the expression of the human light chain constant domains and regulatory elements to express whole IgG light chain in mammalian cells. An OriP fragment was included in the heavy and light chain expression vectors to facilitate use with CHO cells and to allow episomal replication.

To obtain IgGs and peptide antibody fusions, the heavy and light chain expressing vectors were transiently transfected into 30 mL (small scale) or 400 mL (medium scale) of CHO mammalian cells. Table 8 is summarizing the different products that can be obtained by co-transfection of the heavy and light chain expressing vectors with or without a peptide/linker fusion.

TABLE 8
Product description when using heavy and light chain vectors co-transfection

| # | Heavy chain vector | Light chain vector | Product |
|---|---|---|---|
| 1 | VH without peptide fusion | VL without peptide fusion | IgG |
| 2 | VH with peptide fusion | VL without peptide fusion | Peptide/antibody VH fusion |
| | | | (FIG. 1B) |
| 3 | VH without peptide fusion | VL with peptide fusion | Peptide/antibody VL fusion |
| | | | (FIG. 1C) |
| 4 | VH with peptide fusion | VL with peptide fusion | Peptide/antibody VH + VL |
| | | | (shown prophetically in FIG. 1D) |

Compounds were expressed and secreted into the medium. Harvests were pooled and filtered before compound purification using Protein A chromatography. Culture supernatants were loaded on a column of appropriate size of MabSelectSure (GE Healthcare Life Sciences) and washed with 1× DPBS (Gibco). Bound compound was eluted from the column using 0.1 M Sodium Citrate pH 3.0 and neutralised by the addition of Tris-HCl pH 9.0. For small scale transfection, eluted material was buffer exchanged into 1× DPBS using PD10 columns (GE Healthcare). For medium scale transfection, eluted material was further purified by Size Exclusion Chromatography (SEC) using either a HiLoad 16/600 Superdex 200 prep grade column (GE Healthcare) for sample volumes of up to 5 ml or a HiLoad 26/600 Superdex 200 column (GE Healthcare) for sample volumes of up to 12 ml. Isocratic elution was performed using 1× DPBS as the running buffer.

Compound concentration was determined spectrophotometrically using an extinction coefficient based on the amino acid sequence according to the protocol in Mach, H. et al., Statistical determination of the average values of the extinction coefficients of tryptophan and tyrosine in native proteins, Anal Biochem, 200(1):74-80 (1992). Purified compounds were analysed for aggregation and degradation using SEC-HPLC and SDS-PAGE. SEC-HPLC was performed by loading 70 μL of sample onto a TSKgel G3000SWXL 7.8 mm×300 mm column (Tosoh Bioscience) using a flow rate of 1 mL/min and 0.1M Sodium Phosphate Dibasic anhydrous plus 0.1M Sodium Sulphate at pH 6.8 as running buffer. SDS-PAGE is run by loading 2 μg protein on a Nu PAGE® 4%-12% Bis-Tris (Invitrogen) using 1× Nu PAGE® MES SDS Running Buffer (Invitrogen). Compounds from medium scale transfections were further characterized for integrity by Electro-Spray Ionisation Mass Spectrometry (ESI-MS) and tested for endotoxin level using the Limulus Amebocyte Lysate (LAL) Kinetic-QCL (Lonza). For ESI-MS analysis, samples were prepared at 1 mg/mL in 10 mM Tris-HCl pH 8.0. A 30 minutes reduction at 37° C. using 10 mM DTT was carried out prior analysis. Data were acquired using a Waters ACQUITY UPLC® I-Class system coupled to a Waters SYNAPT G1 QTOF Mass Spectrometer, operated using MassLynx software. The mobile phases used were highly purified water plus 0.01% trifluoroacidic acid (TFA), 0.1% formic acid (A) and acetonitrile+0.01% TFA, 0.1% FA (B). Separation between heavy and light chain was achieved using a 2.1 mm×50 mm Waters BEH300 C4 column, heated at 60° C. The flow rate was set at 0.3 mL/min, with 22 minutes total run time. Samples (10 pmol) were injected onto the column and MS data were acquired in positive ion mode, using the following electrospray source parameters: capillary voltage: 3.4 kV, 81° C. source temperature: 81° C., desolvation temperature: 24° C. Data were acquired between 500 and 4500 Da.

### Example 3

### l In Vitro Characterization of a GLP-1 Analogue in Genetic Fusion with Anti-PCSK9 Antibodies

To evaluate the feasibility of generating dual activity molecules by genetic fusion between GLP-1 receptor agonist peptides and anti-PCSK9 antibodies, the GLP-1 analogue peptide of SEQ ID NO:28 was fused using a linker of SEQ ID NO:4 to the heavy or light chain variable domains of seven different previously identified anti-PCSK9 antibodies.

1_PC9#1 with antibody variable heavy chain of SEQ ID NO:10 and antibody variable light chain of SEQ ID NO:11.

2_PC9#2 with antibody variable heavy chain of SEQ ID NO:8 and antibody variable light chain of SEQ ID NO:9.

3_PC9#3 with antibody variable heavy chain of SEQ ID NO: 404 and antibody variable light chain of SEQ ID NO: 405.

4_PC9#4 with antibody variable heavy chain of SEQ ID NO: 406 and antibody variable light chain of SEQ ID NO: 407.

5_PC9#5 with antibody variable heavy chain of SEQ ID NO: 408 and antibody variable light chain of SEQ ID NO: 409.

6_PC9#6 with antibody variable heavy chain of SEQ ID NO: 410 and antibody variable light chain of SEQ ID NO: 411.

7_PC9#7 with antibody variable heavy chain of SEQ ID NO: 412 and antibody variable light chain of SEQ ID NO:413.

PCSK9 activity of each peptide-antibody fusions was assessed using Homogenous Time Resolved Fluorescence (HTRF) epitope competition assays. In these assays a fluorescence resonance energy transfer (FRET) complex is formed between Streptavidin Cryptate, biotinylated-PCSK9 and a fluorescently (DyLight 650) labelled anti-PCSK9 antibody. Unlabeled peptide-antibody fusions that bind the same or overlapping epitopes of PCSK9 as that bound by the fluorescently labelled antibody will compete resulting in a reduction in FRET signal.

Labeling of the anti-PCSK9 antibodies was carried out using a DyLight-650 (Thermo Scientific 84536) according to the manufacturer instructions. For the assay all samples and reagents were prepared in assay buffer containing 1× Phosphate Buffered Saline, 0.1% BSA (Sigma A9576) and 0.4M Potassium Fluoride. Test samples of peptide-antibody fusions or control antibodies were prepared in 384 well polypropylene plates by 3-fold serial dilutions. Samples (5 μl) or assay buffer (total binding control wells) were transferred to a 384-well assay plate (Costar 3676) and incubated for 4 hours at room temperature with 1 nM Streptavidin Cryptate (Cisbio 61SAXLB), 0.05-0.5 nM biotinylated-PCSK9 and 0.1-2 nM Dy650 labelled anti PCSK9 antibody in a 20 μl total assay volume. Non-specific binding (NSB) control wells were set up with the biotinylated PCSK9 omitted. Time resolved fluorescence emission at 665 nm and 620 nm was measured following excitation at 320 nm using the Perkin Elmer Envision. The ratio of the 665 nm counts/620 nm counts was calculated and multiplied by 10000 to give HTRF Counts. Delta F % was then calculated using the following equation.

$$DeltaF \% = ( \frac{Sample HTRF Counts - NSBHTRF Counts}{NSB HTRF Counts} ) \times 100$$

The results were expressed as % specific binding according to the following equation:

$$\% SpecificBinding = ( \frac{( Sample DeltaF \% - NSB DeltaF \% )}{( Total Binding DeltaF \% - NSB DeltaF \% )} ) \times 100$$

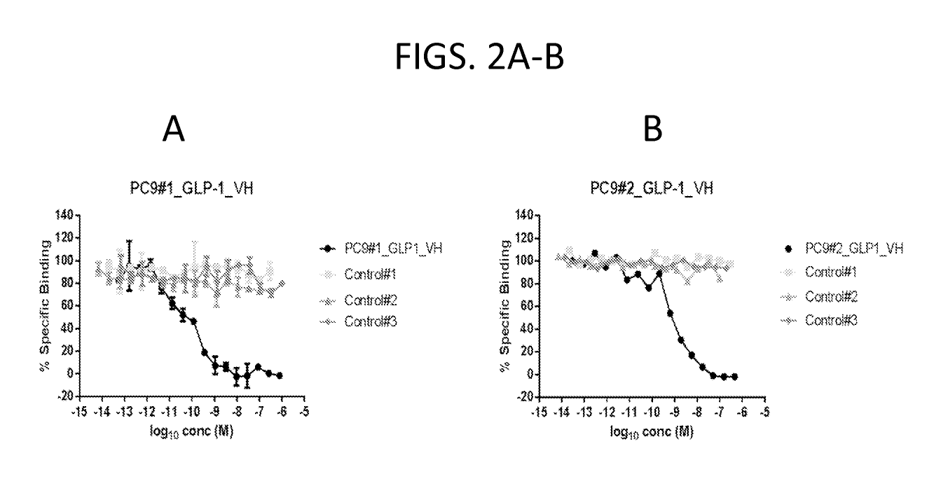

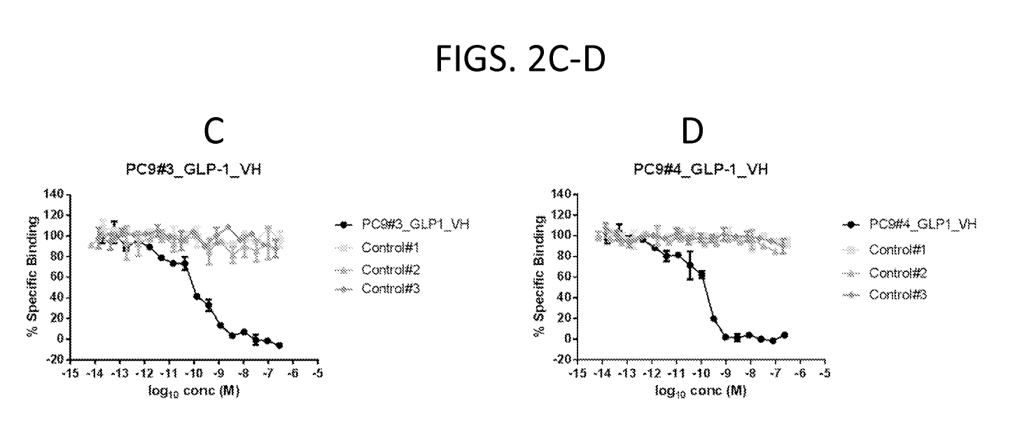

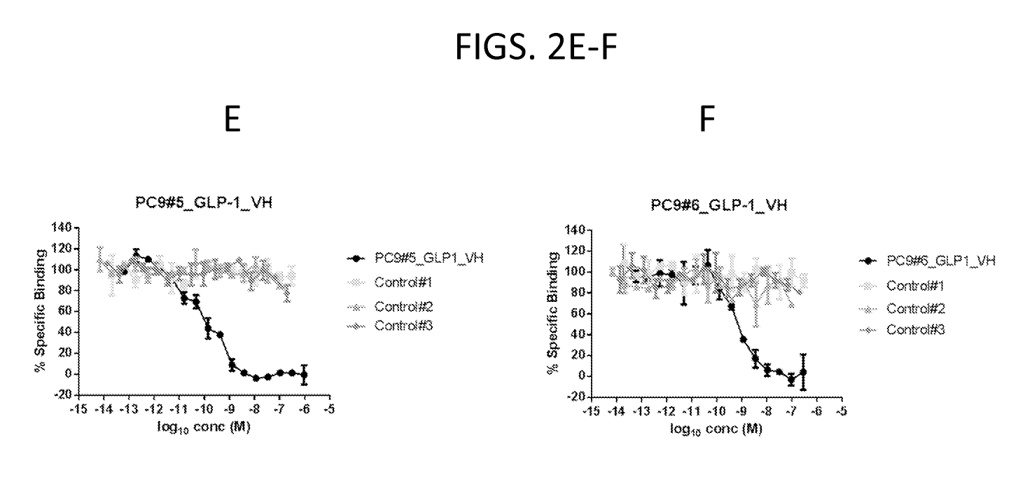

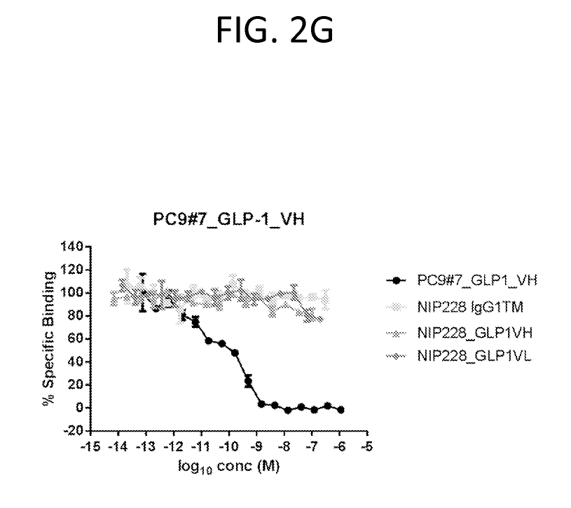

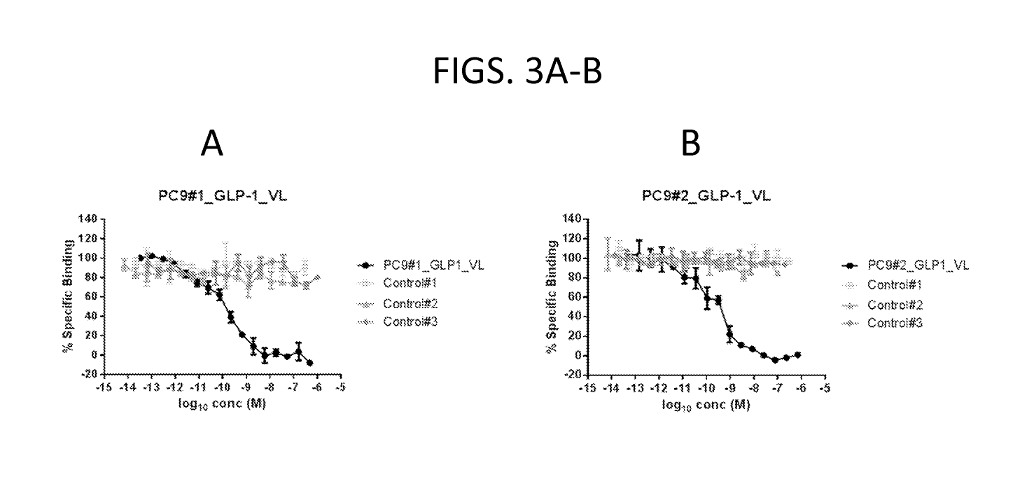

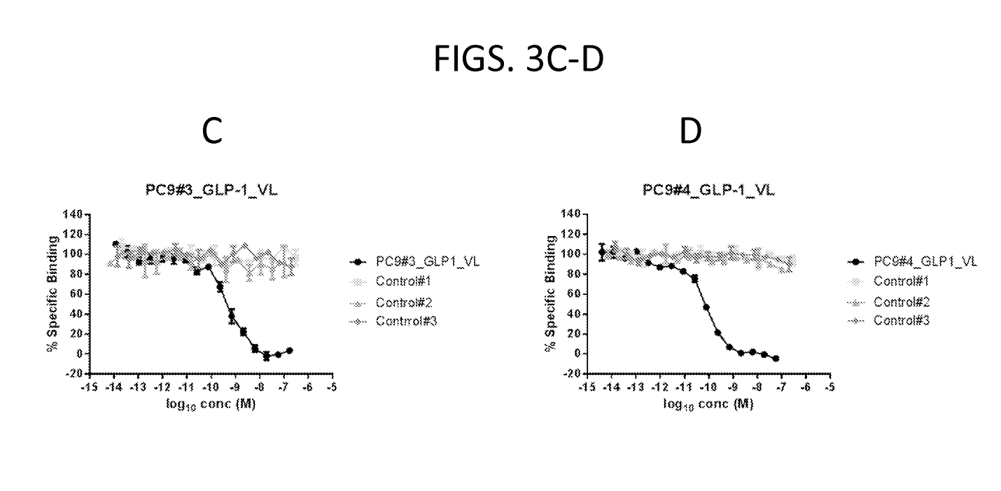

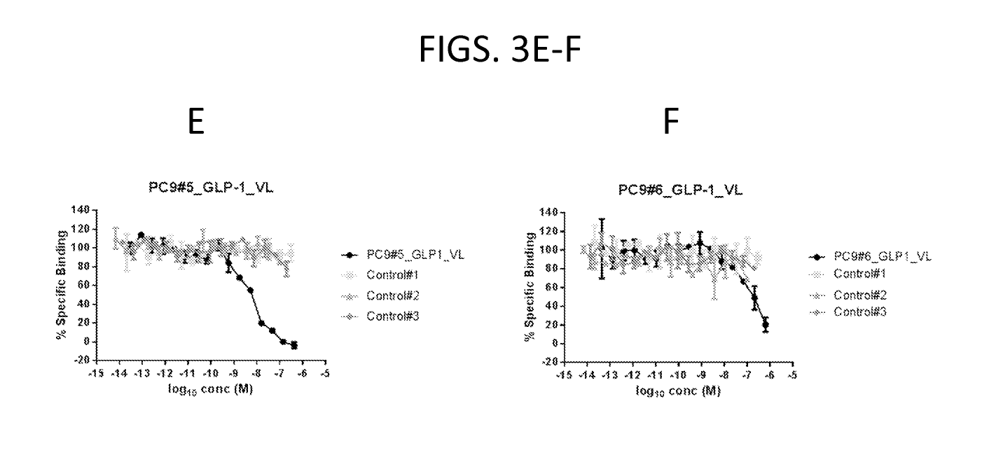

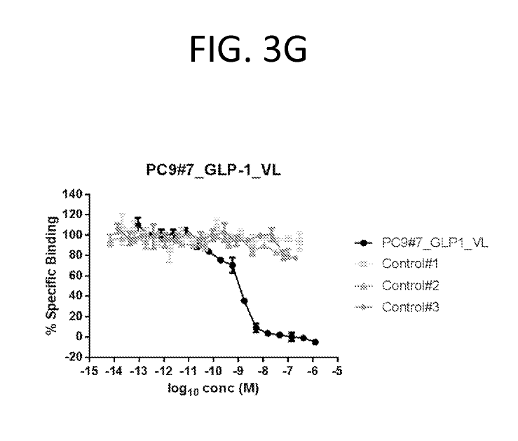

FIGS. 2A-G and FIGS. 3A-G showing the inhibition of human PCSK9 binding to anti-PCSK9 antibodies using a titration of unlabeled heavy chain or light chain peptide antibody fusions respectively. Isotype match irrelevant antibody NIP228 IgG1-TM alone (Control #1) or in fusion to the heavy (Control #2) or light chain (Control #3) with a GLP-1 analogue peptide of SEQ ID NO:28 using a linker of SEQ ID NO:4 were used as negative controls.

Potency of each peptide antibody fusion at human GLP-1 receptor was assessed using a cAMP production assay. Stable cell lines expressing human GLP-1 receptor was generated in CHO cells by standard methods. GLP-1 receptor activation by tested compounds will result in downstream production of cAMP second messenger that can be measured in a functional activity assay. Low protein binding 384-well plates (Greiner) were used to perform eleven 1 in 4 serial dilutions of test compound that were made in assay medium (0.1% bovine serum albumin in Hanks Balanced Salt Solution (GIBCO or Sigma), containing 0.5 mM IBMX (Sigma)). All sample dilutions were made in duplicate. A frozen cryo-vial of cells expressing human GLP-1 receptor was thawed rapidly in a water-bath, transferred to pre-warmed assay media and spun at 240×g for 5 minutes. Cells were then re-suspended in assay buffer at the optimized concentration of 1×10⁵ cells/mL and dispensed at 5 uL per well to black shallow-well u-bottom 384-well plates (Corning). 5 μL of test compound was transferred from the dilution plate to the cell plate and incubated at room temperature for 30 minutes. cAMP levels were measured the cAMP dynamic 2 HTRF kit (Cisbio), following the two step protocol as per manufacturer's recommendations. Briefly, anti-cAMP cryptate (donor fluorophore) and cAMP-d2 (acceptor fluorophore) were made up separately by diluting each 1/20 in conjugate & lysis buffer provided in the kit. 5 uL of anti-cAMP cryptate was added to all wells of the assay plate and 5 uL of cAMP-d2 added to all wells except non-specific binding wells, to which only conjugate and lysis buffer was added. Plates were incubated at room temperature for one hour and then read on an Envision (Perkin Elmer) using excitation wavelength of 320 nm and emission wavelengths of 620 nm and 665 nm. Data was transformed to % Delta F as described in manufacturer's guidelines and analyzed by unconstrained 4-parameter logistic fit of data, curve mid-point to determine EC₅₀.

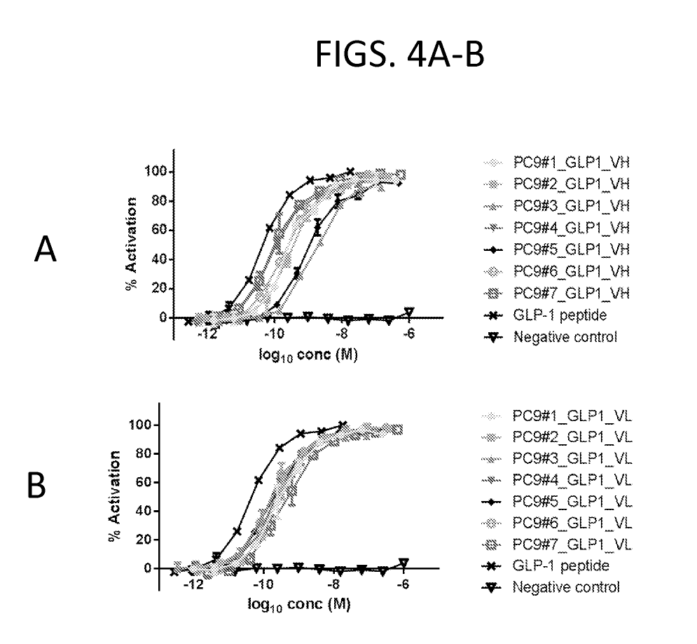

Activation of the human GLP-1 receptor by heavy chain (A) or light chain (B) peptide antibody fusions are shown in FIGS. 4A-B, respectively. Free human GLP-1 peptide (Bachem) and irrelevant isotype match NIP228 human IgG1-TM without peptide were used as positive and negative controls respectively.

To conclude, all tested fusions were able to compete with anti-PCSK9 antibodies for binding to human PCSK9 as well as activating the human GLP-1 receptor. Those fusion molecules between a GLP-1 analogue peptide and different anti-PCSK9 antibodies display dual activity and can be used to provide combined pharmacology.

### Example 4

### In Vivo Stability of Existing GLP-1 Analogues in Antibody Fusion

Without target-mediated clearance, human IgGs have a long circulating half-life in man of around 21 days. This is notably due to the rescue of internalised antibodies by FcRn receptor. After non-specific uptake by cells, antibodies can bind to FcRn in the acidic environment of endosomes and be directed to the cell surface and back into circulation rather than to lysosomes for degradation.

In order to achieve maximum efficacy, GLP-1 analogue peptide in fusion with anti-PCSK9 antibody molecules should display adequate in vivo activity half-life for both activation of the GLP-1 receptor and PCSK9 suppression mediated by the peptide and the antibody moieties respectively. For instance if the peptide is quickly inactivated after injection, a majority of the product will only be functional for PCSK9 suppression and will not properly engage with the GLP-1 receptor over time to provide efficient glucose control through the dosing period.

In order to assess in vivo stability of existing GLP-1 analogue peptides when in antibody fusion, the following fusions were generated:

1: A GLP-1 analogue peptide of SEQ ID NO:28 was fused using a linker of SEQ ID NO:4 to the heavy chain of the irrelevant NIP228 human IgG1-TM antibody of SEQ ID NO: 414 and 415 (compound NIP228_GLP-1_VH).

2: Exendin-4 peptide, a GLP-1 analogue derived from Gila monster' saliva, of SEQ ID NO:12 was fused using a linker of SEQ ID NO:4 to the light chain of the anti-PCSK9 antibody PC9#2 of SEQ ID NO 9 (compound PC9#2_Exe4_VL).

3: A GLP-1 analogue peptide of SEQ ID NO:28 was fused using a linker of SEQ ID NO:4 to human IgG4 Fc fragment of SEQ ID NO: 416 (compound GLP-1-Fc)

4: A GLP-1 analogue peptide of SEQ ID NO:28 was fused using a linker of SEQ ID NO:4 to the light chain of the anti-PCSK9 antibody PC9#2 of SEQ ID NO 9 (compound PC9#2_GLP-1_VL).

All compounds are active at the human GLP-1 receptor in vitro and display EC₅₀ in the cAMP assay described in Example 3 of 2.08E-10 M,1.12E-10 M, 1.12E-10 M and 1.03E-10 M for NIP228_GLP-1_VH, PC9#2_Exe4_VL, GLP-1-Fc and PC9#2_GLP-1_VL respectively.

Compounds NIP228_GLP-1_VH and PC9#2_Exe4_VL were injected intravenously to rat and serum or plasma samples were collected at several timepoints post-injection. Compound concentration in serum or plasma (exposure) and concentration in active compound for GLP-1 activity were determined for each sample. Comparison between decline over time of total compound (exposure) and active compound at GLP-1 receptor provides an assessment of the in vivo stability for the GLP-1 analogue peptide in antibody fusion.

Levels of total human IgG1 antibody in rat plasma or serum were quantified by a generic sandwich enzyme-linked immunosorbent assay (ELISA) method using the Gyrolab platform (Gyros AB). Human IgG1 was captured by a biotinylated monoclonal anti-human IgG1 antibody (clone JDC-10, Southern Biotech, for plasma samples or in-house clone TM446 for serum samples) at 100 ug/mL and detected by an Alexa-labelled monoclonal anti-human IgG1 antibody at 25 nM (BD Pharmingen clone G18-145 for plasma samples) or 10 nM (the Binding Site AU003CUS01 for serum samples) on Gyrolab Bioaffy 200 CD. Standards, controls, plasma or serum samples, wash solution, capture and detection antibodies were added to a 0.2 mL 96-well PCR plate (Thermo Scientific) according to the Gyrolab method and loaded onto the machine with the CD200 plate. All samples were analyzed in duplicate. The mean response of each human IgG standard was plotted against concentration and the points were fit using a 5-parameter weighted logistic model using the Gyrolab Evaluator software.

The concentration of active peptide-antibody fusion at human GLP-1 receptor in rat samples was estimated using an ex-vivo cAMP cell based assay. Reference compounds were spiked into naïve rat serum or plasma at a known concentration to be used as a standard. All samples were serially diluted in assay medium and examined using the cAMP dynamic 2 HTRF kit (Cisbio) as described in Example 3 for serum samples or using the LANCE® Ultra cAMP Detection Kit (Perkin Elmer) for plasma samples.

Test samples were plotted using the same top concentration as the equivalent reference. The EC₅₀ values obtained could then be used to calculate the Sample Ratio (Sample EC₅₀/EC₅₀ reference compound) and then the estimated concentration in active GLP-1 compound (known top concentration of reference compound spiked into rat plasma or serum/Sample Ratio). Rat serum or plasma alone has a quenching effect on the cryptate donor signal and gives concentration-dependent activation of cAMP that can be diluted out. Any tested compound must therefore possess cAMP activity above that of the rat serum or plasma baseline (termed the limit of detection) in order to have an observable effect in the activity assay.

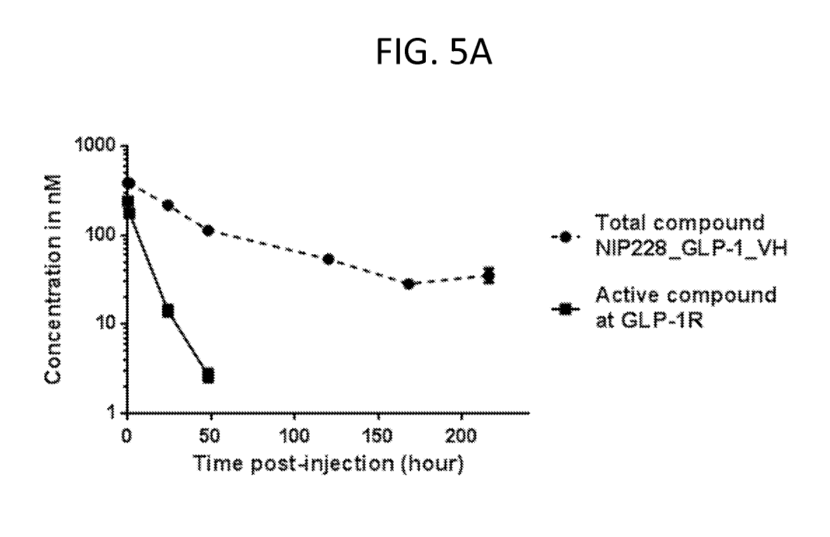

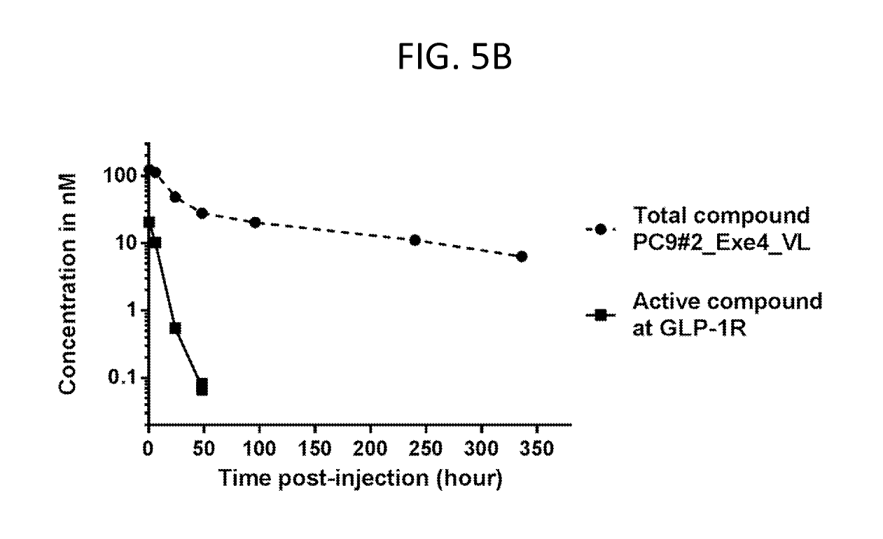

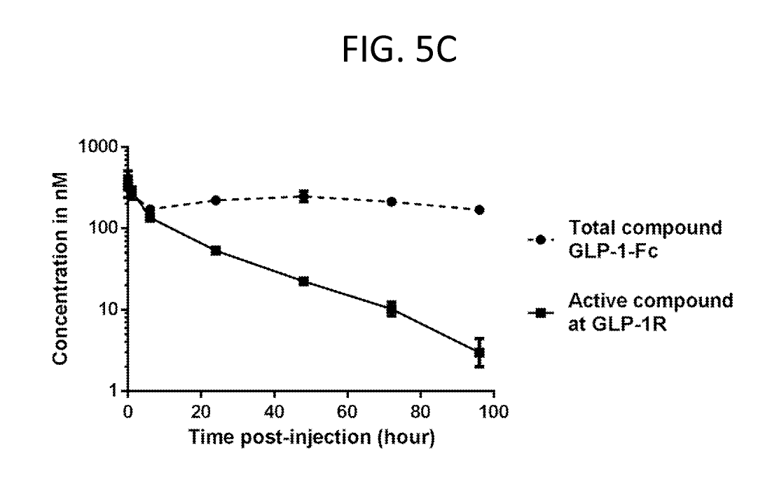

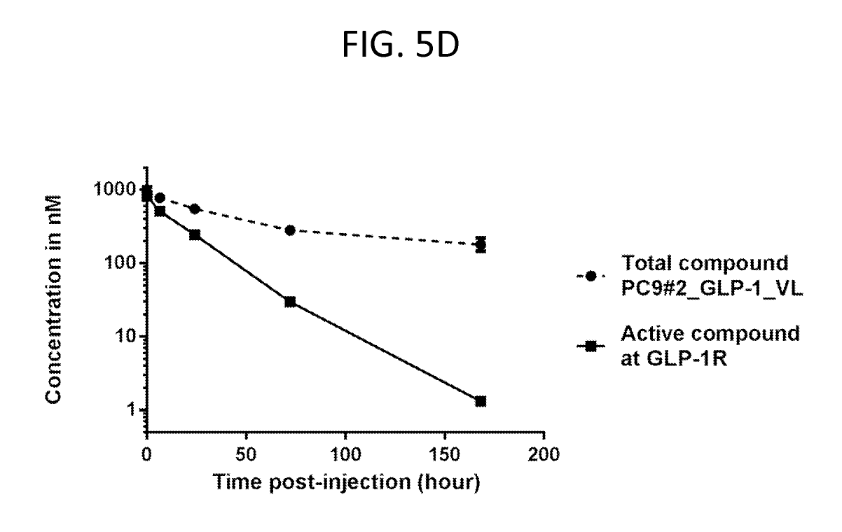

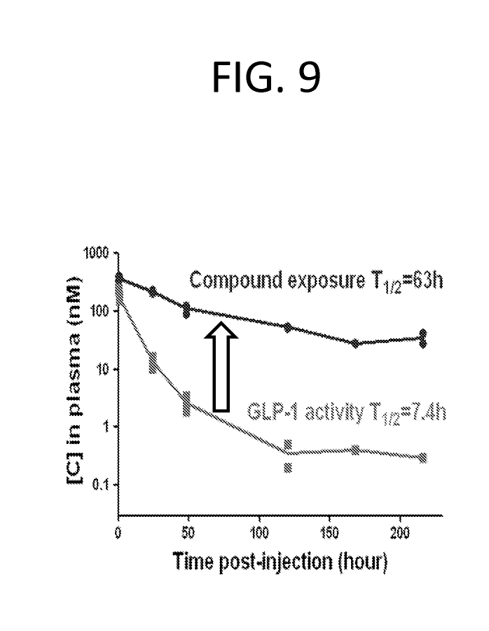

Compound NIP228_GLP-1_VH was injected at 2 mg/kg in three Wistar rats (Charles River) and blood samples for each animal were collected in EDTA tubes containing dPP4 inhibitor at 2 minutes, 1 h, 24 h, 48 h, 120 h, 168 h and 216 h post injection. Total compound and concentration in active GLP-1 compound in rat plasma over time are shown in FIGS. 5A and 9. Concentration of active compound in samples collected after 48 h cannot be determined as they were below the lower limit of quantification of the assay.

Compound PC9#2_Exe4_VL was injected at 1 mg/kg in three CD rats (Charles River) and blood samples were collected in plain tubes containing dPP4 inhibitor at 30 minutes, 6 h, 24 h, 48 h, 96 h, 240 h and 336 h post injection. Serum samples were prepared by leaving the tubes on the bench for 30 minutes followed by 2 minutes centrifugation at 13000 rpm. Exposure and concentration in active GLP-1 compound in rat serum over time are shown in FIG. 5B. Concentration of active compound in samples collected after 48 h cannot be accurately determined as they were below the lower limit of quantification of the assay.

In vivo half-life in rat of NIP228_GLP-1_VH and PC9#2_Exe4_VL for both exposure and active compound at GLP-1 receptor are presented in Table 9.

TABLE 9
Compound and active GLP-1 in vivo half-life in rat
of existing GLP-1 analogues in antibody fusion

| | | | Active |
|---|---|---|---|
| | | Compound | GLP-1 in |
| | Experimental | in vivo | vivo half |
| Compound | design | half life (h) | life (h) |
| NIP228_GLP-1_VH | 2 mg/kg IV in | 63 | 7.4 |
| | Wistar rats | | |
| PC9_2_Exe4_VL | 1 mg/kg IV in | 88 | 5.7 |
| | CD rats | | |

For both NIP228_GLP-1_VH and PC9#2_Exe4_VL compounds, activity at GLP-1 receptor is loss much quicker than the compound itself demonstrating in vivo peptide instability.

Compounds GLP-1-Fc and PC9#2_GLP-1_VL were injected intravenously to healthy C57/B6 mice (7-8 weeks old, females, Charles River) and plasma samples were collected at several timepoints. Compound concentration in plasma (exposure) and concentration in active compound for GLP-1 activity were determined for each sample as described above.

GLP-1-Fc was injected at 1 mg/kg. Groups of three mice were sacrificed at each of the following time points: pre-injection, 2 minutes, 1 h, 6 h, 24 h, 48 h, 72 h and 96 h post injection. Blood for each animal were collected in EDTA tubes containing dPP4 inhibitor. Plasma samples were then centrifuged at 14000 rpm for 5 min at 4° C. and stored at −80° C. pending analysis.

PC9#2_GLP-1_VL was injected at 5 mg/kg and mice were sacrificed at each of the following time points: pre-injection, 5 minutes, 6.5 h, 24 h, 72 h and 168 h post injection. Samples were treated as described above.

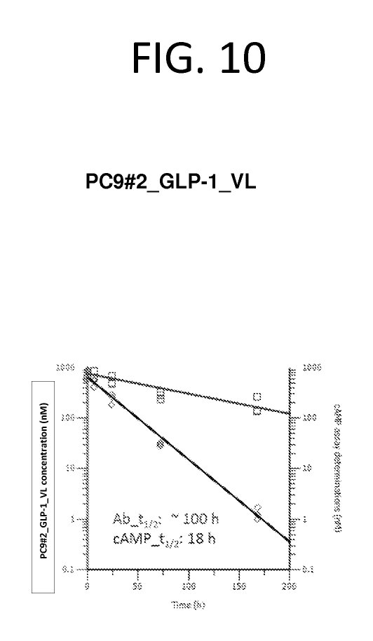

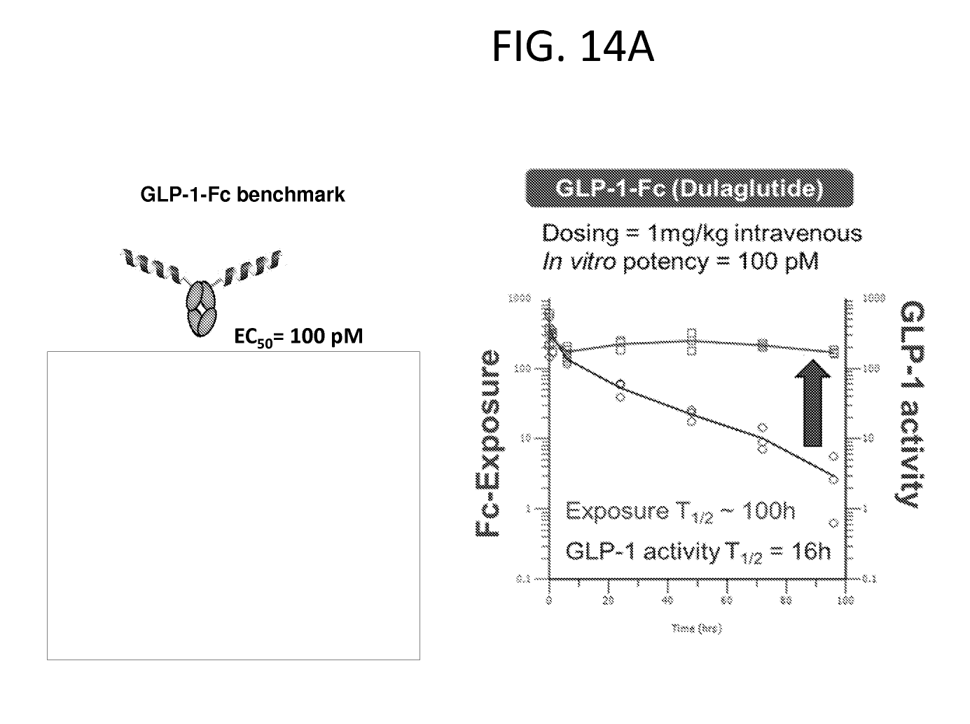

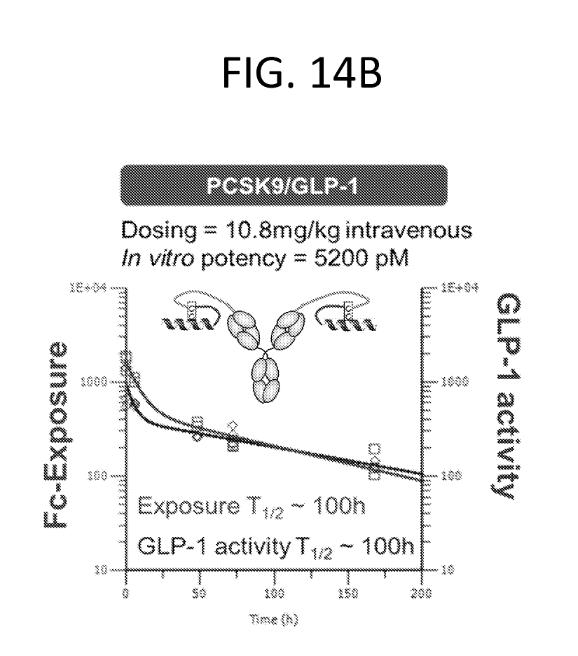

Exposure and concentration in active GLP-1 compound in mouse plasma over time for GLP-1-Fc are shown in FIGS. 5C and 14A and PC9#2_GLP-1_VL are shown in FIGS. 5D and 10, respectively.

In vivo half-life in mice of GLP-1-Fc and PC9#2_GLP-1_VL for both exposure and active GLP-1 are presented in Table 10.

TABLE 10
Compound and active GLP-1 in vivo half-life in mice
of existing GLP-1 analogues in antibody or Fc fusion

| | | Compound | Active GLP- |
|---|---|---|---|
| | Experimental | in vivo | 1 in vivo |
| Compound | design | half life (h) | half life (h) |
| GLP-1-Fc | 1 mg/kg in C57/B6 mice | ~100 | 16 |
| PC9#2_GLP- | 5 mg/kg in C57/B6 mice | ~100 | 18 |
| 1_VL | | | |

As observed for NIP228_GLP-1_VH and PC9#2_Exe4_VL in rat, activity at GLP-1 receptor for both GLP-1-Fc and PC9#2_GLP-1_VL following injection in mice is loss quicker than the compound itself, demonstrating in vivo peptide instability.

Quick inactivation for both GLP-1 analogues of SEQ ID NO:28 and SEQ ID NO:12 will impact efficient glucose control for PCSK9/GLP-1 fusion molecules and GLP-1 analogue peptides with better in vivo stability need to be engineered.

### Example 5

### Affinity for huPCSK9

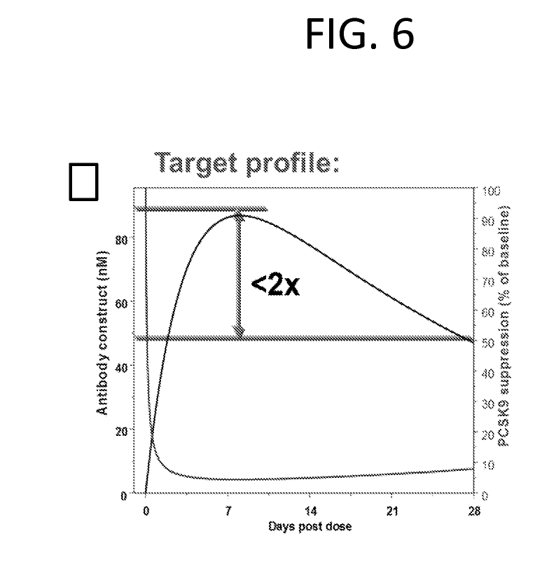

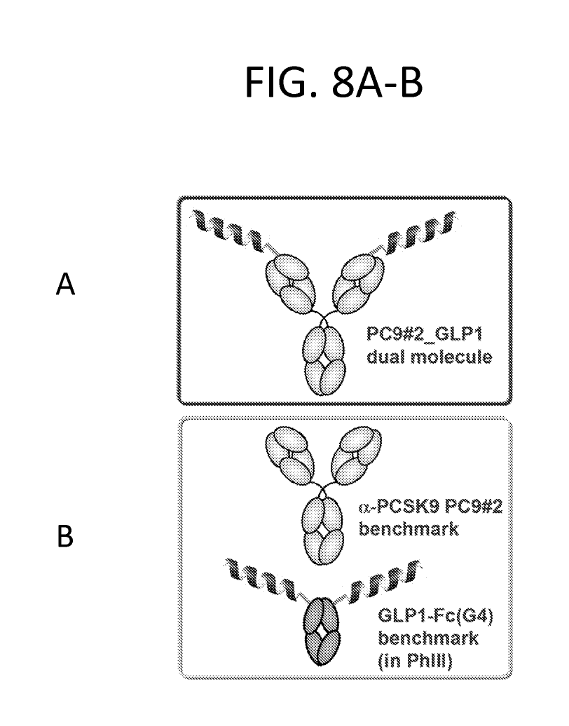

The PCSK9 antibody was used as a benchmark control. Data are presented in Table 11 and a visual representation of the PC9#2_GLP1 molecule and the anti-PC9#2 antibody used as a benchmark control are shown in FIG. 8. A visualization of a potential target profile to guide PCSK9 affinity and GLP-1 potency is provided in FIG. 6.

Association (ka or kon), dissociation (kd or koff) and equilibrium dissociation constants (KD) for PCSK9 binding were determined at 25° C. by Surface Plasmon Resonance (SPR) using the Biacore 2000 biosensor (GE Healthcare).

A Protein G surface was first created on a CM5 sensor chip (GE Healthcare). Human antibodies were then captured on the chip surface before injecting different concentrations of human, cynomolgus or rat PCSK9. Global dissociation rates were first calculated followed by global on-rate calculations both using a 1:1 binding kinetics model.

TABLE 11
Affinity for huPCSK9 (Biacore)

| Test Compound | Kd (nM) | ₖₒₙ (M⁻¹ · s⁻¹) | k_{off} (s⁻¹) |
|---|---|---|---|
| PC9#2 (SEQ ID NO. | 7 | 7.8E+04 | 5.5E−04 |
| 8 & 9) | | | |
| PC9#2_GLP1 (SEQ | 19 | 2.7E+04 | 5.0E−04 |
| ID NO: 8, 9, 4, & 28) | | | |
| (with the light chain | | | |
| fusion SEQ ID NO: | | | |
| 43) | | | |
| x change | 2.7 | 2.9 | 0.9 |

This demonstrates that the fusion is only marginally impacting PCSK9 binding.

Additionally, the dual action fusion molecule HS9_DSB7 was tested in a Biacore assay as described in Example 17 to determine its affinity for human PCSK9. The PCSK9 antibody alone (PC9_2_FG_HS#9 of SEQ ID 1 and 2 for heavy and light chain respectively) was used as a benchmark control. Table 12 provides the data. This data demonstrates that the fusion is only marginally impacting PCSK9 binding across species (human (Hu), cynomolgus monkeys (Cy) and rat).

TABLE 12
Affinity for huPCSK9, CyPCSK9, and RatPCSK9 (Biacore)

| | Hu | | | Cy | | | Rat | | |
|---|---|---|---|---|---|---|---|---|---|
| | ka | kd | KD | ka | kd | KD | ka | kd | KD |
| Summary | (M⁻¹ · s⁻¹) | (s⁻¹) | (M) | (M⁻¹ · s⁻¹) | (s⁻¹) | (M) | (M⁻¹ · s⁻¹) | (s⁻¹) | (M) |
| PC9_2_FG_HS #9 | 3.47E+05 | 3.65E−04 | 1.05E−09 | 3.19E+05 | 3.74E−04 | 1.17E−09 | 9.69E+05 | 3.45E−04 | 3.56E−10 |
| HS9_DSB7 | 1.46E+05 | 2.87E−04 | 1.97E−09 | 9.49E+04 | 2.84E−04 | 2.99E−09 | 5.07E+05 | 2.67E−04 | 5.27E−10 |

### Example 6

### GLP-1 Potency in cAMP Cell-Based Assay

PC9#2_GLP-1 was tested in a cAMP cell-based assay to determine its potency as described in Example 3. The GLP1 peptide alone was used as a control and GLP-1Fc was used as a benchmark. Data are presented in Table 13 and a visual representation of the PC9#2_GLP1 molecule and the GLP-1 Fc used as a benchmark control are shown in FIG. 8.

TABLE 13
GLP-1 Potency in cAMP Cell-Based Assay

| Test Compound | EC50 (pm) | x change |
|---|---|---|
| PC9#2_GLP1 (SEQ | 290 | 2.4 |
| ID NO: 8, 9, 4, & 28) | | |
| GLP1-Fc ( | 120 | 1 |
| GLP1 peptide (SEQ | 15 | 0.1 |
| ID NO: 29) | | |

This data demonstrates that there was no significant loss of GLP-1 potency compared to the benchmark molecule GLP-1-Fc following the fusion of the GLP-1 analogue peptide to the light chain of the anti-PCSK9 antibody PC9#2.

### Example 7

### Dulaglutide: A Benchmark Molecule

FIG. 14A provides stability in rat of GLP-1-Fc benchmark. Benchmark molecule is a GLP-1 moiety fusion to an Fc portion of an antibody, as shown in FIG. 14A.

Compounds GLP-1-Fc was injected intravenously to healthy C57/B6 mice (7-8 weeks old, females, Charles River) and plasma samples were collected at several timepoints. Compound concentration in plasma (exposure) and concentration in active compound for GLP-1 activity were determined for each sample as described in Example 4.

GLP-1-Fc was injected at 1 mg/kg. Groups of three mice were sacrificed at each of the following time points: pre-injection, 2 minutes, 1 h, 6 h, 24 h, 48 h, 72 h and 96 h post injection. Blood for each animal were collected in EDTA tubes containing dPP4 inhibitor. Plasma samples were then centrifuged at 14000 rpm for 5 min at 4° C. and stored at −80° C. pending analysis.

GLP-1 activity is lost at a quicker rate than the compound, demonstrating peptide instability. The line with the squares corresponds to the serum concentration of GLP-1-Fc and the line with the circles corresponds to the activity of GLP-1-Fc for the same samples. See FIG. 14A.

### Example 8

### Fusion Molecules with Enhanced In Vivo Stability Profiles

A) Evaluating GLP-1 Analogue Peptides in Antibody Fusion with Enhanced In Vivo Stability Profiles

To improve in vivo peptide stability of antibody fusion molecules, steric hindrance was engineered around the peptide to protect it from degradation. This was done either by introducing a bulky sugar motif or by engineering an inter molecular disulphide bridge.

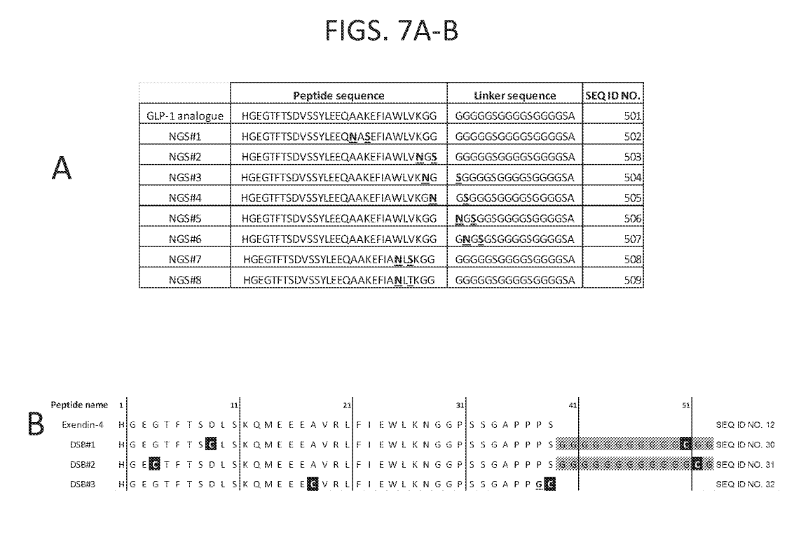

It has been demonstrated that addition of N-glycosylation consensus motifs can increase in vivo stability and duration of action of proteins (Elliott S. et al., Nat. Biotech., 2003, 21, 414-421). GLP-1 analogue peptides incorporating an extra N-glycosylation motif, NxS or NxT where x can be any amino acid except proline, at the C-terminus of the peptide or in the linker between the peptide and the antibody have been engineered in fusion with the light chain of anti-PCSK9 antibody PC9#2 (antibody light chain, SEQ ID NO: 9). Peptide and linker amino acid sequence for eight of those compounds (named NGS for N-Glysosylation Site) are shown in FIG. 7A. Amino acid changes to generate the glycosylation motif are shown in bold underlined.

Among those eight compounds, only PC9_2_GLP-1_NGS#7 shows a high glycosylation yield by SDS-PAGE. This was detected by an increase in the molecular weight of the light chain compared to the control compound without the glycosylation consensus sequence, and with no visible lower molecular band corresponding to the non-glycosylated light chain product. Glycosylation for PC9_2_GLP-1_NGS#7 was further confirmed by ESI mass spectrometry.

It has also been shown that introducing an inter-disulphide bond could be a successful approach to improve in vivo stability of GLP-1 analogues as free peptide (Li Y. et al., Peptides, 2011, 21, 1303-1312).

Exendin-4 peptide variants incorporating two cysteine residues to form the disulphide bridge as well as, if appropriate, a glycine C-terminus cap in order to facilitate the bonding were fused to the light chain of anti-PCSK9 antibody PC9#2 (SEQ ID NO: 9). Peptide amino acid sequence for the three compounds initially generated (named DSB for DiSulphide Bridge) are shown in FIG. 7B (as SEQ ID NOs: 30-32). Cysteine residues are shown in black, other mutated residues are shown as underline and additional glycine residues at the C-terminus cap are shown in grey.

For DSB#1 variant, the first cysteine was engineered in position 9 instead of an aspartic acid and using a C-terminus cap, incorporating the second cysteine, of sequence:

(SEQ ID NO: 401)
GGGGGGGGGGGCGG.

For DSB#2 variant, the first cysteine was engineered in position 4 instead of a glycine and using a C-terminus cap, incorporating the second cysteine, of sequence:

(SEQ ID NO: 402)
GGGGGGGGGGGGCG.

For DSB#3 variant, the first cysteine was engineered in position 18 instead of an alanine but no C-terminus cap was used. The second cysteine was introduced at position 39 of the Exendin-4 sequence instead of a serine. Proline 38 was also changed into a glycine to generate more flexibility in the tryptophan cage of Exendin-4 in order to facilitate the formation of the disulphide bridge.

PC9_2_Exe4_DSB#2 in light chain fusion does not express significantly in mammalian cells and was not further characterised but sufficient amount of PC9_2_Exe4_DSB#1 and PC9_2_Exe4_DSB#3 were obtained. Integrity and identity of the fusions were confirmed by ESI mass spectrometry before in vivo experiments.

In vivo stability of PC9_2_GLP-1_NGS#7, PC9_2_Exe4_DSB#1 and PC9_2_Exe4_DSB#3 was assessed in mouse by following both compound exposure and concentration in active GLP-1 over time as described in Example 4.

Healthy C57/B6 mice (7-8 weeks old, females, Charles River) received one single intravenous (IV) dose of PC9_2_GLP-1_NGS#7 at 40 mg/kg, PC9_2_Exe4_DSB#1 at 10.8 mg/kg or PC9_2_Exe4_DSB#3 at 5 mg/kg. Groups of three mice were sacrificed at each of the following time points: pre-injection, 5 minutes, 6 h, 24 h, 72 h and 168 h post injection and blood for each animal were collected in EDTA tubes containing dPP4 inhibitor. Plasma samples were then centrifuged at 14000 rpm for 5 min at 4° C. and stored at −80° C. pending analysis.

All three compounds are active at the human GLP-1 receptor in vitro but display different EC₅₀ in the cAMP assay: 1.94⁻⁸ M, 5.25⁻⁹ M and 3.25⁻¹⁰ M for PC9_2_GLP-2_NGS#7, PC9_2_Exe4_DSB#1 and PC9_2_Exe4_DSB#3 respectively. Doses were adjusted as much as possible based on potency to generate a signal above the lower limit of quantification in the cAMP ex-vivo assay in order to calculate concentration in active GLP-1 compound over a significant period of time.

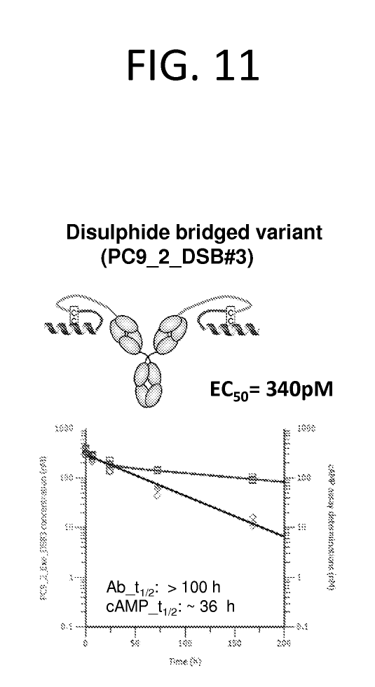

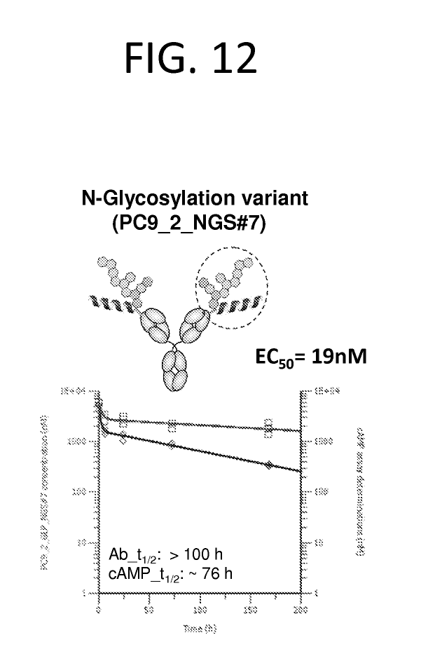

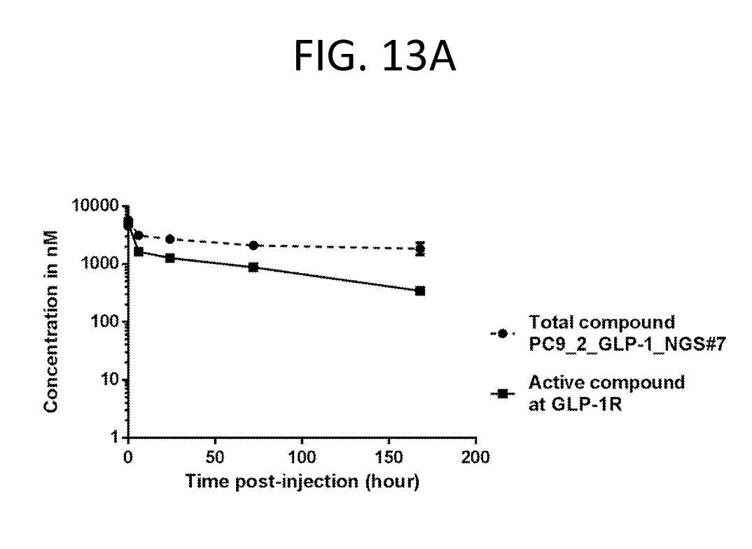

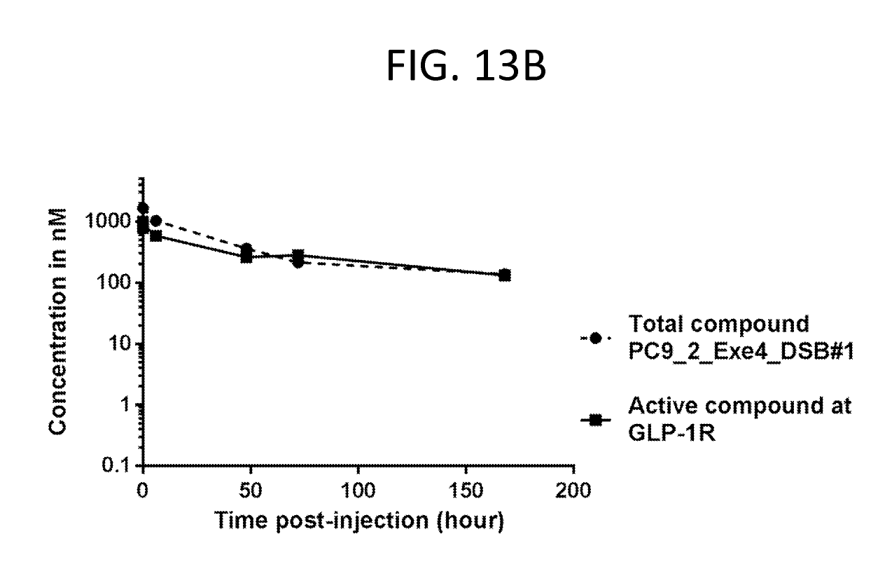

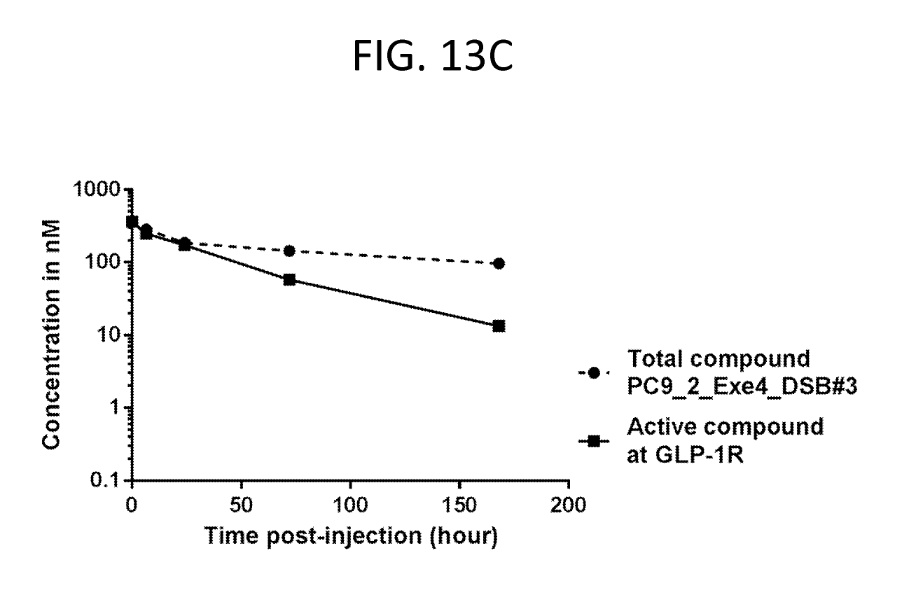

Exposure and concentration in active GLP-1 compound in mouse plasma over time for PC9_2_GLP-2_NGS#7 are shown in FIGS. 12 and 13A, PC9_2_Exe4_DSB#1 are shown in FIGS. 13B and 14B, and PC9_2_Exe4_DSB#3 fusion molecules are shown in FIGS. 11 and 13C, respectively.

In vivo half-life of PC9_2_GLP-1_NGS#7, PC9_2_Exe4_DSB#1 and PC9_2_Exe4_DSB#3 for both exposure and active GLP-1 are presented in Table 14.

TABLE 14
Compound and active GLP-1 in vivo half-life of
engineered GLP-1 analogues in antibody fusion

| | | Compound | Active GLP- |
|---|---|---|---|
| | Experimental | in vivo | 1 in vivo |
| Compound | design | half life (h) | half life (h) |
| PC9_2_GLP- | 40 mg/kg IV in | ~100 | 76 |
| 1_NGS#7 | C57/B6 mice | | |
| PC9_2_Exe4_DSB#l | 10.8 mg/kg IV in | ~100 | ~100 |
| | C57/B6 mice | | |
| PC9_2_Exe4_DSB#3 | 5 mg/kg IV in | ~100 | 36 |
| | C57/B6 mice | | |

Compared to parent molecules NIP228_GLP-1_VH and PC9#2_Exe4_VL (Table 9), all three compounds have improved in vivo stability for GLP-1 activity with half-life of 76 h, around 100 h and 36 h for PC9_2_GLP-1_NGS#7, PC9_2_Exe4_DSB#1 and PC9_2_Exe4_DSB#3 respectively.

Quite interestingly, PC9_2_Exe4_DSB#1 appears fully stable in mice for up to 7 days with no observed loss of GLP-1 activity when compared to compound exposure (FIG. 13B).

Such data are suggesting that generating steric hindrance around GLP-1 analogues can increase in vivo activity half-life of the peptide in antibody fusion.

B) Evaluating Fusion Molecules with Enhanced In Vivo Stability Profiles

The protocol discussed in Example 4 and 8A was followed to administer various compounds to mice and to plot the concentration of the compounds in the plasma over time. Compound potency (EC50) at human GLP-1 receptor was determined using the cAMP assay as described in Example 3.

Fusion molecule optimization was guided through an in vivo PK/stability assessment. As shown in FIGS. 14A, 11 and 12, peptide engineering produced fusion molecules with enhanced in vivo stability profiles. Benefits were seen with disulfide bridge stabilization for which there was longer retention of activity with minimum impact on potency at GLP-1 receptor. NGS#7 also has an improved stability profile but a low potency (19 nM) compared to DSB#3 (340 pM).

FIG. 14A shows a GLP-1-Fc benchmark molecule with an EC₅₀ of 100 pm.

FIG. 11 shows a disulfide bridged variant (PC9_2_DSB#3) (SEQ ID NO: 49 and SEQ ID NO: 8) with an EC₅₀ of 340 pM.

FIG. 12 shows an n-glycosylation variant (PC9_2_NGS#7) (SEQ ID NO: 50 and SEQ ID NO: 8) with an EC₅₀ of 19 nM.

In vivo half-life for those compounds are presented in Table 15.

TABLE 15
Compound and active GLP-1 in vivo half-life of
engineered GLP-1 analogues in antibody fusion

| | | Compound | Active GLP- |
|---|---|---|---|
| | Experimental | in vivo | 1 in vivo |
| Compound | design | half life (h) | half life (h) |
| GLP-1-Fc | 1 mg/kg in | ~100 | 16 |
| | C57/B6 mice | | |
| PC9_2_Exe4_DSB#3 | 5 mg/kg IV in | ~100 | 36 |
| | C57/B6 mice | | |
| PC9_2_GLP- | 40 mg/kg IV in | ~100 | 76 |
| 1_NGS#7 | C57/B6 mice | | |

### Example 9

### A PCSK9/GLP-1 Fusion Demonstrates an Ideal Stability/Activity Profile

The dual action fusion molecule was evaluated for Fc-exposure and GLP-1 activity in a mouse model. Dosing was 10.8 mg/kg intravenously and the in vitro potency was 5200 pM. Protocol was as described in Example 8. The data were compared to the GLP-1 Fc benchmark molecule, with dosing of 1 mg/kg intravenously and an in vitro potency of 100 pM. Similar protocol was here used as described in Example 4. Results are shown in FIGS. 14A (dulaglutide) and 14B (PCSK9/GLP-1 fusion PC9_2_DSB#1) (SEQ ID NO: 48 and SEQ ID NO: 8). There was no loss of GLP-1 activity in the mouse for up to 7 days.

### Example 10

### Production and Purification of Early GLP-1 Analogue Peptide Antibody Fusions with an Intramolecular Disulphide Bridge

Quality of the material post-protein A purification from medium scale batches was poor for PC9_2_Exe4_DSB#1 and PC9_2_Exe4_DSB#3 as lot of aggregates were detected by SEC-HPLC. Preparative Size Exclusion Chromatography using Superdex 200 prep grade columns was then used to further purify the compounds as described in Example 2.

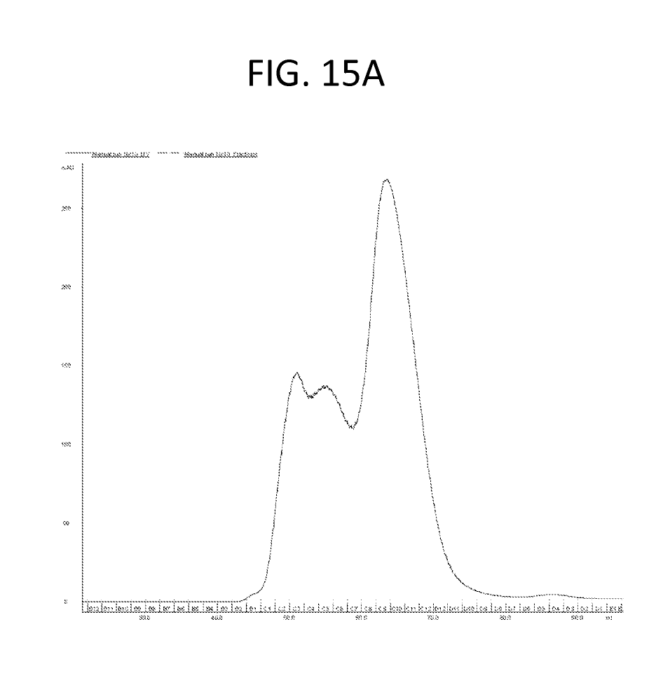

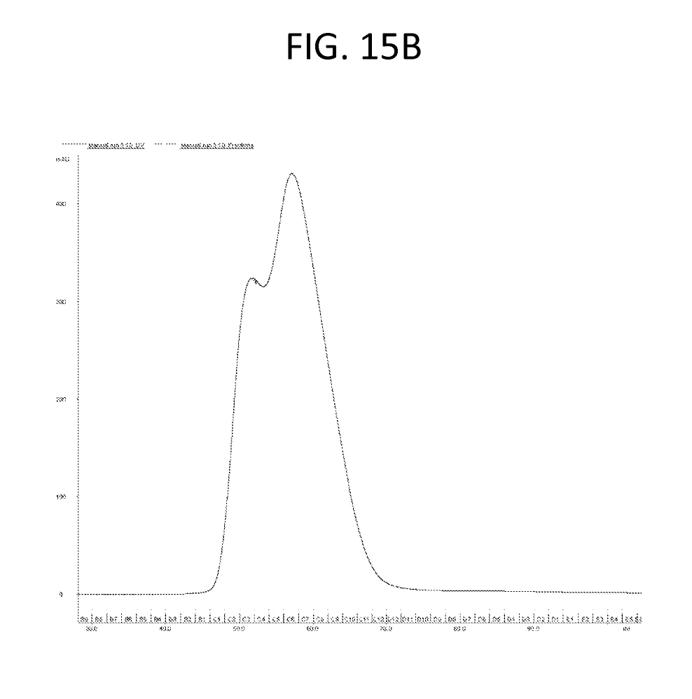

Preparative SEC chromatograms for PC9_2_Exe4_DSB#1 and PC9_2_Exe4_DSB#3 are shown in FIGS. 15A and 15B, respectively. A significant proportion (25-40%) of the material is aggregated as shown by additional peaks at early retention time. Fractions containing the monomeric compound were collected to obtain the material used for in vivo testing. See Example 8 and 9.

Scalability of PC9_2_Exe4_DSB#1 production was further assessed by transiently transfected 48.2L of CHO mammalian cells in wavebags (GE Healthcare). Compound was purified by using a Protein A column followed by two additional purification steps using a mixed-mode resin.

A high level of aggregation (>25%) was detected in the harvest and efficient purification of the monomer product was particularly challenging. Three chromatography steps were required to generate a product at 95.9% purity by SEC-HPLC. This was very detrimental to the purification yield with an overall recovery of around 3.4%. In addition, the titre in harvest was at 104 mg/L which is low compared to monoclonal antibodies using a similar expression system. Production of DSB#1 disulphide bridge GLP-1 analogue in fusion with the light chain of anti-PCSK9 antibody HS9 (SEQ ID NO: 2) gave very similar results.

### Example 11

### Aggregation

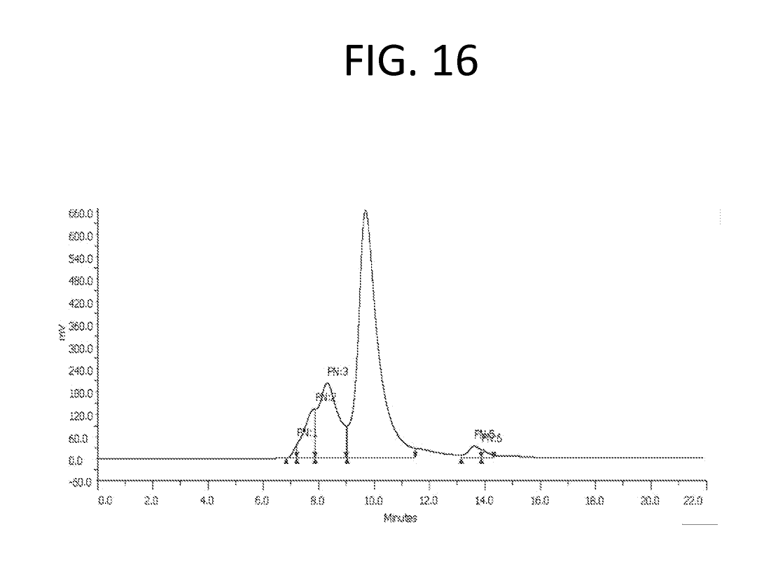

The dual action fusion molecule (PCSK9/GLP-1 fusion PC9_2_DSB#1) (SEQ ID NO: 48) has the potential for aggregation and in some embodiments monomer is desired to be selected. Aggregates, detected during an analytical SEC-HPLC following protein A purification as described in Example 2, are shown in FIG. 16. Thus, in some embodiments additional engineering may be desired to improve the monomeric profile.

### Example 12

### GLP-1 Analogue Peptides in Antibody Fusion with Enhanced Monomeric Profiles

Additional Exendin-4 disulphide bridge peptides (DSB) in fusion with the light chain of PC9#2 (SEQ ID NO: 9) were generated in order to improve the monomeric profile during production of the peptide antibody molecule. Different positions of the cysteine bridge as well as length of the glycine rich C-terminus cap and various glycine point mutations in the C-terminus of the Exendin-4 peptide were engineered in order to facilitate the formation of the disulphide bond and ultimately reduce aggregation during production probably due to disulphide scrambling. Peptide engineering work was guided using the 3-D NMR structure of Exendin-4 (Neidigh, J W et al., Biochemistry, 2001, 40, 13188-200.).

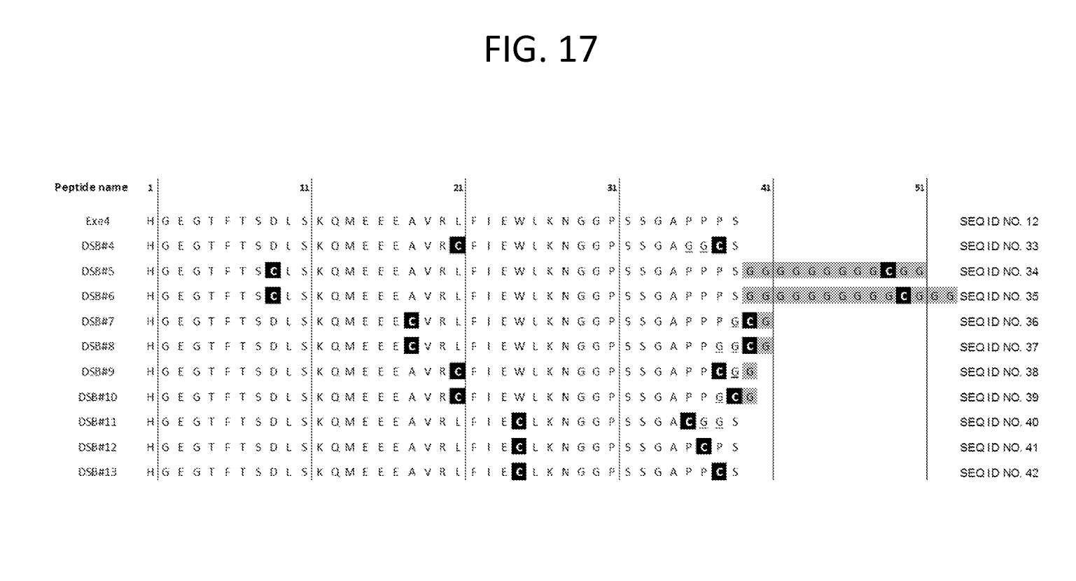

A total of ten DSB peptide anti-PCSK9 fusions were produced at small scale and screened for an improved monomeric profile by SEC-HPLC. Peptide sequences are described in FIG. 17. PC9_2_Exe4_DSB#4 did not significantly express and was not further characterised.

Percentages of aggregate for the nine fusions compared to PC9_2_Exe4_VL and PC9_2_Exe4_DSB#1 determined by analytical SEC-HPLC after protein A purification are described in Table 16.

TABLE 16
Percentage of aggregate post protein A purification
for disulphide bridge exendin-4 variants in fusion with
the light chain of PCSK9 antibody PC9_2

| | | % aggregate by |
|---|---|---|
| # | Compound | analytical SEC |
| 1 | PC9_2_Exe4_VL | 2.9 |
| 2 | PC9_2_Exe4_DSB#1 | 26.5 |
| 3 | PC9_2_Exe4_DSB#5 | 20.5 |
| 4 | PC9_2_Exe4_DSB#6 | 23.3 |
| 5 | PC9_2_Exe4_DSB#7 | 5.1 |
| 6 | PC9_2_Exe4_DSB#8 | 51.1 |
| 7 | PC9_2_Exe4_DSB#9 | 7.8 |
| 8 | PC9_2_Exe4_DSB#10 | 7.3 |
| 9 | PC9_2_Exe4_DSB#11 | 7.6 |
| 10 | PC9_2_Exe4_DSB#12 | 20.1 |
| 11 | PC9_2_Exe4_DSB#13 | 17.1 |

Four compounds among the nine tested achieve a percentage aggregate below 10%: PC9_2_Exe4_DSB#7, PC9_2_Exe4_DSB#9, PC9_2_Exe4_DSB#10 and PC9_2_Exe4_DSB#11. PC9_2_Exe4_DSB#7 has the lowest percentage aggregate at 5.1% compared to 26.5% for the early fusion PC9_2_Exe4_DSB#1. Exendin-4 antibody fusion without a disulphide bridge, PC9_2_Exe4_VL, shows 2.9% aggregate.

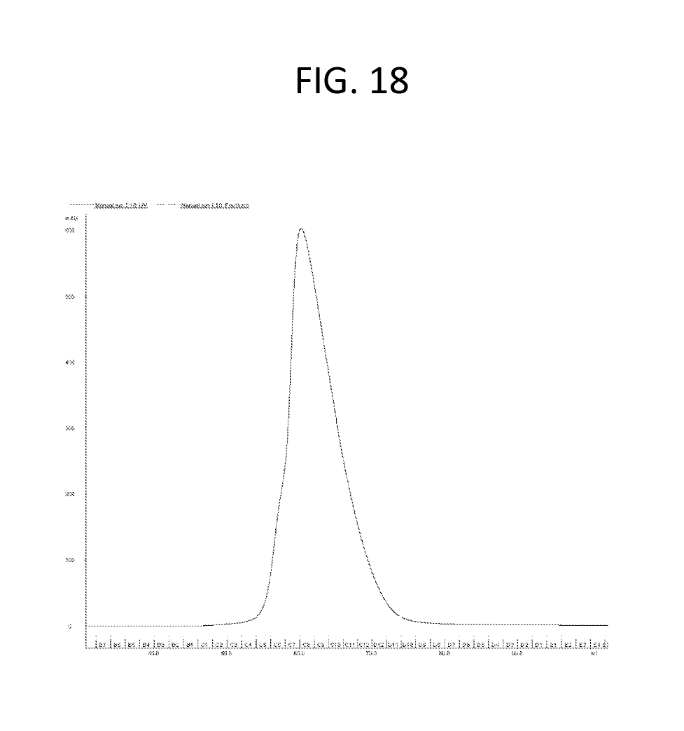

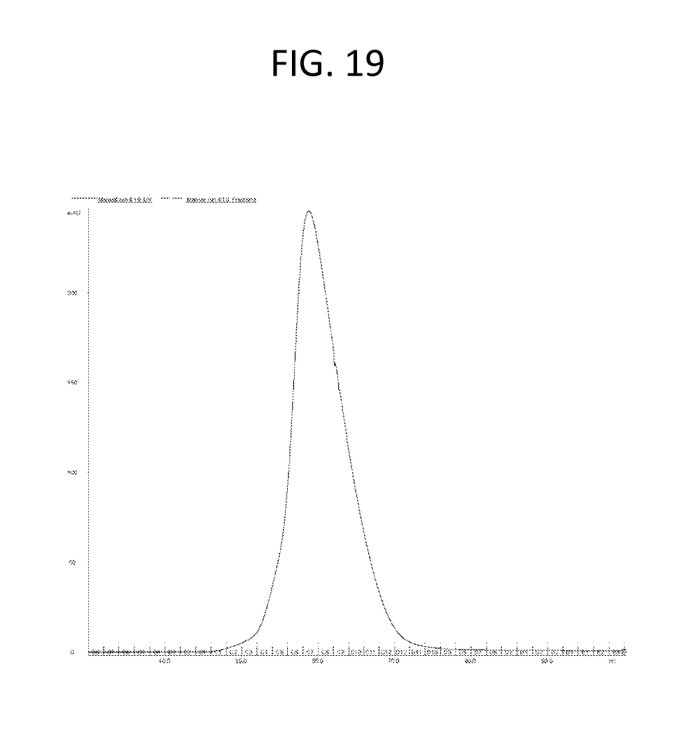

Productions of PC9_2_Exe4_DSB#7 and PC9_2_Exe4_DSB#9 were scaled up to supply material in sufficient quantity to perform in vivo stability experiments. FIGS. 18 and 19 show showing preparative SEC chromatograms of PC9_2_Exe4_DSB#7 and PC9_2_Exe4_DSB#9 respectively following an initial protein A purification as described in Example 2. Unlike PC9_2_Exe4_DSB#1 and PC9_2_Exe4_DSB#3 (FIGS. 15A and B), no significant proportion of aggregates was detected during that purification step.

In order to check that the optimised DSB peptide antibody fusions with an improved monomeric profile do exhibit a superior in vivo stability compared to Exendin-4 antibody fusion, PC9_2_Exe4_DSB#7 and PC9_2_Exe4_DSB#9 were injected intravenously in three CD rats for each compound at 10 mg/kg and 1 mg/kg respectively. Serum samples for each animal were collected at 30 minutes, 6 h, 24 h, 48 h, 96 h, 240 h and 336 h post injection. Compound exposure and concentration in active GLP-1 were measured over time as described in Example 4.

Both compounds are active at the human GLP-1 receptor in the cAMP assay with EC₅₀ of 9.45E-10 M for PC9_2_Exe4_DSB#7 and 1.24E-10 M for PC9_2_Exe4_DSB#9. PC9_2_Exe4_DSB#7 is around eight time less potent than PC9_2_Exe4_DSB#9 and as thus been injected at ten time higher a dose to generate a signal above the lower limit of quantification in the cAMP ex-vivo assay over a significant period of time.

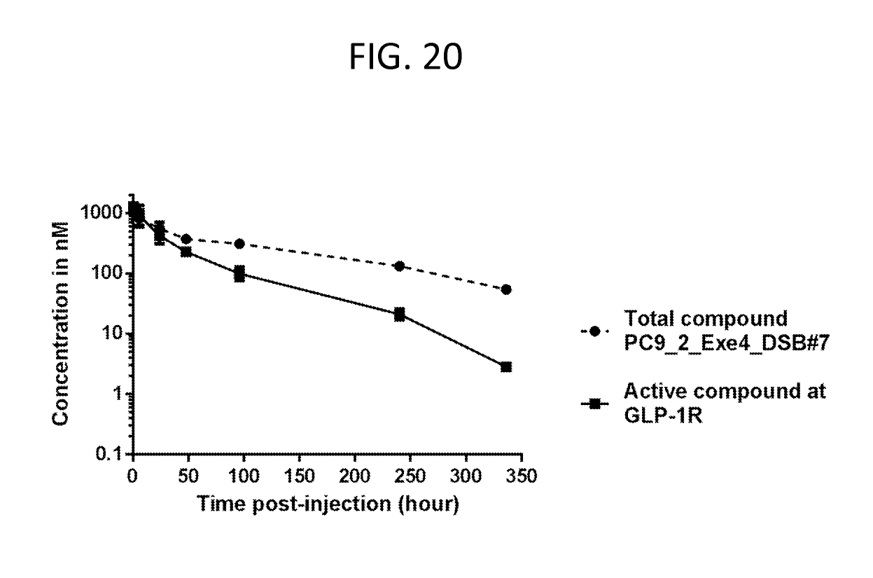

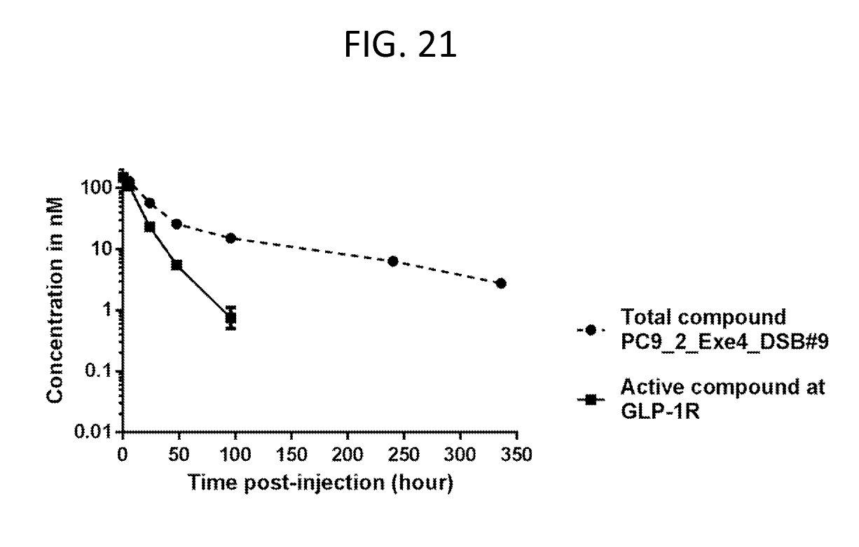

Exposure and concentration in active GLP-1 compound in rat serum over time for PC9_2_Exe4_DSB#7 and PC9_2_Exe4_DSB#9 fusion molecules are shown in FIGS. 20 and 21, respectively. Concentrations in active GLP-1 were normalised to the exposure at the first time point (30 min) to simplify the analysis.

PC9_2_Exe4_DSB#7 and PC9_2_Exe4_DSB#9 have a half-life for GLP-1 activity of 44.2 h and 12.5 h respectively compared to 5.7 h for the parent Exendin-4 fusion molecule PC9_2_Exe4_VL (see Table 9)). Concentrations of active compound for PC9_2_Exe4_DSB#9 samples collected after 96 h cannot be determined as they were below the lower limit of quantification of the assay.

Those data are demonstrating that both PC9_2_Exe4_DSB#7 and PC9_2_Exe4_DSB#9 have an improved in vivo activity half-life of the GLP-1 analogue peptide when in antibody fusion compared to the parent fusion molecule.

As described above peptide engineering can manage the aggregation profile. Mutations were made in the position of the cysteine bridge in the peptide and the composition and length of the peptide/C terminus of the GLP-1 moiety.

The protocol was as described above.

PCSK9/GLP-1 fusion (PC9_2_DSB#7) (SEQ ID NO: 51 and SEQ ID NO: 8) achieved >90% monomer by SEC-HLPC in small-scale batch. Aggregation was still detected in medium scale batch but to a much lower extended than PCSK9/GLP-1 fusion PC9_2_DSB#1 (SEQ ID NO: 48 and SEQ ID NO: 8).

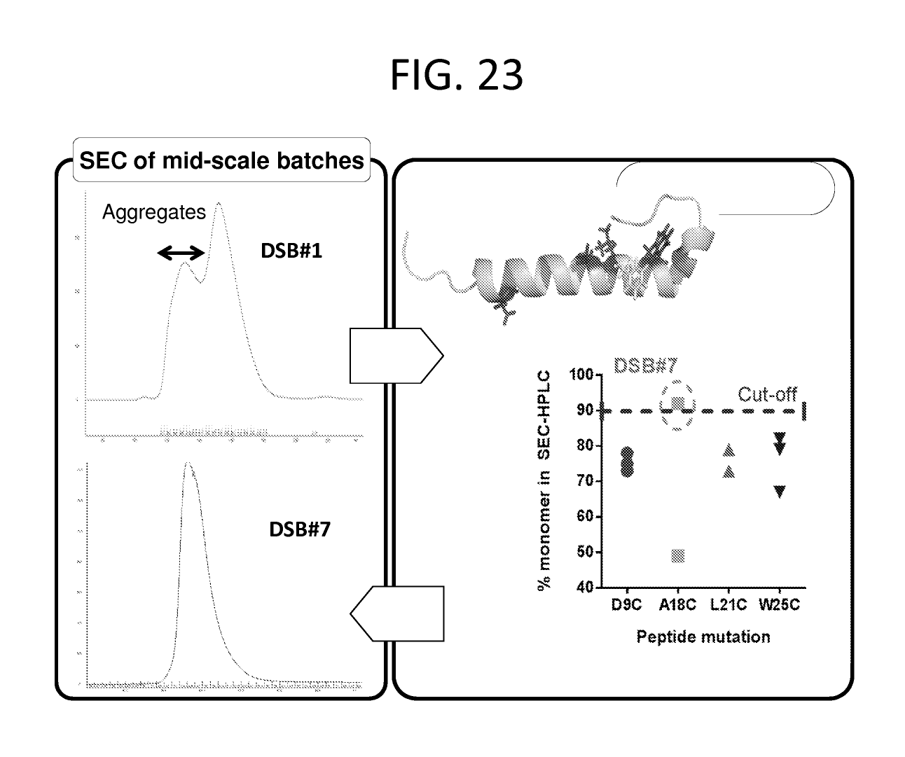

Results are presented in FIGS. 15A, 18, 23 and Table 16.

### Example 13

### Pharmacokinetics and Pharmacodynamics Modeling of GLP-1 Analogue Peptides in Fusion with Anti-PCSK9 Antibodies

To guide the design of PCSK9/GLP-1 fusion molecules, a pharmacokinetic (PK)-pharmacodynamic (PD) model has been developed using prior data on the relationship between PCSK9 suppression and affinity of anti-PCSK9 antibodies tested in the clinic as well as data on the approved GLP-1 receptor agonist molecules Liraglutide and Dulaglutide.

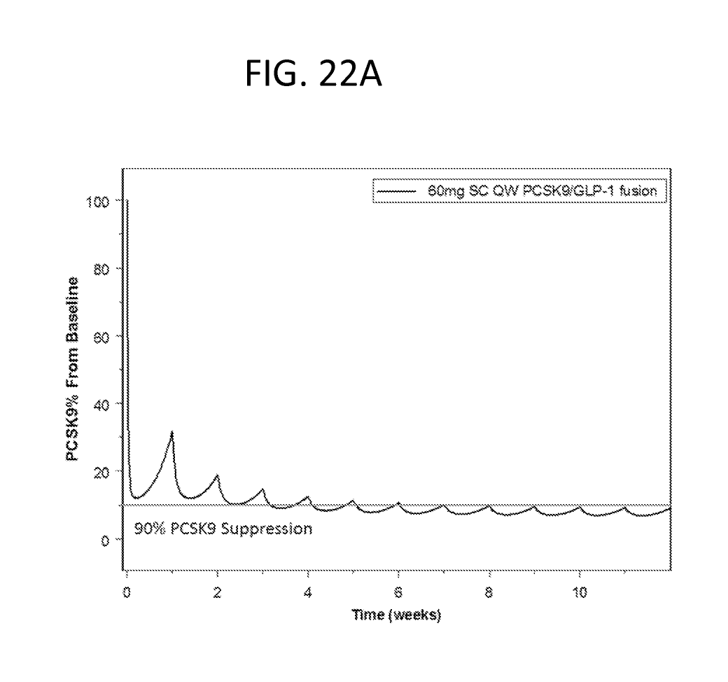

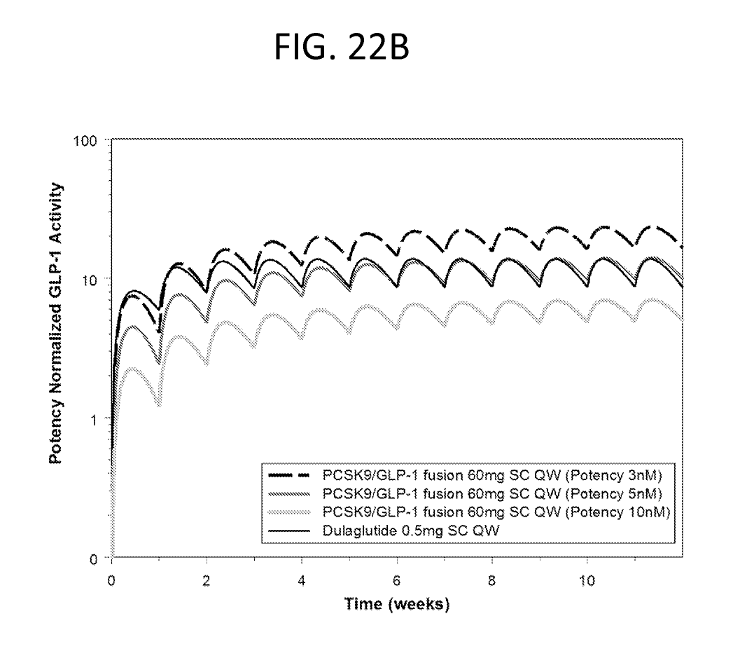

The potency of GLP-1 analogue peptide in fusion with anti-PCSK9 antibody was scanned to identify the optimum range that would result in comparable GLP-1 activity to marketed drugs using a dose able to generate sufficient PCSK9 suppression. Simulations were performed using the pharmacokinetics, plasma protein binding, and receptor affinity properties of Dulaglutide to obtain the potency-normalised GLP-1 activity over time of that compound. For PCSK9/GLP-1 fusion molecule, the PK properties are assumed to be those of a typical human antibody directed towards PCSK9 and the information was derived from compounds in the clinic. These simulations indicate that a 60 mg subcutaneous weekly dose of PCSK9/GLP-1 fusions with an affinity of 3.9 nM for human PCSK9 should result in greater than 90% PCSK9 suppression over the dosing period at steady-state (FIG. 22A).

Using that dosing regimen, simulations indicate that potency of PCSK9/GLP-1 fusion molecules at human GLP-1 receptor should be within 3-5 nM in order to achieve similar GLP-1 activity compared to existing molecules (FIG. 22B). Potency of Dulaglutide in those simulations was set-up at 80 pM suggesting that potency of PCSK9/GLP1 fusion molecules need to be around 30 to 60 fold lower than Dulaglutide in order to manage nausea side effect associated with GLP-1 receptor agonist molecules at the dose required to efficiently suppress PCSK9.

### Example 14

### GLP-1 Analogue Peptides with Reduced Potency at the Human GLP-1 Receptor

Methods for reducing potency of the GLP-1 peptide or GLP-1 analogues are well known in the art as for instance mutating or introducing non-natural amino acids at key residues in the peptide. Critical residues for binding of GLP-1 peptide to the receptor and activity were notably identified by alanine scanning (Adelhorst, K. et al., J. Bio. Chem., 1994, 269, 6276-6278).

To further demonstrate the feasibility to reduce potency at GLP-1 receptor, a panel of GLP-1 analogues with a mutation or non-natural amino acids in position 2 or 3, using the 7-36 GLP-1 sequence, were synthetized as free peptide and tested for activity at the human GLP-1 receptor in the cAMP in vitro assay as described in Example 3. Data are summarised in Table 17 below. Aib is for 2-aminoisobutyric acid and Orn for ornithine.

TABLE 17
Potency at human GLP-1R in the cAMP
assay of GLP-1 point mutant peptides

| | | | | Fold difference |
|---|---|---|---|---|
| | | | Potency at huGLP-1R | in EC50 vs |
| # | Peptide | Modification | in cAMP assay (nM) | GLP-1 |
| 1 | GLP-1 | / | 0.10 | / |
| 2 | g769 | E3D | 0.10 | 1 |
| 3 | g766 | A2Aib | 0.11 | 1.1 |
| 4 | g770 | A2P | 0.15 | 1.5 |
| 5 | g767 | E3Q | 0.28 | 2.8 |
| 6 | g762 | A2S | 0.29 | 2.9 |
| 7 | g755 | A2H | 0.32 | 3.2 |
| 8 | g752 | A2V | 0.56 | 5.6 |
| 9 | g749 | A2G | 0.63 | 6.3 |
| 10 | g768 | E3N | 0.68 | 6.8 |
| 11 | g751 | A2I | 1.78 | 17.8 |
| 12 | g765 | A2Q | 2.98 | 29.8 |
| 13 | g763 | A2T | 4.30 | 43 |
| 14 | g753 | A2F | 4.30 | 43 |
| 15 | g756 | A2W | 5.46 | 54.6 |
| 16 | g754 | A2Y | 14.4 | 144 |
| 17 | g764 | A2N | 21.0 | 210 |
| 18 | g750 | A2L | 80.0 | 800 |
| 19 | g761 | A2Orn | 86.4 | 864 |
| 20 | g757 | A2E | 103.3 | 1033 |
| 21 | g758 | A2D | >100 | >1000 |
| 22 | g760 | A2R | >80 | >800 |
| 23 | g759 | A2K | >100 | >1000 |

Those data demonstrate that EC₅₀ of GLP-1 analogue peptides can be modulated to achieve a defined potency reduction.

### Example 15

### GLP-1 Analogue Peptides in Antibody Fusion with Reduced Potency at the Human GLP-1 Receptor

To reduce the potency of DSB#7 GLP-1 analogue peptide (SEQ ID NO: 13) in fusion with the light chain of the anti-PCSK9 antibody HS9 (SEQ ID NO: 2), point mutations were introduced at position G2, E15, V19, 123 or L26 in the peptide using standard molecular biology techniques. A total of 28 mutants of the peptide antibody fusion molecule were produced and tested for activity at the human GLP-1 receptor using the cAMP assay as described in Example 3. List of the compounds and activity data are presented in Table 18.

Five out of the twenty-eight constructs are inactive. The others compounds display very diverse potency at human GLP1 receptor ranging from 400 pM for HS9_DSB7_V19T to almost 6 uM for HS9_DSB7_L26Q. In addition some compounds, as HS9_DSB7_G2Y or HS9_DSB7_L26Q, are partial agonists: they do not provide the same level of activation at saturating dose compared to GLP-1 peptide.

Based on PKPD modeling in Example 13 the dual activity anti-PCSK9 antibody GLP-1 receptor agonist molecules need to have a potency reduction at human GLP-1 receptor compared to the GLP-1-Fc benchmark of around 30 to 60 fold in order to manage nausea at the dose required to efficiently suppress PCSK9 antigen.

Four peptide antibody fusions (HS9_DSB7_G2V, HS9_DSB7_E15A, HS9_DSB7_V19A and HS9_DSB7_L26I) display a potency between 700 pM and 1.4 nM corresponding to a 25 to 50 fold loss compared to the benchmark GLP1-Fc. All those four compounds are full agonists at human GLP-1 receptor with a percentage of maximum activation greater than 90% compared to the GLP-1 peptide.

### Example 16

### Characterisation of GLP-1 Analogue Peptides in Fusion with Anti-PCSK9 Antibody

A) Developability Assessment of GLP-1 Analogue Peptides in Fusion with Anti-PCSK9 Antibody

The four selected peptide antibody fusions at the desired human GLP-1R potency (HS9_DSB7_G2V, HS9_DSB7_EISA, HS9_DSB7_V19A and HS9_DSB7_L26I) were produced in large scale to support further characterisation. To assess propensity of the compounds to aggregate during production, post protein A purification samples were tested by analytical SEC-HPLC. Data are summarised in Table 19.

TABLE 19
SEC-HPLC analysis of peptide/antibody lead
molecules following Protein A purification

| # | Compound | % Aggregate | % Monomer | % Truncate |
|---|---|---|---|---|
| 1 | HS9_DSB7_G2V | 25.08 | 74.64 | 0.28 |
| 2 | HS9_DSB7_E15A | 21.04 | 78.6 | 0.36 |
| 3 | HS9_DSB7_V19A | 4.33 | 95.43 | 0.24 |
| 4 | HS9_DSB7_L26I | 5.96 | 93.81 | 0.23 |

Only two out of the four fusions (HS9_DSB7_V19A and HS9_DSB7_L26I) achieve a percentage monomer greater than 90% post protein A purification. HS9_DSB7_V19A presents the best profile with more than 95% monomer. HS9_DSB7_G2V and HS9_DSB7_E15A are significantly prone to aggregation during production with percentage aggregate greater than 20% compared to less than 5% for HS9_DSB7_V19A.

Purified compounds were concentrated using centrifugal spin concentrators with a molecular weight cut-off of 30 kDa to achieve a target concentration of 50 mg/mL in default formulation buffer. Concentration of HS9_DSB7_G2V and HS9_DSB7_E15A was stopped at 38.7 and 33.7 mg/mL respectively as it was noticed that further volume reduction leads to a drop in protein concentration. No such issue was observed during the concentration step of HS9_DSB7_V19A and HS9_DSB7_L26I.

Samples were then incubated at 5° C. or 40° C. for 4 weeks followed by analytical SEC-HPLC in order to assess storage stability. Results are summarized in Table 20.

TABLE 20
Purity and aggregation parameters for purified peptide/antibody lead
molecules after 4 weeks incubation at 5° C. or 40° C.

| | | | Purity after | Purity after | Aggregation | Aggregation |
|---|---|---|---|---|---|---|
| | | [C] | 4 weeks at | 4 weeks at | rate per month | rate per month |
| # | Compound | mg/mL | 5° C. (%) | 40° C. (%) | at 5° C. (%) | at 40° C. (%) |
| 1 | HS9_DSB7_G2V | 38.7 * | 88.6 | 77.9 | 1.4 | 9.2 |
| 2 | HS9_DSB7_E15A | 33.7 * | 93.9 | 80.8 | 0.81 | 13.2 |
| 3 | HS9_DSB7_V19A | 52 | 98.7 | 90.1 | 0.3 | 6.3 |
| 4 | HS9_DSB7_L26I | 47.2 | 97 | 87.6 | 0.32 | 7.1 |

Both HS9_DSB7_V19A and HS9_DSB7_L26I display better stability parameters than HS9_DSB7_G2V and HS9_DSB7_E15A. For instance, the first two have an aggregation rate per month at 5° C. of around 0.3% compared to 0.8 and 1.4% for HS9_DSB7_E15A and HS9_DSB7_G2V respectively.

B) Single Intravenous Dose Pharmacokinetics in Rat of GLP-1 Analogue Peptides in Fusion with Anti-PCSK9 Antibody

Pharmacokinetic profile of the four selected peptide antibody fusions at the desired human GLP-1R potency was assessed as described in Example 4 following a single intravenous bolus in three CD rats at 60, 53, 58.5 and 60 mg/kg for HS9_DSB7_G2V, HS9_DSB7_E15A, HS9_DSB7_V19A and HS9_DSB7_L26I respectively. A high dose of the compound (above 50 mg/kg) was used to saturate the PCSK9 sink for a significant period of time to determine PK parameters during the linear phase, without any target mediated drug disposition component.

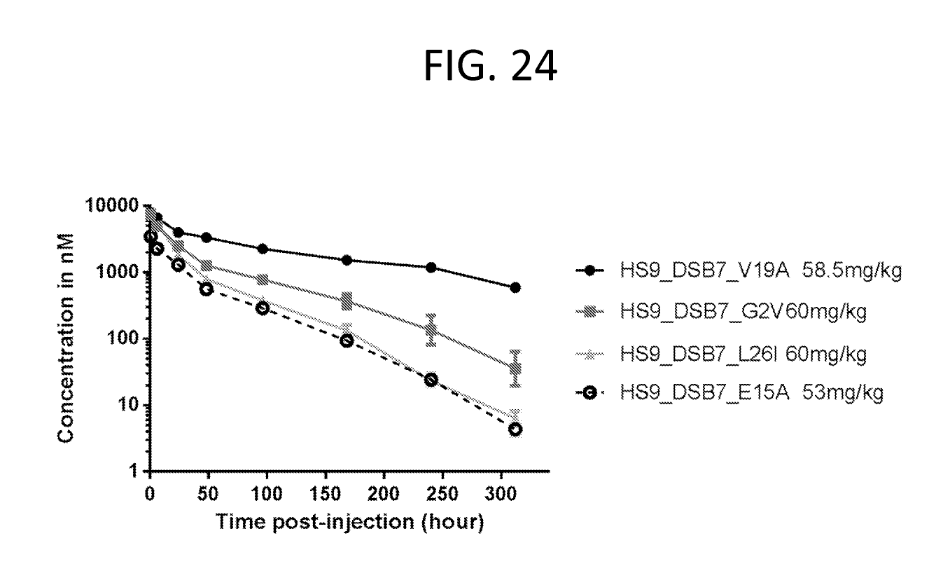

Blood samples were collected at 30 minutes, 6 h, 24 h, 48 h, 96 h, 168 h, 240 h and 336 h post injection. Concentrations for the four compounds over time are shown in FIG. 24 and half-life data are summarized in Table 21.

TABLE 21
In vivo half-life of peptide/antibody lead molecules
in rats after a single i.v. injection.

| | | | Exposure |
|---|---|---|---|
| # | Compound | Design | half-life (h) |
| 1 | HS9_DSB7_G2V | 60 mg/kg iv in CD rats | 48 |
| 2 | HS9_DSB7_E15A | 53 mg/kg iv in CD rats | 41 |
| 3 | HS9_DSB7_V19A | 58.5 mg/kg iv in CD | 128 |
| | | rats | |
| 4 | HS9_DSB7_L26I | 60 mg/kg iv in CD rats | 39 |

The four fusions molecules have significant different profiles despite being very close in sequence and sharing the same antibody backbone. HS9_DSB7_V19A has the longest in vivo half-life of the four fusions, 128 h compared to for instance only 39 h for HS9_DSB7_L26I.

### Example 17

### Affinity and Kinetic Parameters Determination for PCSK9 Across Species of GLP-1 Analogue Peptide in Fusion with Anti-PCSK9 Antibody

Association (ka), dissociation (kd) and equilibrium dissociation constants (KD) for human, cynomolgus and rat PCSK9 binding to the GLP-1 analogue anti-PCSK9 fusion HS9_DSB7_V19A human IgG1-TM were determined at 25° C. by Surface Plasmon Resonance (SPR) using the Biacore 2000 biosensor (GE Healthcare), essentially as described by Karlsson et al. G. Immunol. Methods (1991), vol. 145, p. Dear229-40). Anti-PCSK9 antibody PC9#3 human IgG1-TM was used as benchmark (Variable heavy chain of SEQ ID NO: 404 and variable light chain of SEQ ID NO: 405.

A mouse anti-human IgG monoclonal antibody surface was first created using a Human Antibody Capture Kit and CM5 sensor chip (GE Healthcare). Human antibody compounds were captured at a flow rate of 10 μL/minute for 3 minutes. Recombinant human Avi_PCSK9_Flag_His (in-house), cynomolgus Avi_PCSK9_Flag_His (in-house) and His-tagged rat PCSK9 (SinoBiological) were diluted to concentrations ranging from 1 nM to 200 nM in running buffer (10 mM sodium phosphate pH 7.4, 150 mM sodium chloride, 1 mg/mL BSA, 0.05% Tween20) and injected over the chip surface for 10 minutes, followed by running buffer only for a 10 minutes dissociation phase. The surface of the chip was regenerated using 3 M magnesium chloride between each antibody application. Global dissociation rates were first calculated followed by global on-rate calculations both using a 1:1 binding kinetics model.

Results are shown in Table 22.

TABLE 22
Kinetic parameters determined by Biacore of peptide/antibody
lead molecule HS9_DSB7_V19A for human, cynomolgus
and rat PCSK9 compared to anti-PCSK9 antibody PC9#3

| | | | |
|---|---|---|---|
| kinetic parameters | ka (1/Ms) | kd (1/s) | KD (M) |

Human PCSK9

| | | | |
|---|---|---|---|
| PC9#3 | 6.4E+05 | 4.4E−04 | 7.0E−10 |
| HS9_DSB7_V19A | 1.9E+05 | 1.1E−04 | 6.0E−10 |

Cynomolgus PCSK9

| | | | |
|---|---|---|---|
| PC9#3 | 4.2E+05 | 4.2E−04 | 1.0E−09 |
| HS9_DSB7_V19A | 1.1E+05 | 1.9E−04 | 1.7E−09 |

Rat PCSK9

| | | | |
|---|---|---|---|
| PC9#3 | 9.0E+05 | 4.7E−03 | 5.2E−09 |
| HS9_DSB7_V19A | 3.7E+05 | 2.1E−04 | 5.7E−10 |

Fusion molecule HS9_DSB7_V19A has an affinity at pH7.4 for human PCSK9 of 600 pM very similar to the benchmark anti-PCSK9 antibody PC9#3. Interestingly HS9_DSB7_V19A, with a four time lower dissociation constant, is less prone to dissociate from human PCSK9 compared to PC9#3.

In addition, HS9_DSB7_V19A can strongly bind to cynomolgus and rat PCSK9 at physiological pH with equilibrium dissociation constants close to the human PCSK9 value (1.7 nM and 570 pM respectively).

### Example 18

### Specificity for PCSK9 Compared to Closely Related Human Proteins of GLP-1 Analogue Peptide in Fusion with Anti-PCSK9 Antibody

Specificity for PCSK9 compared to related human proteins of the GLP-1 analogue anti-PCSK9 fusion HS9_DSB7_V19A human IgG1-TM was determined by Dissociation-Enhanced Lanthanide Fluorescent Immunoassay Time Resolved Fluorescence (DELFIA TRF Assay, PerkinElmer). Recombinant human Avi_PCSK9_Flag_His (in-house), GST tagged human PCSK7 (Abnova), GST tagged human MBTPSI (Abnova) and Flag/His tagged human CD86 (in-house) were coated at 10 μg/mL in PBS into 96-well immunoassay plate. After washing, HS9_DSB7_V19A was added to antigen-coated wells at a concentration of 25 μg/mL. Plates were incubated at room temperature for 2 hours before extensive washing. Bound HS9_DSB7_V19A human IgG1-TM was detected using secondary Europium-labelled anti-human IgG antibody (PerkinElmer). Antigen coating to the plates was assessed by using a mouse anti-GST IgG (Abcam) as primary and Europium-labelled anti-mouse IgG (PerkinElmer) as detection for human PCSK7 and human MBTPSI. Human PCSK9 and human CD86 coating was directly assessed using a Europium-labelled anti-His IgG (PerkinElmer).

Results showed that HS9_DSB7_V19A human IgG1-TM binds strongly to human PCSK9 but not to human PCSK7, human MBTPSI (PCSK8) or human CD86 (data not shown).

### Example 19

### Blocking Human PCSK9 Binding to LDL Receptor with GLP-1 Analogue Peptide in Fusion with Anti-PCSK9 Antibody

Ability of the GLP-1 analogue anti-PCSK9 fusion HS9_DSB7_V19A to block the binding of human PCSK9 to human LDL receptor was assessed using an ELISA competition assay. Anti-PCSK9 antibody HS9 and irrelevant isotype match NIP228 human IgG1-TM were used as positive and negative controls respectively.

Binding of biotinylated human PCSK9 (in house) at 5 ug/mL in 1× Phosphate Buffered Saline, 3% skimmed milk to human LDL-R (R&D Systems) coated overnight at 10 ug/mL onto 96 well MaxiSorb plate (NUNC) was detected by ELISA using cryptate labelled streptavidin (Perkin Elmer) diluted at 100 ng/mL in Delfia Buffer (Perkin Elmer). That interaction was challenged using a 3-fold serial dilution, starting at 100 ug/mL, of compounds co-incubated for 2 h at room temperature with the biotinylated PCSK9 reagent in the LDL receptor coated wells. Fluorescence signal was read on the Perkin Elmer Envision machine using a 340 nm excitation and 620 nm emission. Percentage of specific binding was calculated by subtracting the background signal obtained with no LDL receptor coated onto the plate normalized with the maximum specific binding signal obtained with no competitor compound minus background level.

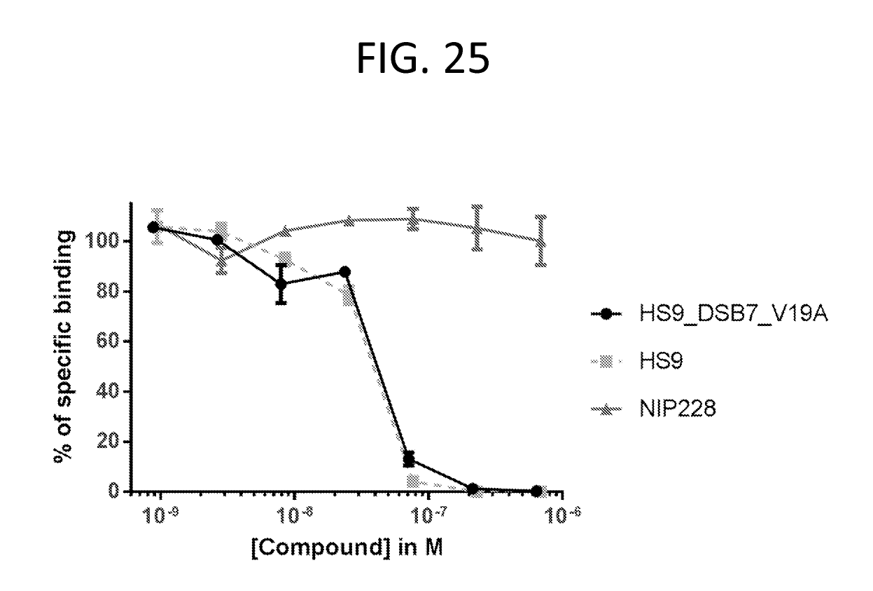

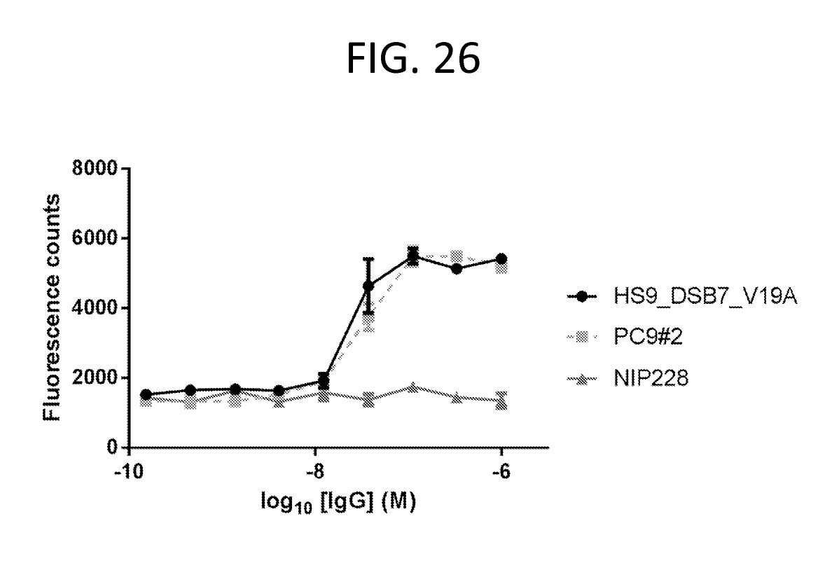

Biochemical inhibition of PCSK9 binding to LDL receptor is presented in FIG. 25.

Fusion molecule HS9_DSB7_V19A can block the binding of biotinylated human PCSK9 to recombinant LDL receptor with similar IC₅₀ compared to the positive control anti-PCSK9 antibody HS9 (4.3E-8 and 3.5E-8 M respectively).

### Example 20

### LDL Uptake by HEPG2 Cells Treated with GLP-1 Analogue Peptide in Fusion with Anti-PCSK9 Antibody

Ability of the GLP-1 analogue anti-PCSK9 fusion HS9_DSB7_V19A to block PCSK9 activity and restore LDL uptake was tested in HepG2 hepatic cells as followed. Anti-PCSK9 antibody PC9#2 and irrelevant isotype match NIP228 human IgG1-TM were used as positive and negative controls respectively.

Human HepG2 cells were seeded in black, clear bottom 96-well Greiner plates at a concentration of 2×10⁴ cells per well in DMEM medium (Gibco) supplemented with 10% lipoprotein deficient serum (Sigma) and incubated at 37° C. (5% CO₂) overnight. To complex PCSK9 with the tested compound, 45 nM of human Avi_PCSK9_FLAG_His (in house) was incubated with or without the tested compound at various concentrations in DMEM+10% LPDS for 1 hour at room temperature. All media was removed from the cell plate and the PCSK9/compound mixtures were transferred to the plate and incubated for 1 hour. Bodipy-LDL (Molecular Probes), diluted in DMEM+10% LPDS to a final concentration of 50 nM, was next transferred to the cells and the plate incubated for 5 hours at 37° C. (5% CO₂). Cells were washed thoroughly with PBS, stained with the nuclear dye Hoescht and fixed using formaldehyde at a final concentration of 3.7% (v/v). Assay plates were read for cell-associated fluorescence using the Cellomics ArrayScan VTi high content imaging system. Hoescht staining was measured in channel 1 using the BGRFR_386_23 filter and Bodipy-LDL in channel 2 using the BGRFR_485_20 filter. Images were analysed using the Compartmental Analysis v4 algorithm.

Inhibition of PCSK9-dependent loss of LDL uptake by HepG2 cells is presented in FIG. 26.

Fusion molecule HS9_DSB7_V19A can restore LDL-uptake by HepG2 cells treated with human PCSK9 with similar IC₅₀ compared to the positive control anti-PCSK9 antibody PC9#2 (2.5E-8 and 3.1E-8 M respectively).

### Example 21

### Potency at GLP-1 Receptors Across Species of GLP-1 Analogue Peptide in Fusion with Anti-PCSK9 Antibody

Cross-reactivity of the GLP-1 analogue anti-PCSK9 fusion HS9_DSB7_V19A at human, cynomolgus, mouse and rat GLP-1 receptors was tested in a cAMP production assay as previously described by using stable cell lines overexpressing the receptor of interest. GLP1-Fc fusion (in house) and GLP-1 peptide were used as positive controls.

Potency at the different GLP-1 receptors is summarised in Table 23.

TABLE 23
Potency of lead molecule HS9_DSB7_V19A
at GLP-1 receptor across species

| | | Potency at GLP-1R in cAMP assay | | | |
|---|---|---|---|---|---|
| | | across species (M) | | | |
| | | | cyno- | | |
| # | Compound | human | molgus | mouse | rat |
| 1 | HS9_DSB7_V19A | 3.73E−09 | 5.83−10 | 5.75−10 | 3.83E−11 |
| 2 | GLP1- | 7.72E−11 | 1.09E−11 | 8.98E−11 | 8.92E−12 |
| | Fc(Gamma4) | | | | |
| 3 | GLP-1 peptide | 1.61E−11 | 1.03E−11 | 2.69E−11 | 9.31E−11 |

Fusion molecule HS9_DSB7_V19A can activate GLP-1 receptor across all the four tested species.

### Example 22

### Several Compounds were Identified by Reducing Potency at the Human GLP-1 Receptor

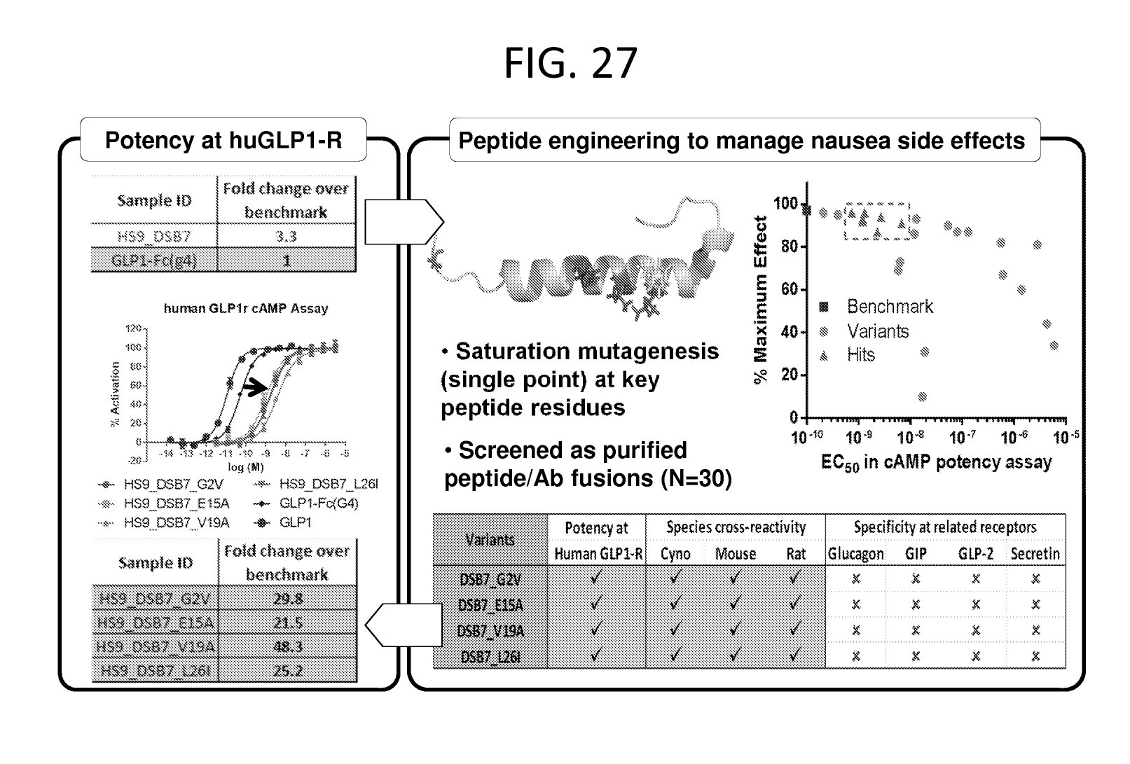

To reduce DSB7 potency, certain residues in the peptide were mutated. Peptide Ab fusions to SEQ ID NO: 2 with linker SEQ ID NO: 4 at the desired potency, here shown in green triangles, were further analyzed for specificity at human GLP1-R and species cross reactivity. See FIG. 27.

The variants are shown as HS9_DSB7_G2V (Gly₂→Val) (SEQ ID NO: 44 and SEQ ID NO: 1); HS9_DSB7_E15A (Glu₁₅→Ala) (SEQ ID NO: 45 and SEQ ID NO: 1); HS9_DSB7_V19A (Va119→Ala) (SEQ ID NO: 47 and SEQ ID NO: 1); HS9_DSB7_L26I (Leu₂₆→Ile) (SEQ ID NO: 46 and SEQ ID NO:1) (compared to Val₁₉→Ala (SEQ ID NO: 3)). These four variants were selected for final characterization. Results are shown in FIG. 27.

### Example 23

### Fusion Molecule with Reduced Potency

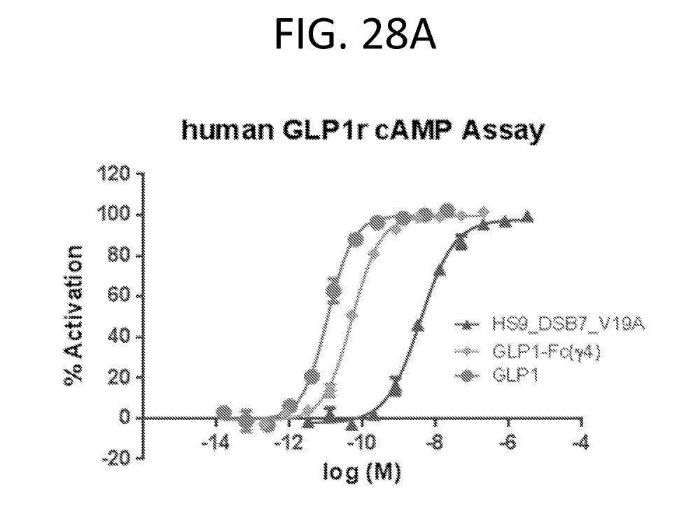

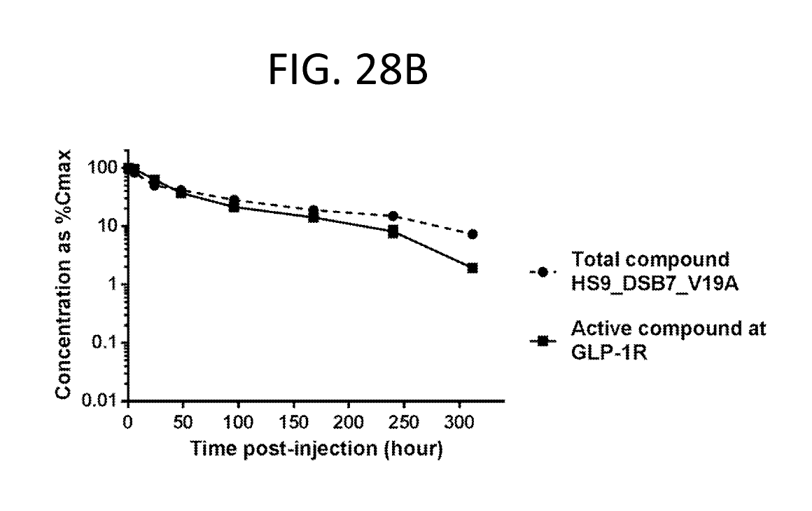

The PCSK9/GLP-1 fusion molecule exhibits the desired potency on the GLP-1 receptor, as shown in FIG. 28A. The potency of this compound has been reduced to minimize nausea. Table 24 shows that HS9_DSB7_V19A has a 57.7 fold reduced potency with respect to dulaglutide. This engineered reduction of potency provides the desired effect of reducing nausea and other untoward effects.

TABLE 24
Fusion Molecule with Reduced Potency

| | Mean | Fold Change Over | Max |
|---|---|---|---|
| Sample ID | EC50 (M) | Benchmark | Activation (%) |
| HS9_DSB7_V19A | 4.4E−09 | 57.7 | 98 |
| (heavy chain of SEQ | | | |
| ID NO: 1 and light | | | |
| chain fusion of SEQ | | | |
| ID NO: 47) | | | |
| GLP-1 (SEQ ID NO: | 1.6E−11 | 0.2 | 100 |
| 29) | | | |
| GLP1-Fc(G4) | 7.7E−11 | 1.0 | 100 |
| (dulaglutide) | | | |

PCSK9/GLP-1 fusion of Example 1 exhibits an Ab exposure/GLP-1 activity profile sufficient to enable weekly dosing, for example, as shown in FIG. 28B. pK stability study in rat of 58.5 mg/kg of HS9_DSB7_V19A injected into rat. Concentration of the fusion compound is measured in the serum over time. Samples taken from rat are analyzed for both activity of the test compound and concentration of the test compound in serum. Data from the GLP-1 activity portion is used to back calculate for concentration of “active” compound. The line with closed circles is the concentration of HS9_DSB7_V19A and line with closed squares is the concentration of active HS9_DSB7_V19A having GLP-1 activity) for the same samples.

### Example 24

### Specificity for GLP-1 Receptor Compared to Closely Related Human Receptors of GLP-1 Analogue Peptide in Fusion with Anti-PCSK9 Antibody

Specificity of the GLP-1 analogue anti-PCSK9 fusion HS9_DSB7_V19A for GLP-1 receptor compared to related human receptors was tested in a cAMP production assay as previously described by using stable cell lines overexpressing the receptor of interest: glucagon, GIP, GLP-2 and secretin receptors. Specific agonist peptides for each of the four receptors were used as positive controls.

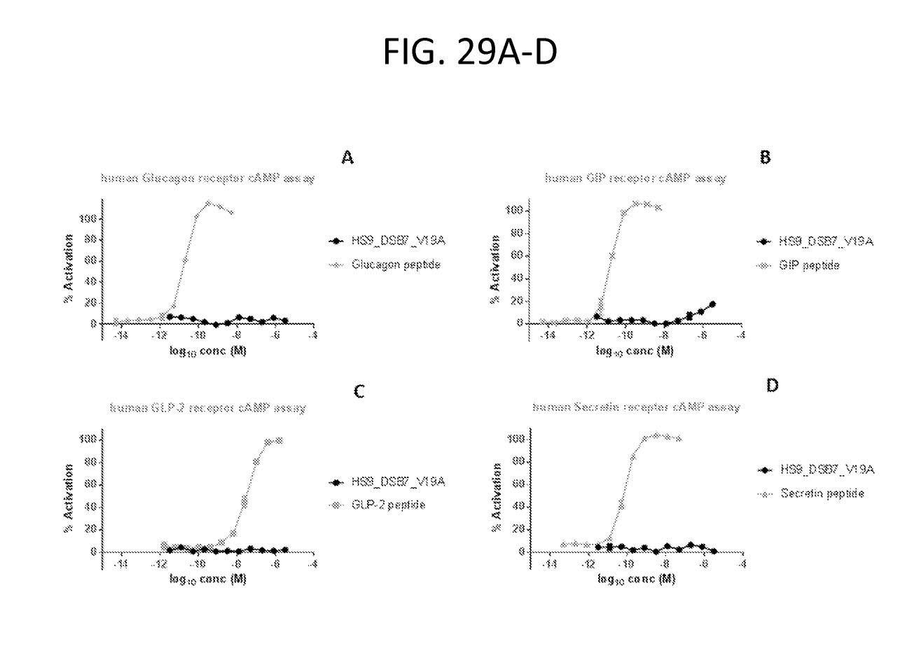

Data are shown in FIG. 29A-D (A: Glucagon receptor, B: GIP receptor, C: GLP-2 receptor, D: Secretin receptor).

Fusion molecule HS9_DSB7_V19A is specific for GLP-1 receptor and does not activate any of the four tested closely related receptors.

### Example 25

### Further Characterization of Fusion Molecule Demonstrates a Favorable 1 n Vivo Profile

The PCSK9/GLP-1 fusion molecule of Example 2 (HS9_DSB7_V19A (heavy chain of SEQ ID NO: 1 and light chain fusion of SEQ ID NO: 47)) shows superior glucose control and weight loss over time, including at day 7 post dose. Data is shown against dulaglutide and PC9_2_VH and VL (SEQ ID NOS: 8 and 9). Animals were dosed at day 0 with the compounds and then their body weight was measured over time to determine a change in body weight.

The fusion molecule has shown that it binds purified PCSK9 with high affinity, it restores LDLc uptake in HEPG2 cells, stimulates GLP-1R at a desired potency, promotes weight loss and demonstrates favorable exposure/activity profile in rat PK to support weekly dosing and sustained GLP-1 activity in vivo.

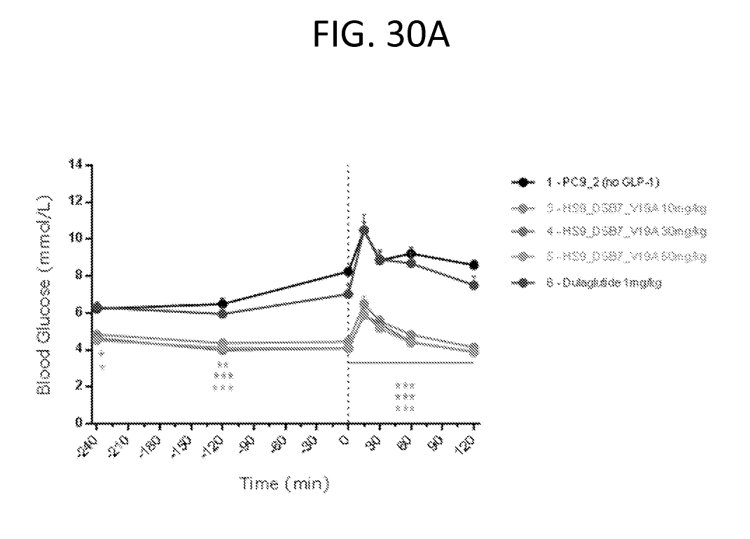

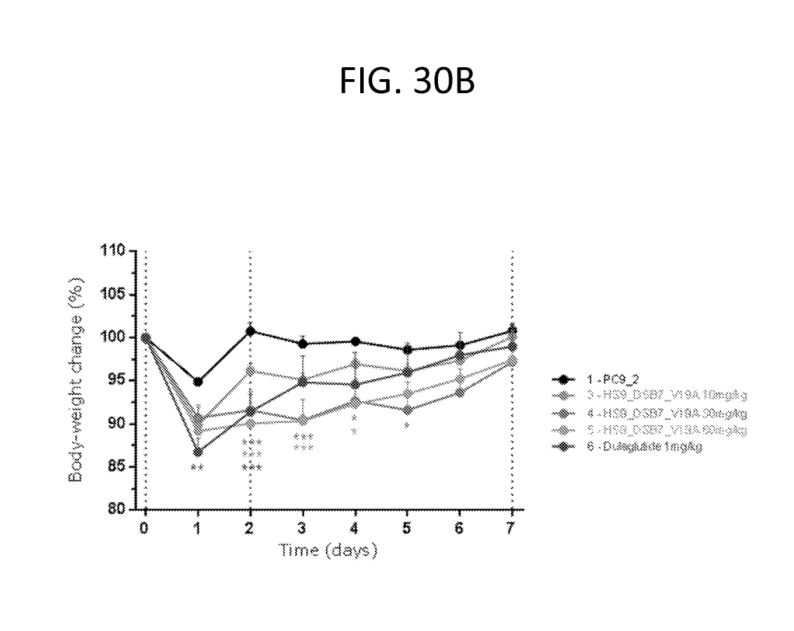

Results are provided in FIGS. 30A-B.

### Example 26

### Impact of the Linker on the Activities of GLP-1 Analogue Peptide in Fusion with Anti-PCSK9 Antibody

The compound HS9_DSB7_V19A has a linker of SEQ ID NO: 4 corresponding to a Gly₄Ser motif repeated three times between the peptide moiety and the antibody light chain. To investigate the impact of the linker length between the GLP-1 analogue peptide DSB7_V19A (SEQ ID NO: 3) when fused to the light chain of anti-PCSK9 antibody HS9 (light chain: SEQ ID NO: 2 and heavy chain: SEQ ID NO: 1), three fusions with a reduced linker length were generated: HS9_DSB7_V19A_L2 (SEQ ID NO: 419) having a linker corresponding to the Gly₄Ser motif repeat two times (linker: SEQ ID NO: 403), HS9_DSB7_V19A_L1 (SEQ ID NO: 420) having a linker corresponding to the Gly₄Ser motif repeat one time (SEQ ID NO. 27) and HS9_DSB7_V19A_L0 with no linker between the peptide and the antibody light chain (SEQ ID NO: 421).

Compounds were tested for both binding to recombinant human PCSK9 by ELISA and activity at the human GLP-1 receptor using the cAMP assay cell based assay as described in Example 4.

Binding ELISA was performed by coating human PCSK9 (in house) at 10 ug/mL in 1× Phosphate Buffered Saline. After plate blocking with 1× Phosphate Buffered Saline, 3% skimmed milk, compounds were added at 100 ug/mL in PBS and incubated for 2 h at room temperature before washing. Bound compounds were detected using cryptate labelled Fc specific anti human IgG (Perkin Elmer) diluted at 100 ng/mL in Delfia Buffer (Perkin Elmer). Fluorescence signal was read on the Perkin Elmer Envision machine using a 340 nm excitation and 620 nm emission. HS9_DSB7_V19A was used as positive control and irrelevant isotype match NIP228 used to determine the background level.

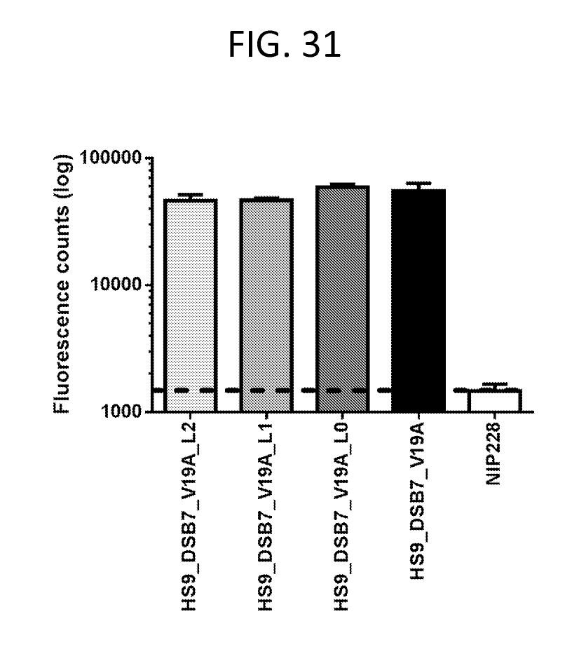

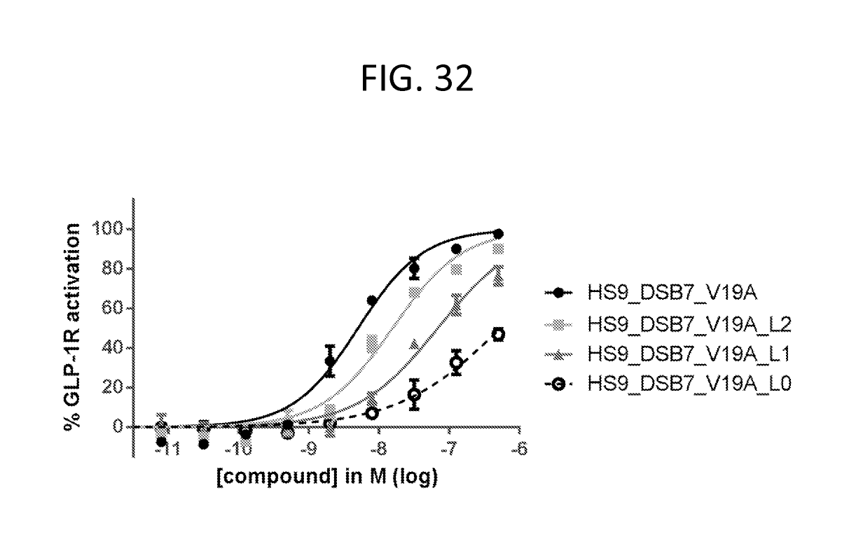

PCSK9 binding and GLP-1 receptor activation data are shown in FIGS. 31 and 32, respectively.

All additional fusion molecules are able to bind human PCSK9 to a similar level compared to HS9_DSB7_V19A. Tested fusion molecules can also activate human GLP-1 receptor in the cAMP cell based assay but reducing linker length is having a negative impact on compound potency. In that assay, EC₅₀ for HS9_DSB7_V19A, HS9_DSB7_V19A_L2, HS9_DSB7_V19A_L1 and HS9_DSB7_V19A_L0 are 5.0 nM, 15.6 nM, 68.7 nM and 537 nM, respectively.

### Example 27

### In Vitro Characterization of Stable GLP-1 Analogue Peptide in Fusion with Anti-PCSK9 Antibodies

The GLP-1 analogue peptide DSB7_V19A of SEQ ID NO: 3) was fused using a linker of SEQ ID NO:4 to the light chain of other anti-PCSK9 antibodies than HS9:

1_PC9#1 with antibody variable heavy chain of SEQ ID NO:10 and antibody variable light chain of SEQ ID NO:11

2_PC9#3 with antibody variable heavy chain of SEQ ID NO:404 and antibody variable light chain of SEQ ID NO:405.

3_PC9#4 with antibody variable heavy chain of SEQ ID NO:406 and antibody variable light chain of SEQ ID NO:407.

4_PC9#5 with antibody variable heavy chain of SEQ ID NO:408 and antibody variable light chain of SEQ ID NO:409.

5_PC9#6 with antibody variable heavy chain of SEQ ID NO:410 and antibody variable light chain of SEQ ID NO:411.

6_PC9#7 with antibody variable heavy chain of SEQ ID NO:412 and antibody variable light chain of SEQ ID NO:413.

Fusions were tested for both binding to recombinant human PCSK9 by ELISA as described in Example 26 and activity at the human GLP-1 receptor using the cAMP cell based assay as described in Example 4. HS9_DSB7_V19A and NIP228 isotype match were used as positive and negative controls respectively.

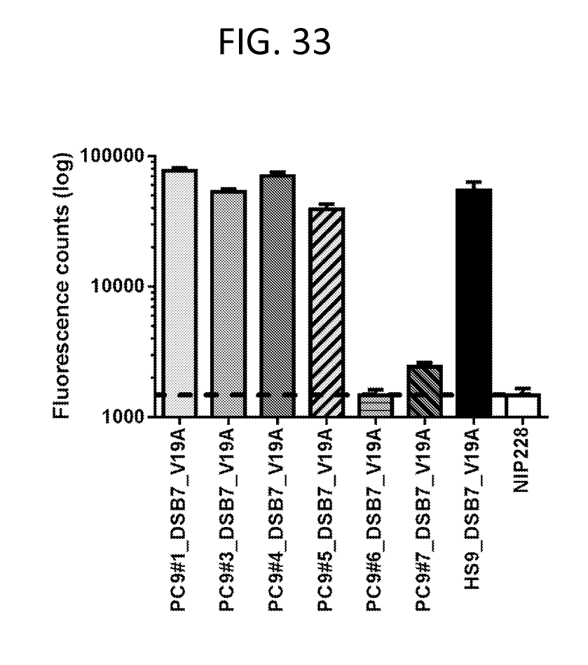

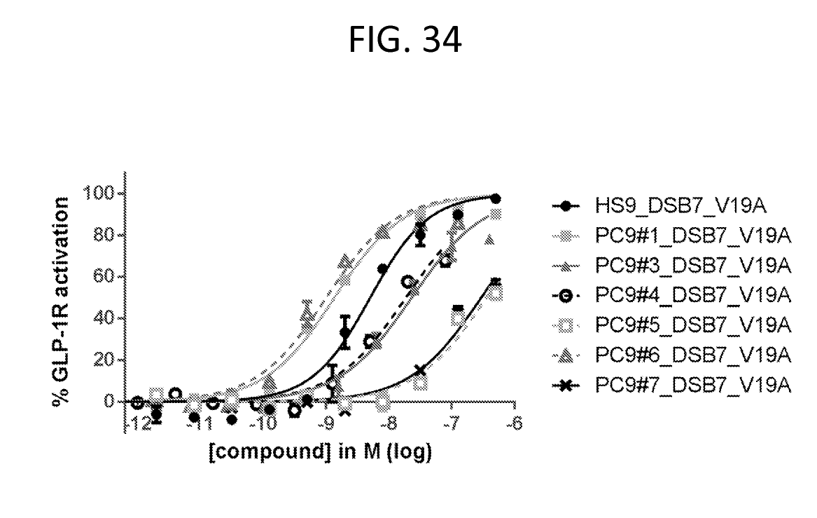

PCSK9 binding and GLP-1 receptor activation data are shown in FIGS. 33 and 34, respectively.

All seven compounds tested in the human GLP-1 receptor cAMP assay are able to activate the receptor. Potency among the panel is ranging from 1 to 350 nM for PC9#6_DSB7_V19A and PC9#5_DSB7_V19A, respectively. HS9_DSB7_V19A has a potency of 5 nM in that assay.

In addition, all tested fusions are also able to bind strongly to human PCSK9 by ELISA at the exception of PC9#7_DSB7_V19A which binds poorly and PC9#6_DSB7_V19A which does not bind.

### Example 28

### In Vitro Characterisation of Stable GLP-1 Analogue Peptide in Fusion with an Anti-B7-H1 Antibody

The GLP-1 analogue peptide DSB7_V19A of SEQ ID NO: 3 was fused using a linker of SEQ ID NO:4 to the light chain of the anti-B7-H1 antibody 2.7A4 described in patent WO2011066389. Anti B7-H1 antibody 2.7A4 has a variable heavy chain of SEQ ID NO:422 and a variable light chain of SEQ ID NO:423.

Fusion was tested for both binding to recombinant human B7-H1 by ELISA and activity at the human GLP-1 receptor using the cAMP assay cell based assay as described in Example 4.

Binding ELISA was performed by coating human B7-H1 (in house) at 5 ug/mL in 1× Phosphate Buffered Saline. After plate blocking with 1× Phosphate Buffered Saline, 3% skimmed milk, compounds were added at 10 ug/mL in PBS and incubated for 2 h at room temperature before washing. Bound compounds were detected using cryptate labelled Fc specific anti human IgG (Perkin Elmer) as described in Example 25. Anti-B7-H1 antibody 2.7A4 and irrelevant isotype match NIP228 were used as positive and negative control respectively.

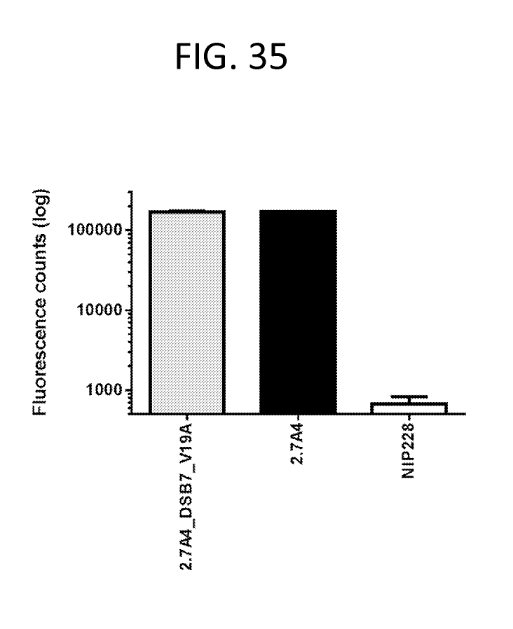

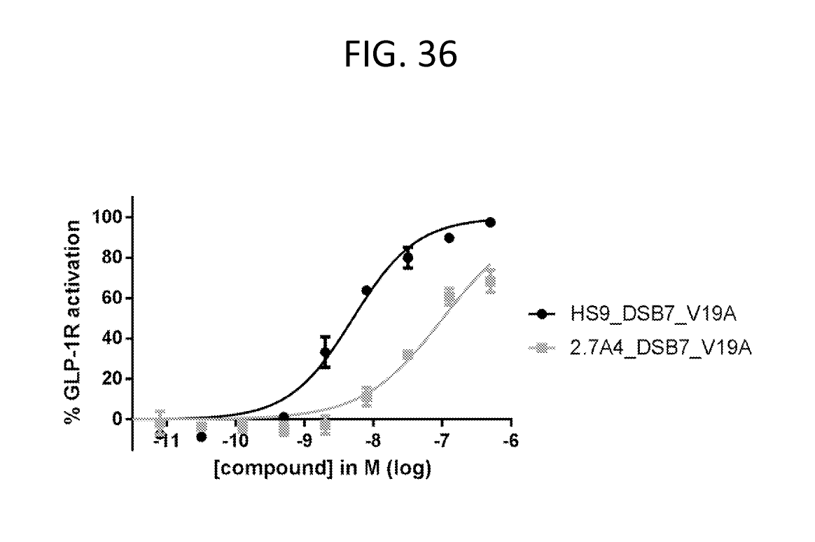

B7-H1 binding and GLP-1 receptor activation data are shown in FIGS. 35 and 36, respectively.

Fusion 2.7A4 DSB7 V19A is able to bind human B7-H1 to a similar level than the positive control 2.7A4 antibody. The fusion can also activate human GLP-1 receptor in the cAMP cell based assay with a potency of 100 nM compared to 5 nM for HS9_DSB7_V19A.

### Example 29

### Pharmacokinetics and Pharmacodynamics in Rat of GLP-1 Analogue Peptide in Fusion with Anti-PCSK9 Antibody Following Single Intravenous Dose

A PKPD study for the peptide antibody fusion HS9_DSB7_V19A following a single intravenous bolus in CD rats was conducted in order to assess its in vivo stability, by measuring both compound exposure and concentration in active GLP-1, as well as target engagement by measuring concentration in free rat PCSK9.

Fusion molecule was injected at 10, 30 and 60 mg/kg. Anti-PCSK9 mAb HS9, without a peptide attached to it, was used as control and injected at 60 mg/kg. Blood samples were collected at:

1: Pre-dose—0.5 h-6 h-24 h-48 h-96 h and 168 h for the 10 mg/kg treatment group;

2: Pre-dose—0.5 h-24 h-48 h-96 h-168 h and 240 h for the 30 mg/kg treatment group; and

3: Pre-dose—0.5 h-24 h-72 h-168 h-336 h and 504 h for the 60 mg/kg treatment groups.

Concentrations of total human IgG1 antibody (exposure) and of active GLP-1 compound in rat serum samples were quantified as described in Example 4.

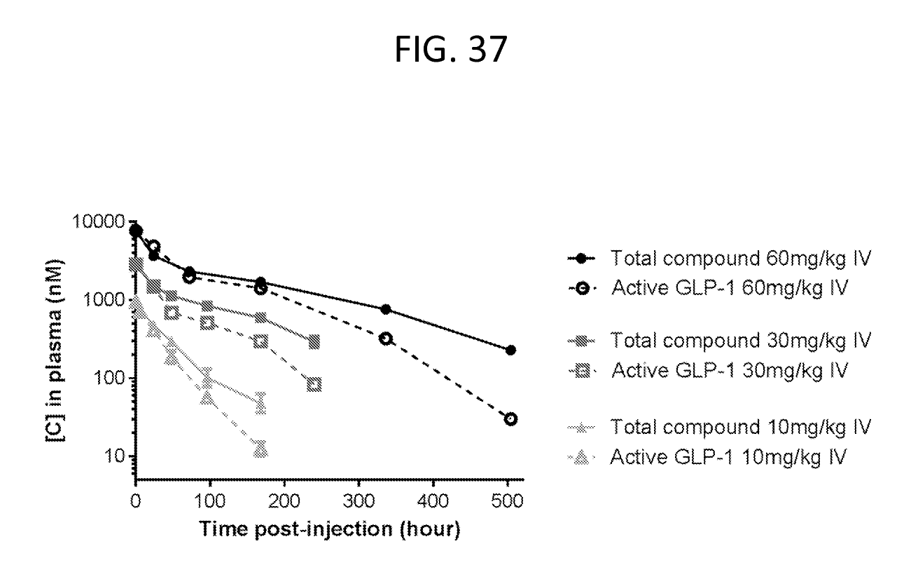

HS9_DSB7_V19A concentration over time in total and active GLP-1 compound in rat serum for the three tested doses (10, 30 and 60 mg/kg) are shown in FIG. 37.

Area under curve (AUC) for both total compound and active GLP-1 are summarised in Table 25. Calculating the ratio between active GLP-1 and total compound AUC is one way to evaluate in vivo stability. A ratio of one is corresponding to a fully stable compound in the tested conditions.

Data for the parent fusion molecule PC9#2_Exe4 comprising Exendin-4 in light chain fusion with the anti-PCSK9 mAb PC9#2 (See Example 4) have also been included for comparison.

TABLE 25
Total and active GLP-1 AUC0-t following a single
IV injection of the fusion molecule HS9_DSB7_V19A
compared to the parent fusion molecule PC9#2_Exe4

| | | | AUC0-t | |
|---|---|---|---|---|
| | | | Active | |
| | | AUC0-t | GLP-1 | Active/Total |
| | | Total (day · | (day · | AUC |
| Compound | Design | nmol) | nmol) | Ratio |
| HS9_DSB7_V19A | 10 mg/kg IV | 1595 | 1216 | 0.76 |
| HS9_DSB7_V19A | 30 mg/kg IV | 8702 | 6230 | 0.72 |
| HS9_DSB7_V19A | 60 mg/kg IV | 24535 | 26494 | 1.08 |
| PC9#2_Exe4 | 1 mg/kg IV | 299 | 40 | 0.13 |

HS9_DSB7_V19A displays a greater Active/Total AUC ratio compared to the parent molecule PC9#2_Exe4 demonstrating an improved in vivo stability profile in rat for activity at GLP-1 receptor.

Determination of free rat PCSK9 concentration in serum samples was based on a sandwich ligand binding assay method using the MSD® platform. Free rat PCSK9 was captured using the anti-PCSK9 mAb HS9 that was first non-specifically adsorbed on to the carbon surface of a Standard Bind MSD® plate. Anti-PCSK9 antibody from Abcam (Product Number ab125251) was labelled in-house with MSD® SULFO-TAG™ and used as detection reagent. Plate was then read using the MSD® Sector Imager 6000 (SI6000) instrument.

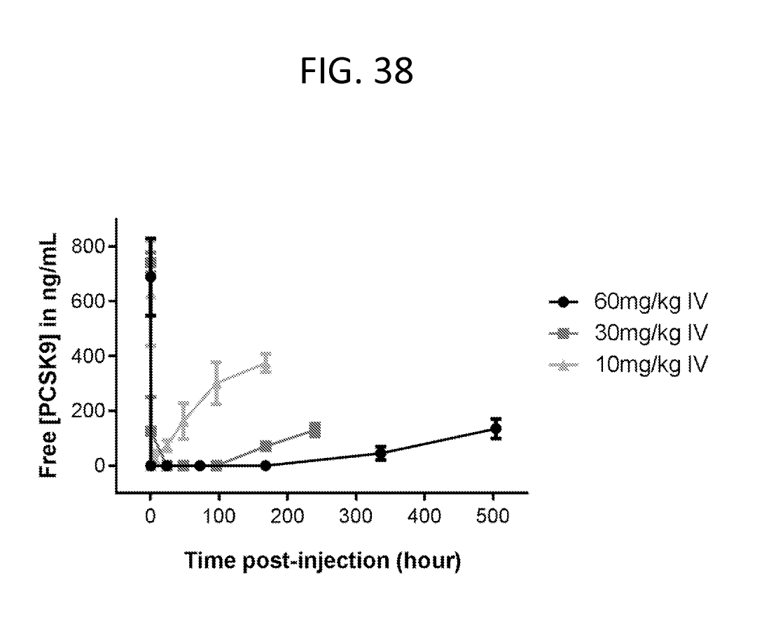

Engagement of HS9_DSB7_V19A to rat PCSK9 over time was evaluated by measuring the concentration of free antigen in the rat serum samples. Data for the three dosing groups (10, 30 and 60 mg/kg) are shown in Table 26 and FIG. 38.

TABLE 26
Concentration of Free Antigen in Rat Plasma Samples Over Time

| 10 mg/kg IV | | 30 mg/kg IV | | 60 mg/kg IV | |
|---|---|---|---|---|---|
| | Mean free | | Mean free | | Mean free |
| | rat | | rat | | rat |
| | [PCSK9] | | [PCSK9] | | [PCSK9] |
| Time (h) | in ng/mL | Time (h) | in ng/mL | Time (h) | in ng/mL |
| 0 | 837.2 | 0 | 741.8 | 0 | 688.5 |
| 0.5 | below | 0.5 | 125.8 | 0.5 | below |
| | LLOQ | | | | LLOQ |
| 6 | 26.7 | 24 | below | 24 | below |
| | | | LLOQ | | LLOQ |
| 24 | 74.1 | 48 | below | 72 | below |
| | | | LLOQ | | LLOQ |
| 48 | 162.5 | 96 | below | 168 | below |
| | | | LLOQ | | LLOQ |
| 96 | 301.0 | 168 | 70.9 | 336 | 45.4 |
| 168 | 374.7 | 240 | 130.3 | 504 | 135.8 |

HS9_DSB7_V19A is able to suppress rat PCSK9 below 90% at all tested doses but the duration of suppression is dose dependent. Free rat PCSK9 is again detectable at 6 h, 168 h and 336 h after injection when HS9_DSB7_V19A is dosed at 10, 30 and 60 mg/kg respectively.

### Example 30

### Pharmacokinetics and Pharmacodynamics in Rat of GLP-1 Analogue Peptide in Fusion with Anti-PCSK9 Antibody Following Single Subcutaneous Dose

A pharmacokinetic study for the GLP-1 analogue peptide antibody fusion HS9_DSB7_V19A following a single subcutaneous bolus at 60 mg/kg in CD rats was performed in order to compare routes of administration and their potential impact on compound exposure and in vivo stability.

Fusion molecule was injected at 40 mg/mL using a 1.5 mL/kg regimen. Blood samples were collected at Pre-dose—6 h-24 h-48 h-96 h-168 h and 240 h.

Concentrations of total human IgG1 antibody and of active GLP-1 compound as well as concentrations in free rat PCSK9 antigen in the serum samples were determined as described in Example 4 and Example 29.

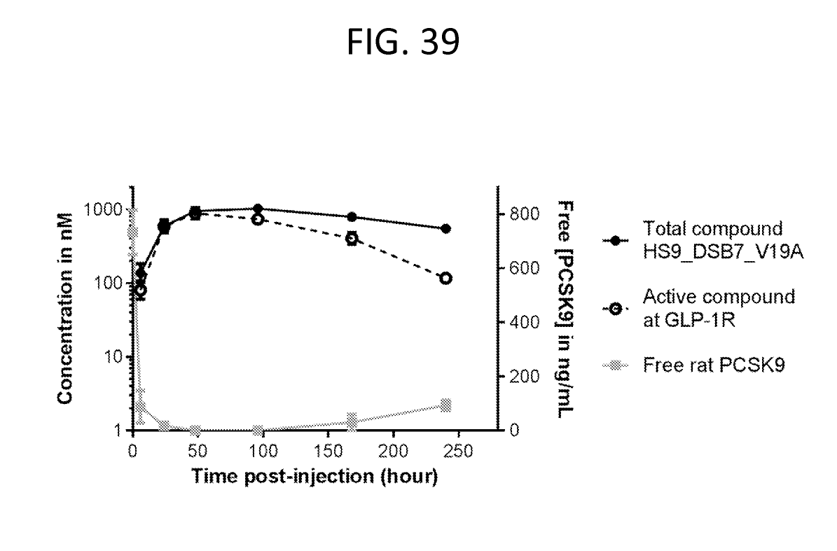

Total and active compound for HS9_DSB7_V19A and free rat PCSK9 concentrations are shown in FIG. 39. A maximum compound concentration of around 1000 nM was observed between 48 h and 96 h post-injection. Concentration in free rat PCSK9 is dropping sharply below the lower limit of quantification of the assay after compound injection and can be detected again at 168 h.

Area under curve (AUC) for both total and active compound were calculated and compared (Table 27) to model prediction using the data from the single intravenous dose injection described in Example 29. Fraction of absorption and absorption rate were set up to 75% and 0.3 d⁻¹ respectively.

TABLE 27
Calculated and predicted Area Under Curve following a single
SC injection of the fusion molecule HS9_DSB7_V19A

| | SC data | Predicted using |
|---|---|---|
| | 60 mg/kg | IV data |
| Exposure AUC0-t (day nmol) | 7815 | 8103 |
| Active GLP-1 AUC0-t (day | 5796 | 5739 |
| nmol) | | |
| Active/Exposure AUC ratio | 0.74 | 0.71 |

Exposure and active GLP-1 AUC of HS9_DSB7_V19A after a single subcutaneous injection at 60 mg/kg are similar to those predicted using the single intravenous injection data. This demonstrates that a subcutaneous route of injection has no significant impact on in vivo compound stability compared to an intravenous route of administration.

### Example 31

### Rodent Pharmacology-Antidiabetic Effects of a GLP-1 Analogue Peptide in Fusion with an Anti-PCSK9 Antibody

In order to confirm that the engineered nature of the GLP-1 analogue peptide portion of the HS9_DSB7_V19A fusion molecule retained antidiabetic activity in vivo, several; rodent pharmacology studies in normal, obese, and diabetic mouse models were performed.

A) Acute and Semi-Acute Effects of HS9_DSB7_V19A on Glucose Tolerance in C57B16 Mice

A single dose of HS9_DSB7_V19A was administered subcutaneously in normal C57B16 mice at either 1 or 50 mg/kg. The efficacy of the GLP-1 analogue component of the fusion molecule was evaluated by multiple glucose challenges (oral glucose tolerance test) at 4, 48, and 168 hours post administration of HS9_DSB7_V19A. Anti-PCSK9 mAb HS9 without a GLP-1 analogue peptide fused to it was administered at 50 mg/kg as a negative control for glucose tolerance, while Liraglutide (Victoza) and a GLP-1 analogue-Fcγ4 fusion (similar to Dulaglutide) were administered at 0.2 and 1 mg/kg, respectively. Due to the short half-life of Liraglutide, this compound was administered 2 hours prior to each glucose challenge while the GLP-1 analogue-Fcγ4 fusion molecule was administered once, at the same time as the HS9_DSB7_V19A test compound.

30 male C57B16 mice from Taconic Denmark were acclimatized for five days before experimentation. On day −1 of the study, animals were randomized into 5 groups based on body weight.

The experimental groups were as follows:

Group 1: Anti-PCSK9 mAb HS9 without GLP-1 analogue peptide component (negative control)—50 mg/kg subcutaneous dose

Group 2: Liraglutide (positive control)—0.2 mg/kg subcutaneous dose

Group 3: HS9_DSB7_V19A—1 mg/kg subcutaneous dose

Group 4: HS9_DSB7_V19A—50 mg/kg subcutaneous dose

Group 5: GLP-1 analogue-Fcγ4 fusion—1 mg/kg subcutaneous dose

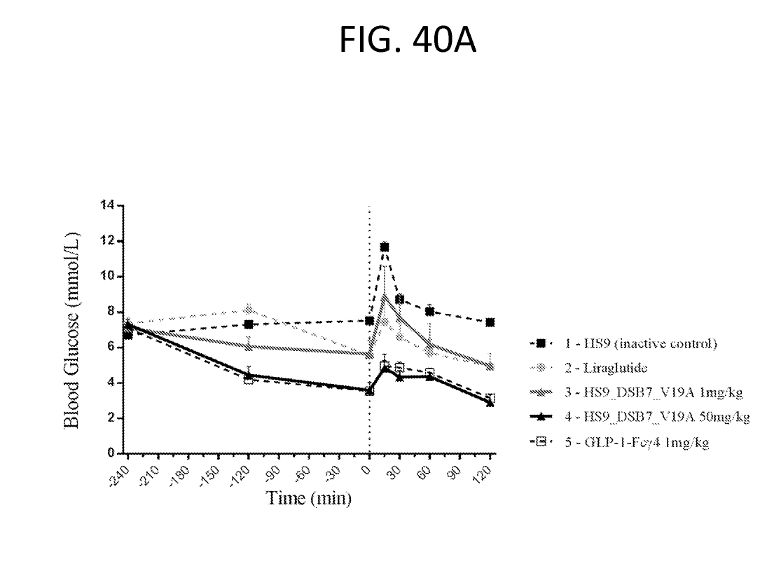

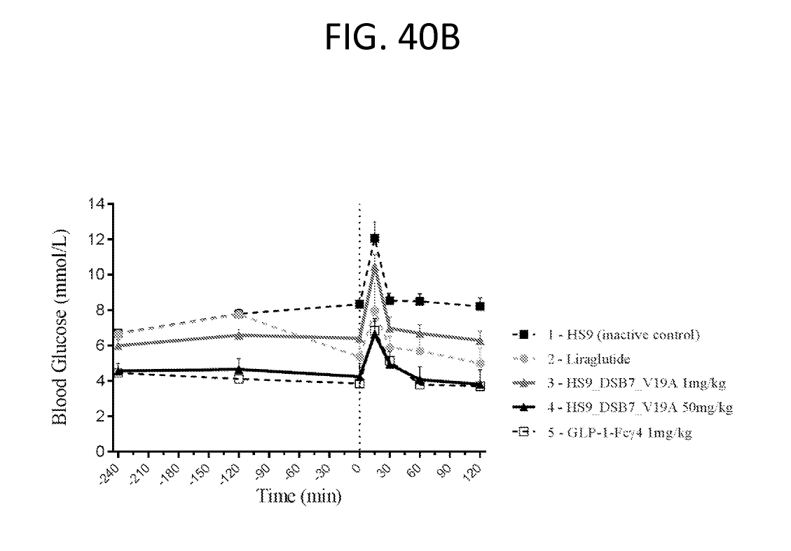

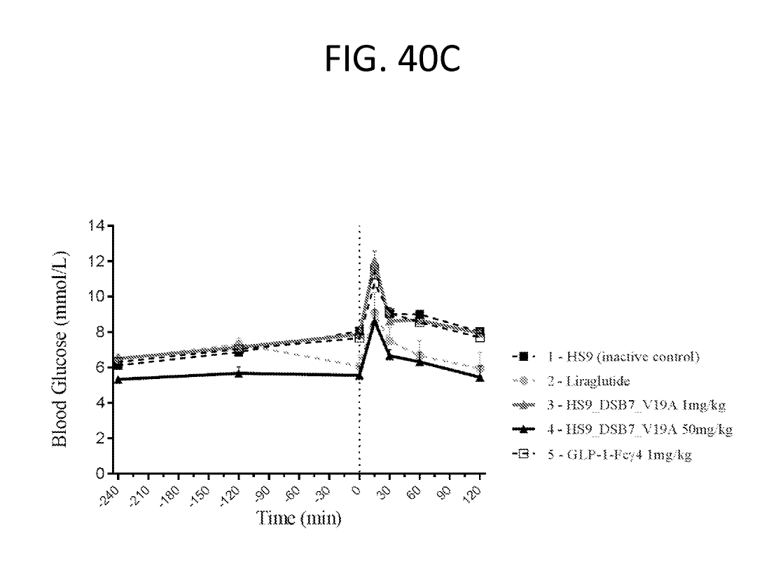

In order to assess efficacy of the GLP-1 analogue component of the fusion molecule over an extended period of time post dosing, 3 Oral Glucose Tolerance Tests (OM I) were performed at days 0, 2 and 7. Animals were fasted for 4 hours prior to the oral glucose challenge(s). Both doses of HS9_DSB7_V19A, inactive control and GLP-1 analogue-Fcγ4 fusion were administered once, 4 hours prior to the Day 0 OGTT. Liraglutide (positive control) was administered 2 hours prior to each glucose challenge at days 0, day 2 and day 7. At t=0 mice all mice are challenged with an oral glucose load of 2 g/kg glucose. Blood glucose is measured at t=−240, −120 and 0 minutes to establish a baseline and at t=15, 30, 60 and 120 minutes to monitor effects on glucose excursion. The results of the day 0, 2 and 7 OGTTs are presented in FIGS. 40A-C (day 0 (A), day 2 (B), day 7 (C)).

This study confirms the ability of a single, subcutaneous administration of HS9_DSB7_V19A at 50 mg/kg to improve glucose tolerance in normal C57B16 mice for at least 7 days.

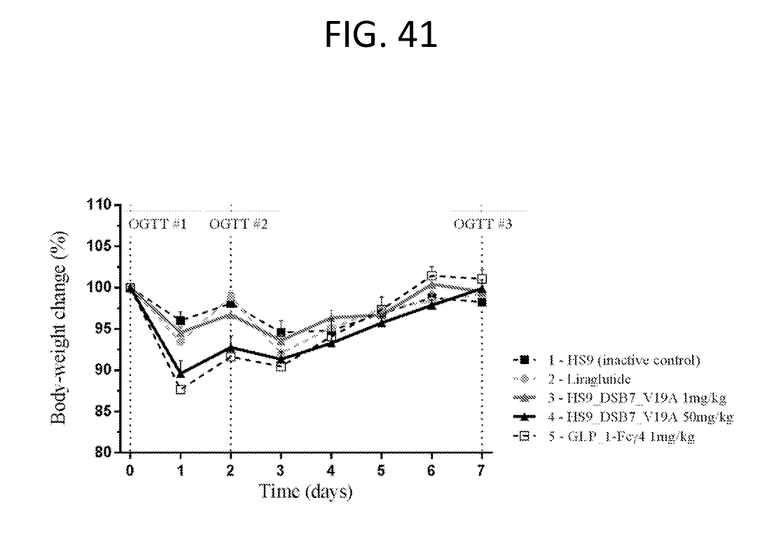

As an additional measure of efficacy of the GLP-1 analogue component of the HS9_DSB7_V19A fusion molecule body weights of all were recorded once daily from day −3 to the end of the study. A single, subcutaneous administration of HS9_DSB7_V19A at 50 mg/kg induced a transient reduction in body weight at days 1 and 2. Percent body-weight change over time is shown in FIG. 41.

B) Acute and Semi-Acute Effects of HS9_DSB7_V19A on Glucose Tolerance in C57B16 Mice-Dose Response

In order to examine a dose response effect and to determine a maximally efficacious dose of HS9_DSB7_V19A on glucose tolerance and body weight reduction in normal C57B16 mice, a study similar in design to that described above was conducted with the following experimental groups and doses:

Group 1: Anti-PCSK9 mAb HS9 without GLP-1 analogue peptide component (negative control)—50 mg/kg subcutaneous dose

Group 2: Liraglutide (positive control)—0.2 mg/kg subcutaneous dose

Group 3: HS9_DSB7_V19A—10 mg/kg subcutaneous dose

Group 4: HS9_DSB7_V19A—30 mg/kg subcutaneous dose

Group 5: HS9_DSB7_V19A—60 mg/kg subcutaneous dose

Group 6: GLP-1 analogue-Fcγ4 fusion—1 mg/kg subcutaneous dose

On day −1 animals were randomized into these 6 groups based on body weight (n=6 animals per group). As previously, OGTTs were performed on Days 0, 2 and 7 and blood glucose was measured at t=−240, −120, 0, 15, 30, 60 and 120 minutes.

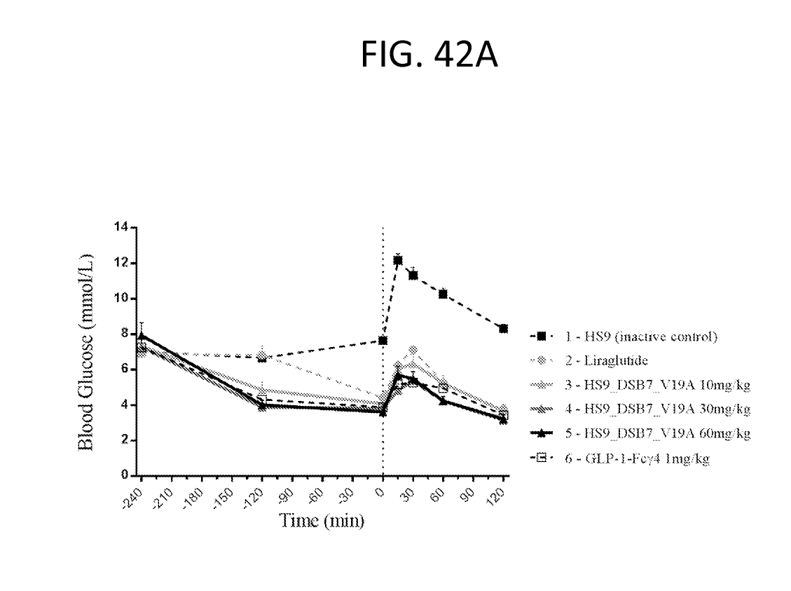

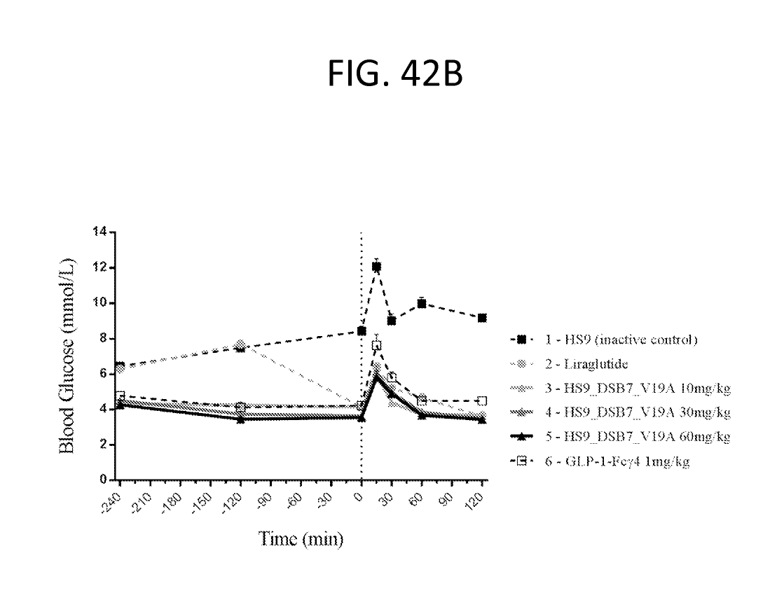

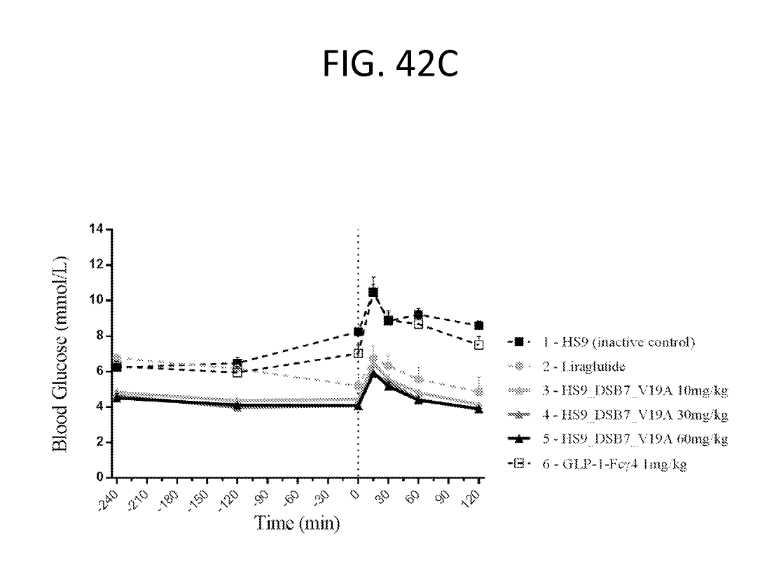

All three doses of HS9_DSB7_V19A resulted in similar levels of improved glucose tolerance at all three time points at which OGTTs were performed. FIGS. 42A-C illustrate the results from this oral glucose tolerance tests (day 0 (A), day 2 (B), day 7 (C)).

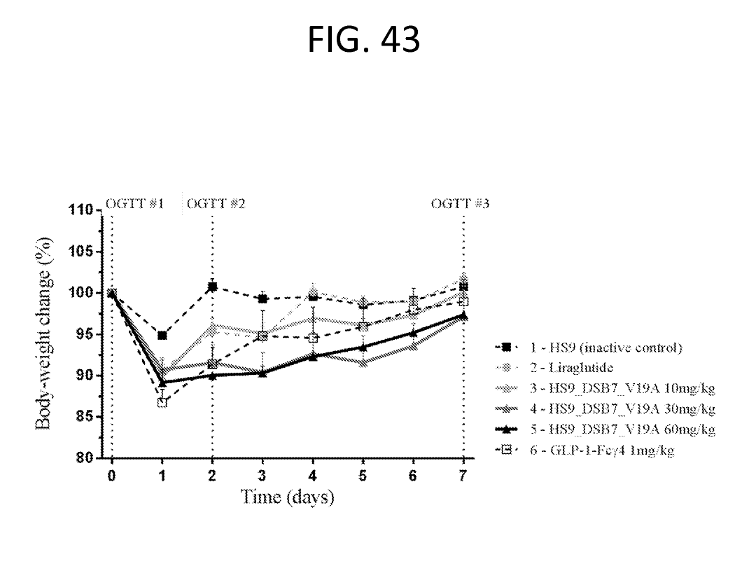

In contrast to the lack of a dose response observed for improvements in glucose homeostasis, a clear dose-dependent effect on body weight reduction was observed in this study. FIG. 43.

In this experimental model a single 10 mg/kg dose of HS9_DSB7_V19A generated a maximally efficacious level of improvement in glucose homeostasis in an OGTT performed 7 days post administration while the same dose did not have a statistically significant effect on body weight reduction at any point during the study.

C) Chronic Metabolic Effects of HS9_DSB7_V19A in a Diabetic db/db Mouse Model

In order to confirm the chronic vivo efficacy of HS9_DSB7_V19A on several metabolic parameters in a diabetic model, db/db (leptin receptor deficient) mice were utilized to examine the effects of weekly administration of HS9_DSB7_V19A on fasting glucose, glucose tolerance, body weight reduction and body mass composition.

In this study we examined the chronic metabolic effects of HS9_DSB7_V19A upon weekly dosing via a subcutaneous route. The vehicle control group and the positive control GLP-1 analogue-Fcγ4 fusion molecule were subcutaneously dosed twice weekly. In order to match the number of dosing manipulations to all animals in the study, the groups receiving a weekly dose of HS9_DSB7_V19A were dosed with vehicle on the days the other animals received their second weekly dose of either vehicle (negative control) or positive control GLP-1 analogue-Fcγ4 fusion molecule.

60 male db/db mice from Charles River, Italy were primarily randomized on body weight and glycosylated hemoglobin (HbA1c) and secondarily on 4 hour fasting blood glucose. These animals were randomized into 5 groups (n=12) as follows:

Group 1: Vehicle control group (Phosphate Buffered Saline)—twice weekly (BIW) subcutaneous dosing

Group 2: GLP-1 analogue-Fcγ4 fusion (positive control)—1 mg/kg subcutaneous dose—twice weekly subcutaneous dosing

Group 3: HS9_DSB7_V19A—30 mg/kg subcutaneous dose—once weekly (QW)

Group 4: HS9_DSB7_V19A—10 mg/kg subcutaneous dose—once weekly (QW)

Group 5: HS9_DSB7_V19A—3 mg/kg subcutaneous dose—once weekly (QW)

The chronic study was run for 28 days post initial dose. Last dose of the BIW groups was on study day 24 whereas the last dose of the QW groups was on study day 21. The major efficacy endpoints of this study are body weight, fasting blood glucose, and glucose tolerance.

Body weight was measured three times weekly during the treatment period.

4 hour fasting blood glucose was measured once weekly at approximately 24 hours post dosing.

Glucose tolerance was measured on study day 22 at approximately 24 hours post dosing by Intraperitoneal glucose tolerance test (IPGTT). Animals were fasted for 4 hours before administration of a 1 g/kg glucose bolus. Blood glucose was measured at t=0, 15, 30, 60, 120, and 180 minutes.

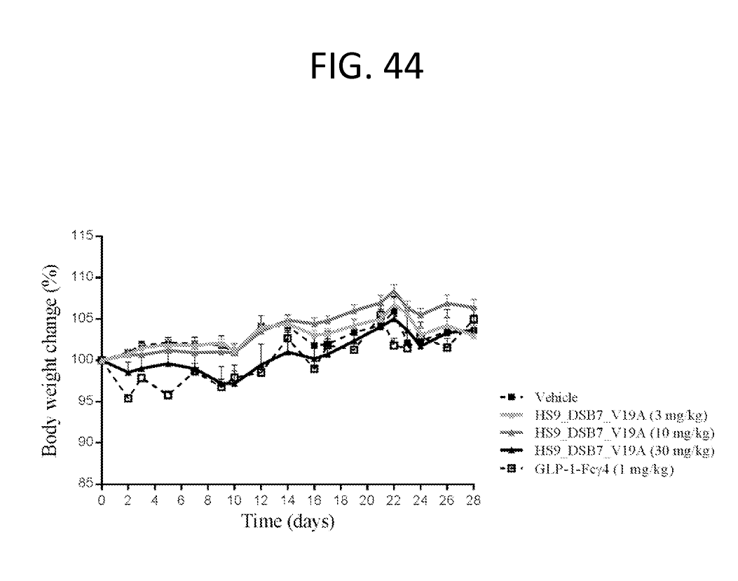

Body weight reduction in this study was not significant with the exception of one time point in the positive control GLP-1 analogue-Fcγ4 fusion molecule group. All three doses of HS9_DSB7_V19A did not result in a significant body weight reduction at any time during the course of the 28 day study. FIG. 44.

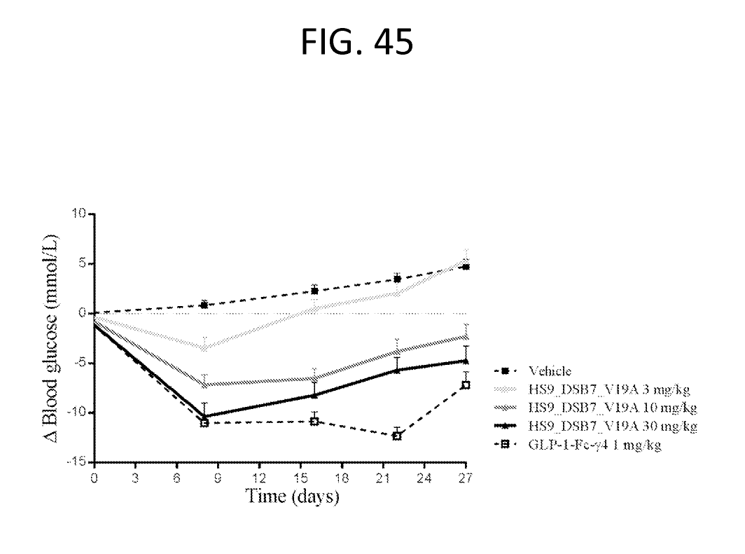

Weekly HS9_DSB7_V19A exhibited a dose dependent reduction in 4 hour fasting blood glucose as compared to vehicle control. FIG. 45.

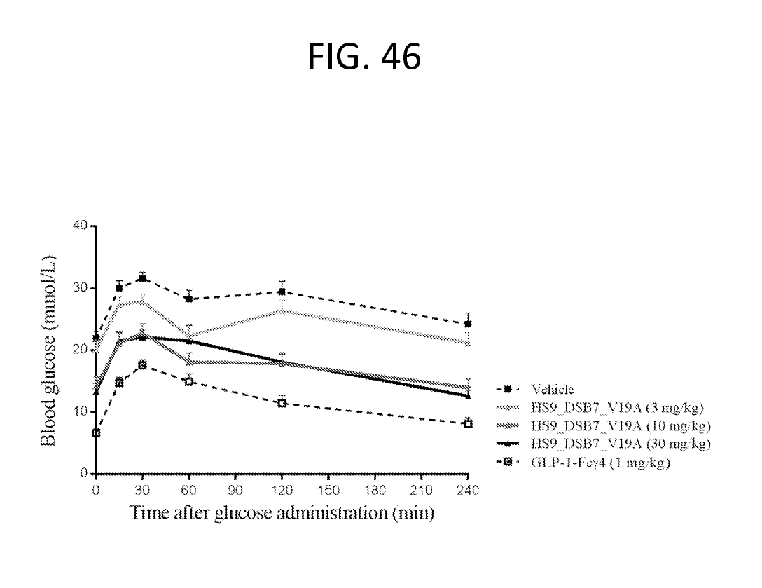

Weekly dosed HS9_DSB7_V19A exhibited a dose dependent improvement in glucose tolerance as assessed by IPGTT at study day 22. FIG. 46.

D) Acute Effect of Single Dose HS9_DSB7_V19A on Glucose Tolerance in a Diet-Induced Obesity (DIO) Mouse Model

A single dose of HS9_DSB7_V19A was administered subcutaneously in Diet-induced obese (DIO) mice at 0.1, 1 or 10 mg/kg. The efficacy of the GLP-1 analogue component of the fusion molecule was evaluated by separate glucose challenges (intraperitoneal glucose tolerance test) at 4 and 168 hours post administration of HS9_DSB7_V19A. Anti-PCSK9 mAb HS9 without a GLP-1 analogue peptide fused to it was administered once at 10 mg/kg as a negative control for effects on glucose tolerance. As a positive control, the GLP-1 analogue-Fcγ4 fusion (similar to Dulaglutide) used in the previous experiments was administered at 1 mg/kg twice weekly (BIW). In order to simulate the dosing regimen of the positive control GLP-1 analogue-Fcγ4 fusion, all animals in the negative control group and the HS9_DSB7_V19A experimental groups were dosed with vehicle BIW. The study duration was for 21 days following the first dose. Primary endpoint was effects on glucose tolerance in IPGTT on day 0 (4 hours post dose) and Day 7. Secondary endpoint was body weight reduction.

50 Male, 21 week old DIO mice on 60% high fat diet for 15 weeks prior to study start were obtained from Jackson labs (JAX: 380050). Just prior to study start, animals were randomized into 5 groups based on body weight.

The experimental groups (n=10 per group) were as follows:

Group 1: Anti-PCSK9 mAb HS9 without GLP-1 analogue peptide component (negative control)—10 mg/kg subcutaneous dose—single dose

Group 2: HS9_DSB7_V19A—0.1 mg/kg subcutaneous dose—single dose

Group 3: HS9_DSB7_V19A—1 mg/kg subcutaneous dose—single dose

Group 4: HS9_DSB7_V19A—10 mg/kg subcutaneous dose—single dose

Group 5: GLP-1 analogue-Fcγ4 fusion—1 mg/kg subcutaneous dose—twice weekly dosing (BIW)

In order to assess efficacy of the GLP-1 analogue component of the fusion molecule over an extended period of time post dosing, two Intraperitoneal Glucose Tolerance Tests (IPGTT) were performed at days 0 and 7. Animals were fasted for 6 hours prior to the IP glucose challenge(s). All three doses of HS9_DSB7_V19A, inactive control and GLP-1 analogue-Fcγ4 fusion were administered once, 4 hours prior to the Day 0 OGTT. The GLP-1 analogue-Fcγ4 fusion was administered on days 0, 3, 7, 10, 14 17 and 21. On day 0 all groups were dosed with test compounds 4 hours prior to the IP glucose challenge while on day 7 the GLP-1 analogue-Fcγ4 fusion was administered 4 hours prior to the IP glucose challenge while all other groups were dosed with vehicle only. For each day in which IPGTTs were performed, at t=0 mice all mice were challenged with an IP glucose load of 1.5 g/kg glucose. Blood glucose was measured at t=−240 and 0 minutes to establish a baseline and at t=15, 30, 45, 60, 90 and 120 minutes to monitor effects on glucose excursion.

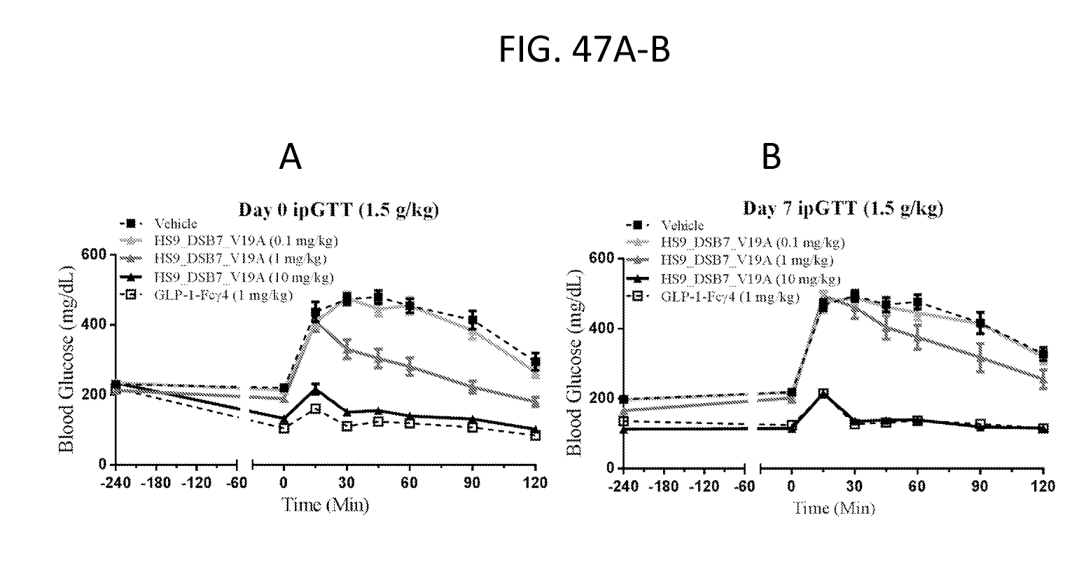

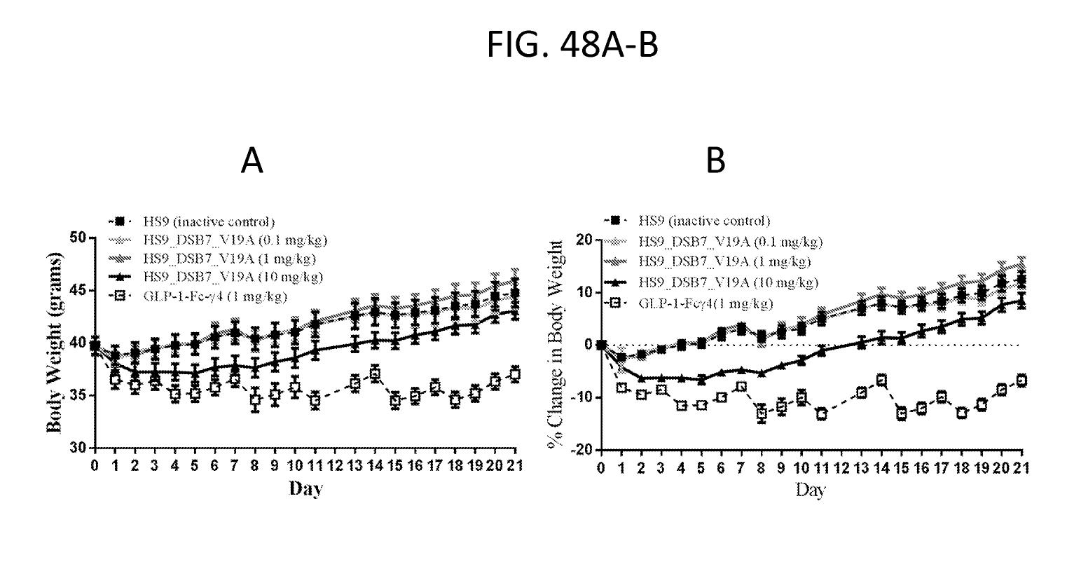

The results of the day 0 and 7 IPGTTs are presented in FIG. 47A-B.

In order to assess effects of the GLP-1 component of HS9_DSB7_V19A on body weight, all animals were weighed daily for the entire course of the study. Effects on body weight are presented in 48A-B.

E) Effects of Multiple Doses of HS9_DSB7_V19A on Body Weight in DIO Mice.

In order to establish a dose-response in body weight reduction HS9_DSB7_V19A was administered subcutaneously to diet-induced obese mice at 3, 10 and 30 mg/kg once weekly (QW). The duration of the study was 28 days and primary endpoint was body weight reduction. Secondary evaluation of glycemic parameters included analyses included fed glucose throughout the course of the study and measurements of terminal fasting glucose. As in our previous studies in both DIO and db/db mice, the GLP-1 analogue-Fcγ4 fusion was administered subcutaneously, twice weekly (BIW) at 1 mg/kg as a positive control for both glucose control and body weight loss.

Male, 21 week old DIO mice on 60% high fat diet for 15 weeks prior to study start were obtained from Jackson labs (JAX: 380050). Just prior to study start, animals were randomized into 5 groups based on body weight (n=8 animals per group).

The experimental groups (n=8 per group) were as follows:

Group 1: Vehicle control

Group 2: HS9_DSB7_V19A—3 mg/kg subcutaneous dose—once weekly (QW)

Group 3: HS9_DSB7_V19A—10 mg/kg subcutaneous dose—once weekly (QW)

Group 4: HS9_DSB7_V19A—30 mg/kg subcutaneous dose—once weekly (QW)

Group 5: GLP-1 analogue-Fcγ4 fusion—1 mg/kg subcutaneous dose—twice weekly dosing (BIW)

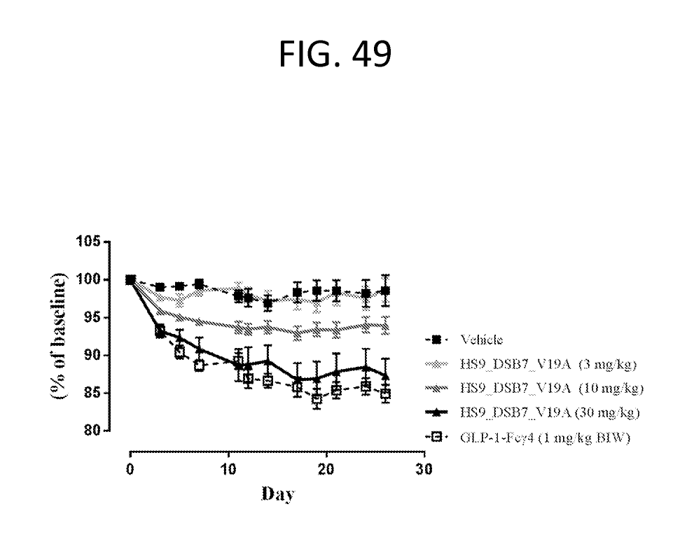

In order to assess effects of the GLP-1 component of HS9_DSB7_V19A on body weight (the primary endpoint of this study), all animals were weighed daily for the entire course of the study. Effects on body weight are presented in FIG. 49.

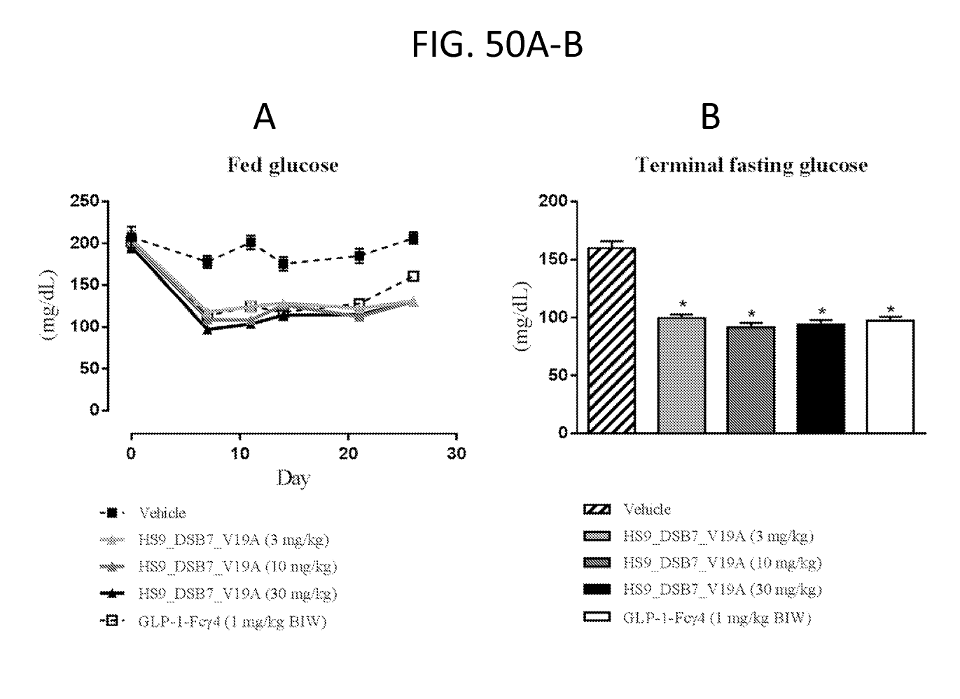

As a secondary endpoint and in order to assess effects of the GLP-1 component of HS9_DSB7_V19A on glycemic control in a weekly dose setting, fed glucose was measured at days 0, 7, 11, 14, 21 and 26 and fasting glucose was measured at just prior to study termination (day 28). Results are presented in FIG. 50A-B.

### Equivalents

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiments may be practiced in many ways and the claims include any equivalents thereof.

1-28. (canceled)

29. A method of controlling glucose in a subject comprising administering to a subject in need thereof, a dual active fusion molecule comprising an anti-PCSK9 antibody stably fused to a GLP-1 peptide, wherein the anti-PCSK9 antibody binds a PCSK9 polypeptide and the GLP-1 peptide binds a GLP-1 receptor.

30. A method of reducing low density lipoprotein (LDL) in a subject comprising administering to a subject in need thereof, a dual active fusion molecule comprising an anti-PCSK9 antibody stably fused to a GLP-1 peptide, wherein the anti-PCSK9 antibody binds a PCSK9 polypeptide and the GLP-1 peptide binds a GLP-1 receptor.

31. A method of controlling glucose and reducing LDL in a subject comprising administering to a subject in need thereof, a dual active fusion molecule comprising an anti-PCSK9 antibody stably fused to a GLP-1 peptide, wherein the anti-PCSK9 antibody binds a PCSK9 polypeptide and the GLP-1 peptide binds a GLP-1 receptor.

31-40. (canceled)


SEQUENCE LISTING (510 sequences)

SEQ ID NO: 1 - protein, 118 aa, Artificial Sequence
  Description of Artificial Sequence Synthetic polypeptide
QVQLVQSGAEVKKPGASVKVSCKASGYTFTSYYMHWVRQAPGQGLEWMGEISPSGGSTSYNQKFQGRVTMTRDTSTSTVYMELSSLRSEDTAVYYCARERPLYASDLWGQGTTVTVSS

SEQ ID NO: 2 - protein, 107 aa, Artificial Sequence
  Description of Artificial Sequence Synthetic polypeptide
DIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 3 - protein, 41 aa, Artificial Sequence
  Description of Artificial Sequence Synthetic polypeptide
HGEGTFTSDLSKQMEEECARLFIEWLKNGGPSSGAPPPGCG

SEQ ID NO: 4 - protein, 16 aa, Artificial Sequence
  Description of Artificial Sequence Synthetic peptide
GGGGSGGGGSGGGGSA

SEQ ID NO: 5 - protein, 118 aa, Artificial Sequence
  Description of Artificial Sequence Synthetic polypeptide
QVQLVQSGAEVKKPGASVKVSCKASGYTFTSYYMHWVRQAPGQGLEWMGEIHPSGGSTSYNQKFQGRVTMTRDTSTSTVYMELSSLRSEDTAVYYCARERPLYASDLWGQGTTVTVSS

SEQ ID NO: 6 - protein, 107 aa, Artificial Sequence
  Description of Artificial Sequence Synthetic polypeptide
DIQMTQSPSSLSASVGDRVTITCQASQDVHTAVAWYQQKPGKAPKLLIYHASYRYTGVPSRFSGSGSGTDFTFTISSLQPEDIATYYCQQRYSLWRTFGQGTKLEIK

SEQ ID NO: 7 - protein, 41 aa, Artificial Sequence
  Description of Artificial Sequence Synthetic polypeptide
HGEGTFTSDLSKQMEEECVRLFIEWLKNGGPSSGAPPPSCG

SEQ ID NO: 8 - protein, 118 aa, Artificial Sequence [sequence omitted]

SEQ ID NO: 9 - protein, 107 aa, Artificial Sequence [sequence omitted]

SEQ ID NO: 10 - protein, 118 aa, Artificial Sequence
  Description of Artificial Sequence Synthetic polypeptide
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 11 - protein, 107 aa, Artificial Sequence
  Description of Artificial Sequence Synthetic polypeptide
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 12 - protein, 39 aa, Artificial Sequence
  Description of Artificial Sequence Synthetic polypeptide
HGEGTFTSDLSKQMEEEAVRLFIEWLKNGGPSSGAPPPS

SEQ ID NO: 13 - protein, 57 aa, Artificial Sequence
  Description of Artificial Sequence Synthetic polypeptide
HGEGTFTSDLSKQMEEECVRLFIEWLKNGGPSSGAPPPGCGGGGGSGGGGSGGGGSA

SEQ ID NO: 14 - protein, 5 aa, Artificial Sequence
  Description of Artificial Sequence Synthetic peptide
SYYMH

SEQ ID NO: 15 - protein, 17 aa, Artificial Sequence
  Description of Artificial Sequence Synthetic peptide
EISPSGGSTSYNQKFQG

SEQ ID NO: 16 - protein, 17 aa, Artificial Sequence
  Description of Artificial Sequence Synthetic peptide
EIHPSGGSTSYNQKFQG

SEQ ID NO: 17 - protein, 17 aa, Artificial Sequence
  Description of Artificial Sequence Synthetic peptide
EIHPSGGRTNYNEKFKS

SEQ ID NO: 18 - protein, 17 aa, Artificial Sequence
  Description of Artificial Sequence Synthetic peptide
EISPFGGRTNYNEKFKS

SEQ ID NO: 19 - protein, 9 aa, Artificial Sequence
  Description of Artificial Sequence Synthetic peptide
ERPLYASDL

SEQ ID NO: 20 - protein, 11 aa, Artificial Sequence
  Description of Artificial Sequence Synthetic peptide
QASQDVKTAVA

SEQ ID NO: 21 - protein, 11 aa, Artificial Sequence
  Description of Artificial Sequence Synthetic peptide
QASQDVHTAVA

SEQ ID NO: 22 - protein, 11 aa, Artificial Sequence
  Description of Artificial Sequence Synthetic peptide
KASQDVHTAVA

SEQ ID NO: 23 - protein, 11 aa, Artificial Sequence
  Description of Artificial Sequence Synthetic peptide
RASQGISSALA

SEQ ID NO: 24 - protein, 7 aa, Artificial Sequence
  Description of Artificial Sequence Synthetic peptide
SASYRYT

SEQ ID NO: 25 - protein, 7 aa, Artificial Sequence
  Description of Artificial Sequence Synthetic peptide
HASYRYT

SEQ ID NO: 26 - protein, 9 aa, Artificial Sequence
  Description of Artificial Sequence Synthetic peptide
QQRYSLWRT

SEQ ID NO: 27 - protein, 5 aa, Artificial Sequence
  Description of Artificial Sequence Synthetic peptide
GGGGS

SEQ ID NO: 28 - protein, 31 aa, Artificial Sequence
  Description of Artificial Sequence Synthetic polypeptide
HGEGTFTSDVSSYLEEQAAKEFIAWLVKGGG

SEQ ID NO: 29 - protein, 31 aa, Homo sapiens
HAEGTFTSDVSSYLEGQAAKEFIAWLVKGRG

SEQ ID NO: 30 - protein, 53 aa, Artificial Sequence
  Description of Artificial Sequence Synthetic polypeptide
HGEGTFTSCLSKQMEEEAVRLFIEWLKNGGPSSGAPPPSGGGGGGGGGGGCGG

SEQ ID NO: 31 - protein, 53 aa, Artificial Sequence
  Description of Artificial Sequence Synthetic polypeptide
HGECTFTSDLSKQMEEEAVRLFIEWLKNGGPSSGAPPPSGGGGGGGGGGGGCG

SEQ ID NO: 32 - protein, 39 aa, Artificial Sequence
  Description of Artificial Sequence Synthetic polypeptide
HGEGTFTSDLSKQMEEECVRLFIEWLKNGGPSSGAPPGC

SEQ ID NO: 33 - protein, 39 aa, Artificial Sequence
  Description of Artificial Sequence Synthetic polypeptide
HGEGTFTSDLSKQMEEEAVRCFIEWLKNGGPSSGAGGCS

SEQ ID NO: 34 - protein, 51 aa, Artificial Sequence
  Description of Artificial Sequence Synthetic polypeptide
HGEGTFTSCLSKQMEEEAVRLFIEWLKNGGPSSGAPPPSGGGGGGGGGCGG

SEQ ID NO: 35 - protein, 53 aa, Artificial Sequence
  Description of Artificial Sequence Synthetic polypeptide
HGEGTFTSCLSKQMEEEAVRLFIEWLKNGGPSSGAPPPSGGGGGGGGGGCGGG

SEQ ID NO: 36 - protein, 41 aa, Artificial Sequence
  Description of Artificial Sequence Synthetic polypeptide
HGEGTFTSDLSKQMEEECVRLFIEWLKNGGPSSGAPPPGCG

SEQ ID NO: 37 - protein, 41 aa, Artificial Sequence
  Description of Artificial Sequence Synthetic polypeptide
HGEGTFTSDLSKQMEEECVRLFIEWLKNGGPSSGAPPGGCG

SEQ ID NO: 38 - protein, 40 aa, Artificial Sequence
  Description of Artificial Sequence Synthetic polypeptide
HGEGTFTSDLSKQMEEEAVRCFIEWLKNGGPSSGAPPCGG

SEQ ID NO: 39 - protein, 40 aa, Artificial Sequence
  Description of Artificial Sequence Synthetic polypeptide
HGEGTFTSDLSKQMEEEAVRCFIEWLKNGGPSSGAPPGCG

SEQ ID NO: 40 - protein, 39 aa, Artificial Sequence
  Description of Artificial Sequence Synthetic polypeptide
HGEGTFTSDLSKQMEEEAVRLFIECLKNGGPSSGACGGS

SEQ ID NO: 41 - protein, 39 aa, Artificial Sequence
  Description of Artificial Sequence Synthetic polypeptide
HGEGTFTSDLSKQMEEEAVRLFIECLKNGGPSSGAPCPS

SEQ ID NO: 42 - protein, 39 aa, Artificial Sequence
  Description of Artificial Sequence Synthetic polypeptide
HGEGTFTSDLSKQMEEEAVRLFIECLKNGGPSSGAPPCS

SEQ ID NO: 43 - protein, 155 aa, Artificial Sequence
  Description of Artificial Sequence Synthetic polypeptide
HGEGTFTSDVSSYLEEQAAKEFIAWLVKGGGGGGGSGGGGSGGGGSADIQMTQSPSSLSASVGDRVTITCKASQDVHTAVAWYQQKPGKAPKLLIYHASYRYTGVPSRFSGSGSGTDFTFTISSLQPEDIATYYCQQRYSLWRTFGQGTKLEIKR

SEQ ID NO: 44 - protein, 41 aa, Artificial Sequence
  Description of Artificial Sequence Synthetic polypeptide
HVEGTFTSDLSKQMEEECVRLFIEWLKNGGPSSGAPPPGCG

SEQ ID NO: 45 - protein, 41 aa, Artificial Sequence
  Description of Artificial Sequence Synthetic polypeptide
HGEGTFTSDLSKQMAEECVRLFIEWLKNGGPSSGAPPPGCG

SEQ ID NO: 46 - protein, 41 aa, Artificial Sequence
  Description of Artificial Sequence Synthetic polypeptide
HGEGTFTSDLSKQMEEECVRLFIEWIKNGGPSSGAPPPGCG

SEQ ID NO: 47 - protein, 164 aa, Artificial Sequence
  Description of Artificial Sequence Synthetic polypeptide
HGEGTFTSDLSKQMEEECARLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 48 - protein, 177 aa, Artificial Sequence
  Description of Artificial Sequence Synthetic polypeptide
HGEGTFTSCLSKQMEEEAVRLFIEWLKNGGPSSGAPPPSGGGGGGGGGGGCGGGGGGSGGGGSGGGGSADIQMTQSPSSLSASVGDRVTITCKASQDVHTAVAWYQQKPGKAPKLLIYHASYRYTGVPSRFSGSGSGTDFTFTISSLQPEDIATYYCQQRYSLWRTFGQGTKLEIKR

SEQ ID NO: 49 - protein, 163 aa, Artificial Sequence
  Description of Artificial Sequence Synthetic polypeptide
HGEGTFTSDLSKQMEEECVRLFIEWLKNGGPSSGAPPGCGGGGSGGGGSGGGGSADIQMTQSPSSLSASVGDRVTITCKASQDVHTAVAWYQQKPGKAPKLLIYHASYRYTGVPSRFSGSGSGTDFTFTISSLQPEDIATYYCQQRYSLWRTFGQGTKLEIKR

SEQ ID NO: 50 - protein, 155 aa, Artificial Sequence
  Description of Artificial Sequence Synthetic polypeptide
HGEGTFTSDVSSYLEEQAAKEFIANLSKGGGGGGGSGGGGSGGGGSADIQMTQSPSSLSASVGDRVTITCKASQDVHTAVAWYQQKPGKAPKLLIYHASYRYTGVPSRFSGSGSGTDFTFTISSLQPEDIATYYCQQRYSLWRTFGQGTKLEIKR

SEQ ID NO: 51 - protein, 165 aa, Artificial Sequence
  Description of Artificial Sequence Synthetic polypeptide
HGEGTFTSDLSKQMEEECVRLFIEWLKNGGPSSGAPPPGCGGGGGSGGGGSGGGGSADIQMTQSPSSLSASVGDRVTITCKASQDVHTAVAWYQQKPGKAPKLLIYHASYRYTGVPSRFSGSGSGTDFTFTISSLQPEDIATYYCQQRYSLWRTFGQGTKLEIKR

SEQ ID NO: 52 - protein, 164 aa, Artificial Sequence
  Description of Artificial Sequence Synthetic polypeptide
HGEGTFTSDLSKQMEEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 53 - protein, 118 aa, Artificial Sequence [sequence omitted]

SEQ ID NO: 54 - protein, 107 aa, Artificial Sequence [sequence omitted]

SEQ ID NO: 55 - protein, 118 aa, Artificial Sequence; identical to SEQ ID NO: 10
  Description of Artificial Sequence Synthetic polypeptide

SEQ ID NO: 56 - protein, 107 aa, Artificial Sequence; identical to SEQ ID NO: 11
  Description of Artificial Sequence Synthetic polypeptide

SEQ ID NO: 57 - protein, 115 aa, Artificial Sequence
  Description of Artificial Sequence Synthetic polypeptide
EVQLQQSGPELVKPGASVKISCKASGYTFTDYYMNWVKQSHGKSLEWIGDINPNNGGTTYNQKFKGKATLTVDKSYSTAYMELRSLTSEDSAVYYCARWLLFAYWGQGTLVTVSA

SEQ ID NO: 58 - protein, 108 aa, Artificial Sequence
  Description of Artificial Sequence Synthetic polypeptide
DIVMTQSQKFMSTSVGDRVSVTCKASQNVGTNVAWYQQKPGQSPKALIYSASYRYSGVPDRFTGSGSGTDFTLTISNVLSEDLAEYFCQQFYSYPYTFGGGTKLEIKR

SEQ ID NO: 59 - protein, 118 aa, Artificial Sequence
  Description of Artificial Sequence Synthetic polypeptide
QVQLQQPGAELVKPGASVKLSCKASGYTFTSYWMHWVKQRPGQGLEWIGEINPSNGRTNYNEKFKSKATLTVDKSSSTAYMQLSSLTSEDSAVYYCARERPLYAMDYWGQGTSVTVSS

SEQ ID NO: 60 - protein, 108 aa, Artificial Sequence
  Description of Artificial Sequence Synthetic polypeptide
DIVMTQSHKFMSTSVGDRVSITCKASQDVSTAVAWYQQKPGQSPKLLIYSASYRYTGVPDRFTGSGSGTDFTFTISSVQAEDLAVYYCQQRYSTPRTFGGGTKLEIKR

SEQ ID NO: 61 - protein, 123 aa, Artificial Sequence
  Description of Artificial Sequence Synthetic polypeptide
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAGGGIYYRYDRNYFDYWGQGTTLTVSS

SEQ ID NO: 62 - protein, 107 aa, Artificial Sequence
  Description of Artificial Sequence Synthetic polypeptide
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPFTFGSGTKLEIK

SEQ ID NO: 63 - protein, 117 aa, Artificial Sequence
  Description of Artificial Sequence Synthetic polypeptide
EVKLVESEGGLVQPGSSMKLSCTASGFTFSDYYMAWVRQVPEKGLEWVANINYDGSNTSYLDSLKSRFIISRDNAKNILYLQMSSLKSEDTATYYCAREKFAAMDYWGQGTSVTVSS

SEQ ID NO: 64 - protein, 108 aa, Artificial Sequence
  Description of Artificial Sequence Synthetic polypeptide
DIVMTQSHKFMSTSFGDRVSITCKASQDVSNALAWYQQKPGHSPKLLIFSASYRYTGVPDRFTGSGSGTDFTFTISSVQAEDLAVYYCQQHYSTPWTFGGGTKLEIKR

SEQ ID NO: 65 - protein, 123 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSDKYYADSVKGRFTISRDNSKNTLYLQMNSLRAEDTAVYYCARETGLPKLYYYGMDVWGQGTTVTVSS

SEQ ID NO: 66 - protein, 122 aa, Artificial Sequence
  Description of Artificial Sequence Synthetic polypeptide
QVQLQESGPGLVKPSQTLSLTCTVSGGSISSSDYYWSWIRQHPGKGLEWIGYIYYSGSTYYNPSLKSRITISVDTSKNLFSLKLSSVTAADTAVYYCARGGVTTYYYAMDVWGQGTTVTVSS

SEQ ID NO: 67 - protein, 121 aa, Artificial Sequence
  Description of Artificial Sequence Synthetic polypeptide
EVQLLESGGGLVQPGGSLRLSCAASGFTFSSYAMNWVRQAPGKGLEWVSTISGSGDNTYYADSVKGRFTISRDNSKNTLYLQMNSLRAEDTAVYYCAKKFVLMVYAMLDYWGQGTLVTVSS

SEQ ID NO: 68 - protein, 121 aa, Artificial Sequence
  Description of Artificial Sequence Synthetic polypeptide
EVQLLESGGGLVQPGGSLRLSCAASGFTFSSYAMNWVRQAPGKGLEWVSTISGSGGNTYYADSVKGRFTISRDNSKNTLYLQMNSLRAEDTAVYYCAKKFVLMVYAMLDYWGQGTLVTVSS

SEQ ID NO: 69 - protein, 123 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 70 - protein, 121 aa, Artificial Sequence
  Description of Artificial Sequence Synthetic polypeptide
QVQLQESGPGLVKPSQTLSLTCTVSGGSISSGGYYWSWIRQHPGKGLEWIGYIYNSGSTYYNPSLKSRVTISVDTSKNQFSLKLSSVTAADTAVYYCAREDTAMVPYFDYWGQGTLVTVSS

SEQ ID NO: 71 - protein, 115 aa, Artificial Sequence
  Description of Artificial Sequence Synthetic polypeptide
QVQLVQSGAEVKKPGASVKVSCKASGYTFPSYGISWVRQAPGQGLEWMGWISAYNGNTNYAEKLQGRVTMTTDTSTSTAYMEVRSLRSDDTAVFYCARGYVMDVWGQGTTVTVSS

SEQ ID NO: 72 - protein, 115 aa, Artificial Sequence
  Description of Artificial Sequence Synthetic polypeptide
QVQLVQSGAEVKRPGASVKVSCKASGYTLTSYGISWVRQAPGQGLEWMGWISVYNGNTNYAQKVQGRVTMTTDTSTSTVYMELRSLSSDDTAVYYCARGYGMDVWGQGTTVTVSS

SEQ ID NO: 73 - protein, 115 aa, Artificial Sequence
  Description of Artificial Sequence Synthetic polypeptide
QVQLVQSGAEVKKPGASVKVSCKASGYTLTSYGISWVRQAPGQGLEWMGWISFYNGNTNYAQKVQGRVTMTTDTSTSTVYMELRSLRSDDTAVYYCARGYGMDVWGQGTTVTVSS

SEQ ID NO: 74 - protein, 115 aa, Artificial Sequence
  Description of Artificial Sequence Synthetic polypeptide
QIQLVQSGAEVKKPGASVKVSCKASGYTLTSYGISWVRQAPGQGLEWMGWISFYNGNTNYAQKVQGRVTMTTDTSTSTVYMELRSLRSDDTAVYFCARGYGMDVWGQGTTVTVSS

SEQ ID NO: 75 - protein, 115 aa, Artificial Sequence
  Description of Artificial Sequence Synthetic polypeptide
QIQLVQSGAEVKKPGASVKVSCKASGYPLTSYGISWVRQAPGQGLEWMGWISAYNGNTNYAQKVQGSVTMTTDTSTSTVYMELRSLRSDDTAVYYCARGYGMDVWGQGTTVTVSS

SEQ ID NO: 76 - protein, 115 aa, Artificial Sequence
  Description of Artificial Sequence Synthetic polypeptide
QVQLVQSGAEVKKPGASLKVSCKASGYSLTSYGISWVRQAPGQGLEWMGWISAYNGNTNYAQKVQGRVTMTTDTSTSTVYMEVRSLRSDDTAVYYCARGYGMDVWGQGTTVTVSS

SEQ ID NO: 77 - protein, 115 aa, Artificial Sequence
  Description of Artificial Sequence Synthetic polypeptide
QVQLVQSGAEVKKPGASVKVSCKASGYPLTSYGISWVRQAPGQGLEWMGWISAYNGNTNYAQKVQGRVTMTTDTSTSTVYMELRSLRSDDTAVYYCARGYGMDVWGQGTTVTVSS

SEQ ID NO: 78 - protein, 115 aa, Artificial Sequence
  Description of Artificial Sequence Synthetic polypeptide
QVQLVQSGAEVKKPGASVKVSCKASGYALTSYGISWVRQAPGQGLEWMGWISAYNGNTNYAQKVQGRVTMTTDTSTSTVYMELRSLRSDDTAVYYCARGYGMDVWGQGTTVTVSS

SEQ ID NO: 79 - protein, 115 aa, Artificial Sequence
  Description of Artificial Sequence Synthetic polypeptide
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 80 - protein, 115 aa, Artificial Sequence
  Description of Artificial Sequence Synthetic polypeptide
QVQLVQSGAEVKKPGASVKVSCKASGYSFTSYGISWVRQAPGQGLEWMGWVSAYNGNTNYAQKFQGRVTMTTDTSTSTAYMELRSLRSDDTAVYYCARGYVMDVWGQGTTVTVSS

SEQ ID NO: 81 - protein, 115 aa, Artificial Sequence; identical to SEQ ID NO: 79
  Description of Artificial Sequence Synthetic polypeptide

SEQ ID NO: 82 - protein, 121 aa, Artificial Sequence
  Description of Artificial Sequence Synthetic polypeptide
QVQLQQSGPGLVKPSQTLSLTCAISGDSVSSNSAAWNWIRQSPSRGLEWLGRTYYRSKWYKNYSVSVKSRITINPDTSKNQFSLQLNSVTPGDTAVYYCARGGPTAAFDYWGQGTLVTVSS

SEQ ID NO: 83 - protein, 119 aa, Artificial Sequence
  Description of Artificial Sequence Synthetic polypeptide
EVQLVESGGGLVQPGGSLRLSCVVSGFTFSSYWMSWVRQAPGKGLEWVANIKQDGSEKYYVDSVKGRFTISRDNAKNSLYLQMNSLRAEDTAVYYCARESNWGFAFDIWGQGTMVTVSS

SEQ ID NO: 84 - protein, 119 aa, Artificial Sequence
  Description of Artificial Sequence Synthetic polypeptide
EVQLVESGGGLVQPGGSLRLSCAASGFTFSRYWMSWVRQAPGKGLEWVANIKHDGSEKYYVDSVKGRFTISRDNAKNSLYLQMNSLRAEDTAVYYCARESNWGFAFDVWGHGTMVTVSS

SEQ ID NO: 85 - protein, 116 aa, Artificial Sequence
  Description of Artificial Sequence Synthetic polypeptide
QVQLQQWGAGLLKPSETLSLTCAVYGGSFSAYYWNWIRQPPGKGLEWIGEINHSGRTDYNPSLKSRVTISVDTSKKQFSLKLNSVTAADTAVYYCARGQLVPFDYWGQGTLVTVSS

SEQ ID NO: 86 - protein, 119 aa, Artificial Sequence
  Description of Artificial Sequence Synthetic polypeptide
EVQLVESGGGLVQPGGSLRLSCAASGLTFSNFWMSWVRQAPGKGLEWVANIKQDGSEKYYVDSVKGRFTISRDNAKNSLYLQMNSLRAEDTAVYSCTRESNWGFAFDIWGQGTMVTVSS

SEQ ID NO: 87 - protein, 122 aa, Artificial Sequence
  Description of Artificial Sequence Synthetic polypeptide
QVHLVESGGGVVQPGRSLRLSCAASGFTFNSFGMHWVRQAPGKGLEWVALIWSDGSDEYYADSVKGRFTISRDNSKNTLYLQMNSLRAEDTAVYYCARAIAALYYYYGMDVWGQGTTVTVSS

SEQ ID NO: 88 - protein, 122 aa, Artificial Sequence
  Description of Artificial Sequence Synthetic polypeptide
QVQLVESGGGVVQPGRSLRLSCAASGFTFSSFGMHWVRQAPGKGLEWVALIWNDGSNKYYADSVKGRFTISRDNSKNTLYLQMNSLRAEDTAVYYCARAIAALYYYYGMDVWGQGTTVTVSS

SEQ ID NO: 89 - protein, 122 aa, Artificial Sequence
  Description of Artificial Sequence Synthetic polypeptide
QVHLVESGGGVVQPGRSLRLSCAASGFTFNSFGMHWVRQAPGKGLEWVALIWSDGSDKYYADSVKGRFTISRDNSKNTLYLQMNSLRAEDTAVYYCARAIAALYYYYGMDVWGQGTTVTVSS

SEQ ID NO: 90 - protein, 122 aa, Artificial Sequence
  Description of Artificial Sequence Synthetic polypeptide
QVQLVESGGGVVQPGRSLRLSCAASGFTFSSFGMHWVRQAPGKGLEWVALIWNDGSNKYYADSVKGRFTISRDNSKNTLYLQMNSLRAEDTAVYYCARAIAALYYYYGMDVWGHGTTVTVSS

SEQ ID NO: 91 - protein, 122 aa, Artificial Sequence; identical to SEQ ID NO: 88
  Description of Artificial Sequence Synthetic polypeptide

SEQ ID NO: 92 - protein, 122 aa, Artificial Sequence
  Description of Artificial Sequence Synthetic polypeptide
QVQLVESGGGVVQPGRSLRLSCAASGFTFRSYGMHWVRQAPGKGLEWVALIWHDGSNTYYVDSVKGRFTISRDNSKNTLYLQMNSLRAEDTAVYYCARGIAVAYYYYGMDVWGQGTTVTVSS

SEQ ID NO: 93 - protein, 117 aa, Artificial Sequence
  Description of Artificial Sequence Synthetic polypeptide
EVQLLESGGGLVQPGGSLRLSCAASGFTFSSYAMSWVRQAPGKGLEWVSTISGSGGRTYYADSVKGRFTISRDNSKNTLYLQMNSLRAEDTAVYYCAKEVGSPFDYWGQGTLVTVSS

SEQ ID NO: 94 - protein, 122 aa, Artificial Sequence
  Description of Artificial Sequence Synthetic polypeptide
QVQLVESGGGVVQPGRSLRLSCAASGFTFSSYGMHWVRQAPGKGLEWVAIIWYDGSNKYYADSVKGRFTISRDNSKNTLYLQMNSLRAEDTAVYYCARRGGLAARPGGMDVWGQGTTVTVSS

SEQ ID NO: 95 - protein, 115 aa, Artificial Sequence
  Description of Artificial Sequence Synthetic polypeptide
QVQLVQSGAEVKKPGASVKVSCKASGYTFTSYGISWVRQAPGQGLEWMGWISTYNGNTNYAQKVQGRVTMTTDTSTSTAYMELRSLRSDDTAVYYCARGYTRDYWGQGTLVTVSS

SEQ ID NO: 96 - protein, 112 aa, Artificial Sequence
  Description of Artificial Sequence Synthetic polypeptide
DIVMTQSPLSLSVTPGEPPSISCRSSQSLLHSNGYNFLNWYLQKPGQSPQLLIYLGSHRASGVPDRFSGSGSGTDFTLEISRVEAEDVGVYYCMQVLQTPFTFGPGTKVDIK

SEQ ID NO: 97 - protein, 107 aa, Artificial Sequence
  Description of Artificial Sequence Synthetic polypeptide
DIQMTQSPSSLSASVGDRVTITCRASQRISNYLSWYLQKPGIAPKLLIYAASSLQSGVPSRFSGSGSGTDFTLTISSLQSEDFATYYCQQSYSTPLIFGGGTKVEIK

SEQ ID NO: 98 - protein, 107 aa, Artificial Sequence
  Description of Artificial Sequence Synthetic polypeptide
DILMTQSPSSLSASVGDRVTITCRASQSISSYLNWYQQKPGKAPKVLIYAASSLQSGVPSRFSGSGSGTDFTLTINSLQPEDFATYYCQQSYSSPITFGQGTRLEIK

SEQ ID NO: 99 - protein, 107 aa, Artificial Sequence
  Description of Artificial Sequence Synthetic polypeptide
DIQMTQSPSSLSASVGDRVTITCRASQSISIYLNWYQQKPGKAPYLLIYAAASLQSGVPSRFSGSGSGTDFTLTISSLQPEDFATYYCQQSYSAPITFGQGTRLEIK

SEQ ID NO: 100 - protein, 111 aa, Artificial Sequence
  Description of Artificial Sequence Synthetic polypeptide
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 101 - protein, 111 aa, Artificial Sequence
  Description of Artificial Sequence Synthetic polypeptide
QSVLTQPPSVSGAPGQRVTISCTGSSSNIGAHYDVHWYQQVPGTAPKLLIYGNTYRPSGVPDRFSGSKSGTSASLAITGLQAEDEADYYCQSYDNSLSGVVFGGGTKLTVL

SEQ ID NO: 102 - protein, 109 aa, Artificial Sequence
  Description of Artificial Sequence Synthetic polypeptide
QSALTQPASVSGSPGQSITISCTGTSSDVGRYNSVSWYQHHPGKAPKVMIYEVSNRPSGVSTRFSGSKSGNTASLTISGLQAEDEADYYCSSYTSSSVVFGGGTKLTVL

SEQ ID NO: 103 - protein, 109 aa, Artificial Sequence
  Description of Artificial Sequence Synthetic polypeptide
QSALTQPASVSGSPGQSITISCTGTSSDVGGYNSVSWYQQHPGKPPKLMIYEVSNRPSGVSIRFSGSKSGNTASLTISGLQAEDEADYFCSSYTSTSMVFGGGTKLTVL

SEQ ID NO: 104 - protein, 109 aa, Artificial Sequence; identical to SEQ ID NO: 103
  Description of Artificial Sequence Synthetic polypeptide

SEQ ID NO: 105 - protein, 109 aa, Artificial Sequence
  Description of Artificial Sequence Synthetic polypeptide
QSALTQPASVSGSPGQSITISCTGTSSDVGGYNSVSWYQQHPGKPPKLMIYEVSNRPSGVSNRFSGSKSGNTASLTISGLQAEDEADYFCSSYTSTSMVFGGGTKLAVL

SEQ ID NO: 106 - protein, 109 aa, Artificial Sequence
  Description of Artificial Sequence Synthetic polypeptide
QSALTQPASVSGSPGQSITISCTGTSSDVGGYNSVSWYQQYPGKPPKLKIYEVSNRPSGVSNRFSGSKSGNTASLTISGLQAEDEADYFCSSYTSTSMVFGGGTKLTVL

SEQ ID NO: 107 - protein, 109 aa, Artificial Sequence
  Description of Artificial Sequence Synthetic polypeptide
QSALTQPASVSGSPGQSITISCTGTSSDVGGYNSVSWYQQHPGKPPKLMIYEVSNRPSGVSNRFSGSKSGNTASLTISGLQAEDEADYFCSSYTSTSMVFGGGTKLTVL

SEQ ID NO: 108 - protein, 109 aa, Artificial Sequence; identical to SEQ ID NO: 107
  Description of Artificial Sequence Synthetic polypeptide

SEQ ID NO: 109 - protein, 109 aa, Artificial Sequence
  Description of Artificial Sequence Synthetic polypeptide
QSALTQPASVSGSPGQSITISCTGTNSDVGGYNSVSWYQQHPGKPPKLMIYEVSNRPSGISNRFSGSKSGNTASLTISGLQAEDEADYFCSSYTSTSMVFGGGTKLTVL

SEQ ID NO: 110 - protein, 109 aa, Artificial Sequence
  Description of Artificial Sequence Synthetic polypeptide
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 111 - protein, 109 aa, Artificial Sequence
  Description of Artificial Sequence Synthetic polypeptide
QSALTQPASVSGSPGQSITISCTGTSSDVGAYNSVSWYQQHPGKAPKRMIYEVSNRPSGVSNRFSGSKSGNTASLTISGLQAEDEADYYCSSYTSTNMVFGGGTKLTVL

SEQ ID NO: 112 - protein, 109 aa, Artificial Sequence
  Description of Artificial Sequence Synthetic polypeptide
QSALTQPASVSGSPGQSITISCTGTSSDVGGYNSVSWYQQHPGKAPKLMIYEVTNRPSGVSNRFSGSKSGNTASLTISGLQAEDEADYYCNSYTSTSMVFGGGTKLTVL

SEQ ID NO: 113 - protein, 110 aa, Artificial Sequence
  Description of Artificial Sequence Synthetic polypeptide
LSALTQPASVSGSPGQSITISCTGTSSDVGNYNLVSWYQQYSGKAPKLMIYEVSKRPSGVSNRFSGSKSGNTASLTISGLQAEDEADYYCCSYAGSSTLVFGGGTKLTVL

SEQ ID NO: 114 - protein, 109 aa, Artificial Sequence
  Description of Artificial Sequence Synthetic polypeptide
QSVLTQPPSASGTPGQRVTISCSGSSSNIGSKTVNWYQQVPGTAPKLLIYRNNQRPLGVPDRFSGSKSGTSASLAISGLQSEDEADYYCAAWDDSLNWVFGGGTKLTVL

SEQ ID NO: 115 - protein, 109 aa, Artificial Sequence
  Description of Artificial Sequence Synthetic polypeptide
QSVLTQPPSASGPPGQRVTISCSGSSSNIGSNTVNWYQQLPGTAPKLLIYSNNRRPSGVPDRFSGSKSGTSASLAISGLQSEDEADYYCAAWDDSLNWVFGGGTKLTVL

SEQ ID NO: 116 - protein, 110 aa, Artificial Sequence
  Description of Artificial Sequence Synthetic polypeptide
QSVLTQPPSASGTPGQRVTISCSGSSSNIGSNTVNWYQQLPGTAPKLLIYSNNQRPSGVPDRFSGSKSGTSASLAISGLQSEDEADYYCAVWDDSLNGWVFGGGTKLTVL

SEQ ID NO: 117 - protein, 109 aa, Artificial Sequence
  Description of Artificial Sequence Synthetic polypeptide
QSVLTQPPSASGTPGQRVTISCSGSSSNIGSKTVNWYQQFPGTAPKLLIYSNNRRPSGVPDRFSGSKSGTSASLAISGLQSEDEADYYCAAWDDSLNWVFGAGTKLTVL

SEQ ID NO: 118 - protein, 110 aa, Artificial Sequence
  Description of Artificial Sequence Synthetic polypeptide
QSVLTQPPSVSAAPGQKVTISCSGSSSNIGNNFVSWYQQLPGTAPKLLIYDYNKRPSGIPDRFSGSKSGTSATLGITGLQTGDEADYYCGTWDSSLSAYVFGTGTRVTVL

SEQ ID NO: 119 - protein, 110 aa, Artificial Sequence
  Description of Artificial Sequence Synthetic polypeptide
QSVLTQPPSVSAAPGQKVTISCSGSSSNIGNNFVSWYQQLPGTAPKLLIYDYNKRPSGIPDRFSGSKSGTSATLGITGLQTGDEADYYCGTWDSSLSGYVFGTGTRVTVL

SEQ ID NO: 120 - protein, 110 aa, Artificial Sequence
  Description of Artificial Sequence Synthetic polypeptide
QSVLTQPPSVSAAPGQKVTISCSGSSSNIGNNFVSWYQQFPGTAPKLLIYDYNKRPSGIPDRFSGSKSGTSATLGITGLQTGDEADYYCGTWDSSLSSYVFGTGTRVTVL

SEQ ID NO: 121 - protein, 110 aa, Artificial Sequence; identical to SEQ ID NO: 119
  Description of Artificial Sequence Synthetic polypeptide

SEQ ID NO: 122 - protein, 110 aa, Artificial Sequence
  Description of Artificial Sequence Synthetic polypeptide
QSVLTQPPTVSAAPGQKVTISCSGSSSNIGNNFVSWYQQLPGTAPKLLIYDYNKRPSGIPDRFSGSKSGTSATLGITGLQTGDEADYYCGTWDSSLSGYVFGTGTRVTVL

SEQ ID NO: 123 - protein, 110 aa, Artificial Sequence
  Description of Artificial Sequence Synthetic polypeptide
QSVLTQPPSVSAAPGQKVTISCSGSSSNIGNNFVSWYQQLPGTAPKLLIYDSNKRPSGIPDRFSGSKSGTSATLDITGLQTGDEADYYCGTWDSSLSAYVFGTGTKVTVL

SEQ ID NO: 124 - protein, 110 aa, Artificial Sequence
  Description of Artificial Sequence Synthetic polypeptide
QSVLTQPPSVSAAPGQKVTISCSGSNSNIGNNYVSWYQQLPGTAPKLLIYDNNKRPSGIPDRFSGSNSGTSATLGITGLQTGDEADYYCGTWDSSLSAVVFGGGTKLTVL

SEQ ID NO: 125 - protein, 106 aa, Artificial Sequence
  Description of Artificial Sequence Synthetic polypeptide
SYELTQPPSVSVSPGQTARITCSGDKLGDKYACWYQQKPGQSPVLVIYQNTKWPLGIPERFSGSKSGNTVTLTISGTQAMDEADYYCQAWDSSTVVFGGGTKLTVL

SEQ ID NO: 126 - protein, 116 aa, Artificial Sequence
  Description of Artificial Sequence Synthetic polypeptide
QPVLTQPLFASASLGASVTLTCTLSSGYSSYEVDWYQQRPGKGPRFVMRVDTGGIVGSKGEGIPDRFSVLGSGLNRYLTIKNIQEEDESDYHCGADHGSGTNFVVVFGGGTKLTVL

SEQ ID NO: 127 - protein, 114 aa, Artificial Sequence
  Description of Artificial Sequence Synthetic polypeptide
QVQLVESGGGVVQPGRSLRLSCAASGFTFSSYGMHWVRQAPGKGLEWVAFIGFDGSNIYYGDSVRGRIIISRDNSENTLYLEMNSLRAEDTAVYYCAREKGLDWGQGTLVTVSS

SEQ ID NO: 128 - protein, 118 aa, Artificial Sequence [sequence omitted]

SEQ ID NO: 129 - protein, 128 aa, Artificial Sequence
  Description of Artificial Sequence Synthetic polypeptide
QVQLQESGPGLVKPSETLSLTCTVSGDSINTYYWSWFRQPPGKGLEWIGYIYYSGTTNYNPSLKSRVTISIDTPRNQFSLKLISVTAADTAVYYCARERITMIRGVTLYYYSYGMDVWGQGTTVTVSS

SEQ ID NO: 130 - protein, 127 aa, Artificial Sequence
  Description of Artificial Sequence Synthetic polypeptide
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 131 - protein, 117 aa, Artificial Sequence
  Description of Artificial Sequence Synthetic polypeptide
QVQLVQSGGGLVQPGGSLRLSCAASGFTLSSYDMHWVRQSTGKGLEWVSAIGSTGDTYYPGSVKGRFTITREKAKNSVYLQMNSLRAGDTAVYYCVREGWEVPFDYWGQGTLVTVSS

SEQ ID NO: 132 - protein, 117 aa, Artificial Sequence
  Description of Artificial Sequence Synthetic polypeptide
EVQLVESGGGLVQPGGSLRLSCAASGFTLSSYDMHWVRQSTGKGLEWVSAIGSTGDTYYPGSVKGRFTITREKAKNSVYLQMNSLRAGDTAVYYCVREGWEVPFDYWGQGTLVTVSS

SEQ ID NO: 133 - protein, 117 aa, Artificial Sequence
  Description of Artificial Sequence Synthetic polypeptide
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCVREGWEVPFDYWGQGTLVTVSS

SEQ ID NO: 134 - protein, 114 aa, Artificial Sequence
  Description of Artificial Sequence Synthetic polypeptide
QVQLVQSGGGVVQPGRSLRLSCAASGFTFSSYGMHWVRQAPGKGLEWVAFIGFDGSNIHYGDSVRGRIIISRDNSENTLYLEMNSLRAEDTAMYYCAREKGLDWGQGTTVTVSS

SEQ ID NO: 135 - protein, 114 aa, Artificial Sequence
  Description of Artificial Sequence Synthetic polypeptide
QVQLVESGGGVVQPGRSLRLSCAASGFTFSSYGMHWVRQAPGKGLEWVAFIGFDGSNIHYGDSVRGRIIISRDNSENTLYLEMNSLRAEDTAMYYCAREKGLDWGQGTLVTVSS

SEQ ID NO: 136 - protein, 114 aa, Artificial Sequence
  Description of Artificial Sequence Synthetic polypeptide
QVQLVESGGGVVQPGRSLRLSCAASGFTFSSYGMHWVRQAPGKGLEWVAVIGFDGSNIYYADSVKGRFTISRDNSKNTLYLQMNSLRAEDTAVYYCAREKGLDWGQGTLVTVSS

SEQ ID NO: 137 - protein, 114 aa, Artificial Sequence
  Description of Artificial Sequence Synthetic polypeptide
QVQLQESGGGVVQPGRSLRLSCAASGFTFSSYGMHWVRQAPGKGLEWVAFIGFDGSNIYYGDSVRGRIIISRDNSENTLYLEMNSLRAEDTAVYYCAREKGLDWGQGTLVTVSS

SEQ ID NO: 138 - protein, 114 aa, Artificial Sequence; identical to SEQ ID NO: 136
  Description of Artificial Sequence Synthetic polypeptide

SEQ ID NO: 139 - protein, 118 aa, Artificial Sequence
  Description of Artificial Sequence Synthetic polypeptide
EVQLVESGGGLVQPGGSLRLSCAASGFTFNNYAMSWVRQAPGKGLEWVSAISGSGGTTYYADSVKGRFTISRDNSKNTLYLQMNSLRAEDTAVYYCAKDSNWGNFDLWGRGTLVTVSS

SEQ ID NO: 140 - protein, 117 aa, Artificial Sequence
  Description of Artificial Sequence Synthetic polypeptide
QVQLVQSGGGLVQPGGSLRLSCAVSGFTLSSYDMHWVRQPTGKGLEWVSAIGSTGDTYYPGSVKGRFTISRENAKNSLYLQMNSLRAGDTAVYYCAREGWDVPFDFWGQGTLVTVSS

SEQ ID NO: 141 - protein, 117 aa, Artificial Sequence
  Description of Artificial Sequence Synthetic polypeptide
EVQLVESGGGLVQPGGSLRLSCAVSGFTLSSYDMHWVRQPTGKGLEWVSAIGSTGDTYYPGSVKGRFTISRENAKNSLYLQMNSLRAGDTAVYYCAREGWDVPFDFWGQGTLVTVSS

SEQ ID NO: 142 - protein, 117 aa, Artificial Sequence
  Description of Artificial Sequence Synthetic polypeptide
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCAREGWDVPFDFWGQGTLVTVSS

SEQ ID NO: 143 - protein, 128 aa, Artificial Sequence; identical to SEQ ID NO: 129
  Description of Artificial Sequence Synthetic polypeptide

SEQ ID NO: 144 - protein, 128 aa, Artificial Sequence
  Description of Artificial Sequence Synthetic polypeptide
QVQLQESGPGLVKPSETLSLTCTVSGDSINTYYWSWIRQPPGKGLEWIGYIYYSGTTNYNPSLKSRVTISVDTSKNQFSLKLSSVTAADTAVYYCARERITMIRGVTLYYYSYGMDVWGQGTTVTVSS

SEQ ID NO: 145 - protein, 126 aa, Artificial Sequence
  Description of Artificial Sequence Synthetic polypeptide
QVQLVQSGAEVKKPGASVKVSCKASGYTFTNYGISWVRQAPGQGLELMGWISGYNGNTNYAQELQARVTMTTDTSTSTAYMELRNLRSDDTAVYYCARDRVVVAAANYYFYSMDVWGQGTTVTVSS

SEQ ID NO: 146 - protein, 126 aa, Artificial Sequence; identical to SEQ ID NO: 145
  Description of Artificial Sequence Synthetic polypeptide

SEQ ID NO: 147 - protein, 126 aa, Artificial Sequence
  Description of Artificial Sequence Synthetic polypeptide
QVQLVQSGAEVKKPGASVKVSCKASGYTFTNYGISWVRQAPGQGLEWMGWISGYNGNTNYAQKLQGRVTMTTDTSTSTAYMELRSLRSDDTAVYYCARDRVVVAAANYYFYSMDVWGQGTTVTVSS

SEQ ID NO: 148 - protein, 125 aa, Artificial Sequence
  Description of Artificial Sequence Synthetic polypeptide
QVHLKESGPTLVKPTQTLTLTCTFSGFSLITSGVGVGWIRQPPGKALEWLALIYWNGDKRYSPSLKSRLTITKDTSKNQVVLTMTNMDPVDTATYYCAHRITETSYYFYYGMDVWGQGTTVTVSS

SEQ ID NO: 149 - protein, 125 aa, Artificial Sequence
  Description of Artificial Sequence Synthetic polypeptide
QITLKESGPTLVKPTQTLTLTCTFSGFSLITSGVGVGWIRQPPGKALEWLALIYWNGDKRYSPSLKSRLTITKDTSKNQVVLTMTNMDPVDTATYYCAHRITETSYYFYYGMDVWGQGTTVTVSS

SEQ ID NO: 150 - protein, 125 aa, Artificial Sequence; identical to SEQ ID NO: 149
  Description of Artificial Sequence Synthetic polypeptide

SEQ ID NO: 151 - protein, 125 aa, Artificial Sequence
  Description of Artificial Sequence Synthetic polypeptide
QITLKESGPTLVKPSQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGITVTVSS

SEQ ID NO: 152 - protein, 125 aa, Artificial Sequence
  Description of Artificial Sequence Synthetic polypeptide
QITLKESGPTLVKPSQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGTTVTVSS

SEQ ID NO: 153 - protein, 125 aa, Artificial Sequence
  Description of Artificial Sequence Synthetic polypeptide
QITLKESGPTLVKPTQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGTTVTVSS

SEQ ID NO: 154 - protein, 127 aa, Artificial Sequence
  Description of Artificial Sequence Synthetic polypeptide
EVQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 155 - protein, 127 aa, Artificial Sequence
  Description of Artificial Sequence Synthetic polypeptide
EVQLVESGGGLVQPGGSLRLSCAASGFTFSSHWMSWVRQAPGKGLEWVANINQDGSEKYYVDSVKGRFTISRDNAKNSLYLQMNSLRAEDTAVYYCARDIVLMVYDMDYYYYGMDVWGQGTTVTVSS

SEQ ID NO: 156 - protein, 127 aa, Artificial Sequence
  Description of Artificial Sequence Synthetic polypeptide
QVQLVESGGGVVQPGRSLRLSCAVSGFTFSSYGMHWVRQAPGKGLEWVAAISYDGSNKYYVDSVKGRFTISRDNSKKTLYLQMNSLRAEDTAVYNCAKNIVLVMYDIDYHYYGMDVWGQGTTVTVSS

SEQ ID NO: 157 - protein, 127 aa, Artificial Sequence; identical to SEQ ID NO: 156
  Description of Artificial Sequence Synthetic polypeptide

SEQ ID NO: 158 - protein, 127 aa, Artificial Sequence
  Description of Artificial Sequence Synthetic polypeptide
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKNIVLVMYDIDYHYYGMDVWGQGTTVTVSS

SEQ ID NO: 159 - protein, 127 aa, Artificial Sequence; identical to SEQ ID NO: 156
  Description of Artificial Sequence Synthetic polypeptide

SEQ ID NO: 160 - protein, 127 aa, Artificial Sequence; identical to SEQ ID NO: 156
  Description of Artificial Sequence Synthetic polypeptide

SEQ ID NO: 161 - protein, 127 aa, Artificial Sequence; identical to SEQ ID NO: 158
  Description of Artificial Sequence Synthetic polypeptide

SEQ ID NO: 162 - protein, 124 aa, Artificial Sequence
  Description of Artificial Sequence Synthetic polypeptide
QITLKESGPTLVKPTQTLTLTCTFSGFSLSASGVGVGWFRQPPGKALEWLALIYWNDDKRYSPSLKNSLTITKDTSKNQVVLTMTNMDPVDTATYYCAHRIHLWSYFYYGMDVWGQGTTVTVSS

SEQ ID NO: 163 - protein, 124 aa, Artificial Sequence; identical to SEQ ID NO: 162
  Description of Artificial Sequence Synthetic polypeptide

SEQ ID NO: 164 - protein, 124 aa, Artificial Sequence
  Description of Artificial Sequence Synthetic polypeptide
QITLKESGPTLVKPTQTLTLTCTFSGFSLSASGVGVGWIRQPPGKALEWLALIYWNDDKRYSPSLKSRLTITKDTSKNQVVLTMTNMDPVDTATYYCAHRIHLWSYFYYGMDVWGQGTTVTVSS

SEQ ID NO: 165 - protein, 127 aa, Artificial Sequence
  Description of Artificial Sequence Synthetic polypeptide
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALNYSYYVMDVWGQGTTVTVSS

SEQ ID NO: 166 - protein, 127 aa, Artificial Sequence; identical to SEQ ID NO: 165
  Description of Artificial Sequence Synthetic polypeptide

SEQ ID NO: 167 - protein, 127 aa, Artificial Sequence
  Description of Artificial Sequence Synthetic polypeptide
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALNYSYYVMDVWGQGTTVTVSS

SEQ ID NO: 168 - protein, 118 aa, Artificial Sequence
  Description of Artificial Sequence Synthetic polypeptide
EVQLVESGGGLVKPGGSLRLSCAASGFTFSSYSMDWVRQAPGKGLEWVSSISSSSSYIYYADSVKGRFTISRDTAKNSLYLQMNSLRDEDTAVYYCAREGSSRLFDYWGQGTLVTVSS

SEQ ID NO: 169 - protein, 118 aa, Artificial Sequence; identical to SEQ ID NO: 168
  Description of Artificial Sequence Synthetic polypeptide

SEQ ID NO: 170 - protein, 118 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAREGSSRLFDYWGQGTLVTVSS

SEQ ID NO: 171 - protein, 128 aa, Artificial Sequence
  Description of Artificial Sequence Synthetic polypeptide
QVHLVESGGGLVKPGGSLRLSCAASGFTFSDHYMSWIRQAPGKGLEWISYISNDGGTKYYVDSVEGRFIISRDNAKNSLYLHMNSLRADDTAVYYCARDQGYIGYDSYYYYSYGMDVWGQGTTVTVAS

SEQ ID NO: 172 - protein, 128 aa, Artificial Sequence
  Description of Artificial Sequence Synthetic polypeptide
QVQLVESGGGLVKPGGSLRLSCAASGFTFSDHYMSWIRQAPGKGLEWISYISNDGGTKYYVDSVEGRFIISRDNAKNSLYLHMNSLRADDTAVYYCARDQGYIGYDSYYYYSYGMDVWGQGTTVTVSS

SEQ ID NO: 173 - protein, 128 aa, Artificial Sequence
  Description of Artificial Sequence Synthetic polypeptide
QVQLVESGGGLVKPGGSLRLSCAASGFTFSDHYMSWIRQAPGKGLEWVSYISNDGGTKYYADSVKGRFTISRDNAKNSLYLQMNSLRAEDTAVYYCARDQGYIGYDSYYYYSYGMDVWGQGTTVTVSS

SEQ ID NO: 174 - protein, 120 aa, Artificial Sequence
  Description of Artificial Sequence Synthetic polypeptide
EVQKVESGGGLVKPGGSLRLSCTASGFTFSTYNMNWVRQAPGKGLEWVSSIRSSSNYIYYADSVKGRFTISRDNAKNSLYLQMNSLRADDTAVYYCARDGSSWYDYSDYWGQGTLVTVSS

SEQ ID NO: 175 - protein, 120 aa, Artificial Sequence
  Description of Artificial Sequence Synthetic polypeptide
EVQLVESGGGLVKPGGSLRLSCTASGFTFSTYNMNWVRQAPGKGLEWVSSIRSSSNYIYYADSVKGRFTISRDNAKNSLYLQMNSLRADDTAVYYCARDGSSWYDYSDYWGQGTLVTVSS

SEQ ID NO: 176 - protein, 120 aa, Artificial Sequence
  Description of Artificial Sequence Synthetic polypeptide
EVQLVESGGGLVKPGGSLRLSCAASGFTFSTYNMNWVRQAPGKGLEWVSSIRSSSNYIYYADSVKGRFTISRDNAKNSLYLQMNSLRAEDTAVYYCARDGSSWYDYSDYWGQGTLVTVSS

SEQ ID NO: 177 - protein, 120 aa, Artificial Sequence; identical to SEQ ID NO: 175
  Description of Artificial Sequence Synthetic polypeptide

SEQ ID NO: 178 - protein, 120 aa, Artificial Sequence; identical to SEQ ID NO: 175
  Description of Artificial Sequence Synthetic polypeptide

SEQ ID NO: 179 - protein, 120 aa, Artificial Sequence; identical to SEQ ID NO: 176
  Description of Artificial Sequence Synthetic polypeptide

SEQ ID NO: 180 - protein, 120 aa, Artificial Sequence
  Description of Artificial Sequence Synthetic polypeptide
EVQLVESGGGLVKPGGSLRLSCTASGFTFSTYNMNWVRQAPGKGLEWVSSIRSSSNYIYYADSVKGRFTISRDNAKSSLYLQMNSLRAEDTAVYYCARDGSSWYDYSDYWGQGTLVTVSS

SEQ ID NO: 181 - protein, 120 aa, Artificial Sequence; identical to SEQ ID NO: 180
  Description of Artificial Sequence Synthetic polypeptide

SEQ ID NO: 182 - protein, 120 aa, Artificial Sequence; identical to SEQ ID NO: 176
  Description of Artificial Sequence Synthetic polypeptide

SEQ ID NO: 183 - protein, 120 aa, Artificial Sequence; identical to SEQ ID NO: 175
  Description of Artificial Sequence Synthetic polypeptide

SEQ ID NO: 184 - protein, 120 aa, Artificial Sequence; identical to SEQ ID NO: 175
  Description of Artificial Sequence Synthetic polypeptide

SEQ ID NO: 185 - protein, 120 aa, Artificial Sequence; identical to SEQ ID NO: 176
  Description of Artificial Sequence Synthetic polypeptide

SEQ ID NO: 186 - protein, 118 aa, Artificial Sequence
  Description of Artificial Sequence Synthetic polypeptide
EVQLVESGGGLVQPGGSLRLSCVVSGFTFGDYDMHWVRQATGRGLEWVSGIAPAGDTSYTGSVKGRFTISRENAKNSLHLQMNSLTTGDTAIYYCAREDIAVPGFDYWGQGTLVTVSS

SEQ ID NO: 187 - protein, 118 aa, Artificial Sequence; identical to SEQ ID NO: 186
  Description of Artificial Sequence Synthetic polypeptide

SEQ ID NO: 188 - protein, 118 aa, Artificial Sequence
  Description of Artificial Sequence Synthetic polypeptide
EVQLVESGGGLVQPGGSLRLSCAASGFTFGDYDMHWVRQATGKGLEWVSAIAPAGDTYYPGSVKGRFTISRENAKNSLYLQMNSLRAGDTAVYYCAREDIAVPGFDYWGQGTLVTVSS

SEQ ID NO: 189 - protein, 126 aa, Artificial Sequence
  Description of Artificial Sequence Synthetic polypeptide
QILLVQSGPEVKEPGASVKVSCKASGYTFTNYAISWVRQVPGQGLEWMGWVSAYNGHTNYAHEVQGRVTMTTDTSTTTAYMELRSLRSDDTAMYYCARGGVVVPVAPHFYNGMDVWGQGTTVTVSS

SEQ ID NO: 190 - protein, 126 aa, Artificial Sequence
  Description of Artificial Sequence Synthetic polypeptide
QVQLVQSGPEVKEPGASVKVSCKASGYTFTNYAISWVRQVPGQGLEWMGWVSAYNGHTNYAHEVQGRVTMTTDTSTTTAYMELRSLRSDDTAMYYCARGGVVVPVAPHFYNGMDVWGQGTTVTVSS

SEQ ID NO: 191 - protein, 126 aa, Artificial Sequence
  Description of Artificial Sequence Synthetic polypeptide
QVQLVQSGAEVKKPGASVKVSCKASGYTFTNYAISWVRQAPGQGLEWMGWVSAYNGHTNYAQKLQGRVTMTTDTSTSTAYMELRSLRSDDTAVYYCARGGVVVPVAPHFYNGMDVWGQGTTVTVSS

SEQ ID NO: 192 - protein, 117 aa, Artificial Sequence
  Description of Artificial Sequence Synthetic polypeptide
EVQLVESGGGLVQPGGSLRLSCAASGFTLSSYDMHWVRQATGKGLEWVSAIGSTGDTYYTGSVMGRFTISRDAAKNSFYLEMNSLRVGDTAVYYCAREGIRTPYDYWGQGARVTVSS

SEQ ID NO: 193 - protein, 117 aa, Artificial Sequence
  Description of Artificial Sequence Synthetic polypeptide
EVQLVESGGGLVQPGGSLRLSCAASGFTLSSYDMHWVRQATGKGLEWVSAIGSTGDTYYTGSVMGRFTISRDAAKNSFYLEMNSLRVGDTAVYYCAREGIRTPYDYWGQGTLVTVSS

SEQ ID NO: 194 - protein, 117 aa, Artificial Sequence
  Description of Artificial Sequence Synthetic polypeptide
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCAREGIRTPYDYWGQGTLVTVSS

SEQ ID NO: 195 - protein, 117 aa, Artificial Sequence; identical to SEQ ID NO: 192
  Description of Artificial Sequence Synthetic polypeptide

SEQ ID NO: 196 - protein, 117 aa, Artificial Sequence; identical to SEQ ID NO: 193
  Description of Artificial Sequence Synthetic polypeptide

SEQ ID NO: 197 - protein, 117 aa, Artificial Sequence; identical to SEQ ID NO: 194
  Description of Artificial Sequence Synthetic polypeptide

SEQ ID NO: 198 - protein, 121 aa, Artificial Sequence
  Description of Artificial Sequence Synthetic polypeptide
EVQLVESGGGLVQPGRSLRLSCAASGFTFDDYAMHWVRQAPGKGLEWVSGINWNSGSIGYADSVKGRFTISRDNAKHSLYLQMNSLRPEDTALYYCVKEVTTGYYYGMDVWGQGTTVTVSS

SEQ ID NO: 199 - protein, 121 aa, Artificial Sequence; identical to SEQ ID NO: 198
  Description of Artificial Sequence Synthetic polypeptide

SEQ ID NO: 200 - protein, 121 aa, Artificial Sequence
  Description of Artificial Sequence Synthetic polypeptide
EVQLVESGGGLVQPGRSLRLSCAASGFTFDDYAMHWVRQAPGKGLEWVSGINWNSGSIGYADSVKGRFTISRDNAKNSLYLQMNSLRAEDTALYYCVKEVTTGYYYGMDVWGQGTTVTVSS

SEQ ID NO: 201 - protein, 122 aa, Artificial Sequence
  Description of Artificial Sequence Synthetic polypeptide
EVQLLESGGGLVQPGGSLRLSCAASGFTFSSYAMNWVRQAPGKGLDWVSGISGNGGSTYYADSVKGRFTISRDISKNTLYVQMHSLRVEDTAVYYCAKARYYDFWGGNFDLWGRGTQVTVSS

SEQ ID NO: 202 - protein, 122 aa, Artificial Sequence
  Description of Artificial Sequence Synthetic polypeptide
EVQLLESGGGLVQPGGSLRLSCAASGFTFSSYAMNWVRQAPGKGLDWVSGISGNGGSTYYADSVKGRFTISRDISKNTLYVQMHSLRVEDTAVYYCAKARYYDFWGGNFDLWGRGTLVTVSS

SEQ ID NO: 203 - protein, 122 aa, Artificial Sequence
  Description of Artificial Sequence Synthetic polypeptide
EVQLLESGGGLVQPGGSLRLSCAASGFTFSSYAMSWVRQAPGKGLEWVSAISGNGGSTYYADSVKGRFTISRDNSKNTLYLQMNSLRAEDTAVYYCAKARYYDFWGGNFDLWGRGTLVTVSS

SEQ ID NO: 204 - protein, 127 aa, Artificial Sequence
  Description of Artificial Sequence Synthetic polypeptide
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALYYSYYVMDVWGQGTTVTVSS

SEQ ID NO: 205 - protein, 127 aa, Artificial Sequence; identical to SEQ ID NO: 204
  Description of Artificial Sequence Synthetic polypeptide

SEQ ID NO: 206 - protein, 127 aa, Artificial Sequence
  Description of Artificial Sequence Synthetic polypeptide
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALYYSYYVMDVWGQGTTVTVSS

SEQ ID NO: 207 - protein, 127 aa, Artificial Sequence
  Description of Artificial Sequence Synthetic polypeptide
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALNYYYYVMDVWGQGTTVTVSS

SEQ ID NO: 208 - protein, 127 aa, Artificial Sequence; identical to SEQ ID NO: 207
  Description of Artificial Sequence Synthetic polypeptide

SEQ ID NO: 209 - protein, 127 aa, Artificial Sequence
  Description of Artificial Sequence Synthetic polypeptide
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALNYYYYVMDVWGQGTTVTVSS

SEQ ID NO: 210 - protein, 127 aa, Artificial Sequence
  Description of Artificial Sequence Synthetic polypeptide
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALYYYYYVMDVWGQGTTVTVSS

SEQ ID NO: 211 - protein, 127 aa, Artificial Sequence; identical to SEQ ID NO: 210
  Description of Artificial Sequence Synthetic polypeptide

SEQ ID NO: 212 - protein, 127 aa, Artificial Sequence
  Description of Artificial Sequence Synthetic polypeptide
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALYYYYYVMDVWGQGTTVTVSS

SEQ ID NO: 213 - protein, 128 aa, Artificial Sequence; identical to SEQ ID NO: 171
  Description of Artificial Sequence Synthetic polypeptide

SEQ ID NO: 214 - protein, 128 aa, Artificial Sequence; identical to SEQ ID NO: 172
  Description of Artificial Sequence Synthetic polypeptide

SEQ ID NO: 215 - protein, 128 aa, Artificial Sequence; identical to SEQ ID NO: 173
  Description of Artificial Sequence Synthetic polypeptide

SEQ ID NO: 216 - protein, 126 aa, Artificial Sequence; identical to SEQ ID NO: 189
  Description of Artificial Sequence Synthetic polypeptide

SEQ ID NO: 217 - protein, 126 aa, Artificial Sequence; identical to SEQ ID NO: 190
  Description of Artificial Sequence Synthetic polypeptide

SEQ ID NO: 218 - protein, 126 aa, Artificial Sequence; identical to SEQ ID NO: 191
  Description of Artificial Sequence Synthetic polypeptide

SEQ ID NO: 219 - protein, 113 aa, Artificial Sequence
  Description of Artificial Sequence Synthetic polypeptide
DIVMTQSPDSLAVSLGERATINCKSSQSVFHTSNNKNYLVWYQQKPGQPPKLLLYWASTRESGVPDRFSGSGSGTDFTLTISSLQAEDVANYYCHQYYSIPWTFGQGTKVEIK

SEQ ID NO: 220 - protein, 113 aa, Artificial Sequence [sequence omitted]

SEQ ID NO: 221 - protein, 107 aa, Artificial Sequence
  Description of Artificial Sequence Synthetic polypeptide
DIQMTQSPSFLSASVGDRVTITCWASQDISSYLAWYQQKPGIAPKLLIYAASTLQSGVPSRFGGSGSGTEFTLTISSLQPEDFATYYCQQLNSYPRTFGQGTKVEIK

SEQ ID NO: 222 - protein, 112 aa, Artificial Sequence
  Description of Artificial Sequence Synthetic polypeptide
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK

SEQ ID NO: 223 - protein, 109 aa, Artificial Sequence
  Description of Artificial Sequence Synthetic polypeptide
DIQMTQSPATLSVSPGERAALSCRASQSVSSNLAWYHQKPGQAPRLLIYGASTRATGIPARFSGIGSGTEFTLIISSLQSEDFAFYFCQQYNNWPPFTFGPGTKVEIKR

SEQ ID NO: 224 - protein, 108 aa, Artificial Sequence
  Description of Artificial Sequence Synthetic polypeptide
EIVMTQSPATLSVSPGERAALSCRASQSVSSNLAWYHQKPGQAPRLLIYGASTRATGIPARFSGIGSGTEFTLIISSLQSEDFAFYFCQQYNNWPPFTFGPGTKVDIK

SEQ ID NO: 225 - protein, 108 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPPFTFGPGTKVDIK

SEQ ID NO: 226 - protein, 107 aa, Artificial Sequence
  Description of Artificial Sequence Synthetic polypeptide
AIQMTQSPSTLSASVGDRVTITCRASQSISSWLAWYQQKPGKAPKLLIYKASSLESGVPSRFSGSGSGTEFTLTISSLQPDDFATYYCQQYNSYYTFGQGTKVEIKR

SEQ ID NO: 227 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYYTFGQGTKLEIK

SEQ ID NO: 228 - protein, 106 aa, Artificial Sequence; identical to SEQ ID NO: 227
  Description of Artificial Sequence Synthetic polypeptide

SEQ ID NO: 229 - protein, 114 aa, Artificial Sequence
  Description of Artificial Sequence Synthetic polypeptide
AIQMTQSPDSLAVSLGERATINCKSSQSVFHTSNNKNYLVWYQQKPGQPPKLLLYWASTRESGVPDRFSGSGSGTDFTLTISSLQAEDVANYYCHQYYSIPWTFGQGTKVEIKR

SEQ ID NO: 230 - protein, 113 aa, Artificial Sequence
  Description of Artificial Sequence Synthetic polypeptide
DIVMTQSPDSLAVSLGERATINCKSSQSVFHTSNNKNYLAWYQQKPGQPPKLLIYWASTRESGVPDRFSGSGSGTDFTLTISSLQAEDVAVYYCHQYYSIPWTFGQGTKVEIK

SEQ ID NO: 231 - protein, 114 aa, Artificial Sequence
  Description of Artificial Sequence Synthetic polypeptide
DIQMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKVEIKR

SEQ ID NO: 232 - protein, 113 aa, Artificial Sequence
  Description of Artificial Sequence Synthetic polypeptide
DIVMTQSPDSLAVSLGERATINCKSSQSVLYRSNNRNFLAWYQQKPGQPPKLLIYWASTRESGVPDRFSGSGSGTDFTLTISSLQAEDVAVYYCQQYYTTPYTFGQGTKLEIK

SEQ ID NO: 233 - protein, 108 aa, Artificial Sequence
  Description of Artificial Sequence Synthetic polypeptide
AIQLTQSPSSLSASVGDRVTITCRASQDIRNDLGWYQQKPGKAPKLLIYAASSLQSGVPSRFSGSGSGTDFTLTISSLQPEDFATYYCLQDYNYPWTFGQGTKVEIKR

SEQ ID NO: 234 - protein, 107 aa, Artificial Sequence
  Description of Artificial Sequence Synthetic polypeptide
AIQMTQSPSSLSASVGDRVTITCRASQDIRNDLGWYQQKPGKAPKLLIYAASSLQSGVPSRFSGSGSGTDFTLTISSLQPEDFATYYCLQDYNYPWTFGQGTKVEIK

SEQ ID NO: 235 - protein, 107 aa, Artificial Sequence; identical to SEQ ID NO: 234
  Description of Artificial Sequence Synthetic polypeptide

SEQ ID NO: 236 - protein, 108 aa, Artificial Sequence
  Description of Artificial Sequence Synthetic polypeptide
DIQMTQSPSFLSASVGDRVTITCWASQDISSYLAWYQQKPGIAPKLLIYAASTLQSGVPSRFGGSGSGTEFTLTISSLQPEDFATYYCQQLNSYPRTFGQGTKVEIKR

SEQ ID NO: 237 - protein, 107 aa, Artificial Sequence
  Description of Artificial Sequence Synthetic polypeptide
DIQMTQSPSSLSASVGDRVTITCRASQDISSYLGWYQQKPGKAPKRLIYAASSLQSGVPSRFSGSGSGTEFTLTISSLQPEDFATYYCQQLNSYPRTFGQGTKVEIK

SEQ ID NO: 238 - protein, 113 aa, Artificial Sequence
  Description of Artificial Sequence Synthetic polypeptide
AIQMTQSPLSLSVTLGQPASISCRSSQSLVYSDGDTYLNWFQQRPGQSPRRLIYKVSNRDSGVPDRFSGSGSGTAFTLKISGVEAEDVGVYYCMQATHWPRTFGQGTKVEIKR

SEQ ID NO: 239 - protein, 112 aa, Artificial Sequence
  Description of Artificial Sequence Synthetic polypeptide
DVVMTQSPLSLSVTLGQPASISCRSSQSLVYSDGDTYLNWFQQRPGQSPRRLIYKVSNRDSGVPDRFSGSGSGTAFTLKISGVEAEDVGVYYCMQATHWPRTFGQGTKVEIK

SEQ ID NO: 240 - protein, 112 aa, Artificial Sequence
  Description of Artificial Sequence Synthetic polypeptide
DVVMTQSPLSLPVTLGQPASISCRSSQSLVYSDGDTYLNWFQQRPGQSPRRLIYKVSNRDSGVPDRFSGSGSGTDFTLKISRVEAEDVGVYYCMQATHWPRTFGQGTKVEIK

SEQ ID NO: 241 - protein, 113 aa, Artificial Sequence
  Description of Artificial Sequence Synthetic polypeptide
DIQMTQSPLSLPVTPGEPASISCRSSQSLLHSHGYDYLDWYLQKPGQSPQLLIYLGSNRASGVPDRFSGSGSGTDFTLKISRVEAEDVGVYYCMQALQTPLTFGGGTKVEIKR

SEQ ID NO: 242 - protein, 112 aa, Artificial Sequence
  Description of Artificial Sequence Synthetic polypeptide
DIVMTQSPLSLPVTPGEPASISCRSSQSLLHSHGYDYLDWYLQKPGQSPQLLIYLGSNRASGVPDRFSGSGSGTDFTLKISRVEAEDVGVYYCMQALQTPLTFGGGTKVEIK

SEQ ID NO: 243 - protein, 112 aa, Artificial Sequence; identical to SEQ ID NO: 242
  Description of Artificial Sequence Synthetic polypeptide

SEQ ID NO: 244 - protein, 113 aa, Artificial Sequence
  Description of Artificial Sequence Synthetic polypeptide
DIQMTQSPLSLPVTPGEPASISCRSSQSLLHSHGYNYLDWYLQKPGQSPQLLIYLGSNRASGVPDRFSGGGSGTDFTLKISRVEAEDVGIYYCMQALQTPLTFGGGTKVEIKR

SEQ ID NO: 245 - protein, 112 aa, Artificial Sequence
  Description of Artificial Sequence Synthetic polypeptide
DIVMTQSPLSLPVTPGEPASISCRSSQSLLHSHGYNYLDWYLQKPGQSPQLLIYLGSNRASGVPDRFSGGGSGTDFTLKISRVEAEDVGIYYCMQALQTPLTFGGGTKVEIK

SEQ ID NO: 246 - protein, 112 aa, Artificial Sequence
  Description of Artificial Sequence Synthetic polypeptide
DIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQALQTPLTFGGGTKVEIK

SEQ ID NO: 247 - protein, 112 aa, Artificial Sequence; identical to SEQ ID NO: 222
  Description of Artificial Sequence Synthetic polypeptide

SEQ ID NO: 248 - protein, 112 aa, Artificial Sequence; identical to SEQ ID NO: 222
  Description of Artificial Sequence Synthetic polypeptide

SEQ ID NO: 249 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGFNRASGVPDRFSGSGSGTDFTLKISRVEAEDVGVYYCMQALQTPLTFGGGTKVEIR

SEQ ID NO: 250 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGFNRASGVPDRFSGSGSGTDFTLKISRVEAEDVGVYYCMQALQTPLTFGGGTKVEIK

SEQ ID NO: 251 - protein, 112 aa, Artificial Sequence; identical to SEQ ID NO: 250
  Description of Artificial Sequence Synthetic polypeptide

SEQ ID NO: 252 - protein, 112 aa, Artificial Sequence; identical to SEQ ID NO: 249
  Description of Artificial Sequence Synthetic polypeptide

SEQ ID NO: 253 - protein, 112 aa, Artificial Sequence; identical to SEQ ID NO: 250
  Description of Artificial Sequence Synthetic polypeptide

SEQ ID NO: 254 - protein, 112 aa, Artificial Sequence; identical to SEQ ID NO: 250
  Description of Artificial Sequence Synthetic polypeptide

SEQ ID NO: 255 - protein, 112 aa, Artificial Sequence
  Description of Artificial Sequence Synthetic polypeptide
DIVMTQSPLSLPVTPGEPASISCRSSQTLLHSNGYNYFDWYLQKPGQSPQLLIYLGSNRASGVPDRFSGSGSGTDFTLKISRVEAEDVGIYYCMQALQTPLTFGGGTKVEIR

SEQ ID NO: 256 - protein, 112 aa, Artificial Sequence
  Description of Artificial Sequence Synthetic polypeptide
DIVMTQSPLSLPVTPGEPASISCRSSQTLLHSNGYNYFDWYLQKPGQSPQLLIYLGSNRASGVPDRFSGSGSGTDFTLKISRVEAEDVGIYYCMQALQTPLTFGGGTKVEIK

SEQ ID NO: 257 - protein, 112 aa, Artificial Sequence
  Description of Artificial Sequence Synthetic polypeptide
DIVMTQSPLSLPVTPGEPASISCRSSQTLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 258 - protein, 112 aa, Artificial Sequence
  Description of Artificial Sequence Synthetic polypeptide
DVVMTQSPLSLPVTLGQPASISCRSSQSLVYSDGNTYLNWSQQRPGQSPRRLIYKVSNRDSGVPDRFSGSGSGTDFTLKISRVEAEDVGVYYCMQGTHWPYTFGQGTKLEIK

SEQ ID NO: 259 - protein, 112 aa, Artificial Sequence; identical to SEQ ID NO: 258
  Description of Artificial Sequence Synthetic polypeptide

SEQ ID NO: 260 - protein, 112 aa, Artificial Sequence
  Description of Artificial Sequence Synthetic polypeptide
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPYTFGQGTKLEIK

SEQ ID NO: 261 - protein, 107 aa, Artificial Sequence
  Description of Artificial Sequence Synthetic polypeptide
DIQMTQSPSTLSASVGDRVTITCRASQSISSWLAWYQQRPGKAPKLLIYKASSLEGGVPSRFSGSGSGTEFTLTISSLQPEDFATYYCQQYNSYWYTFGQGTKLEIK

SEQ ID NO: 262 - protein, 107 aa, Artificial Sequence; identical to SEQ ID NO: 261
  Description of Artificial Sequence Synthetic polypeptide

SEQ ID NO: 263 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYWYTFGQGTKLEIK

SEQ ID NO: 264 - protein, 107 aa, Artificial Sequence
  Description of Artificial Sequence Synthetic polypeptide
KIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTLGGGTKVEIK

SEQ ID NO: 265 - protein, 107 aa, Artificial Sequence
  Description of Artificial Sequence Synthetic polypeptide
EIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTLGGGTKVEIK

SEQ ID NO: 266 - protein, 107 aa, Artificial Sequence
  Description of Artificial Sequence Synthetic polypeptide
EIVLTQSPGTLSLSPGERATLSCRASQSVNNKFLAWYQQKPGQAPRLLIYGASSRATGIPDRFSGSGSGTDFTLTISRLEPEDFAVYYCQVYGNSLTFGGGTKVEIK

SEQ ID NO: 267 - protein, 107 aa, Artificial Sequence
  Description of Artificial Sequence Synthetic polypeptide
DIQMTQSPSTLSASVGDRVTITCRASQSISSWLAWYQQIPGKAPKLLIYKASSLENGVPSRFSGSGSGTEFTLIISSLQPDDFATYYCQQYISYSRTFGQGTKVEIK

SEQ ID NO: 268 - protein, 107 aa, Artificial Sequence; identical to SEQ ID NO: 267
  Description of Artificial Sequence Synthetic polypeptide

SEQ ID NO: 269 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYISYSRTFGQGTKVEIK

SEQ ID NO: 270 - protein, 107 aa, Artificial Sequence; identical to SEQ ID NO: 267
  Description of Artificial Sequence Synthetic polypeptide

SEQ ID NO: 271 - protein, 107 aa, Artificial Sequence; identical to SEQ ID NO: 267
  Description of Artificial Sequence Synthetic polypeptide

SEQ ID NO: 272 - protein, 107 aa, Artificial Sequence; identical to SEQ ID NO: 269
  Description of Artificial Sequence Synthetic polypeptide

SEQ ID NO: 273 - protein, 107 aa, Artificial Sequence
  Description of Artificial Sequence Synthetic polypeptide
DIQMTQSPSTLSASVGDRVTITCRASQSISSWLAWYQQVPGKAPKLLIYKASSLENGVPSRFSGSGSGTEFTLIISSLQPDDFATYYCQQYISYSRTFGQGTKVEIK

SEQ ID NO: 274 - protein, 107 aa, Artificial Sequence; identical to SEQ ID NO: 273
  Description of Artificial Sequence Synthetic polypeptide

SEQ ID NO: 275 - protein, 107 aa, Artificial Sequence; identical to SEQ ID NO: 269
  Description of Artificial Sequence Synthetic polypeptide

SEQ ID NO: 276 - protein, 107 aa, Artificial Sequence; identical to SEQ ID NO: 267
  Description of Artificial Sequence Synthetic polypeptide

SEQ ID NO: 277 - protein, 107 aa, Artificial Sequence; identical to SEQ ID NO: 267
  Description of Artificial Sequence Synthetic polypeptide

SEQ ID NO: 278 - protein, 107 aa, Artificial Sequence; identical to SEQ ID NO: 269
  Description of Artificial Sequence Synthetic polypeptide

SEQ ID NO: 279 - protein, 108 aa, Artificial Sequence
  Description of Artificial Sequence Synthetic polypeptide
EIVMTQSPATLSVSPGERGTLSCRASQSVSSNLAWYQQKPGQAPRLLIYGASTRATGFPARFSGSGSGTEFTLTISSLQSEDFAVYYCQQYNKWPPFTFGPGTKVDFK

SEQ ID NO: 280 - protein, 108 aa, Artificial Sequence
  Description of Artificial Sequence Synthetic polypeptide
EIVMTQSPATLSVSPGERGTLSCRASQSVSSNLAWYQQKPGQAPRLLIYGASTRATGFPARFSGSGSGTEFTLTISSLQSEDFAVYYCQQYNKWPPFTFGPGTKVDIK

SEQ ID NO: 281 - protein, 108 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNKWPPFTFGPGTKVDIK

SEQ ID NO: 282 - protein, 112 aa, Artificial Sequence
  Description of Artificial Sequence Synthetic polypeptide
DIVMTQFPLSLPVTPGEPASISCRSSQSLLHINEYNYLDWYLKKPGQSPQLLIYLGFNRASGVPDRFSGSGSGTDFTLKISRVEAEDVGVYYCMQALQTPWTLGQGTKVEIK

SEQ ID NO: 283 - protein, 112 aa, Artificial Sequence
  Description of Artificial Sequence Synthetic polypeptide
DIVMTQSPLSLPVTPGEPASISCRSSQSLLHINEYNYLDWYLKKPGQSPQLLIYLGFNRASGVPDRFSGSGSGTDFTLKISRVEAEDVGVYYCMQALQTPWTLGQGTKVEIK

SEQ ID NO: 284 - protein, 112 aa, Artificial Sequence
  Description of Artificial Sequence Synthetic polypeptide
DIVMTQSPLSLPVTPGEPASISCRSSQSLLHINEYNYLDWYLQKPGQSPQLLIYLGSNRASGVPDRFSGSGSGTDFTLKISRVEAEDVGVYYCMQALQTPWTFGQGTKVEIK

SEQ ID NO: 285 - protein, 108 aa, Artificial Sequence
  Description of Artificial Sequence Synthetic polypeptide
EIVMTQSPATLSVSPGERATLSCRASQSVSSNVAWYQQKPGQAPRLLIYGASTRATGIPARFSGSGSGTEFTLTISSLQSEDFAVYYCQQYNNWPPFTFGPGTKVDIK

SEQ ID NO: 286 - protein, 108 aa, Artificial Sequence; identical to SEQ ID NO: 285
  Description of Artificial Sequence Synthetic polypeptide

SEQ ID NO: 287 - protein, 108 aa, Artificial Sequence; identical to SEQ ID NO: 225
  Description of Artificial Sequence Synthetic polypeptide

SEQ ID NO: 288 - protein, 108 aa, Artificial Sequence; identical to SEQ ID NO: 285
  Description of Artificial Sequence Synthetic polypeptide

SEQ ID NO: 289 - protein, 108 aa, Artificial Sequence; identical to SEQ ID NO: 285
  Description of Artificial Sequence Synthetic polypeptide

SEQ ID NO: 290 - protein, 108 aa, Artificial Sequence; identical to SEQ ID NO: 225
  Description of Artificial Sequence Synthetic polypeptide

SEQ ID NO: 291 - protein, 107 aa, Artificial Sequence
  Description of Artificial Sequence Synthetic polypeptide
DIQLTQSPSFLSASVGDRVTITCWASQGISSYLAWYQKKPGKAPNLLIYDASTLQSGVPSRFSGSGSGTEFTLTLSSLQPEDFATYYCQQLNIYPFTFGPGTKVDIK

SEQ ID NO: 292 - protein, 107 aa, Artificial Sequence; identical to SEQ ID NO: 291
  Description of Artificial Sequence Synthetic polypeptide

SEQ ID NO: 293 - protein, 107 aa, Artificial Sequence
  Description of Artificial Sequence Synthetic polypeptide
DIQLTQSPSFLSASVGDRVTITCRASQGISSYLAWYQQKPGKAPKLLIYDASTLQSGVPSRFSGSGSGTEFTLTISSLQPEDFATYYCQQLNIYPFTFGPGTKVDIK

SEQ ID NO: 294 - protein, 108 aa, Artificial Sequence
  Description of Artificial Sequence Synthetic polypeptide
EIVLTQSPGTLSLSPGERATLSCRASQSVSIRYLAWYQQKPGQAPRLLIYGASSRATGIPDRFSVSVSGTDFTLTITRLEPEDFAVYYCQQYGSSPLTFGGGTKVEIK

SEQ ID NO: 295 - protein, 108 aa, Artificial Sequence; identical to SEQ ID NO: 294
  Description of Artificial Sequence Synthetic polypeptide

SEQ ID NO: 296 - protein, 108 aa, Artificial Sequence
  Description of Artificial Sequence Synthetic polypeptide
EIVLTQSPGTLSLSPGERATLSCRASQSVSIRYLAWYQQKPGQAPRLLIYGASSRATGIPDRFSGSGSGTDFTLTISRLEPEDFAVYYCQQYGSSPLTFGGGTKVEIK

SEQ ID NO: 297 - protein, 112 aa, Artificial Sequence; identical to SEQ ID NO: 260
  Description of Artificial Sequence Synthetic polypeptide

SEQ ID NO: 298 - protein, 112 aa, Artificial Sequence; identical to SEQ ID NO: 260
  Description of Artificial Sequence Synthetic polypeptide

SEQ ID NO: 299 - protein, 112 aa, Artificial Sequence; identical to SEQ ID NO: 260
  Description of Artificial Sequence Synthetic polypeptide

SEQ ID NO: 300 - protein, 112 aa, Artificial Sequence; identical to SEQ ID NO: 260
  Description of Artificial Sequence Synthetic polypeptide

SEQ ID NO: 301 - protein, 112 aa, Artificial Sequence; identical to SEQ ID NO: 260
  Description of Artificial Sequence Synthetic polypeptide

SEQ ID NO: 302 - protein, 112 aa, Artificial Sequence; identical to SEQ ID NO: 260
  Description of Artificial Sequence Synthetic polypeptide

SEQ ID NO: 303 - protein, 112 aa, Artificial Sequence; identical to SEQ ID NO: 260
  Description of Artificial Sequence Synthetic polypeptide

SEQ ID NO: 304 - protein, 112 aa, Artificial Sequence; identical to SEQ ID NO: 260
  Description of Artificial Sequence Synthetic polypeptide

SEQ ID NO: 305 - protein, 112 aa, Artificial Sequence; identical to SEQ ID NO: 260
  Description of Artificial Sequence Synthetic polypeptide

SEQ ID NO: 306 - protein, 107 aa, Artificial Sequence
  Description of Artificial Sequence Synthetic polypeptide
KIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTFGGGTKVEIK

SEQ ID NO: 307 - protein, 107 aa, Artificial Sequence
  Description of Artificial Sequence Synthetic polypeptide
EIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTFGGGTKVEIK

SEQ ID NO: 308 - protein, 107 aa, Artificial Sequence; identical to SEQ ID NO: 266
  Description of Artificial Sequence Synthetic polypeptide

SEQ ID NO: 309 - protein, 112 aa, Artificial Sequence
  Description of Artificial Sequence Synthetic polypeptide
DIVMTQFPLSLPVTPGEPASISCRSSQSLLHINEYNYLDWYLKKPGQSPQLLIYLGFNRASGVPDRFSGSGSGTDFTLKISRVEAEDVGVYYCMQALQTPWTFGQGTKVEIK

SEQ ID NO: 310 - protein, 112 aa, Artificial Sequence
  Description of Artificial Sequence Synthetic polypeptide
DIVMTQSPLSLPVTPGEPASISCRSSQSLLHINEYNYLDWYLKKPGQSPQLLIYLGFNRASGVPDRFSGSGSGTDFTLKISRVEAEDVGVYYCMQALQTPWTFGQGTKVEIK

SEQ ID NO: 311 - protein, 112 aa, Artificial Sequence; identical to SEQ ID NO: 284
  Description of Artificial Sequence Synthetic polypeptide

SEQ ID NO: 312 - protein, 121 aa, Artificial Sequence
  Description of Artificial Sequence Synthetic polypeptide
EVQLVESGGGLVQPGGSLRLSCAASGFTFTGYAIHWVRQAPGKGLEWVGRISPANGNTNYADSVKGRFTISADTSKNTAYLQMNSLRAEDTAVYYCARWIGSRELYIMDYWGQGTLVTVSS

SEQ ID NO: 313 - protein, 121 aa, Artificial Sequence; identical to SEQ ID NO: 312
  Description of Artificial Sequence Synthetic polypeptide

SEQ ID NO: 314 - protein, 121 aa, Artificial Sequence; identical to SEQ ID NO: 312
  Description of Artificial Sequence Synthetic polypeptide

SEQ ID NO: 315 - protein, 121 aa, Artificial Sequence
  Description of Artificial Sequence Synthetic polypeptide
EVQLVESGGGLVQPGGSLRLSCAASGFTFTRHTIHWVRQAPGKGLEWVGRISPANGNTNYADSVKGRFTISADTSKNTAYLQMNSLRAEDTAVYYCARWIGSRELYIMDYWGQGTLVTVSS

SEQ ID NO: 316 - protein, 121 aa, Artificial Sequence
  Description of Artificial Sequence Synthetic polypeptide
EVQLVESGGGLVQPGGSLRLSCAASGFTFSSTAIHWVRQAPGKGLEWVGRISPANGNTNYADSVKGRFTISADTSKNTAYLQMNSLRAEDTAVYYCARWIGSRELYIMDYWGQGTLVTVSS

SEQ ID NO: 317 - protein, 121 aa, Artificial Sequence; identical to SEQ ID NO: 312
  Description of Artificial Sequence Synthetic polypeptide

SEQ ID NO: 318 - protein, 108 aa, Artificial Sequence
  Description of Artificial Sequence Synthetic polypeptide
DIQMTQSPSSLSASVGDRVTITCRASQDVSSAVAWYQQKPGKAPKLLIYSASSLYSGVPSRFSGSRSGTDFTLTISSLQPEDFATYYCQQSYTTPPTFGQGTKVEIKR

SEQ ID NO: 319 - protein, 108 aa, Artificial Sequence
  Description of Artificial Sequence Synthetic polypeptide
DIQMTQSPSSLSASVGDRVTITCRASQDVSSAVAWYQQKPGKAPKLLIYSASSLYSGVPSRFSGSRSGTDFTLTISSLQPEDFATYYCQQSYPAPATFGQGTKVEIKR

SEQ ID NO: 320 - protein, 108 aa, Artificial Sequence
  Description of Artificial Sequence Synthetic polypeptide
DIQMTQSPSSLSASVGDRVTITCRASQDVSSAVAWYQQKPGKAPKLLIYSASSLYSGVPSRFSGSRSGTDFTLTISSLQPEDFATYYCQQSYPSPATFGQGTKVEIKR

SEQ ID NO: 321 - protein, 108 aa, Artificial Sequence
  Description of Artificial Sequence Synthetic polypeptide
DIQMTQSPSSLSASVGDRVTITCRASQDVSSAVAWYQQKPGKAPKLLIYSASSLYSGVPSRFSGSRSGTDFTLTISSLQPEDFATYYCQQSYRIQPTFGQGTKVEIKR

SEQ ID NO: 322 - protein, 108 aa, Artificial Sequence
  Description of Artificial Sequence Synthetic polypeptide
DIQMTQSPSSLSASVGDRVTITCRASQDVSSAVAWYQQKPGKAPKLLIYSASSLYSGVPSRFSGSRSGTDFTLTISSLQPEDFATYYCQQSYPALHTFGQGTKVEIKR

SEQ ID NO: 323 - protein, 108 aa, Artificial Sequence
  Description of Artificial Sequence Synthetic polypeptide
DIQMTQSPSSLSASVGDRVTITCRASQDVSSAVAWYQQKPGKAPKLLIYSASSLYSGVPSRFSGSRSGTDFTLTISSLQPEDFATYYCQQSYPAPSTFGQGTKVEIKR

SEQ ID NO: 324 - protein, 125 aa, Artificial Sequence
  Description of Artificial Sequence Synthetic polypeptide
EVQLVESGGGLVKPGGSLRLSCAASGFPFSKLGMVWVRQAPGKGLEWVSTISSGGGYTYYPDSVKGRFTISRDNAKNSLYLQMNSLRAEDTAVYYCAREGISFQGGTYTYVMDYWGQGTLVTVSS

SEQ ID NO: 325 - protein, 112 aa, Artificial Sequence
  Description of Artificial Sequence Synthetic polypeptide
DIVMTQSPLSLPVTPGEPASISCRSSKSLLHRNGITYSYWYLQKPGQSPQLLIYQLSNLASGVPDRFSGSGSGTDFTLKISRVEAEDVGVYYCYQNLELPLTFGQGTKVEIK

SEQ ID NO: 326 - protein, 119 aa, Artificial Sequence
  Description of Artificial Sequence Synthetic polypeptide
QVQLVQSGAEVKKPGESLKISCKGSGYSFTNYWISWVRQMPGKGLEWMGIIYPGDSYTNYSPSFQGQVTISADKSISTAYLQWSSLKASDTAMYYCARDYWYKPLFDIWGQGTLVTVSS

SEQ ID NO: 327 - protein, 114 aa, Artificial Sequence
  Description of Artificial Sequence Synthetic polypeptide
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYSSFPITFGQGTKVEIKR

SEQ ID NO: 328 - protein, 10 aa, Artificial Sequence
  Description of Artificial Sequence Synthetic peptide
  MOD_RES (6)..(6): Asn or Asp
  MOD_RES (8)..(8): Trp or Tyr
  MOD_RES (10)..(10): Ser, Thr or Ala
GYSFTXYXIX

SEQ ID NO: 329 - protein, 20 aa, Artificial Sequence
  Description of Artificial Sequence Synthetic peptide
  MOD_RES (4)..(4): Ile, Arg, Trp, Leu or Met
  MOD_RES (11)..(11): Tyr or Asp
  MOD_RES (13)..(13): Asn, Arg, His, Ser, Lys or Gln
  MOD_RES (15)..(15): Ser or Asn
  MOD_RES (16)..(16): Pro, Gln or His
  MOD_RES (17)..(17): Ser, Lys, Asn or Arg
WMGXIYPGDSXTXYXXXFQG

SEQ ID NO: 330 - protein, 10 aa, Artificial Sequence
  Description of Artificial Sequence Synthetic peptide
  MOD_RES (2)..(2): Tyr, Arg or His
  MOD_RES (3)..(3): Trp, Tyr, Gly, Ala or Phe
  MOD_RES (4)..(4): Tyr or Ser
  MOD_RES (5)..(5): Lys, Arg, Thr, Gly, Ser, Asp, Glu, His or Asn
  MOD_RES (6)..(6): Pro, Ser, Gly, Asp, Ala, His, Arg or Tyr
  MOD_RES (7)..(7): Leu, Tyr, Phe, Ala, Asp, His, Pro or Ser
  MOD_RES (8)..(8): Phe or Ser
  MOD_RES (10)..(10): Ile, Val, Tyr, Phe or Asn
DXXXXXXXDX

SEQ ID NO: 331 - protein, 20 aa, Artificial Sequence
  Description of Artificial Sequence Synthetic peptide
  MOD_RES (4)..(4): Ile or Arg
  MOD_RES (13)..(13): Asn, Arg, His or Ser
  MOD_RES (16)..(16): Pro or Gln
WMGXIYPGDSYTXYSXSFQG

SEQ ID NO: 332 - protein, 10 aa, Artificial Sequence
  Description of Artificial Sequence Synthetic peptide
  MOD_RES (5)..(5): Lys, Gly, Arg, Ser, Asp, Glu, His, Asn or Gln
  MOD_RES (6)..(6): Pro, Gly, Asp, Ser, Ala, His, Arg or Tyr
  MOD_RES (7)..(7): Leu, Ala, Tyr, Asp, Phe, His, Pro, Ser or Val
  MOD_RES (10)..(10): Ile, Tyr, Phe or Asn
DYWYXXXFDX

SEQ ID NO: 333 - protein, 17 aa, Artificial Sequence
  Description of Artificial Sequence Synthetic peptide
  MOD_RES (1)..(1): Arg, Lys, His, Asn, Gln or Ser
  MOD_RES (7)..(7): Leu or Phe
  MOD_RES (8)..(8): Tyr or His
  MOD_RES (10)..(10): Ser, Arg or Gly
  MOD_RES (11)..(11): Asn or Thr
  MOD_RES (12)..(12): Asn, Arg, His or Ser
  MOD_RES (15)..(15): Tyr or Phe
  MOD_RES (17)..(17): Ala or Thr
XSSQSVXXSXXXKNXLX

SEQ ID NO: 334 - protein, 11 aa, Artificial Sequence
  Description of Artificial Sequence Synthetic peptide
  MOD_RES (5)..(5): Trp, Phe or Leu
  MOD_RES (6)..(6): Ala or Thr
  MOD_RES (8)..(8): Thr, Ile, Ala or Val
  MOD_RES (10)..(10): Glu, Ala or Lys
  MOD_RES (11)..(11): Ser or Thr
LLIYXXSXRXX

SEQ ID NO: 335 - protein, 8 aa, Artificial Sequence
  Description of Artificial Sequence Synthetic peptide
  MOD_RES (4)..(4): Ser or Tyr
  MOD_RES (5)..(5): Ser or Thr
  MOD_RES (6)..(6): Phe, Tyr, Leu, Thr, His, Ile, Asn, Pro or Ser
  MOD_RES (8)..(8): Ile, Arg, Val, Tyr, Asp, Phe, Gly, His, Leu,      Asn or Ser
QQYXXXPX

SEQ ID NO: 336 - protein, 17 aa, Artificial Sequence
  Description of Artificial Sequence Synthetic peptide
  MOD_RES (15)..(15): Tyr or Phe
  MOD_RES (17)..(17): Ala or Thr
RSSQSVLYSSNNKNXLX

SEQ ID NO: 337 - protein, 11 aa, Artificial Sequence
  Description of Artificial Sequence Synthetic peptide
  MOD_RES (5)..(5): Trp, Phe or Leu
  MOD_RES (10)..(10): Glu or Lys
  MOD_RES (11)..(11): Ser or Thr
LLIYXASTRXX

SEQ ID NO: 338 - protein, 8 aa, Artificial Sequence
  Description of Artificial Sequence Synthetic peptide
  MOD_RES (6)..(6): Phe, Tyr, Thr, Ile, Asn or Ser
  MOD_RES (8)..(8): Ile, Tyr, Arg, Phe, His, Leu, Asn or Ser
QQYSSXPX

SEQ ID NO: 339 - protein, 119 aa, Artificial Sequence
  Description of Artificial Sequence Synthetic polypeptide
  MOD_RES (31)..(31): Asn or Asp
  MOD_RES (33)..(33): Trp or Tyr
  MOD_RES (35)..(35): Ser, Thr or Ala
  MOD_RES (50)..(50): Ile, Arg, Trp, Leu or Met
  MOD_RES (57)..(57): Tyr or Asp
  MOD_RES (59)..(59): Asn, Arg, His, Ser, Lys or Gln
  MOD_RES (61)..(61): Ser or Asn
  MOD_RES (62)..(62): Pro, Gln or His
  MOD_RES (63)..(63): Ser, Lys, Asn or Arg
  MOD_RES (100)..(100): Tyr, Arg or His
  MOD_RES (101)..(101): Trp, Tyr or Gly
  MOD_RES (102)..(102): Tyr or Ser
  MOD_RES (103)..(103): Lys, Arg, Thr, Gly, Ser, Asp, Glu, His, Asn or      Gln
  MOD_RES (104)..(104): Pro, Ser, Gly, Asp, Ala, His, Arg or Tyr
  MOD_RES (105)..(105): Leu, Tyr, Phe, Ala, Asp, His, Pro, Ser or Val
  MOD_RES (106)..(106): Phe or Ser
  MOD_RES (108)..(108): Ile, Val, Tyr, Phe or Asn
QVQLVQSGAEVKKPGESLKISCKGSGYSFTXYXIXWVRQMPGKGLEWMGXIYPGDSXTXYXXXFQGQVTISADKSISTAYLQWSSLKASDTAMYYCARDXXXXXXXDXWGQGTLVTVSS

SEQ ID NO: 340 - protein, 119 aa, Artificial Sequence
  Description of Artificial Sequence Synthetic polypeptide
  MOD_RES (31)..(31): Asn or Asp
  MOD_RES (33)..(33): Trp or Tyr
  MOD_RES (35)..(35): Ser, Thr or Ala
  MOD_RES (50)..(50): Ile or Arg
  MOD_RES (59)..(59): Asn, Arg, His, Ser, Lys or Gln
  MOD_RES (62)..(62): Pro or Gln
  MOD_RES (103)..(103): Lys, Gly, Arg, Ser, Asp, Glu, His, Asn or Gln
  MOD_RES (104)..(104): Pro, Gly, Asp, Ser, Ala, His, Arg or Tyr
  MOD_RES (105)..(105): Leu, Ala, Tyr, Asp, Phe, His, Pro, Ser or Val
  MOD_RES (108)..(108): Ile, Tyr, Phe or Asn
QVQLVQSGAEVKKPGESLKISCKGSGYSFTXYXIXWVRQMPGKGLEWMGXIYPGDSYTXYSXSFQGQVTISADKSISTAYLQWSSLKASDTAMYYCARDWYWXXXFDXWGQGTLVTVSS

SEQ ID NO: 341 - protein, 114 aa, Artificial Sequence
  Description of Artificial Sequence Synthetic polypeptide
  MOD_RES (24)..(24): Arg, Lys, His, Asn, Gln or Ser
  MOD_RES (30)..(30): Leu or Phe
  MOD_RES (31)..(31): Tyr or His
  MOD_RES (33)..(33): Ser, Arg or Gly
  MOD_RES (34)..(34): Asn or Thr
  MOD_RES (35)..(35): Asn, Arg, His or Ser
  MOD_RES (38)..(38): Tyr or Phe
  MOD_RES (40)..(40): Ala or Thr
  MOD_RES (56)..(56): Trp, Phe or Leu
  MOD_RES (57)..(57): Ala or Thr
  MOD_RES (59)..(59): Thr, Ile, Ala or Val
  MOD_RES (61)..(61): Glu, Ala or Lys
  MOD_RES (62)..(62): Ser or Thr
  MOD_RES (98)..(98): Ser or Tyr
  MOD_RES (99)..(99): Ser or Thr
  MOD_RES (100)..(100): Phe, Tyr, Leu, Thr, His, Ile, Asn, Pro or Ser
  MOD_RES (102)..(102): Ile, Arg, Val, Tyr, Asp, Phe, Gly, His, Leu,      Asn or Ser
DIVMTQSPDSLAVSLGERATINCXSSQSVXXSXXXKNXLXWYQQKPGQPPKLLIYXXSXRXXGVPDRFSGSGSGTDFTLTISSLQAEDVAVYYCQQYXXXPXTFGQGTKVEIKR

SEQ ID NO: 342 - protein, 114 aa, Artificial Sequence
  Description of Artificial Sequence Synthetic polypeptide
  MOD_RES (38)..(38): Tyr or Phe
  MOD_RES (40)..(40): Ala or Thr
  MOD_RES (56)..(56): Trp, Phe or Leu
  MOD_RES (61)..(61): Glu or Lys
  MOD_RES (62)..(62): Ser or Thr
  MOD_RES (100)..(100): Phe, Tyr, Thr, Ile, Asn or Ser
  MOD_RES (102)..(102): Ile, Tyr, Arg, Phe, His, Leu, Asn or Ser
DIVMTQSPDSLAVSLGERATINCRSSQSVLYSSNNKNXLXWYQQKPGQPPKLLIYXASTRXXGVPDRFSGSGSGTDFTLTISSLQAEDVAVYYCQQYSSXPXTFGQGTKVEIKR

SEQ ID NO: 343 - protein, 119 aa, Artificial Sequence
  Description of Artificial Sequence Synthetic polypeptide
QVQLVQSGAEVKKPGESLKISCKGSGYSFTNYYISWVRQMPGKGLEWMGLIYPGDSYTRYNPSFQGQVTISADKSISTAYLQWSSLKASDTAMYYCARDYYSRPFSDIWGQGTLVTVSS

SEQ ID NO: 344 - protein, 119 aa, Artificial Sequence
  Description of Artificial Sequence Synthetic polypeptide
QVQLVQSGAEVKKPGESLKISCKGSGYSFTNYYISWVRQMPGKGLEWMGLIYPGDSYTRYNPKFQGQVTISADKSISTAYLQWSSLKASDTAMYYCARDYYSRPYSDVWGQGTLVTVSS

SEQ ID NO: 345 - protein, 119 aa, Artificial Sequence
  Description of Artificial Sequence Synthetic polypeptide
QVQLVQSGAEVKKPGESLKISCKGSGYSFTDYYISWVRQMPGKGLEWMGLIYPGDSYTRYSPNFQGQVTISADKSISTAYLQWSSLKASDTAMYYCARDHGYRPYSDIWGQGTLVTVSS

SEQ ID NO: 346 - protein, 119 aa, Artificial Sequence
  Description of Artificial Sequence Synthetic polypeptide
QVQLVQSGAEVKKPGESLKISCKGSGYSFTNYYISWVRQMPGKGLEWMGLIYPGDSYTNYNPNFQGQVTISADKSISTAYLQWSSLKASDTAMYYCARDHYSTPFFDVWGQGTLVTVSS

SEQ ID NO: 347 - protein, 119 aa, Artificial Sequence
  Description of Artificial Sequence Synthetic polypeptide
QVQLVQSGAEVKKPGESLKISCKGSGYSFTNYYISWVRQMPGKGLEWMGLIYPGDSYTRYNRKFQGQVTISADKSISTAYLQWSSLKASDTAMYYCARDYYSRPLSDVWGQGTLVTVSS

SEQ ID NO: 348 - protein, 119 aa, Artificial Sequence
  Description of Artificial Sequence Synthetic polypeptide
QVQLVQSGAEVKKPGESLKISCKGSGYSFTNYYISWVRQMPGKGLEWMGLIYPGDSYTRYSPRFQGQVTISADKSISTAYLQWSSLKASDTAMYYCARDHGYKPYSDIWGQGTLVTVSS

SEQ ID NO: 349 - protein, 119 aa, Artificial Sequence
  Description of Artificial Sequence Synthetic polypeptide
QVQLVQSGAEVKKPGESLKISCKGSGYSFTDYYISWVRQMPGKGLEWMGLIYPGDSYTRYSPSFQGQVTISADKSISTAYLQWSSLKASDTAMYYCARDYYSKPLFDVWGQGTLVTVSS

SEQ ID NO: 350 - protein, 119 aa, Artificial Sequence
  Description of Artificial Sequence Synthetic polypeptide
QVQLVQSGAEVKKPGESLKISCKGSGYSFTNYYISWVRQMPGKGLEWMGIIYPGDSYIHYNQNFQGQVTISADKSISTAYLQWSSLKASDTAMYYCARDYYSRPFSDIWGQGTLVTVSS

SEQ ID NO: 351 - protein, 119 aa, Artificial Sequence
  Description of Artificial Sequence Synthetic polypeptide
QVKLVQSGAEVKKPGESLKISCKGSGYSFTNYYIAWVRQMPGKGLEWMGIIYPGDSYTHYNPKFQGQVTISADKSISTAYLQWSSLKASDTAMYYCARDHGYKPFSDIWGQGTLVTVSS

SEQ ID NO: 352 - protein, 119 aa, Artificial Sequence
  Description of Artificial Sequence Synthetic polypeptide
QVQLVQSGAEVKKPGESLKISCKGSGYSFTNYYIAWVRQMPGKGLEWMGVIYPGDSYTRYNPKFQGQVTISADKSISTAYLQWSSLKASDTAMYYCARDYWSRPYFDIWGQGTLVTVSS

SEQ ID NO: 353 - protein, 119 aa, Artificial Sequence
  Description of Artificial Sequence Synthetic polypeptide
QVQLVQSGAEVKKPGESLKISCKGSGYSFTNYYIAWVRQMPGKGLEWMGIIYPGDSYTHYNPKFQGQVTISADKSISTAYLQWSSLKASDTAMYYCARDHWSRPFSDVWGQGTLVTVSS

SEQ ID NO: 354 - protein, 119 aa, Artificial Sequence
  Description of Artificial Sequence Synthetic polypeptide
QVQLVQSGAEVKKPGESLKISCKGSGYSFTNYYISWVRQMPGKGLEWMGIIYPGDSYTRYNPNFQGQVTISADKSISTAYLQWSSLKASDTAMYYCARDYWSKPLSDVWGQGTLVTVSS

SEQ ID NO: 355 - protein, 119 aa, Artificial Sequence
  Description of Artificial Sequence Synthetic polypeptide
QVQLVQSGAEVKKPGESLKISCKGSGYSFTDYYISWVRQMPGKGLEWMGLIYPGDSYTHYNPNFQGQVTISADKSISTAYLQWSSLKASDTAMYYCARDHWSKPYFDVWGQGTLVTVSS

SEQ ID NO: 356 - protein, 119 aa, Artificial Sequence
  Description of Artificial Sequence Synthetic polypeptide
QVQLVQSGAEVKKPGESLKISCKGSGYSFTNYYITWVRQMPGRGLEWMGIIYPGDSYTRYNPKFQGQVTISADKSISTAYLQWSSLKASDTAMYYCARDHWSKPFSDVWGQGTLVTVSS

SEQ ID NO: 357 - protein, 119 aa, Artificial Sequence
  Description of Artificial Sequence Synthetic polypeptide
QVQLVQSGAEVKKPGESLKISCKGSGYSFTNYYITWVRQMPGKGLEWMGLIYPGDSYTRYSPRFQGQVTISADKSISTAYLQWSSLKASDTAMYYCARDHWSRPYSDVWGQGTLVTVSS

SEQ ID NO: 358 - protein, 119 aa, Artificial Sequence
  Description of Artificial Sequence Synthetic polypeptide
QVQLVQSGAEVKKPGESLKISCKGSGYSFTDYYIAWVRQMPGKGLEWMGIIYPGDSYTRYNPKFQGQVTISADKSISTAYLQWSSLKASDTAMYYCARDYWSRPFSDVWGQGTLVTVSS

SEQ ID NO: 359 - protein, 119 aa, Artificial Sequence
  Description of Artificial Sequence Synthetic polypeptide
QVQLVQSGAEVKKPGESLKISCKGSGYSFTDYYISWVRQMPGKGLEWMGLIYPGDSYTHYNPNFQGQVTISADKSISTAYLQWSSLKASDTAMYYCARDYWSKPLFDVWGQGTLVTVSS

SEQ ID NO: 360 - protein, 119 aa, Artificial Sequence
  Description of Artificial Sequence Synthetic polypeptide
QVQLVQSGAEVKKPGESLKISCKGSGYSFTDYYIAWVRQMPGKGLEWMGIIYPGDSYTRYNPRFQGQVTISADKSISTAYLQWSSLKASDTAMYYCARDYWSKPLSDIWGQGTLVTVSS

SEQ ID NO: 361 - protein, 119 aa, Artificial Sequence
  Description of Artificial Sequence Synthetic polypeptide
QVKLVQSGAEVKKPGESLKISCKGSGYSFTDYYISWVRQMPGKGLEWMGIIYPGDSYTHYNPKFQGQVTISADKSISTAYLQWSSLKASDTAMYYCARDRWSKPLFDVWGQGTLVTVSS

SEQ ID NO: 362 - protein, 119 aa, Artificial Sequence
  Description of Artificial Sequence Synthetic polypeptide
QVQLVQSGAEVKKPGESLKISCKGSGYSFTDYYISWVRQMPGKGLEWMGLIYPGDSYTRYNPSFQGQVTISADKSISTAYLQWSSLKASDTAMYYCARDYWSKPLSDIWGQGTLVTVSS

SEQ ID NO: 363 - protein, 119 aa, Artificial Sequence
  Description of Artificial Sequence Synthetic polypeptide
QVQLVQSGAEVKKPGESLKISCKGSGYSFTDYYISWVRQMPGKGLEWMGSIYPGDSYTHYSHKFQGQVTISADKSISTAYLQWSSLKASDTAIYYCARDHWSRPFFDVWGQGTLVTVSS

SEQ ID NO: 364 - protein, 119 aa, Artificial Sequence
  Description of Artificial Sequence Synthetic polypeptide
QVQLVQSGAEVKKPGESLKISCKGSGYSFTDYYIAWVRQMPGKGLEWMGLIYPGDSYTSYNPRFQGQVTISADKSISTAYLQWSSLKASDTAMYYCARDYWSTPYFDIWGQGTLVTVSS

SEQ ID NO: 365 - protein, 119 aa, Artificial Sequence
  Description of Artificial Sequence Synthetic polypeptide
QVQLVQSGAEVKKPGESLKISCKGSGYSFTDYYISWVRQMPGKGLEWMGIIYPGDSYTSYSPRFQGQVTISADKSISTAYLQWSSLKASDTAMYYCARDYWSRPLFDVWGQGTLVTVSS

SEQ ID NO: 366 - protein, 119 aa, Artificial Sequence
  Description of Artificial Sequence Synthetic polypeptide
QVQLVQSGAEVKKPGESLKISCKGSGYSFTNYYISWVRQMPGKGLEWMGLIYPGDSYTHYNPSFQGQVTISADKSISTAYLQWSSLKASDTAMYYCARDHWYKPFSDIWGQGTLVTVSS

SEQ ID NO: 367 - protein, 119 aa, Artificial Sequence
  Description of Artificial Sequence Synthetic polypeptide
QVQLVQSGAEVKKPGESLKISCKGSGYSFTDYYISWVRQMPGKGLEWMGIIYPGDSYTNYNPSFQGQVTISADKSISTAYLQWSSLKASDTAMYYCARDYWYRPFSDIWGQGTLVTVSS

SEQ ID NO: 368 - protein, 119 aa, Artificial Sequence
  Description of Artificial Sequence Synthetic polypeptide
QVQLVQSGAEVKKPGESLKISCKGSGYSFTDYYISWVRQMPGKGLEWMGIIYPGDSYTHYSPNFQGQVTISADKSISTAYLQWSSLKASDTAMYYCARDYWYTPFFDVWGQGTLVTVSS

SEQ ID NO: 369 - protein, 119 aa, Artificial Sequence
  Description of Artificial Sequence Synthetic polypeptide
QVQLVQSGAEVKKPGESLKISCKGSGYSFTDYYISWVRQMPGKGLEWMGLIYPGDSYTNYSPKFQGQVTISADKSISTAYLQWSSLKASDTAMYYCARDHWSKPLSDVWGQGTLVTVSS

SEQ ID NO: 370 - protein, 119 aa, Artificial Sequence
  Description of Artificial Sequence Synthetic polypeptide
QVQLVQSGAEVKKPGESLKISCKGSGYSFTNYYISWVRQMPGKGLEWMGMIYPGDSYTHYSPNFQGQVTISADKSISTAYLQWSSLKASDTAMYYCARDYWSKPYFDVWGQGTLVTVSS

SEQ ID NO: 371 - protein, 119 aa, Artificial Sequence
  Description of Artificial Sequence Synthetic polypeptide
QVQLVQSGAEVKKPGESLKISCKGSGYSFTNYYISWVRQMPGKGLEWMGMIYPGDSYTSYNPKFQGQVTISADKSISTAYLQWSSLKASDTAMYYCARDHWSKPYFDIWGQGTLVTVSS

SEQ ID NO: 372 - protein, 119 aa, Artificial Sequence
  Description of Artificial Sequence Synthetic polypeptide
QVQLVQSGAEVKKPGESLKISCKGSGYSFTNYYISWVRQMPGKGLEWMGMIYPGDSYTNYNQKFQGQVTISADKSISTAYLQWSSLKASDTAMYYCARDYWSRPYSDIWGQGTLVTVSS

SEQ ID NO: 373 - protein, 119 aa, Artificial Sequence
  Description of Artificial Sequence Synthetic polypeptide
QVQLVQSGAEVKKPGESLKISCKGSGYSFTDYYISWVRQMPGKGLEWMGMIYPGDSYTHYSQNFQGQVTISADKSISTAYLQWSSLKASDTAMYYCARDHWSRPFFDVWGQGTLVTVSS

SEQ ID NO: 374 - protein, 119 aa, Artificial Sequence
  Description of Artificial Sequence Synthetic polypeptide
QVQLVQSGAEVKKPGESLKISCKGSGYSFTDYYISWVRQMPGKGLEWMGMIYPGDSYTRYSPSFQGQVTISADKSISTAYLQSSSLKASDTAMYYCARDYWYKPFSDVWGQGTLVTVSS

SEQ ID NO: 375 - protein, 119 aa, Artificial Sequence
  Description of Artificial Sequence Synthetic polypeptide
QVQLVQSGAEVKKPGESLKISCKGSGYSFTNYYISWVRQMPGKGLEWMGMIYPGDSYTSYNPNFQGQVTISADKSISTAYLQWSSLKASDTAMYYCARDHWSKPFFDIWGQGTLVTVSS

SEQ ID NO: 376 - protein, 119 aa, Artificial Sequence
  Description of Artificial Sequence Synthetic polypeptide
QVQLVQSGAEVKKPGESLKISCKGSGYSFTNYYITWVRQMPGKGLEWMGMIYPGDSYTRYSPKFQGQVTISADKSISTAYLQWSSLKASDTAMYYCARDHWSKPLSDVWGQGTLVTVSS

SEQ ID NO: 377 - protein, 119 aa, Artificial Sequence
  Description of Artificial Sequence Synthetic polypeptide
QVQLVQSGAEVKKPGESLKISCKGSGYSFTNYYISWVRQMPGKGLEWMGMIYPGDSYTRYSPKFQGQVTISADKSISTAYLQWSSLKASDTAMYYCARDHWYRPYSDIWGQGTLVTVSS

SEQ ID NO: 378 - protein, 119 aa, Artificial Sequence
  Description of Artificial Sequence Synthetic polypeptide
QVQLVQSGAEVKKPGESLKISCKGSGYSFTNYYITWVRQMPGKGLEWMGMIYPGDSYTHYSQRFQGQVTISADKSISTAYLQWSSLKASDTAIYYCARDHWSRPLFDVWGQGTLVTVSS

SEQ ID NO: 379 - protein, 119 aa, Artificial Sequence; identical to SEQ ID NO: 377
  Description of Artificial Sequence Synthetic polypeptide

SEQ ID NO: 380 - protein, 119 aa, Artificial Sequence
  Description of Artificial Sequence Synthetic polypeptide
QVQLVQSGAEVKKPGESLKISCKGSGYSFTDYYISWVRQMPGKGLEWMGWIYPGDSYTHYSPSFQGQVTISADKSISTAYLQWSSLKASDTAMYYCARDHWSKPYFDVWGQGTLVTVSS

SEQ ID NO: 381 - protein, 119 aa, Artificial Sequence
  Description of Artificial Sequence Synthetic polypeptide
QVQLVQSGAEVKKPGESLKISCKGSGYSFTNYYITWVRQMPGKGLEWMGMIYPGDSYTHYNPSFQGQVTISADKSISTAYLQWSSLKASDTAMYYCARDHWYKPLFDIWGQGTLVTVSS

SEQ ID NO: 382 - protein, 119 aa, Artificial Sequence
  Description of Artificial Sequence Synthetic polypeptide
QVQLVQSGAEVKKPGESLKISCKGSGYSFTDYYINWVRQMPGKGLEWMGMIYPGDSYTNYNPKFQGQVTISADKSISTAYLQWSSLKASDTAIYYCARDHWSRPFSDVWGQGTLVTVSS

SEQ ID NO: 383 - protein, 119 aa, Artificial Sequence
  Description of Artificial Sequence Synthetic polypeptide
QVQLVQSGAEVKKPGESLKISCKGSGYSFTNYYISWVRQMPGKGLEWMGWIYPGDSYTSYNQKFQGQVTISADKSISTAYLQWSSLKASDTAMYYCARDYWSKPYSDVWGQGTLVTVSS

SEQ ID NO: 384 - protein, 119 aa, Artificial Sequence
  Description of Artificial Sequence Synthetic polypeptide
QVQLVQSGAEVKKPGESLKISCKGSGYSFTDYYITWVRQMPGKGLEWMGMIYPGDSYTNYRHNFQGQVTISADKSISTAYLQWSSLKASDTAMYYCARDYWSRPYFDIWGQGTLVTVSS

SEQ ID NO: 385 - protein, 119 aa, Artificial Sequence
  Description of Artificial Sequence Synthetic polypeptide
QVQLVQSGAEVKKPGESLKISCKGSGYSFTDYYISWVRQMPGKGLEWMGWIYPGDSYTHYSHNFQGQVTISADKSISTAYLQWSSLKASDTAMYYCARDHWSRPFSDVWGQGTLVTVSS

SEQ ID NO: 386 - protein, 119 aa, Artificial Sequence
  Description of Artificial Sequence Synthetic polypeptide
QVQLVQSGAEVKKPGESLKISCKGSGYSFTDYYISWVRQMPGKGLEWMGMIYPGDSYTSYSPRFQGQVTISADKSISTAYLQWSSLKASDTAMYYCARDYWYRPFSDIWGQGTLVTVSS

SEQ ID NO: 387 - protein, 119 aa, Artificial Sequence
  Description of Artificial Sequence Synthetic polypeptide
QVQLVQSGAEVKKPGESLKISCKGSGYSFTDYYITWVRQMPGKGLEWMGMIYPGDSYTRYSPKFQGQVTISADKSISTAYLQWSSLKASDTAMYYCARDYWSKPFFDVWGQGTLVTVSS

SEQ ID NO: 388 - protein, 119 aa, Artificial Sequence
  Description of Artificial Sequence Synthetic polypeptide
QVQLVQSGAEVKKPGESLKISCKGSGYSFTDYYISWVRQMPGKGLEWMGMIYPGDSYTHYSQSFQGQVTISADKSISTAYLQWSSLKASDTAMYYCARDHWYRPLFDIWGQGTLVTVSS

SEQ ID NO: 389 - protein, 119 aa, Artificial Sequence
  Description of Artificial Sequence Synthetic polypeptide
QVQLVQSGAEVKKPGESLKISCKGSGYSFTNYYIAWVRQMPGKGLEWMGMIYPGDSYTSYSHRFQGQVTISADKSISTAYLQWSSLKASDTAMYYCARDHWYRPYFDIWGQGTLVTVSS

SEQ ID NO: 390 - protein, 119 aa, Artificial Sequence
  Description of Artificial Sequence Synthetic polypeptide
QVQLVQSGAEVKKPGESLKISCKGSGYSFTDYYISWVRQMPGKGLEWMGWIYPGDSYTHYNPNFQGQVTISADKSISTAYLQWSSLKASDTAMYYCARDRWSTPYFDVWGQGTLVTVSS

SEQ ID NO: 391 - protein, 119 aa, Artificial Sequence; identical to SEQ ID NO: 370
  Description of Artificial Sequence Synthetic polypeptide

SEQ ID NO: 392 - protein, 119 aa, Artificial Sequence
  Description of Artificial Sequence Synthetic polypeptide
QVQLVQSGAEVKKPGESLKISCKGSGYSFTDYYISWVRQMPGKGPEWMGMIYPGDSYTRYSQKFQGQVTISADKSISTAYLQWSSLKASDTAIYYCARDHWSRPLSDIWGQGTLVTVSS

SEQ ID NO: 393 - protein, 119 aa, Artificial Sequence
  Description of Artificial Sequence Synthetic polypeptide
QVQLVQSGAEVKKPGESLKISCKGSGYSFTDYYISWVRQMPGKGLEWMGWIYPGDSYTHYNPMFQGQVTISADKSISTAYLQWSSLKASDTAMYYCARDRWSRPYFDIWGQGTLVTVSS

SEQ ID NO: 394 - protein, 119 aa, Artificial Sequence
  Description of Artificial Sequence Synthetic polypeptide
QVQLVQSGAEVKKPGESLKISCKGSGYSFTDYYISWVRQMPGKGLEWMGMIYPGDSYTRYSPNFQGQVTISADKSISTAYLQWSSLKASDTAMYYCARDHWYRPLSDIWGQGTLVTVSS

SEQ ID NO: 395 - protein, 119 aa, Artificial Sequence
  Description of Artificial Sequence Synthetic polypeptide
QVQLVQSGAEVKKPGESLKISCKGSGYSFTNYWISWVRQMPGKGLEWMGIIYPGDSYTKYSPSFQGQVTISADKSISTAYLQWSSLKASDTAMYYCARDYWYKPLFDIWGQGTLVTVSS

SEQ ID NO: 396 - protein, 119 aa, Artificial Sequence
  Description of Artificial Sequence Synthetic polypeptide
QVQLVQSGAEVKKPGESLKISCKGSGYSFTNYWISWVRQMPGKGLEWMGIIYPGDSYTQYSPSFQGQVTISADKSISTAYLQWSSLKASDTAMYYCARDYWYKPLFDIWGQGTLVTVSS

SEQ ID NO: 397 - protein, 119 aa, Artificial Sequence
  Description of Artificial Sequence Synthetic polypeptide
QVQLVQSGAEVKKPGESLKISCKGSGYSFTNYWISWVRQMPGKGLEWMGIIYPGDSYTRYSPSFQGQVTISADKSISTAYLQWSSLKASDTAMYYCARDYWYKPLFDIWGQGTLVTVSS

SEQ ID NO: 398 - protein, 119 aa, Artificial Sequence
  Description of Artificial Sequence Synthetic polypeptide
QVQLVQSGAEVKKPGESLKISCKGSGYSFTNYWISWVRQMPGKGLEWMGIIYPGDSYTNYSPSFQGQVTISADKSISTAYLQWSSLKASDTAMYYCARDYAYKPLFDIWGQGTLVTVSS

SEQ ID NO: 399 - protein, 119 aa, Artificial Sequence
  Description of Artificial Sequence Synthetic polypeptide
QVQLVQSGAEVKKPGESLKISCKGSGYSFTNYWISWVRQMPGKGLEWMGIIYPGDSYTNYSPSFQGQVTISADKSISTAYLQWSSLKASDTAMYYCARDYFYKPLFDIWGQGTLVTVSS

SEQ ID NO: 400 - protein, 119 aa, Artificial Sequence
  Description of Artificial Sequence Synthetic polypeptide
QVQLVQSGAEVKKPGESLKISCKGSGYSFTNYWISWVRQMPGKGLEWMGIIYPGDSYTNYSPSFQGQVTISADKSISTAYLQWSSLKASDTAMYYCARDYYYKPLFDIWGQGTLVTVSS

SEQ ID NO: 401 - protein, 14 aa, Artificial Sequence
  Description of Artificial Sequence Synthetic peptide
GGGGGGGGGGGCGG

SEQ ID NO: 402 - protein, 14 aa, Artificial Sequence
  Description of Artificial Sequence Synthetic peptide
GGGGGGGGGGGGCG

SEQ ID NO: 403 - protein, 10 aa, Artificial Sequence
  Description of Artificial Sequence Synthetic peptide
GGGGSGGGGS

SEQ ID NO: 404 - protein, 118 aa, Artificial Sequence [sequence omitted]

SEQ ID NO: 405 - protein, 113 aa, Artificial Sequence [sequence omitted]

SEQ ID NO: 406 - protein, 123 aa, Artificial Sequence; identical to SEQ ID NO: 69
  Description of Artificial Sequence Synthetic polypeptide

SEQ ID NO: 407 - protein, 111 aa, Artificial Sequence; identical to SEQ ID NO: 100
  Description of Artificial Sequence Synthetic polypeptide

SEQ ID NO: 408 - protein, 127 aa, Artificial Sequence; identical to SEQ ID NO: 130
  Description of Artificial Sequence Synthetic polypeptide

SEQ ID NO: 409 - protein, 112 aa, Artificial Sequence; identical to SEQ ID NO: 222
  Description of Artificial Sequence Synthetic polypeptide

SEQ ID NO: 410 - protein, 131 aa, Artificial Sequence
  Description of Artificial Sequence Synthetic polypeptide
QVQLVQSGAEVKKPGSSVKVSCKASGGTFNSHAISWVRQAPGQGLEWMGGINPILGIANYAQKFQGRVTITADESTSTAYMELSSLRSEDTAVYYCARHYEIQIGRYGMNVYYLMYRFASWGQGTLVTVSS

SEQ ID NO: 411 - protein, 106 aa, Artificial Sequence
  Description of Artificial Sequence Synthetic polypeptide
DIQMTQSPSSLSASVGDRVTITCRASQGIRSALNWYQQKPGKAPKLLIYNGSTLQSGVPSRFSGSGSGTDFTLTISSLQPEDFAVYYCQQFDGDPTFGQGTKVEIK

SEQ ID NO: 412 - protein, 119 aa, Artificial Sequence; identical to SEQ ID NO: 326
  Description of Artificial Sequence Synthetic polypeptide

SEQ ID NO: 413 - protein, 113 aa, Artificial Sequence
  Description of Artificial Sequence Synthetic polypeptide
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYSSFPITFGQGTKVEIK

SEQ ID NO: 414 - protein, 118 aa, Artificial Sequence
  Description of Artificial Sequence Synthetic polypeptide
QVNLRESGGGVVQPGRSLRLSCAASGFTFSSYGMHWVRQAPGEGLEWVSAISGSGGSTYYADSVKGRFTISRDNSKNTLYLQMNSLRAEDTAVYYCAKRFGEFAFDIWGRGTTVTVSS

SEQ ID NO: 415 - protein, 107 aa, Artificial Sequence
  Description of Artificial Sequence Synthetic polypeptide
AIRMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 416 - protein, 228 aa, Homo sapiens
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 417 - protein, 99 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARD

SEQ ID NO: 418 - protein, 90 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQ

SEQ ID NO: 419 - protein, 266 aa, Artificial Sequence
  Description of Artificial Sequence Synthetic polypeptide
HGEGTFTSDLSKQMEEECARLFIEWLKNGGPSSGAPPPGCGGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 420 - protein, 261 aa, Artificial Sequence
  Description of Artificial Sequence Synthetic polypeptide
HGEGTFTSDLSKQMEEECARLFIEWLKNGGPSSGAPPPGCGGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 421 - protein, 256 aa, Artificial Sequence
  Description of Artificial Sequence Synthetic polypeptide
HGEGTFTSDLSKQMEEECARLFIEWLKNGGPSSGAPPPGCGADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 422 - protein, 120 aa, Artificial Sequence
  Description of Artificial Sequence Synthetic polypeptide
EVQLVESGGGLVKPGGSLRLSCAASGFTFSTYSMNWVRQAPGKGLEWVSSISSSGDYIYYADSVKGRFTISRDNAKNSLYLQMNSLRAEDTAVYYCARDLVTSMVAFDYWGQGTLVTVSS

SEQ ID NO: 423 - protein, 108 aa, Artificial Sequence
  Description of Artificial Sequence Synthetic polypeptide
SYELTQPPSVSVSPGQTARITCSGDALPQKYVFWYQQKSGQAPVLVIYEDSKRPSGIPERFSGSSSGTMATLTISGAQVEDEADYYCYSTDRSGNHRVFGGGTKLTVL

SEQ ID NO: 424 - protein, 118 aa, Artificial Sequence
  Description of Artificial Sequence Synthetic polypeptide
QVQLVQSGAEVKKPGASVKVSCKASGYTFTSYYMHWVRQAPGQGLEWMGEIKPSGGSTSYNQKFQGRVTMTRDTSTSTVYMELSSLRSEDTAVYYCARERPLYASDLWGQGTTVTVSS

SEQ ID NO: 425 - protein, 107 aa, Artificial Sequence
  Description of Artificial Sequence Synthetic polypeptide
DIQMTQSPSSLSASVGDRVTITCQASQDVYTAVAWYQQKPGKAPKLLIYYASYRYTGVPSRFSGSGSGTDFTFTISSLQPEDIATYYCQQRYSLWRTFGQGTKLEIK

SEQ ID NO: 426 - protein, 118 aa, Artificial Sequence; identical to SEQ ID NO: 424
  Description of Artificial Sequence Synthetic polypeptide

SEQ ID NO: 427 - protein, 107 aa, Artificial Sequence
  Description of Artificial Sequence Synthetic polypeptide
DIQMTQSPSSLSASVGDRVTITCQASQDVYTAVAWYQQKPGKAPKLLIYSASYRYTGVPSRFSGSGSGTDFTFTISSLQPEDIATYYCQQRYSLWRTFGQGTKLEIK

SEQ ID NO: 428 - protein, 118 aa, Artificial Sequence; identical to SEQ ID NO: 424
  Description of Artificial Sequence Synthetic polypeptide

SEQ ID NO: 429 - protein, 107 aa, Artificial Sequence
  Description of Artificial Sequence Synthetic polypeptide
DIQMTQSPSSLSASVGDRVTITCQASQDVKTAVAWYQQKPGKAPKLLIYYASYRYTGVPSRFSGSGSGTDFTFTISSLQPEDIATYYCQQRYSLWRTFGQGTKLEIK

SEQ ID NO: 430 - protein, 118 aa, Artificial Sequence; identical to SEQ ID NO: 424
  Description of Artificial Sequence Synthetic polypeptide

SEQ ID NO: 431 - protein, 107 aa, Artificial Sequence; identical to SEQ ID NO: 2
  Description of Artificial Sequence Synthetic polypeptide

SEQ ID NO: 432 - protein, 118 aa, Artificial Sequence; identical to SEQ ID NO: 424
  Description of Artificial Sequence Synthetic polypeptide

SEQ ID NO: 433 - protein, 107 aa, Artificial Sequence
  Description of Artificial Sequence Synthetic polypeptide
DIQMTQSPSSLSASVGDRVTITCQASQDVSTAVAWYQQKPGKAPKLLIYSASYRYTGVPSRFSGSGSGTDFTFTISSLQPEDIATYYCQQRYSLWRTFGQGTKLEIK

SEQ ID NO: 434 - protein, 118 aa, Artificial Sequence; identical to SEQ ID NO: 1
  Description of Artificial Sequence Synthetic polypeptide

SEQ ID NO: 435 - protein, 107 aa, Artificial Sequence; identical to SEQ ID NO: 425
  Description of Artificial Sequence Synthetic polypeptide

SEQ ID NO: 436 - protein, 118 aa, Artificial Sequence; identical to SEQ ID NO: 1
  Description of Artificial Sequence Synthetic polypeptide

SEQ ID NO: 437 - protein, 107 aa, Artificial Sequence; identical to SEQ ID NO: 427
  Description of Artificial Sequence Synthetic polypeptide

SEQ ID NO: 438 - protein, 118 aa, Artificial Sequence; identical to SEQ ID NO: 1
  Description of Artificial Sequence Synthetic polypeptide

SEQ ID NO: 439 - protein, 107 aa, Artificial Sequence; identical to SEQ ID NO: 429
  Description of Artificial Sequence Synthetic polypeptide

SEQ ID NO: 440 - protein, 118 aa, Artificial Sequence; identical to SEQ ID NO: 1
  Description of Artificial Sequence Synthetic polypeptide

SEQ ID NO: 441 - protein, 107 aa, Artificial Sequence; identical to SEQ ID NO: 2
  Description of Artificial Sequence Synthetic polypeptide

SEQ ID NO: 442 - protein, 118 aa, Artificial Sequence; identical to SEQ ID NO: 1
  Description of Artificial Sequence Synthetic polypeptide

SEQ ID NO: 443 - protein, 107 aa, Artificial Sequence; identical to SEQ ID NO: 433
  Description of Artificial Sequence Synthetic polypeptide

SEQ ID NO: 444 - protein, 164 aa, Artificial Sequence
  Description of Artificial Sequence Synthetic polypeptide
HYEGTFTSDLSKQMEEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 445 - protein, 164 aa, Artificial Sequence
  Description of Artificial Sequence Synthetic polypeptide
HVEGTFTSDLSKQMEEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 446 - protein, 164 aa, Artificial Sequence
  Description of Artificial Sequence Synthetic polypeptide
HTEGTFTSDLSKQMEEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 447 - protein, 164 aa, Artificial Sequence
  Description of Artificial Sequence Synthetic polypeptide
HQEGTFTSDLSKQMEEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 448 - protein, 164 aa, Artificial Sequence
  Description of Artificial Sequence Synthetic polypeptide
HNEGTFTSDLSKQMEEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 449 - protein, 164 aa, Artificial Sequence
  Description of Artificial Sequence Synthetic polypeptide
HIEGTFTSDLSKQMEEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 450 - protein, 164 aa, Artificial Sequence
  Description of Artificial Sequence Synthetic polypeptide
HFEGTFTSDLSKQMEEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 451 - protein, 164 aa, Artificial Sequence
  Description of Artificial Sequence Synthetic polypeptide
HGEGTFTSDLSKQMGEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 452 - protein, 164 aa, Artificial Sequence
  Description of Artificial Sequence Synthetic polypeptide
HGEGTFTSDLSKQMAEECVRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 453 - protein, 164 aa, Artificial Sequence
  Description of Artificial Sequence Synthetic polypeptide
HGEGTFTSDLSKQMEEECTRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 454 - protein, 164 aa, Artificial Sequence
  Description of Artificial Sequence Synthetic polypeptide
HGEGTFTSDLSKQMEEECSRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 455 - protein, 164 aa, Artificial Sequence
  Description of Artificial Sequence Synthetic polypeptide
HGEGTFTSDLSKQMEEECGRLFIEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 456 - protein, 164 aa, Artificial Sequence; identical to SEQ ID NO: 47
  Description of Artificial Sequence Synthetic polypeptide

SEQ ID NO: 457 - protein, 164 aa, Artificial Sequence
  Description of Artificial Sequence Synthetic polypeptide
HGEGTFTSDLSKQMEEECVRLFTEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 458 - protein, 164 aa, Artificial Sequence
  Description of Artificial Sequence Synthetic polypeptide
HGEGTFTSDLSKQMEEECVRLFSEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 459 - protein, 164 aa, Artificial Sequence
  Description of Artificial Sequence Synthetic polypeptide
HGEGTFTSDLSKQMEEECVRLFGEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 460 - protein, 164 aa, Artificial Sequence
  Description of Artificial Sequence Synthetic polypeptide
HGEGTFTSDLSKQMEEECVRLFAEWLKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 461 - protein, 164 aa, Artificial Sequence
  Description of Artificial Sequence Synthetic polypeptide
HGEGTFTSDLSKQMEEECVRLFIEWTKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 462 - protein, 164 aa, Artificial Sequence
  Description of Artificial Sequence Synthetic polypeptide
HGEGTFTSDLSKQMEEECVRLFIEWSKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 463 - protein, 164 aa, Artificial Sequence
  Description of Artificial Sequence Synthetic polypeptide
HGEGTFTSDLSKQMEEECVRLFIEWPKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 464 - protein, 164 aa, Artificial Sequence
  Description of Artificial Sequence Synthetic polypeptide
HGEGTFTSDLSKQMEEECVRLFIEWNKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 465 - protein, 164 aa, Artificial Sequence
  Description of Artificial Sequence Synthetic polypeptide
HGEGTFTSDLSKQMEEECVRLFIEWQKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 466 - protein, 164 aa, Artificial Sequence
  Description of Artificial Sequence Synthetic polypeptide
HGEGTFTSDLSKQMEEECVRLFIEWMKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 467 - protein, 164 aa, Artificial Sequence
  Description of Artificial Sequence Synthetic polypeptide
HGEGTFTSDLSKQMEEECVRLFIEWIKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 468 - protein, 164 aa, Artificial Sequence
  Description of Artificial Sequence Synthetic polypeptide
HGEGTFTSDLSKQMEEECVRLFIEWHKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 469 - protein, 164 aa, Artificial Sequence
  Description of Artificial Sequence Synthetic polypeptide
HGEGTFTSDLSKQMEEECVRLFIEWGKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 470 - protein, 164 aa, Artificial Sequence
  Description of Artificial Sequence Synthetic polypeptide
HGEGTFTSDLSKQMEEECVRLFIEWEKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 471 - protein, 164 aa, Artificial Sequence
  Description of Artificial Sequence Synthetic polypeptide
HGEGTFTSDLSKQMEEECVRLFIEWDKNGGPSSGAPPPGCGGGGGSGGGGSGGGGSADIQMTQSPSSLSASVGDRVTITCQASQDVKTAVAWYQQKPGKAPKLLIYSASYRYTGVPSRFSGSGSGTDFTFTISSLQPEDIATYYCQQRYSLWRTFGQGTKLEIK

SEQ ID NO: 472 - protein, 164 aa, Artificial Sequence; identical to SEQ ID NO: 52
  Description of Artificial Sequence Synthetic polypeptide

SEQ ID NO: 473 - protein, 30 aa, Artificial Sequence
  Description of Artificial Sequence Synthetic polypeptide
HGEGTFTSDVSSYLEEQAAKEFIAWLVKGG

SEQ ID NO: 474 - protein, 30 aa, Artificial Sequence
  Description of Artificial Sequence Synthetic polypeptide
HGEGTFTSDVSSYLEEQNASEFIAWLVKGG

SEQ ID NO: 475 - protein, 30 aa, Artificial Sequence
  Description of Artificial Sequence Synthetic polypeptide
HGEGTFTSDVSSYLEEQAAKEFIAWLVNGS

SEQ ID NO: 476 - protein, 30 aa, Artificial Sequence
  Description of Artificial Sequence Synthetic polypeptide
HGEGTFTSDVSSYLEEQAAKEFIAWLVKNG

SEQ ID NO: 477 - protein, 30 aa, Artificial Sequence
  Description of Artificial Sequence Synthetic polypeptide
HGEGTFTSDVSSYLEEQAAKEFIAWLVKGN

SEQ ID NO: 478 - protein, 30 aa, Artificial Sequence; identical to SEQ ID NO: 473
  Description of Artificial Sequence Synthetic polypeptide

SEQ ID NO: 479 - protein, 30 aa, Artificial Sequence; identical to SEQ ID NO: 473
  Description of Artificial Sequence Synthetic polypeptide

SEQ ID NO: 480 - protein, 30 aa, Artificial Sequence
  Description of Artificial Sequence Synthetic polypeptide
HGEGTFTSDVSSYLEEQAAKEFIANLSKGG

SEQ ID NO: 481 - protein, 30 aa, Artificial Sequence
  Description of Artificial Sequence Synthetic polypeptide
HGEGTFTSDVSSYLEEQAAKEFIANLTKGG

SEQ ID NO: 482 - protein, 17 aa, Artificial Sequence
  Description of Artificial Sequence Synthetic peptide
GGGGGSGGGGSGGGGSA

SEQ ID NO: 483 - protein, 17 aa, Artificial Sequence
  Description of Artificial Sequence Synthetic peptide
SGGGGSGGGGSGGGGSA

SEQ ID NO: 484 - protein, 17 aa, Artificial Sequence
  Description of Artificial Sequence Synthetic peptide
GSGGGSGGGGSGGGGSA

SEQ ID NO: 485 - protein, 17 aa, Artificial Sequence
  Description of Artificial Sequence Synthetic peptide
NGSGGSGGGGSGGGGSA

SEQ ID NO: 486 - protein, 17 aa, Artificial Sequence
  Description of Artificial Sequence Synthetic peptide
GNGSGSGGGGSGGGGSA

SEQ ID NO: 487 - protein, 176 aa, Artificial Sequence
  Description of Artificial Sequence Synthetic polypeptide
HGEGTFTSCLSKQMEEEAVRLFIEWLKNGGPSSGAPPPSGGGGGGGGGGGCGGGGGGSGGGGSGGGGSADIQMTQSPSSLSASVGDRVTITCKASQDVHTAVAWYQQKPGKAPKLLIYHASYRYTGVPSRFSGSGSGTDFTFTISSLQPEDIATYYCQQRYSLWRTFGQGTKLEIK

SEQ ID NO: 488 - protein, 162 aa, Artificial Sequence
  Description of Artificial Sequence Synthetic polypeptide
HGEGTFTSDLSKQMEEECVRLFIEWLKNGGPSSGAPPGCGGGGSGGGGSGGGGSADIQMTQSPSSLSASVGDRVTITCKASQDVHTAVAWYQQKPGKAPKLLIYHASYRYTGVPSRFSGSGSGTDFTFTISSLQPEDIATYYCQQRYSLWRTFGQGTKLEIK

SEQ ID NO: 489 - protein, 154 aa, Artificial Sequence
  Description of Artificial Sequence Synthetic polypeptide
HGEGTFTSDVSSYLEEQAAKEFIANLSKGGGGGGGSGGGGSGGGGSADIQMTQSPSSLSASVGDRVTITCKASQDVHTAVAWYQQKPGKAPKLLIYHASYRYTGVPSRFSGSGSGTDFTFTISSLQPEDIATYYCQQRYSLWRTFGQGTKLEIK

SEQ ID NO: 490 - protein, 164 aa, Artificial Sequence
  Description of Artificial Sequence Synthetic polypeptide
HGEGTFTSDLSKQMEEECVRLFIEWLKNGGPSSGAPPPGCGGGGGSGGGGSGGGGSADIQMTQSPSSLSASVGDRVTITCKASQDVHTAVAWYQQKPGKAPKLLIYHASYRYTGVPSRFSGSGSGTDFTFTISSLQPEDIATYYCQQRYSLWRTFGQGTKLEIK

SEQ ID NO: 491 - protein, 125 aa, Artificial Sequence; identical to SEQ ID NO: 324
  Description of Artificial Sequence Synthetic polypeptide

SEQ ID NO: 492 - protein, 112 aa, Artificial Sequence; identical to SEQ ID NO: 325
  Description of Artificial Sequence Synthetic polypeptide

SEQ ID NO: 493 - protein, 10 aa, Artificial Sequence
  Description of Artificial Sequence Synthetic peptide
GFPFSKLGMV

SEQ ID NO: 494 - protein, 16 aa, Artificial Sequence
  Description of Artificial Sequence Synthetic peptide
TISSGGGYTYYPDSVK

SEQ ID NO: 495 - protein, 16 aa, Artificial Sequence
  Description of Artificial Sequence Synthetic peptide
EGISFQGGTYTYVMDY

SEQ ID NO: 496 - protein, 16 aa, Artificial Sequence
  Description of Artificial Sequence Synthetic peptide
RSSKSLLHRNGITYSY

SEQ ID NO: 497 - protein, 7 aa, Artificial Sequence
  Description of Artificial Sequence Synthetic peptide
QLSNLAS

SEQ ID NO: 498 - protein, 9 aa, Artificial Sequence
  Description of Artificial Sequence Synthetic peptide
YQNLELPLT

SEQ ID NO: 499 - protein, 98 aa, Artificial Sequence
  Description of Artificial Sequence Synthetic polypeptide
QVQLVQSGAEVKKPGASVKVSCKASGYTFTSYYMHWVRQAPGQGLEWMGEIHPSGGRTNYNEKFKSRVTMTRDTSTSTVYMELSSLRSEDTAVYYCAR

SEQ ID NO: 500 - protein, 90 aa, Artificial Sequence
  Description of Artificial Sequence Synthetic polypeptide
DIQMTQSPSSLSASVGDRVTITCKASQDVHTAVAWYQQKPGKAPKLLIYHASYRYTGVPSRFSGSGSGTDFTFTISSLQPEDIATYYCQQ

SEQ ID NO: 501 - protein, 47 aa, Artificial Sequence
  Description of Artificial Sequence Synthetic polypeptide
HGEGTFTSDVSSYLEEQAAKEFIAWLVKGGGGGGGSGGGGSGGGGSA

SEQ ID NO: 502 - protein, 47 aa, Artificial Sequence
  Description of Artificial Sequence Synthetic polypeptide
HGEGTFTSDVSSYLEEQNASEFIAWLVKGGGGGGGSGGGGSGGGGSA

SEQ ID NO: 503 - protein, 47 aa, Artificial Sequence
  Description of Artificial Sequence Synthetic polypeptide
HGEGTFTSDVSSYLEEQAAKEFIAWLVNGSGGGGGSGGGGSGGGGSA

SEQ ID NO: 504 - protein, 47 aa, Artificial Sequence
  Description of Artificial Sequence Synthetic polypeptide
HGEGTFTSDVSSYLEEQAAKEFIAWLVKNGSGGGGSGGGGSGGGGSA

SEQ ID NO: 505 - protein, 47 aa, Artificial Sequence
  Description of Artificial Sequence Synthetic polypeptide
HGEGTFTSDVSSYLEEQAAKEFIAWLVKGNGSGGGSGGGGSGGGGSA

SEQ ID NO: 506 - protein, 47 aa, Artificial Sequence
  Description of Artificial Sequence Synthetic polypeptide
HGEGTFTSDVSSYLEEQAAKEFIAWLVKGGNGSGGSGGGGSGGGGSA

SEQ ID NO: 507 - protein, 47 aa, Artificial Sequence
  Description of Artificial Sequence Synthetic polypeptide
HGEGTFTSDVSSYLEEQAAKEFIAWLVKGGGNGSGSGGGGSGGGGSA

SEQ ID NO: 508 - protein, 47 aa, Artificial Sequence
  Description of Artificial Sequence Synthetic polypeptide
HGEGTFTSDVSSYLEEQAAKEFIANLSKGGGGGGGSGGGGSGGGGSA

SEQ ID NO: 509 - protein, 47 aa, Artificial Sequence
  Description of Artificial Sequence Synthetic polypeptide
HGEGTFTSDVSSYLEEQAAKEFIANLTKGGGGGGGSGGGGSGGGGSA

SEQ ID NO: 510 - protein, 15 aa, Artificial Sequence
  Description of Artificial Sequence Synthetic peptide
GGGGSGGGGSGGGGS